SEQUENCE LISTING (153 sequences)

SEQ ID NO: 1 - protein, 300 aa, E. coli
MKRVITLFAVLLMGWSVNAWSFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 2 - protein, 279 aa, E. coli
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 3 - protein, 28 aa, E. coli
ASATIQAADVTITVNGKVVAKPCTVSTT

SEQ ID NO: 4 - protein, 36 aa, E. coli
PSMDKSKLTENTLQLAIISRIKLYYRPAKLALPPDQ

SEQ ID NO: 5 - protein, 14 aa, E. coli
ADVTITVNGKVVAK

SEQ ID NO: 6 - protein, 18 aa, E. coli
ENTLQLAIISRIKLYYRP

SEQ ID NO: 7 - protein, 5 aa, Artificial
  Linker
PGDGN

SEQ ID NO: 8 - protein, 4 aa, Artificial
  Linker
DNKQ

SEQ ID NO: 9 - protein, 21 aa, Artificial
  Leader
METDTLLLWVLLLWVPGSTGD

SEQ ID NO: 10 - protein, 36 aa, Artificial
  Leader
METDTLLLWVLLLWVPGSTGDAAQPARRARRTKLAL

SEQ ID NO: 11 - protein, 18 aa, Artificial
  Leader
MRLLAKIICLMLWAICVA

SEQ ID NO: 12 - protein, 19 aa, Artificial
  Leader
MGWSCIILFLVATATGVHS

SEQ ID NO: 13 - protein, 20 aa, Artificial
  Leader
METPAELLFLLLLWLPDTTG

SEQ ID NO: 14 - protein, 19 aa, Artificial
  Leader
MEFGLSWVFLVAILEGVHC

SEQ ID NO: 15 - protein, 162 aa, H. pylori
DIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 16 - protein, 164 aa, E. coli
LKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDTQN

SEQ ID NO: 17 - protein, 56 aa, Artificial
  iMX313
KKQGDADVCGEVAYIQSVVSDCHVPTAELRTLLEIRKLFLEIQKLKVELQGLSKEG

SEQ ID NO: 18 - protein, 205 aa, Artificial
  mi3
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 19 - protein, 265 aa, Artificial [sequence omitted]

SEQ ID NO: 20 - protein, 475 aa, Artificial
  FIMH_DG_PGDGN_HIS-Ferritin 536
MFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 21 - protein, 259 aa, Artificial
  LS-FIMHL-IMX313-HIS
MKYLLPTAAAGLLLLAAQPAMAFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGSSGSGSGSKKQGDADVCGEVAYIQSVVSDCHVPTAELRTLLEIRKLFLEIQKLKVELQGLSKEGGGSGSHHHHHHHH

SEQ ID NO: 22 - protein, 236 aa, Artificial
  FIMHL-S24S65- IMX313
MFASKTANGTAIPIGGGSANVYVNLAPVVNVGQNLVVDLSTQIFSHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGSSGSGSGSKKQGDADVCGEVAYIQSVVSDCHVPTAELRTLLEIRKLFLEIQKLKVELQGLSKEGGGSGSHHHHHH

SEQ ID NO: 23 - protein, 362 aa, Artificial
  FIMHL-S24S65-foldon -ferritin
MFASKTANGTAIPIGGGSANVYVNLAPAVNVGQNLVVDLSTQIFSHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGSGYIPEAPRDGQAYVRKDGEWVLLSTFLGSGHHHHHHGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 24 - protein, 389 aa, Artificial
  FIMHL-S24S65-Mi3
MFASKTANGTAIPIGGGSANVYVNLAPAVNVGQNLVVDLSTQIFSHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGSGGSGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGSGSGSHHHHHH

SEQ ID NO: 25 - protein, 389 aa, Artificial
  FIMHL-mI3
MFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGSGGSGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGSGSGSHHHHHH

SEQ ID NO: 26 - protein, 394 aa, Artificial
  FimHL-NOCYS -MI3
MFASKTANGTAIPIGGGSANVYVNLAPAVNVGQNLVVDLSTQIFSHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGGGSGGSGGSGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGSGSGSHHHHHH

SEQ ID NO: 27 - protein, 524 aa, Artificial
  FimHdeltaGG_PGDGN_DG_mi3
MFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGGGGMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGSGSGSHHHHHH

SEQ ID NO: 28 - protein, 343 aa, Artificial
  FimHL-GSG4- Ferritin
MGSSHHHHHHENLYFQGFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGSGGGGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 29 - protein, 404 aa, Artificial
  pelBLS-FimHL-mI3
MKYLLPTAAAGLLLLAAQPAMAFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGGSGMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGSGSHHHHHH

SEQ ID NO: 30 - protein, 331 aa, Artificial
  FimH_DG_Ferritin (GSGGGG)
MGSSHHHHHHENLYFQGDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGGGGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 31 - protein, 394 aa, Artificial
  FimHL-C-C- MI3
MFACKTANGTAIPIGGGSANVYVNLAPVCNVGQNCVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGGGSGGSGGSGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGSGSGSHHHHHH

SEQ ID NO: 32 - protein, 305 aa, Artificial
  FimHL-C-C-qBeta
MFACKTANGTAIPIGGGSANVYVNLAPVCNVGQNCVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGGGSGGSGGSGGSAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 33 - protein, 401 aa, Artificial
  FIMHL-HIS-Mi3 J96
METDTLLLWVLLLWVPGSTGDFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGGSGSHHHHHHHHGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 34 - protein, 358 aa, Artificial
  FIMHL-HIS-Fer 536
METDTLLLWVLLLWVPGSTGDFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGGSGSHHHHHHHHGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 35 - protein, 395 aa, Artificial
  FIMH_DG_PGDGN_IMX313_HIS J96
METDTLLLWVLLLWVPGSTGDFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSSGSGSGSKKQGDADVCGEVAYIQSVVSDCHVPTAELRTLLEIRKLFLEIQKLKVELQGLSKEGGGSGSHHHHHH

SEQ ID NO: 36 - protein, 495 aa, Artificial
  FimH_PGDGN_DG_HIS-Ferritn j96
METDTLLLWVLLLWVPGSTGDFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 37 - protein, 495 aa, Artificial
  FIMH_DG_PGDGN-HIS-Ferritin 536
METDTLLLWVLLLWVPGSTGDFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 38 - protein, 510 aa, Artificial
  FimH_PGDGN_DG-HIS-Ferritin J96
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 39 - protein, 553 aa, Artificial
  FimH_PGDGN_DG-HIS-MI3 j96
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 40 - protein, 410 aa, Artificial
  FimH_PGDGN_DG-HIS-IMX313 j96
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSSGSGSGSKKQGDADVCGEVAYIQSVVSDCHVPTAELRTLLEIRKLFLEIQKLKVELQGLSKEGGGSGSHHHHHH

SEQ ID NO: 41 - protein, 510 aa, Artificial
  FimH_PGDGN_DG_Ferritin  (536)
METDTLLLWVLLLWVPGSTGDAAQPARRARRTKLALFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 42 - protein, 389 aa, Artificial
  FimH-IMX313 j96
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQGSSGSGSGSKKQGDADVCGEVAYIQSVVSDCHVPTAELRTLLEIRKLFLEIQKLKVELQGLSKEGGGSGSHHHHHH

SEQ ID NO: 43 - protein, 542 aa, Artificial
  FimH_mi3 j96
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQGSGGGGMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEGSGSGSGSGSHHHHHHHH

SEQ ID NO: 44 - protein, 415 aa, Artificial
  FIMHL-HIS-mI3 j96
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGSGGHHHHHHHHGSGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 45 - DNA, 1497 bp, Artificial
  FIMH_DG_536-1EUM_0_5
atggaaaccgatacactgctgctgtgggtgctgttgctctgggttccaggctctacaggcgattttgcctgcaagaccgccaacggcacagccattcctattggcggaggcagcgccaatgtgtacgttaacctggctcctgccgtgaacgtgggccagaatctggtggtggatctgagcacccagatcttttgccacaacgactaccccgagacaatcaccgactacgtgacactgcagagaggctctgcttacggcggcgtgctgtctagctttagcggcaccgtgaagtacaacggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagagccagaacctgggctactacctgtctggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaacgccgatgtgaccatcacagtgaatggcaaggtggtggccaagagcggaagccaccaccatcatcaccatcaccacggcggctctatgctgaagcccgagatgatcgagaagctgaacgagcagatgaacctggaactgtacagctccctgctgtaccagcagatgagcgcctggtgtagctatcacggatttgagggcgctgccgcctttctgagaaggcacgcccaagaggaaatgacccacatgcagcggctgttcgactacctgaccgataccggcaatctgcccagaatcgacacaatcccatctccattcgccgagtacagcagcctggacgagctgttccaagaaacctacaagcacgagcagctgatcacccagaagatcaacgaactggcccatgccgccatgaccaaccaggactaccctaccttcaacttcctgcagtggtacgtggccgagcagcacgaggaagagaagctgttcaagagcatcatcgacaagctgagcctggccggaaagtctggcgagggcctgtactttatcgacaaagagctgagcacactggatacccagaactga

SEQ ID NO: 46 - DNA, 1617 bp, Artificial
  FIMH_DG_PGDGN_536-MI3
atggaaaccgatacactgctgctgtgggtgctgttgctctgggttccaggctctacaggcgattttgcctgcaagaccgccaacggcacagccattcctattggcggaggcagcgccaatgtgtacgttaacctggctcctgccgtgaacgtgggccagaatctggtggtggatctgagcacccagatcttttgccacaacgactaccccgagacaatcaccgactacgtgacactgcagagaggctctgcttacggcggcgtgctgtctagctttagcggcaccgtgaagtacaacggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagagccagaacctgggctactacctgtctggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaacgccgatgtgaccatcacagtgaatggcaaggtggtggccaagagcggaagccaccaccatcatcaccatcaccacggcggcagcatgaagatggaagaactgttcaagaagcacaagatcgtcgccgtgctgcgggccaattctgtggaagaggccaaaaaaaaggccctggccgtgtttcttggcggagtgcacctgatcgagatcacctttaccgtgcctgacgccgacaccgtgatcaaagagctgagcttcctgaaagagatgggcgccatcatcggagccggcacagtgacatctgttgagcaggccagaaaggccgtggaatctggcgccgagtttatcgtgtcccctcacctggatgaggaaatcagccagttcgccaaagaaaagggcgtgttctacatgcccggcgtgatgacacctacagagctggtcaaagccatgaagctgggccacaccatcctgaagctgtttccaggcgaagtcgtgggccctcagttcgtgaaagctatgaagggcccatttccaaacgtgaagttcgtgcccactggcggcgtgaacctggataatgtgtgcgagtggttcaaggctggcgtgctggctgttggagttggctctgctctggtcaagggcacacctgtggaagtggctgagaaggccaaggccttcgtggaaaagatcagaggctgcaccgagtga

SEQ ID NO: 47 - DNA, 1797 bp, Artificial
  FIMH_DG_PGDGN_536-encapsuline
atggaaaccgatacactgctgctgtgggtgctgttgctctgggttccaggctctacaggcgattttgcctgcaagaccgccaacggcacagccattcctattggcggaggcagcgccaatgtgtacgttaacctggctcctgccgtgaacgtgggccagaatctggtggtggatctgagcacccagatcttttgccacaacgactaccccgagacaatcaccgactacgtgacactgcagagaggctctgcttacggcggcgtgctgtctagctttagcggcaccgtgaagtacaacggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagagccagaacctgggctactacctgtctggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaacgccgatgtgaccatcacagtgaatggcaaggtggtggccaagagcggaagccaccaccatcatcaccatcaccacggcggcagcatggaatttctgaagagaagcttcgccccactgaccgagaagcagtggcaagagatcgacaaccgggccagagagatcttcaagacccagctgtacggccggaagttcgtggatgtggaaggcccttatggctgggagtatgccgctcatcctctgggcgaagtggaagtgctgagcgacgagaatgaggtcgtgaagtggggcctgagaaagagcctgcctctgatcgagctgagagccaccttcacactggacctgtgggaactcgacaacctggaaaggggcaagcccaatgtggacctgagcagcctggaagagacagtgcggaaggtggccgagttcgaggacgaagtgatcttcagaggctgcgagaagtctggcgtgaagggcctgctgagcttcgaggaacggaagatcgagtgtggcagcacccctaaggatctgctggaagccatcgtgcgggccctgagcatcttctctaaggatggcatcgagggcccctacacactggtcatcaacaccgaccggtggatcaacttcctgaaagaggaagccggccactatcctctggaaaagcgcgtggaagagtgcctgagaggcggcaagatcatcacaacccctagaatcgaggacgccctggtggtttctgagagaggcggagacttcaagctgatccttggccaggacctgtccatcggctacgaggacagagaaaaagacgccgtgcggctgttcatcaccgaaaccttcaccttccaagtggtcaaccccgaggctctgattctgctgaagttctga

SEQ ID NO: 48 - DNA, 1053 bp, Artificial
  HBcFIMHLJ96
atggaaaccgatacactgctgctgtgggtgctgttgctctgggttccaggatctaccggcgacgacatcgacccctacaaagagtttggcgccagcgtcgagctgctgagcttcctgcctagcgacttcttcccttccatccgggatctgctggataccgctagcgccctgtatagagaggccctggaaagccctgagcactgctctccacatcacacagccctgagacaggccatcctgtgttggggcgaactgatgaatctggccacctgggtcggaagcaacctggaagatcctggttctggcggcggaggctttgcctgtaaaacagccaatggcaccgccattcctatcggaggcggcagcgccaatgtgtacgttaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgctggcggagtggccatcaaggccggatctctgattgccgtgctgatcctgcggcagaccaacaactacaacagcgacgacttccagttcgtgtggaacatctacgccaacaacgacgtggtggtgcctacaggcggaggatctggcggagcttctagagaactggtcgtgtcctacgtgaacgtgaacatgggcctgaagatccggcagctgctctggtttcacatcagctgtctgaccttcggccgggaaaccgtgctggaatacctggtgtccttcggcgtgtggatcagaacccctcctgcctatagacctcctaacgctcccatcctgagcacactgcctgagacaacagttgttggaagcggaggcggaggccaccaccatcaccatcat

SEQ ID NO: 49 - DNA, 1473 bp, Artificial
  HBcFIMHDGJ96
atggagaccgacaccctgctgctgtgggtgctgctgctgtgggtgcccggcagcaccggcgacgacatcgacccctacaaggagttcggcgccagcgtggagctgctgagcttcctgcccagcgacttcttccccagcatccgggacctgctggacaccgccagcgccctgtaccgggaggccctggagagccccgagcactgcagcccccaccacaccgccctgcggcaggccatcctgtgctggggcgagctgatgaacctggccacctgggtgggcagcaacctggaggaccccggcagcggcggcggcggcttcgcctgcaagaccgccaacggcaccgccatccccatcggcggcggcagcgccaacgtgtacgtgaacctggcccccgtggtgaacgtgggccagaacctggtggtggacctgagcacccagatcttctgccacaacgactaccccgagaccatcaccgactacgtgaccctgcagcggggcagcgcctacggcggcgtgctgagcaacttcagcggcaccgtgaagtacagcggcagcagctaccccttccccaccaccagcgagaccccccgggtggtgtacaacagccggaccgacaagccctggcccgtggccctgtacctgacccccgtgagcagcgccggcggcgtggccatcaaggccggcagcctgatcgccgtgctgatcctgcggcagaccaacaactacaacagcgacgacttccagttcgtgtggaacatctacgccaacaacgacgtggtggtgcccaccggcggctgcgacgtgagcgcccgggacgtgaccgtgaccctgcccgactaccccggcagcgtgcccatccccctgaccgtgtactgcgccaagagccagaacctgggctactacctgagcggcaccaccgccgacgccggcaacagcatcttcaccaacaccgccagcttcagccccgcccagggcgtgggcgtgcagctgacccggaacggcaccatcatccccgccaacaacaccgtgagcctgggcgccgtgggcaccagcgccgtgagcctgggcctgaccgccaactacgcccggaccggcggccaggtgaccgccggcaacgtgcagagcatcatcggcgtgaccttcgtgtaccagcccggcgacggcaacgccgacgtgaccatcaccgtgaacggcaaggtggtggccaagggcagcggcggcggcggcgccagccgggagctggtggtgagctacgtgaacgtgaacatgggcctgaagatccggcagctgctgtggttccacatcagctgcctgaccttcggccgggagaccgtgctggagtacctggtgagcttcggcgtgtggatccggaccccccccgcctaccggccccccaacgcccccatcctgagcaccctgcccgagaccaccgtggtgggcagcggcggcggcggccaccaccaccaccaccac

SEQ ID NO: 50 - DNA, 1197 bp, Artificial
  FIMHL-HIS-Mi3 J96
atggaaaccgatacactgctgctgtgggtgctgttgctctgggttccaggctctacaggcgattttgcctgcaagaccgccaacggcacagccattcctattggcggaggcagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaagcggcagccaccaccatcatcaccatcaccacggcggcagcatgaagatggaagaactgttcaagaagcacaagatcgtcgccgtgctgcgggccaattctgtggaagaggccaaaaaaaaggccctggccgtgtttcttggcggagtgcacctgatcgagatcacctttaccgtgcctgacgccgacaccgtgatcaaagagctgagcttcctgaaagagatgggcgccatcatcggagccggaaccgtgacatctgttgagcaggccagaaaggccgtggaatctggcgccgagtttatcgtgtcccctcacctggatgaggaaatcagccagttcgccaaagaaaagggcgtgttctacatgcccggcgtgatgacacctacagagctggtcaaagccatgaagctgggccacaccatcctgaagctgtttccaggcgaagtcgtgggccctcagttcgtgaaagctatgaagggcccatttccaaacgtgaagttcgtgcccactggcggcgtcaacctggataatgtgtgcgagtggttcaaggctggcgtgctggctgttggagtgggatctgctctggtcaagggcacacctgtggaagtggctgagaaggccaaggccttcgtggaaaagatcagaggctgcaccgagtga

SEQ ID NO: 51 - DNA, 1068 bp, Artificial
  FIMHL-HIS-Fer 536
atggaaaccgatacactgctgctgtgggtgctgttgctctgggttccaggctctacaggcgattttgcctgcaagaccgccaacggcacagccattcctattggcggaggcagcgccaatgtgtacgttaacctggctcctgccgtgaacgtgggccagaatctggtggtggatctgagcacccagatcttttgccacaacgactaccccgagacaatcaccgactacgtgacactgcagagaggctctgcttacggcggcgtgctgtctagctttagcggcaccgtgaagtacaacggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaagcggcagccaccaccatcatcaccatcaccacggcggcagcgacatcatcaagctgctgaacgagcaagtgaacaaagagatgaacagcagcaacctgtacatgagcatgagcagctggtgctacacacacagcctggatggcgccggactgttcctgtttgatcatgccgccgaggaatacgagcacgccaagaagctgatcatcttcctgaacgagaacaacgtgcccgtgcagctgacatctatcagcgcccctgagcacaagttcgagggcctgacacagatcttccagaaggcctacgaacacgagcagcacatcagcgagagcatcaacaacatcgtggaccacgccatcaagagcaaggatcacgccaccttcaactttctgcagtggtacgtggccgaacagcacgaggaagaggtgctgttcaaggacatcctggacaagatcgagctgatcggcaacgagaaccacggcctgtacctggccgatcagtatgtgaagggaatcgccaagagccggaagtga

SEQ ID NO: 52 - DNA, 1191 bp, Artificial
  FIMH_DG_PGDGN_IMX313_HIS J96
atggaaaccgatacactgctgctgtgggtgctgttgctctgggttccaggatctacaggggattttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaagggcagctcaggctctggctctggatctaaaaaacagggcgacgccgatgtgtgtggcgaggtggcatatatccagagcgtggtgtccgattgtcacgtgccaaccgccgagctgagaaccctgctggaaatccggaagctgttcctcgaaattcagaagctgaaggtcgagctgcagggcctgtctaaagaaggcggaggaagcggatctcaccaccaccatcaccactgatga

SEQ ID NO: 53 - DNA, 1620 bp, Artificial
  FimH_PGDGN_DG_HIS-Ferritn j96
atggaaaccgatacactgctgctgtgggtgctgttgctctgggttccaggatctacaggggattttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaagagcggaagccaccaccatcatcaccatcaccacggcggcagcatgaagatggaagaactgttcaagaagcacaagatcgtcgccgtgctgcgggccaattctgtggaagaggccaaaaaaaaggccctggccgtgtttcttggcggagtgcacctgatcgagatcacctttaccgtgcctgacgccgacaccgtgatcaaagagctgagcttcctgaaagagatgggcgccatcatcggagccggaaccgtgacatctgttgagcaggccagaaaggccgtggaatctggcgccgagtttatcgtgtcccctcacctggatgaggaaatcagccagttcgccaaagaaaagggcgtgttctacatgcccggcgtgatgacacctacagagctggtcaaagccatgaagctgggccacaccatcctgaagctgtttccaggcgaagtcgtgggccctcagttcgtgaaagctatgaagggcccatttccaaacgtgaagttcgtgcccactggcggcgtcaacctggataatgtgtgcgagtggttcaaggctggcgtgctggctgttggagttggctctgctctggtcaagggcacacctgtggaagtggctgagaaggccaaggccttcgtggaaaagatcagaggctgcaccgagtgatga

SEQ ID NO: 54 - DNA, 1489 bp, Artificial
  FIMH_DG_PGDGN-HIS-Ferritin 536
atggaaaccgatacactgctgctgtgggtgctgttgctctgggttccaggatctacaggggatgtttgcctgcaagaccgccaatggcacagccattcctattggcggcggaagcgccaatgtgtacgttaacctggctcctgccgtgaacgtgggccagaatctggtggtggatctgagcacccagatcttttgccacaacgactaccccgagacaatcaccgactacgtgacactgcagagaggctctgcttacggcggcgtgctgtctagctttagcggcaccgtgaagtacaacggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaagagcggaagccaccaccatcatcaccatcaccacggcggcagcgacatcatcaagctgctgaacgagcaagtgaacaaagagatgaacagcagcaacctgtacatgagcatgagcagctggtgctacacacacagcctggatggcgccggactgttcctgtttgatcatgccgccgaggaatacgagcacgccaagaagctgatcatcttcctgaacgagaacaacgtgcccgtccagctgacatctatcagcgcccctgagcacaagttcgagggcctgacacagatcttccagaaggcctacgaacacgagcagcacatcagcgagagcatcaacaacatcgtggaccacgccatcaagagcaaggatcacgccaccttcaactttctgcagtggtacgtggccgaacagcacgaggaagaggtgctgttcaaggacatcctggacaagatcgagctgatcggcaacgagaaccacggcctgtacctggccgatcagtatgtgaagggaatcgccaagagccgcaagtga

SEQ ID NO: 55 - DNA, 1533 bp, Artificial
  FimH_PGDGN_DG-HIS-Ferritin J96
atggaaaccgatacactgctgctgtgggtgctgttgctctgggttccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaagagcggaagccaccaccatcatcaccatcaccacggcggcagcgacatcatcaagctgctgaacgagcaagtgaacaaagagatgaacagcagcaacctgtacatgagcatgagcagctggtgctacacacacagcctggatggcgccggactgttcctgtttgatcatgccgccgaggaatacgagcacgccaagaagctgatcatcttcctgaacgagaacaacgtgcccgtccagctgacatctatcagcgcccctgagcacaagttcgagggcctgacacagatcttccagaaggcctacgaacacgagcagcacatcagcgagagcatcaacaacatcgtggaccacgccatcaagagcaaggatcacgccaccttcaactttctgcagtggtacgtggccgaacagcacgaggaagaggtgctgttcaaggacatcctggacaagatcgagctgatcggcaacgagaaccacggcctgtacctggccgatcagtatgtgaagggaatcgccaagagccgcaagtga

SEQ ID NO: 56 - DNA, 1665 bp, Artificial
  FimH_PGDGN_DG-HIS-MI3 j96
atggaaaccgatacactgctgctgtgggtgctgttgctctgggttccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaagagcggaagccaccaccatcatcaccatcaccacggcggcagcatgaagatggaagaactgttcaagaagcacaagatcgtcgccgtgctgcgggccaattctgtggaagaggccaaaaaaaaggccctggccgtgtttcttggcggagtgcacctgatcgagatcacctttaccgtgcctgacgccgacaccgtgatcaaagagctgagcttcctgaaagagatgggcgccatcatcggagccggaaccgtgacatctgttgagcaggccagaaaggccgtggaatctggcgccgagtttatcgtgtcccctcacctggatgaggaaatcagccagttcgccaaagaaaagggcgtgttctacatgcccggcgtgatgacacctacagagctggtcaaagccatgaagctgggccacaccatcctgaagctgtttccaggcgaagtcgtgggccctcagttcgtgaaagctatgaagggcccatttccaaacgtgaagttcgtgcccactggcggcgtcaacctggataatgtgtgcgagtggttcaaggctggcgtgctggctgttggagttggctctgctctggtcaagggcacacctgtggaagtggctgagaaggccaaggccttcgtggaaaagatcagaggctgcaccgagtgatga

SEQ ID NO: 57 - DNA, 1237 bp, Artificial
  FimH_PGDGN_DG-HIS-IMX313 j96
atggaaaccgatacactgctgctgtgggtgctgttgctctgggttccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaagggcagctcaggctctggctctggatctaaaaaacagggcgacgccgatgtgtgtggcgaggtggcatatatccagagcgtggtgtccgattgtcacgtgccaaccgccgagctgagaaccctgctggaaatccggaagctgttcctcgaaattcagaagctgaaggtcgagctgcagggcctgtctaaagaaggcggaggaagcggatctcaccaccaccatcaccactgatgac

SEQ ID NO: 58 - DNA, 1533 bp, Artificial
  FimH_PGDGN_DG_Ferritin  (536)
atggaaaccgatacactgctgctgtgggtgctgttgctctgggttccaggatctacaggggatgccgctcaacctgctcggagagccagaagaacaaagctggccctgtttgcctgcaagaccgccaatggcacagccattcctattggcggcggaagcgccaatgtgtacgttaacctggctcctgccgtgaacgtgggccagaatctggtggtggatctgagcacccagatcttttgccacaacgactaccccgagacaatcaccgactacgtgacactgcagagaggctctgcttacggcggcgtgctgtctagctttagcggcaccgtgaagtacaacggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtgtctcttggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaagagcggaagccaccaccatcatcaccatcaccacggcggcagcgacatcatcaagctgctgaacgagcaagtgaacaaagagatgaacagcagcaacctgtacatgagcatgagcagctggtgctacacacacagcctggatggcgccggactgttcctgtttgatcatgccgccgaggaatacgagcacgccaagaagctgatcatcttcctgaacgagaacaacgtgcccgtccagctgacatctatcagcgcccctgagcacaagttcgagggcctgacacagatcttccagaaggcctacgaacacgagcagcacatcagcgagagcatcaacaacatcgtggaccacgccatcaagagcaaggatcacgccaccttcaactttctgcagtggtacgtggccgaacagcacgaggaagaggtgctgttcaaggacatcctggacaagatcgagctgatcggcaacgagaaccacggcctgtacctggccgatcagtatgtgaagggaatcgccaagagccgcaagtga

SEQ ID NO: 59 - DNA, 1170 bp, Artificial
  FimH-IMX313 j96
atggaaaccgatacactgctgctgtgggtgctgttgctctgggttccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcccacctgtgatgtgtccgctagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacatctgcagtttctctgggcctgacagccaactatgccagaacaggcggacaagtgacagccggcaatgtgcagtctatcatcggcgtgacattcgtgtatcagggcagctctggcagcggctctggatctaaaaaacagggcgacgccgatgtgtgtggcgaggtggcatatatccagagcgtggtgtccgattgtcacgtgccaaccgccgagctgagaaccctgctggaaatccggaagctgttcctcgaaattcagaagctgaaggtcgagctgcagggcctgtctaaagaaggcggaggaagcggatctcaccaccaccatcaccactga

SEQ ID NO: 60 - DNA, 1629 bp, Artificial
  FimH_mi3 j96
atggaaaccgatacactgctgctgtgggtgctgttgctctgggttccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcccacctgtgatgtgtccgctagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacatctgcagtttctctgggcctgacagccaactatgccagaacaggcggacaagtgacagccggcaatgtgcagtctatcatcggcgtgaccttcgtgtaccaaggatctggcggaggcggcatgaagatggaagaactgttcaagaaacacaagatcgtggccgtgctgcgggccaattctgtggaagaggccaaaaaaaaggccctggccgtgtttctcggaggcgtgcacctgatcgagatcacctttaccgtgcctgacgccgacaccgtgatcaaagagctgagcttcctgaaagagatgggcgccatcatcggagccggaaccgtgacatctgttgagcaggccagaaaggccgtggaatctggcgccgagtttatcgtgtcccctcacctggatgaggaaatcagccagttcgccaaagaaaagggcgtgttctacatgcccggcgtgatgacacctacagagctggtcaaagccatgaagctgggccacaccatcctgaagctgtttccaggcgaagtcgtgggccctcagttcgtgaaagctatgaagggcccatttccaaacgtgaagttcgtgcccactggcggagtgaatctggacaacgtgtgcgagtggttcaaggctggcgtgctggctgttggagttggctctgctctggtcaagggcacacctgtggaagtggctgagaaggccaaggccttcgtggaaaagatcagaggctgtaccgaaggcagcggctctggaagcggatctggatctcaccaccatcatcaccatcaccactga

SEQ ID NO: 61 - DNA, 1248 bp, Artificial
  FIMHL-HIS-mI3 j96
atggaaaccgatacactgctgctgtgggtgctgttgctctgggttccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggatctggcggacaccaccatcatcaccatcaccacggcagcggctccatgaagatggaagaactgttcaagaagcacaagatcgtcgccgtgctgcgggccaattctgtggaagaggccaaaaaaaaggccctggccgtgtttcttggcggagtgcacctgatcgagatcacctttaccgtgcctgacgccgacaccgtgatcaaagagctgagcttcctgaaagagatgggcgccatcatcggagccggaaccgtgacatctgttgagcaggccagaaaggccgtggaatctggcgccgagtttatcgtgtcccctcacctggatgaggaaatcagccagttcgccaaagaaaagggcgtgttctacatgcccggcgtgatgacacctacagagctggtcaaagccatgaagctgggccacaccatcctgaagctgtttccaggcgaagtcgtgggccctcagttcgtgaaagctatgaagggcccatttccaaacgtgaagttcgtgcccactggcggcgtcaacctggataatgtgtgcgagtggttcaaggctggcgtgctggctgttggagtgggatctgctctggtcaagggcacacctgtggaagtggctgagaaggccaaggccttcgtggaaaagatcagaggctgcaccgagtga

SEQ ID NO: 62 - DNA, 1428 bp, Artificial
  FIMH_DG_PGDGN_HIS-Ferritin 536
atgtttgcatgtaaaaccgcaaatggcaccgcaattccgattggtggtggtagcgcaaatgtttatgttaatctggcaccggcagttaatgttggtcagaatctggttgttgatctgagcacccagattttttgccataatgattatccggaaaccatcaccgattatgttaccctgcagcgtggtagtgcatatggtggtgttctgagcagctttagcggcaccgtgaaatataacggtagcagctatccgtttccgaccaccagtgaaacaccgcgtgttgtgtataatagccgtaccgataaaccgtggcctgttgcactgtatctgacaccggttagcagtgccggtggtgttgcaattaaagcaggtagcctgattgcagttctgattctgcgtcagaccaataactataactccgatgattttcagtttgtgtggaacatctatgccaataatgatgttgttgttccgaccggtggttgtgatgttagcgcacgtgatgttaccgttacactgccggattatcctggtagcgttccgattccgctgaccgtttattgtgcaaaaagccagaacctgggttattatctgagcggcaccaccgcagatgcaggtaatagcatttttaccaataccgccagctttagtccggcacaaggtgttggtgttcagctgacccgtaatggcaccattattccggcaaataataccgttagcctgggtgcagttggcaccagcgcagtgagcctgggtctgaccgccaattatgcacgtaccggtggtcaggttaccgcaggtaatgttcagagcattattggtgttacctttgtgtatcagcctggtgatggtaatgcagatgtgaccattaccgtgaatggtaaagttgttgccaaaagcggtagtcatcatcaccaccatcatcatcacggtggtagcgatatcatcaaactgctgaatgaacaggtgaacaaagaaatgaatagcagcaacctgtatatgagcatgagcagctggtgttatacccatagcctggatggtgcaggtctgtttctgtttgatcatgcagccgaagaatatgagcacgcaaaaaaactgatcatcttcctgaatgaaaataatgttccggtgcagctgaccagcattagcgctccggaacataaatttgaaggtctgacacagatttttcagaaagcctatgaacatgaacagcacattagcgaaagcattaacaacattgtggatcacgccatcaaaagcaaagatcatgcaacctttaactttctgcagtggtatgttgcagaacagcatgaagaagaagtgctgtttaaagacatcctggataaaattgaactgatcggcaacgaaaatcatggtctgtatctggcagatcagtatgttaaaggtattgcgaaaagccgcaaataa

SEQ ID NO: 63 - DNA, 774 bp, Artificial
  LS-FIMHL-IMX313-HIS
atgaagtatctgctgccgaccgcagcagcgggtctgctgctgctggcagcacagcctgcaatggcatttgcatgtaaaaccgcaaatggcaccgcaattccgattggtggtggtagcgcaaatgtttatgttaatctggcaccggttgttaatgttggtcagaatctggttgttgatctgagcacccagattttttgccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcactgtatctgacaccggtgagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtagcagcggtagcggttcaggtagcaaaaaacagggtgatgcagatgtttgtggtgaagttgcatatattcagagcgttgttagcgattgtcatgttccgacagcagaactgcgtaccctgctggaaattcgtaaactgtttctggaaatccagaagctgaaagttgaactgcagggtctgagcaaagaaggtggcggaagcggtagccatcaccatcaccatcactga

SEQ ID NO: 64 - DNA, 714 bp, Artificial
  FIMHL-S24S65-IMX313
atgtttgcaagcaaaaccgcaaatggcaccgcaattccgattggtggtggtagcgcaaatgtttatgttaatctggcaccggttgttaatgttggtcagaatctggttgttgatctgagcacccagatttttagccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcactgtatctgacaccggttagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtagtagcggtagtggtagcggttcaaaaaaacagggtgatgcagatgtttgtggtgaagttgcatatattcagagcgttgttagcgattgtcatgtgccgaccgcagaactgcgtaccctgctggaaattcgtaaactgtttctggaaatccagaagctgaaagttgaactgcagggtctgagtaaagaaggtggtggtagtggtagccatcaccatcatcatcactaataa

SEQ ID NO: 65 - DNA, 1092 bp, Artificial
  FIMHL-S24S65-foldon-ferritin
atgtttgcaagcaaaaccgcaaatggcaccgcaattccgattggtggtggtagcgcaaatgtttatgttaatctggcaccggcagttaatgttggtcagaatctggttgttgatctgagcacccagatttttagccataatgattatccggaaaccatcaccgattatgttaccctgcagcgtggtagtgcatatggtggtgttctgagcagctttagcggcaccgtgaaatataacggtagcagctatccgtttccgaccaccagtgaaacaccgcgtgttgtgtataatagccgtaccgataaaccgtggcctgttgcactgtatctgacaccggttagcagtgccggtggtgttgcaattaaagcaggtagcctgattgcagttctgattctgcgtcagaccaataactataactccgatgattttcagtttgtgtggaacatctatgccaataatgatgttgttgttccgaccggttcaggttatattccggaagcaccgcgtgatggtcaggcatatgttcgtaaagatggtgaatgggttctgctgagcacctttttaggtagcggtcatcatcaccatcatcatggtagcggtgatatcattaaactgctgaatgaacaggtgaacaaagagatgaatagcagcaatctgtatatgagcatgagcagctggtgttatacccatagcctggatggtgcaggtctgtttctgtttgatcatgcagccgaagaatatgagcacgcaaaaaaactgatcatcttcctgaatgaaaataatgttccggttcagctgaccagcattagcgctccggaacataaatttgaaggtctgacacagatttttcagaaagcctatgaacatgaacagcacattagcgaaagcattaacaacattgtggatcacgccatcaaaagcaaagatcatgcaacctttaactttctgcagtggtatgttgcagaacagcatgaagaagaagtgctgtttaaagacatcctggataaaattgaactgatcggcaacgaaaatcatggtctgtatctggcagatcagtatgttaaaggtattgccaaaagccgcaagtaataa

SEQ ID NO: 66 - DNA, 1173 bp, Artificial
  FIMHL-S24S65-Mi3
atgtttgcaagcaaaaccgcaaatggcaccgcaattccgattggtggtggtagcgcaaatgtttatgttaatctggcaccggcagttaatgttggtcagaatctggttgttgatctgagcacccagatttttagccataatgattatccggaaaccatcaccgattatgttaccctgcagcgtggtagtgcatatggtggtgttctgagcagctttagcggcaccgtgaaatataacggtagcagctatccgtttccgaccaccagtgaaacaccgcgtgttgtgtataatagccgtaccgataaaccgtggcctgttgcactgtatctgacaccggttagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtagtggtggttcaggtggtagcatgaaaatggaagaactgttcaaaaagcacaaaattgttgccgttctgcgtgcaaatagcgttgaagaagcaaaaaagaaagcactggccgtttttttaggtggtgtgcatctgattgaaatcacctttaccgttccggatgcagataccgttattaaagaactgagctttctgaaagaaatgggtgcaattattggtgcaggcaccgttaccagcgttgaacaggcacgtaaagcagttgaaagcggtgcagaatttattgttagtccgcatctggatgaagaaattagccagtttgcaaaagaaaagggcgtgttttatatgcctggtgttatgaccccgaccgaactggttaaagcaatgaaactgggtcataccatcctgaaactgtttccgggtgaagttgttggtccgcagtttgtgaaagccatgaaaggtccttttccgaacgttaaatttgtgccgacaggtggcgtgaatctggataatgtttgtgaatggtttaaagccggtgttctggccgttggtgttggtagtgccctggtgaaaggtacaccggttgaagttgcagaaaaagcaaaagcctttgtggaaaaaattcgtggttgtaccgaaggtagcggtagcggttcaggtagtggtagccatcaccatcatcatcactaataa

SEQ ID NO: 67 - DNA, 1173 bp, Artificial
  FIMHL-mI3
atgtttgcatgtaaaaccgcaaatggcaccgcaattccgattggtggtggtagcgcaaatgtttatgttaatctggcaccggcagttaatgttggtcagaatctggttgttgatctgagcacccagattttttgccataatgattatccggaaaccatcaccgattatgttaccctgcagcgtggtagtgcatatggtggtgttctgagcagctttagcggcaccgtgaaatataacggtagcagctatccgtttccgaccaccagtgaaacaccgcgtgttgtgtataatagccgtaccgataaaccgtggcctgttgcactgtatctgacaccggttagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtagtggtggttcaggtggtagcatgaaaatggaagaactgttcaaaaagcacaaaattgttgccgttctgcgtgcaaatagcgttgaagaagcaaaaaagaaagcactggccgtttttttaggtggtgtgcatctgattgaaatcacctttaccgttccggatgcagataccgttattaaagaactgagctttctgaaagaaatgggtgcaattattggtgcaggcaccgttaccagcgttgaacaggcacgtaaagcagttgaaagcggtgcagaatttattgttagtccgcatctggatgaagaaattagccagtttgcaaaagaaaagggcgtgttttatatgcctggtgttatgaccccgaccgaactggttaaagcaatgaaactgggtcataccatcctgaaactgtttccgggtgaagttgttggtccgcagtttgtgaaagccatgaaaggtccttttccgaacgttaaatttgtgccgacaggtggcgtgaatctggataatgtttgtgaatggtttaaagccggtgttctggccgttggtgttggtagtgccctggtgaaaggtacaccggttgaagttgcagaaaaagcaaaagcctttgtggaaaaaattcgtggttgtaccgaaggtagcggtagcggttcaggtagtggtagccatcaccatcatcatcactaataa

SEQ ID NO: 68 - DNA, 1185 bp, Artificial
  FimHL-NOCYS-MI3
atgttcgcaagcaaaaccgcaaatggcaccgcaattccgattggtggtggtagcgcaaatgtttatgttaatctggcaccggcagttaatgttggtcagaatctggttgttgatctgagcacccagatttttagccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcactgtatctgacaccggttagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtggtggcagtggtggttcaggcggtagcggtggtagcatgaaaatggaagaactgttcaaaaagcacaaaattgttgccgttctgcgtgcaaatagcgttgaagaagcaaaaaagaaagcactggccgtttttttaggtggtgtgcatctgattgaaatcacctttaccgttccggatgcagataccgttattaaagaactgagctttctgaaagaaatgggtgcaattattggtgcaggcaccgttaccagcgttgaacaggcacgtaaagcagttgaaagcggtgcagaatttattgttagtccgcatctggatgaagaaattagccagtttgcaaaagaaaagggcgtgttttatatgcctggtgttatgaccccgaccgaactggttaaagcaatgaaactgggtcataccatcctgaaactgtttccgggtgaagttgttggtccgcagtttgtgaaagccatgaaaggtccttttccgaacgttaaatttgtgccgacaggtggcgtgaatctggataatgtttgtgaatggtttaaagccggtgttctggcagttggtgttggtagtgccctggtgaaaggtacaccggttgaagttgcagaaaaagcaaaagcctttgtggaaaaaattcgtggttgtaccgaaggtagcggtagtggtagcggttcaggtagccatcaccatcaccatcactga

SEQ ID NO: 69 - DNA, 1575 bp, Artificial
  FimHdeltaGG_PGDGN_DG_mi3
atgttcgcctgcaaaaccgcaaatggcaccgcaattccgattggtggtggtagcgcaaatgtttatgttaatctggcaccggttgttaatgttggtcagaatctggttgttgatctgagcacccagattttttgccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcactgtatctgacaccggtgagcagtgccggtggtgttgcaattaaagcaggtagcctgattgcagttctgattctgcgtcagaccaataactataactccgatgattttcagtttgtgtggaacatctatgccaataatgatgttgttgttccgacctgtgatgttagcgcacgtgatgttaccgttacactgccggattatcctggtagcgttccgattccgctgaccgtttattgtgcaaaaagccagaacctgggttattatctgagcggcaccaccgcagatgcaggtaatagcatttttaccaataccgcaagctttagtccggcacaaggtgttggtgttcagctgacccgtaatggcaccattattccggcaaataataccgttagcctgggtgcagttggcaccagcgcagtgagcctgggtctgaccgccaattatgcacgtaccggtggtcaggttaccgcaggtaatgttcagagcattattggtgttacctttgtgtatcagcctggtgatggtaatgcagatgtgaccattaccgtgaatggtaaagttgttgcaaaaggtagcggtggtggtggcatgaaaatggaagaactgttcaaaaaacacaagattgttgccgttctgcgtgcaaatagcgttgaagaagcaaaaaagaaagcactggccgtttttttaggtggtgtgcatctgattgaaatcacctttaccgttccggatgcagataccgttattaaagaactgagctttctgaaagaaatgggtgcaattattggcgcaggcaccgttaccagcgttgaacaggcacgtaaagcagttgaaagcggtgcagaatttattgttagtccgcatctggatgaagaaattagccagtttgcaaaagaaaagggcgtgttttatatgcctggtgttatgaccccgaccgaactggttaaagcaatgaaactgggtcataccatcctgaaactgtttccgggtgaagttgttggtccgcagtttgtgaaagccatgaaaggtccttttccgaacgttaaatttgtgccgaccggtggcgtgaatctggataatgtttgtgaatggtttaaagccggtgttctggcagttggtgttggtagtgccctggtgaaaggtacaccggttgaagttgcagaaaaagcaaaagcctttgtggaaaaaattcgtggttgtaccgaaggtagtggtagcggcagcggtagcggttcacatcaccatcaccatcactga

SEQ ID NO: 70 - DNA, 1029 bp, Artificial
  FimHL-GSG4-Ferritin
atgggcagcagccatcatcatcatcatcacgaactgtacttccagggctttgcatgtaaaaccgcaaatggcaccgcaattccgattggtggtggtagcgcaaatgtttatgttaatctggcaccggcagttaatgttggtcagaatctggttgttgatctgagcacccagattttttgccataatgattatccggaaaccatcaccgattatgttaccctgcagcgtggtagtgcatatggtggtgttctgagcagctttagcggcaccgtgaaatataacggtagcagctatccgtttccgaccaccagtgaaacaccgcgtgttgtgtataatagccgtaccgataaaccgtggcctgttgcactgtatctgacaccggttagcagtgccggtggtgttgcaattaaagcaggtagcctgattgcagttctgattctgcgtcagaccaataactataactccgatgattttcagtttgtgtggaacatctatgccaataatgatgttgttgttccgaccggtagcggtggtggtggcgatattatcaaactgctgaatgaacaggtgaacaaagaaatgaatagcagcaacctgtatatgagcatgagcagctggtgttatacccatagcctggatggtgcaggtctgtttctgtttgatcatgcagccgaagaatatgagcacgcaaaaaaactgatcatcttcctgaatgaaaataatgttccggtgcagctgaccagcattagcgctccggaacataaatttgaaggtctgacacagatttttcagaaagcctatgaacatgaacagcacattagcgaaagcattaacaacattgtggatcacgccatcaaaagcaaagatcatgcaacctttaactttctgcagtggtatgttgcagaacagcatgaagaagaagtgctgtttaaagacatcctggataaaattgaactgatcggcaatgaaaatcacggtctgtatctggcagatcagtatgttaaaggtattgccaaaagccgcaaataa

SEQ ID NO: 71 - DNA, 1215 bp, Artificial
  pelBLS-FimHL-mI3
atgaaatacctgctgccgaccgctgctgctggtctgctgctcctcgctgcccagccggcgatggcctttgcatgtaaaaccgcaaatggcaccgcaattccgattggtggtggtagcgcaaatgtttatgttaatctggcaccggttgttaatgttggtcagaatctggttgttgatctgagcacccagattttttgccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcactgtatctgacaccggtgagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtggttcaggtatgaaaatggaagaactgttcaaaaagcacaagattgttgccgttctgcgtgcaaatagcgttgaagaagcaaaaaagaaagcactggccgtttttttaggtggtgtgcatctgattgaaatcacctttaccgttccggatgcagataccgttattaaagaactgagctttctgaaagaaatgggtgcaattattggcgcaggcaccgttaccagcgttgaacaggcacgtaaagcagttgaaagcggtgcagaatttattgttagtccgcatctggatgaagaaattagccagtttgcaaaagaaaagggcgtgttttatatgcctggtgttatgaccccgaccgaactggttaaagcaatgaaactgggtcataccatcctgaaactgtttccgggtgaagttgttggtccgcagtttgtgaaagccatgaaaggtccttttccgaacgttaaatttgtgccgacaggtggcgtgaatctggataatgtttgtgaatggtttaaagccggtgttctggcagttggtgttggtagtgccctggtgaaaggtacaccggttgaagttgcagaaaaagcaaaagcctttgtggaaaaaattcgtggttgtaccgaaggtagtggtagcggttcaggtagccaccaccaccaccaccactga

SEQ ID NO: 72 - DNA, 1449 bp, Artificial
  FimH_DG_Ferritin (GSGGGG)
atgggcagcagccatcatcatcatcatcacgaactgtacttccagggctttgcatgtaaaaccgcaaatggcaccgcaattccgattggtggtggtagcgcaaatgtttatgttaatctggcaccggcagttaatgttggtcagaatctggttgttgatctgagcacccagattttttgccataatgattatccggaaaccatcaccgattatgttaccctgcagcgtggtagtgcatatggtggtgttctgagcagctttagcggcaccgtgaaatataacggtagcagctatccgtttccgaccaccagtgaaacaccgcgtgttgtgtataatagccgtaccgataaaccgtggcctgttgcactgtatctgacaccggttagcagtgccggtggtgttgcaattaaagcaggtagcctgattgcagttctgattctgcgtcagaccaataactataactccgatgattttcagtttgtgtggaacatctatgccaataatgatgttgttgttccgaccggtggttgtgatgttagcgcacgtgatgttaccgttacactgccggattatcctggtagcgttccgattccgctgaccgtttattgtgcaaaaagccagaacctgggttattatctgagcggcaccaccgcagatgcaggtaatagcatttttaccaataccgcaagctttagtccggcacaaggtgttggtgttcagctgacccgtaatggcaccattattccggcaaataataccgttagcctgggtgcagttggcaccagcgcagtgagcctgggtctgaccgccaattatgcacgtaccggtggtcaggttaccgcaggtaatgttcagagcattattggtgttacctttgtgtatcagcctggtgatggtaatgcagatgtgaccattaccgtgaatggtaaagttgttgcaaaaggtagcggtggtggtggcgatattatcaaactgctgaatgaacaggtgaacaaagaaatgaatagcagcaacctgtatatgagcatgagcagctggtgttatacccatagcctggatggtgcaggtctgtttctgtttgatcatgcagccgaagaatatgagcacgcaaaaaaactgatcatcttcctgaatgaaaataatgttccggtgcagctgaccagcattagcgctccggaacataaatttgaaggtctgacacagatttttcagaaagcctatgaacatgaacagcacattagcgaaagcattaacaacattgtggatcacgccatcaaaagcaaagatcatgcaacctttaactttctgcagtggtatgttgcagaacagcatgaagaagaagtgctgtttaaagacatcctggataaaattgaactgatcggcaatgaaaatcacggtctgtatctggcagatcagtatgttaaaggtattgccaaaagccgcaaataa

SEQ ID NO: 73 - DNA, 1185 bp, Artificial
  FimHL-C-C-MI3
atgttcgcctgcaaaaccgcaaatggcaccgcaattccgattggtggtggtagcgcaaatgtttatgttaatctggcaccggtttgtaatgttggtcagaattgtgttgttgatctgagcacccagattttttgccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcactgtatctgacaccggtgagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtggtggcagtggtggttcaggcggtagcggtggtagcatgaaaatggaagaactgttcaaaaagcacaaaattgttgccgttctgcgtgcaaatagcgttgaagaagcaaaaaagaaagcactggccgtttttttaggtggtgtgcatctgattgaaatcacctttaccgttccggatgcagataccgttattaaagaactgagctttctgaaagaaatgggtgcaattattggtgcaggcaccgttaccagcgttgaacaggcacgtaaagcagttgaaagcggtgcagaatttattgttagtccgcatctggatgaagaaattagccagtttgcaaaagaaaagggcgtgttttatatgcctggtgttatgaccccgaccgaactggttaaagcaatgaaactgggtcataccatcctgaaactgtttccgggtgaagttgttggtccgcagtttgtgaaagccatgaaaggtccttttccgaacgttaaatttgtgccgacaggtggcgtgaatctggataatgtttgtgaatggtttaaagccggtgttctggcagttggtgttggtagtgccctggtgaaaggtacaccggttgaagttgcagaaaaagcaaaagcctttgtggaaaaaattcgtggttgtaccgaaggtagcggtagtggtagcggttcaggtagccatcaccatcaccatcactga

SEQ ID NO: 74 - DNA, 904 bp, Artificial
  FimHL-C-C-qBeta
atgttcgcctgcaaaaccgcaaatggcaccgcaattccgattggtggtggtagcgcaaatgtttatgttaatctggcaccggtttgtaatgttggtcagaattgtgttgttgatctgagcacccagattttttgccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcactgtatctgacaccggtgagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtggtggcagtggtggttcaggcggtagcggtggcagcgccaaactggaaaccgttacactgggtaatattggtaaagatggtaaacagaccctggttctgaatccgcgtggtgttaatccgaccaatggtgttgccagcctgagccaggcaggcgcagttccggcactggaaaaacgtgttaccgttagcgttagccagccgagccgtaatcgtaaaaactataaagttcaggtgaaaatccagaatccgaccgcatgtaccgccaatggtagctgtgatccgagcgttacccgtcaggcatatgcagatgttacctttagttttacccagtatagcaccgatgaagaacgtgcatttgttcgtaccgaactggcagcactgctggcaagtccgctgctgattgatgcaattgatcagctga

SEQ ID NO: 75 - DNA, 965 bp, Artificial
  HBcAgNC_fimHL splitted
atggatatcgatccgtataaagaatttggtgcaagcgttgaactgctgagctttctgccgagcgatttttttccgagcattcgtgatctgctggataccgcaagcgcactgtatcgtgaagcactggaaagtccggaacattgtagtccgcatcataccgcactgcgtcaggcaattctgtgttggggtgaactgatgaatctggcaacctgggttggtagcaatctggaagatccgtagaaggagatatacatatgtttgcatgtaaaaccgcaaatggcaccgcaattccgattggtggtggtagcgcaaatgtttatgttaatctggcaccggttgttaatgttggtcagaatctggttgttgatctgagcacccagattttttgccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcgctgtatctgacaccggtgagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtggttcaggtgccagccgtgaactggttgttagctatgttaatgtgaatatgggcctgaaaattcgtcagctgctgtggtttcatatttcatgtctgacctttggtcgtgaaaccgttctggaatatctggttagctttggtgtttggattcgtacccctccggcatatcgtccgcctaatgcaccgattctgagtaccctgccggaaacaaccgttgtttgaggatcc

SEQ ID NO: 76 - DNA, 1123 bp, Artificial
  HBcAgNC_fimHL-LS
atgaaatatctgctgccgaccgcagcagcgggtctgctgctgctggcagcacagcctgcaatggcaggtcatcatcaccatcatcatagcggtggtatggatattgatccgtataaagaatttggtgccagcgttgaactgctgagctttctgccgagcgatttttttccgagcattcgtgatctgctggataccgcaagcgcactgtatcgtgaagcactggaaagtccggaacattgtagtccgcatcataccgcactgcgtcaggcaattctgtgttggggtgaactgatgaatctggcaacctgggttggtagcaatctggaagatccgtagaaggagatatacatatgaaatacctgttaccgacagccgcagcaggcctgttactgttagcagcccagccagccatggcatttgcatgtaaaaccgcaaatggcaccgcaattccgattggtggtggtagcgcaaatgtttatgttaatctggcaccggttgttaatgttggtcagaatctggttgttgatctgagcacccagattttttgccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcgctgtatctgacaccggtgagcagtgccggtggtgttgcaattaaagcaggtagcctgattgcagttctgattctgcgtcagaccaataactataactccgatgattttcagtttgtgtggaacatctatgccaataatgatgttgttgttccgaccggtggtggttcaggtgcaagccgtgaactggttgttagctatgttaatgtgaatatgggcctgaaaattcgtcagctgctgtggtttcatatttcatgtctgacctttggtcgtgaaaccgttctggaatatctggttagctttggtgtttggattcgtacccctccggcatatcgtccgcctaatgcaccgattctgagtaccctgccggaaacaaccgttgtttgactcga

SEQ ID NO: 77 - DNA, 1161 bp, Artificial
  FIMHL-MI3
atgttcgcctgcaaaaccgcaaatggcaccgcaattccgattggtggtggtagcgcaaatgtttatgttaatctggcaccggttgttaatgttggtcagaatctggttgttgatctgagcacccagattttttgccataatgattatccggaaaccatcaccgattatgttaccctgcagcgtggtagtgcatatggtggtgttctgagcaattttagcggcaccgtgaaatatagcggtagcagctatccgtttccgaccaccagtgaaacaccgcgtgttgtgtataatagccgtaccgataaaccgtggcctgttgcactgtatctgacaccggtgagcagtgccggtggtgttgcaattaaagcaggtagcctgattgcagttctgattctgcgtcagaccaataactataactccgatgattttcagtttgtgtggaacatctatgccaataatgatgttgttgttccgaccggtagcggtggtggtggcatgaaaatggaagaactgttcaaaaaacacaagattgttgccgttctgcgtgcaaatagcgttgaagaagcaaaaaagaaagcactggccgtttttttaggtggtgtgcatctgattgaaatcacctttaccgttccggatgcagataccgttattaaagaactgagctttctgaaagaaatgggtgcaattattggcgcaggcaccgttaccagcgttgaacaggcacgtaaagcagttgaaagcggtgcagaatttattgttagtccgcatctggatgaagaaattagccagtttgcaaaagaaaagggcgtgttttatatgcctggtgttatgaccccgaccgaactggttaaagcaatgaaactgggtcataccatcctgaaactgtttccgggtgaagttgttggtccgcagtttgtgaaagccatgaaaggtccttttccgaacgttaaatttgtgccgaccggtggcgtgaatctggataatgtttgtgaatggtttaaagccggtgttctggcagttggtgttggtagtgccctggtgaaaggtacaccggttgaagttgcagaaaaagcaaaagcctttgtggaaaaaattcgtggttgtaccgaaggtagtggtagcggcagcggtagcggttcacatcaccatcaccatcactga

SEQ ID NO: 78 - protein, 15 aa, Artificial
  Leader
AAQPARRARRTKLAL

SEQ ID NO: 79 - protein, 342 aa, Artificial
  FimH_DNKQ_DG_deglyc
METDTLLLWVLLLWVPGSTGDAAQPARRARRTKLALFACKTASGTAIPIGGGSANVYVNLAPVVNVGQNLVVDLSTQIFCHNDYPETITDYVTLQRGSAYGGVLSDFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRDGTIIPADNTVSLGAVGTSAVSLGLTANYARTGGQVTAGNVQSIIGVTFVYQDNKQADVTITVNGKVVAKGSGHHHHHH

SEQ ID NO: 80 - protein, 343 aa, Artificial
  FimH_PGDGN_DG
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGHHHHHH

SEQ ID NO: 81 - protein, 342 aa, Artificial
  FimH_DNKQ_DG
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQDNKQADVTITVNGKVVAKGSGHHHHHH

SEQ ID NO: 82 - protein, 341 aa, Artificial
  FimH_DeltaGG_PGDGN_DG
METDTLLLWVLLLWVPGSTGDAAQPARRARRTKLALFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGHHHHHH

SEQ ID NO: 83 - protein, 326 aa, Artificial
  FimH_DGG_sl
METDTLLLWVLLLWVPGSTGDFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGHHHHHH

SEQ ID NO: 84 - protein, 328 aa, Artificial
  FimH_PGDGN_sl
METDTLLLWVLLLWVPGSTGDFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGHHHHHH

SEQ ID NO: 85 - protein, 327 aa, Artificial
  FimH_DNKQ_sl
METDTLLLWVLLLWVPGSTGDFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQDNKQADVTITVNGKVVAKGSGHHHHHH

SEQ ID NO: 86 - protein, 538 aa, Artificial
  FIMH_DG_PGDGN_536-MI3
METDTLLLWVLLLWVPGSTGDFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 87 - protein, 598 aa, Artificial
  FIMH_DG_PGDGN_536-encapsuline
METDTLLLWVLLLWVPGSTGDFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSMEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKF

SEQ ID NO: 88 - protein, 351 aa, Artificial
  HBcFIMHLJ96
METDTLLLWVLLLWVPGSTGDDIDPYKEFGASVELLSFLPSDFFPSIRDLLDTASALYREALESPEHCSPHHTALRQAILCWGELMNLATWVGSNLEDPGSGGGGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGGSGGASRELVVSYVNVNMGLKIRQLLWFHISCLTFGRETVLEYLVSFGVWIRTPPAYRPPNAPILSTLPETTVVGSGGGGHHHHHH

SEQ ID NO: 89 - protein, 491 aa, Artificial
  HBcFIMHDGJ96
METDTLLLWVLLLWVPGSTGDDIDPYKEFGASVELLSFLPSDFFPSIRDLLDTASALYREALESPEHCSPHHTALRQAILCWGELMNLATWVGSNLEDPGSGGGGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGGGGASRELVVSYVNVNMGLKIRQLLWFHISCLTFGRETVLEYLVSFGVWIRTPPAYRPPNAPILSTLPETTVVGSGGGGHHHHHH

SEQ ID NO: 90 - DNA, 1028 bp, Artificial
  FimH_DNKQ_DG_deglyc
atggaaaccgatacactgctgctgtgggtgctgttgctctgggttccaggatctacaggggatgccgctcaacctgctcggagagccagaagaacaaagctggccctgtttgcctgtaaaaccgccagcggcacagccattcctattggcggaggcagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcgatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgaccagagatggcacaatcatccccgccgacaataccgtgtctctgggcgctgttggcacatctgcagtttctctgggcctgaccgccaactatgccagaacaggtggacaagtgaccgccggcaatgtgcagtctatcatcggcgtgacattcgtgtatcaggacaacaagcaggccgacgtgaccatcaccgtgaatggcaaagtggtggccaaaggctctggccatcaccaccaccatcactg

SEQ ID NO: 91 - DNA, 1011 bp, Artificial
  FimH_PGDGN_DG
atggaaaccgacacactgctgctgtgggtgctgcttttgtgggtgccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtttctctgggcctgacagccaactatgccagaacaggcggacaagtgacagccggcaatgtgcagtctatcatcggcgtgacattcgtgtatcaggacaacaagcaggccgacgtgaccatcaccgtgaatggcaaagtggtggccaaaggctctggccat

SEQ ID NO: 92 - DNA, 1031 bp, Artificial
  FimH_DNKQ_DG
atggaaaccgacacactgctgctgtgggtgctgcttttgtgggtgccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtttctctgggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaaaggctctggacaccaccaccatcaccactg

SEQ ID NO: 93 - DNA, 1025 bp, Artificial
  FimH_DeltaGG_PGDGN_DG
atggaaaccgacacactgctgctgtgggtgctgcttttgtgggtgccaggatctacaggggatgccgctcaacctgctcggagagccagaagaacaaagctggccctgtttgcctgcaagaccgccaatggcacagccattcctattggcggcggaagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcccacctgtgatgtgtccgctagagatgtgacagtgaccctgcctgattaccccggctctgtgcctattcctctgaccgtgtactgcgccaagtctcagaacctgggctactacctgagcggcacaacagccgatgccggcaacagcatctttaccaacaccgccagcttcagccctgctcaaggtgttggagtgcagctgacccggaacggaacaatcatccccgccaacaataccgtgtctctgggagctgtgggcacatctgctgtttctctgggcctgacagccaactatgccagaacaggcggacaagtgacagccggcaatgtgcagtctatcatcggcgtgaccttcgtgtatcagcctggcgacggaaatgccgacgtgaccatcacagtgaatggcaaggtggtggccaaaggctctggacaccaccaccatcaccactg

SEQ ID NO: 94 - DNA, 981 bp, Artificial
  FimH_DGG_sl
atggaaaccgacacactgctgctgtgggtgctgcttttgtgggtgccaggctctacaggcgattttgcctgcaagaccgccaacggcacagccattcctattggcggaggcagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcccacctgtgatgtgtccgctagagatgtgacagtgaccctgcctgattaccccggctctgtgcctattcctctgaccgtgtactgcgccaagagccagaacctgggctactacctgtctggcacaacagccgatgccggcaacagcatctttaccaacaccgccagcttcagccctgctcaaggtgttggagtgcagctgacccggaacggaacaatcatccccgccaacaataccgtgtctctgggagctgtgggcacatctgctgtttctctgggcctgaccgccaattatgccagaacaggcggacaagtgaccgccggcaatgtgcagtctatcatcggcgtgaccttcgtgtatcagcctggcgacggaaacgccgatgtgaccatcacagtgaatggcaaggtggtggccaaaggctctggacaccaccaccatcaccactga

SEQ ID NO: 95 - DNA, 993 bp, Artificial
  FimH_PGDGN_sl
atggaaaccgacacactgctgctgtgggtgctgcttttgtgggtgccaggctctacaggcgattttgcctgcaagaccgccaacggcacagccattcctattggcggaggcagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagagccagaacctgggctactacctgtctggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtttctctgggcctgacagccaactatgccagaacaggcggacaagtgacagccggcaatgtgcagtctatcatcggcgtgaccttcgtgtatcagcctggcgacggaaacgccgatgtgaccatcacagtgaatggcaaggtggtggccaaaggctctggacaccaccaccatcaccactgactcgag

SEQ ID NO: 96 - DNA, 990 bp, Artificial
  FimH_DNKQ_sl
atggaaaccgacacactgctgctgtgggtgctgcttttgtgggtgccaggctctacaggcgattttgcctgcaagaccgccaacggcacagccattcctattggcggaggcagcgccaatgtgtacgtgaacctggctcctgtggtcaacgtgggccagaatctggtggtggacctgagcacccagatcttttgccacaacgactaccccgagacaatcaccgactacgtgacactgcagagaggctctgcttacggcggcgtgctgagcaatttttccggcacagtgaagtacagcggcagcagctacccatttcctaccaccagcgagacacccagagtggtgtacaacagcagaaccgacaagccctggcctgtggctctgtacctgacacctgttagttctgccggcggagtggccattaaggccggatctctgattgccgtgctgatcctgcggcagaccaacaactacaacagcgacgacttccagttcgtgtggaacatctacgccaacaacgacgtggtggtgcctacaggcggatgtgatgtgtccgccagagatgtgacagtgaccctgcctgattaccccggctctgtgcctattcctctgaccgtgtactgcgccaagagccagaacctgggctactacctgtctggcacaacagccgatgccggcaacagcatctttaccaacaccgccagcttcagccctgctcaaggtgttggagtgcagctgacccggaacggaacaatcatccccgccaacaataccgtgtctctgggagctgtgggcacctctgctgtttctctgggcctgacagccaactatgccagaacaggcggacaagtgacagccggcaatgtgcagtctatcatcggcgtgacattcgtgtatcaggacaacaagcaggccgacgtgaccatcaccgtgaatggcaaagtggtggccaaaggctctggccatcaccaccaccatcactgactcgag

SEQ ID NO: 97 - DNA, 1617 bp, Artificial; identical to SEQ ID NO: 46
  FIMH_DG_PGDGN_536-MI3

SEQ ID NO: 98 - DNA, 1053 bp, Artificial; identical to SEQ ID NO: 48
  HBcFIMHLJ96

SEQ ID NO: 99 - DNA, 1473 bp, Artificial; identical to SEQ ID NO: 49
  HBcFIMHDGJ96

SEQ ID NO: 100 - protein, 303 aa, E. coli
MIVMKRVITLFAVLLMGWSVNAWSFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 101 - protein, 279 aa, E. coli
FACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 102 - protein, 303 aa, E. coli
MIVMKRVITLFAVLLMGWSVNAWSFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDASARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 103 - protein, 279 aa, E. coli
FACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDASARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 104 - protein, 303 aa, E. coli
MIVMKRVITLFAVLLMGWSVNAWSFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 105 - protein, 279 aa, E. coli; identical to SEQ ID NO: 2

SEQ ID NO: 106 - protein, 300 aa, E. coli
MKRVITLFAVLLMGWSVNAWSFACKTANGTAIPIGGGSANVYVNLAPAVNVGQNLVVDLSTQIFCHNDYPETITDYVTLQRGAAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 107 - protein, 279 aa, E. coli
FACKTANGTAIPIGGGSANVYVNLAPAVNVGQNLVVDLSTQIFCHNDYPETITDYVTLQRGAAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 108 - protein, 20 aa, Artificial
  Leader
METDTLLLWVLLLWVPGSTG

SEQ ID NO: 109 - protein, 163 aa, H. pylori
DIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 110 - protein, 148 aa, Artificial
  HBC
MDIDPYKEFGATVELLSFLPSDFFPSVRDLLDTASALYREALESPEHCSPHHTALRQAILCWGELMTLATWVGNNLEDASRDLVVNYVNTNMGLKIRQLLWFHISCLTFGRETVLEYLVSFGVWIRTPPAYRPPNAPILSTLPETTVV

SEQ ID NO: 111 - protein, 133 aa, Artificial
  Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 112 - protein, 9 aa, Artificial
  Linker
GSSGSGSGS

SEQ ID NO: 113 - protein, 5 aa, Artificial
  Linker
GGSGS

SEQ ID NO: 114 - protein, 14 aa, Artificial
  Linker
SGSHHHHHHHHGGS

SEQ ID NO: 115 - protein, 13 aa, Artificial
  Linker
AKFVAAWTLKAAA

SEQ ID NO: 116 - protein, 16 aa, Artificial
  Linker
GGGGSLVPRGSGGGGS

SEQ ID NO: 117 - protein, 16 aa, Artificial
  Linker
EAAAKEAAAKEAAAKA

SEQ ID NO: 118 - protein, 17 aa, Artificial
  Linker
SGSFVAAWTLKAAAGGS

SEQ ID NO: 119 - protein, 12 aa, Artificial
  Linker
SGSGSGGGGGGS

SEQ ID NO: 120 - protein, 307 aa, Artificial
  FimH_DNKQ_DG citopl pET22b+
MFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQDNKQADVTITVNGKVVAKGSGHHHHHH

SEQ ID NO: 121 - protein, 308 aa, Artificial
  FimH_PGDGN_DG citopl pET22b+
MFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGHHHHHH

SEQ ID NO: 122 - protein, 306 aa, Artificial
  FimH_DGG_PGDGN_DG citopl pET22b+
MFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGHHHHHH

SEQ ID NO: 123 - protein, 299 aa, Artificial
  FimH_PGDGN_DG citopl PET24 Tagless
MFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAK

SEQ ID NO: 124 - protein, 298 aa, Artificial
  FimH_PGDGN_DG citopl tagless no Met
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAK

SEQ ID NO: 125 - protein, 188 aa, Artificial
  VH of mAb 926
QVQLQQSGAELATPGASVKMSCKASGYTSTNYWIHWVKQRPGQGLEWIGYINPTSGYTEYNQNFKDKATLTADKSSSTAYMQLTSLTSEDSAVYYCARGVIRDFWGQGTTLTVSSAKTTAPSVYPLAPVCGDTTGSSVTLGCLVKGYFPEPVTLTWNSGSLSSGVHTFPAVLQSDLYTLSSSVTVTSS

SEQ ID NO: 126 - protein, 112 aa, Artificial
  VL (kappa) of mAb 926
DVLMTQTPLSLPVSLGDQASISCRSSQNIVHNNGNTYLEWYLQSPGQSPKLLIYKVSNRFSGVPDRFSGSGSGTDFTLKISRVEAEDLGVYYCFQGSHVPFTFGSGTKLEIK

SEQ ID NO: 127 - protein, 191 aa, Artificial
  VH of mAb 475
QVQLQQSGAELVRPGSSVKISCKASGYAFSSYWMNWVKQRPGQGLEWIGQIYPRDGDTNYNGKFMDKVTLTADKSSNTAYMQLSSLTSEDSAVYFCEVGRGFYGMDYWGQGTSVTVSSAKTTAPSVYPLAPVCGDTTGSSVTLGCLVKGYFPEPVTLTWNSGSLSSGVHTFPAVLQSDLYTLSSSVTVTSS

SEQ ID NO: 128 - protein, 106 aa, Artificial
  VL (kappa) of mAb 475
DIVMTQSPKFMSTSVGDRVSVTCKASQNVSNVAWYQQKPGQSPKAMIYSASYRYSGVPGRFTGSGSGTDFTLTINNVQSEDLATYFCQQNSSFPFTFGGGTKLEIK

SEQ ID NO: 129 - protein, 487 aa, Artificial
  D_FimHDG_Fer_GSG4
METDTLLLWVLLLWVPGSTGDFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGSGGGGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 130 - protein, 492 aa, Artificial
  D_FimHDG_Fer0.5_deglyc_tagless
METDTLLLWVLLLWVPGSTGDFACKTAQGTAIPIGGGSANVYVNLAPVVNVGQNLVVDLSTQIFCHNDYPETITDYVTLQRGSAYGGVLSQFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPAQNTVSLGAVGTSAVSLGLTANYARTGGQVTAGNVQSIIGVTFVYQPGDGNADVTITVNGKVVAKGSGGGGGGMLKPEMIEKLNEQMNLELYSSLLYQQMSAWCSYHGFEGAAAFLRRHAQEEMTHMQRLFDYLTDTGNLPRIDTIPSPFAEYSSLDELFQETYKHEQLITQKINELAHAAMTNQDYPTFNFLQWYVAEQHEEEKLFKSIIDKLSLAGKSGEGLYFIDKELSTLDTQN

SEQ ID NO: 131 - protein, 318 aa, Artificial
  FimHDG_deglyc_tagless
METDTLLLWVLLLWVPGSTGFACKTAQGTAIPIGGGSANVYVNLAPVVNVGQNLVVDLSTQIFCHNDYPETITDYVTLQRGSAYGGVLSQFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPAQNTVSLGAVGTSAVSLGLTANYARTGGQVTAGNVQSIIGVTFVYQPGDGNADVTITVNGKVVAK

SEQ ID NO: 132 - protein, 319 aa, Artificial
  D_FimHDG_deglyc_tagless
METDTLLLWVLLLWVPGSTGDFACKTAQGTAIPIGGGSANVYVNLAPVVNVGQNLVVDLSTQIFCHNDYPETITDYVTLQRGSAYGGVLSQFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPAQNTVSLGAVGTSAVSLGLTANYARTGGQVTAGNVQSIIGVTFVYQPGDGNADVTITVNGKVVAK

SEQ ID NO: 133 - protein, 318 aa, Artificial
  FimHDG_N7Q_tagless
METDTLLLWVLLLWVPGSTGFACKTAQGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAK

SEQ ID NO: 134 - protein, 493 aa, Artificial
  D_FimHDG_Fer_deglyc_tagless
METDTLLLWVLLLWVPGSTGDFACKTAQGTAIPIGGGSANVYVNLAPVVNVGQNLVVDLSTQIFCHNDYPETITDYVTLQRGSAYGGVLSQFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPAQNTVSLGAVGTSAVSLGLTANYARTGGQVTAGNVQSIIGVTFVYQPGDGNADVTITVNGKVVAKSGSGSGGGGGGSDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 135 - protein, 493 aa, Artificial
  FimH_PGDGN_DG_GGS4-Ferritn j96
METDTLLLWVLLLWVPGSTGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGGSGGSGGSGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 136 - protein, 318 aa, Artificial
  FimH_PGDGN_DG j96
METDTLLLWVLLLWVPGSTGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAK

SEQ ID NO: 137 - protein, 594 aa, Artificial
  FIMHDG_PADRE-encapsuline536
METDTLLLWVLLLWVPGSTGFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKFVAAWTLKAAAMEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKF

SEQ ID NO: 138 - protein, 494 aa, Artificial
  FIMH_NOD-FERRITN_J96
METDTLLLWVLLLWVPGSTGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHGSGGGGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 139 - protein, 497 aa, Artificial
  FIMH_DG_NOALFA-ferritin_J96
METDTLLLWVLLLWVPGSTGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGGGGSLVPRGSGGGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 140 - protein, 495 aa, Artificial
  FIMH_NOD_S_HIS_FERRITN_J96
METDTLLLWVLLLWVPGSTGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 141 - protein, 498 aa, Artificial
  FIMH_DG_PGDGN-PADRE-Ferritin536
METDTLLLWVLLLWVPGSTGFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSFVAAWTLKAAAGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 142 - protein, 498 aa, Artificial
  fimh-DG_ferritina-linkerAlpha
METDTLLLWVLLLWVPGSTGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKAEAAAKEAAAKEAAAKADIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 143 - protein, 492 aa, Artificial
  FIMH-PADRE-Ferritin536_noSpaces
METDTLLLWVLLLWVPGSTGFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKFVAAWTLKAAADIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 144 - protein, 493 aa, Artificial
  FIMH_DG_GSG4-ferritin_J96
METDTLLLWVLLLWVPGSTGDFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSGSGGGGGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 145 - protein, 498 aa, Artificial
  FIMH_DG_PGDGN-SGS_PADRE-Ferritinj96
METDTLLLWVLLLWVPGSTGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSFVAAWTLKAAAGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 146 - protein, 497 aa, Artificial; identical to SEQ ID NO: 139
  fimh-ferritina-linkerNONa

SEQ ID NO: 147 - protein, 492 aa, Artificial
  FIMH-Ferritinj96_noSpaces
METDTLLLWVLLLWVPGSTGFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKFVAAWTLKAAADIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 148 - protein, 493 aa, Artificial
  FIMH_DG_PGDGN-GGS4-Ferritin536
METDTLLLWVLLLWVPGSTGDFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKGGSGGSGGSGGSDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 149 - protein, 164 aa, Artificial
  1EUM_0_5 - stabilized E. coli ferritin
LKPEMIEKLNEQMNLELYSSLLYQQMSAWCSYHGFEGAAAFLRRHAQEEMTHMQRLFDYLTDTGNLPRIDTIPSPFAEYSSLDELFQETYKHEQLITQKINELAHAAMTNQDYPTFNFLQWYVAEQHEEEKLFKSIIDKLSLAGKSGEGLYFIDKELSTLDTQN

SEQ ID NO: 150 - protein, 164 aa, Artificial
  1EUM_2 - stabilized E. coli ferritin
LKPEMIEKLNEQMNLELYSSLLYQQMSAWCSYHGFEGAAAFLRRHAQEEMTHMQRLFDYLTDTGNLPRINTIPSPFAEYSSLDELFQETYKHEQLITQKINELAHAAMTNQDYPTFNFLQWYVAEQHEEEKLFKSIIDKLSLAGKSGEGLYFIDKELSTLDTQN

SEQ ID NO: 151 - protein, 164 aa, Artificial
  1EUM_2_5 - stabilized E. coli ferritin
LKPEMIEKLNEQMNLELYSSLLYQQMSAWCSYHGFEGAAAFLRRHAQEEMTHMQRLFDYLTDTGNLPRINTVPSPFAEYSSLDELFQETYKHEQLITQKINELAHAAMTNQDYPTFNFLQWYVAEQHEEEKLFKSIIDKLSLAGKSGEGLYFIDKELSTLDTQN

SEQ ID NO: 152 - protein, 164 aa, Artificial
  1EUM_6- stabilized E. coli ferritin
LKPEMIEKLNEQMNLELYSSLLYQQMSAWCSYHGFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDTQN

SEQ ID NO: 153 - protein, 498 aa, Artificial
  Fusion of FimH_DG to E.Coli Ferritin stabilized 0.5
METDTLLLWVLLLWVPGSTGDFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQPGDGNADVTITVNGKVVAKSGSHHHHHHHHGGSMLKPEMIEKLNEQMNLELYSSLLYQQMSAWCSYHGFEGAAAFLRRHAQEEMTHMQRLFDYLTDTGNLPRIDTIPSPFAEYSSLDELFQETYKHEQLITQKINELAHAAMTNQDYPTFNFLQWYVAEQHEEEKLFKSIIDKLSLAGKSGEGLYFIDKELSTLDTQN

(19) United States
(12) Patent Application Publication
(10) Pub. No.: US 2024/0115688 A1
(43) Pub. Date: Apr. 11, 2024

(54) Novel Antigens

(71) Applicant: GLAXOSMITHKLINE BIOLOGICALS SA, Rixensart (BE)

(72) Inventors: Roberto ADAMO, Siena (IT); Roberta COZZI, Siena (IT); Adele FANTONI, Siena (IT); Sanjay PHOGAT, Rockville, MD (US); Roberto ROSINI, Siena (IT); Maria SCARSELLI, Siena (IT); Newton WAHOME, Rockville, MD (US)

(73) Assignee: GLAXOSMITHKLINE BIOLOGICALS SA, Rixensart (BE)

(21) Appl. No.: 18/255,442

(22) PCT Filed: Nov. 30, 2021
(86) PCT No.: PCT/EP2021/083659

§ 371 Date: Jun. 1, 2023

(30) Foreign Application Priority Data
Dec. 2, 2020 (EP) 20211337.9
Dec. 17, 2020 (EP) 20214942.3

(51) Int. Cl. A61K 39/12; C07K 14/005; C07K 14/245

(52) U.S. Cl. CPC A61K 39/12; C07K 14/005; C07K 14/245; A61K 2039/55572

(57) ABSTRACT

The present invention is directed to novel, modified FimH polypeptides, nucleic acids encoding them, and the use of the polypeptides and nucleic acids in the treatment and/or prevention of disease, in particular, urinary tract infection (UTI).

### SEQUENCE LISTING

The instant application contains an electronically submitted Sequence Listing in ASCII text file format (VB67013 FF Seq List_ST25.txt; Size: 356.838 bytes; and Date of Creation: 27 Oct. 2021) which is hereby incorporated by reference in its entirety.

### TECHNICAL FIELD OF THE INVENTION

The present invention is directed to novel, modified FimH polypeptides, nucleic acids encoding them, and the use of the polypeptides and nucleic acids in the treatment and/or prevention of disease, in particular, urinary tract infection (UTI).

### BACKGROUND

Uropathogenic Escherichia coli (UPEC) account for approximately 85% of all urinary tract infections (UTIs) (A. R. Ronald, Urinary tract infection in adults: Research priorities and strategies. Int. J. Antimicrob. Agents 17, 343-348; 2001). The tip-localized adhesin FimH of the type 1 pili allows UPEC to colonize the bladder epithelium during UTIs by binding to mannosylated receptors on the urothelial surface (M. A. Mulvey, Induction and evasion of host defences by type 1-piliated uropathogenic Escherichia coli. Science 282, 1494-1497; 1998).

FimH is phase variable and environmental signals influence its expression, allowing bacteria to attach and avoid being eliminated by micturition (Infect. Immun. 1998, 66, 3303). Anti-FimH IgGs are known to inhibit bacterial adhesion to the bladder in mice and monkeys and the protective effect was associated with the presence of anti-FimH IgGs in the urine (Langermann S, et al. Science. 1997 Apr. 25; 276(5312):607-11; Langermann S, et al. J Infect Dis. 2000 February; 181(2):774-8). Transudation of serum functional IgGs in the urogenital tract seems responsible for inhibiting bacterial adhesion.

FimH protein is composed of an N-terminal lectin domain (FimH_{L}), which binds mannose via a pocket formed by three loops, a 5-amino acids linker and the C-terminal pilin domain (FimH_{P}) that attaches FimH to the pilus.

Crystal structures of FimH in different stages of pilus assembly showed that FimH_{P} is constituted by an incomplete immunoglobulin (Ig)-like fold which is stabilized via a donor strand complementation interaction with the chaperone FimC in the periplasm, and with FimG when the pilus assembles. FimH_{P} adopts a single conformation, but FimH_{L} can assume at least two conformational states with different affinities for mannose
the high-affinity conformation, the relaxed (R) state, and the low-affinity conformation, the tense (T) state (D. Choudhury, X-ray structure of the FimC-FimH chaperone-adhesin complex from uropathogenic Escherichia coli. Science 285, 1061-1066 (1999); C.-S. Hung, Structural basis of tropism of Escherichia coli to the bladder during urinary tract infection. Mol. Microbiol. 44, 903-915 (2002); I. Le Trong, Structural basis for mechanical force regulation of the adhesin FimH via finger trap-like 3 sheet twisting. Cell 141, 645-655 (2010); G. Phan, Crystal structure of the FimD usher bound to its cognate FimC-FimH substrate. Nature 474, 49-53 (2011); S. Geibel, Structural and energetic basis of folded-protein transport by the FimD usher. Nature 496, 243-246 (2013)).

When FimH binds to FimC, FimH adopts an elongated conformation in which FimH_{L} and FimH_{P} do not interact with each other, and FimH_{L} is in a high-affinity mannose-binding state. When FimH is bound to FimG, FimH adopts a compact conformation, wherein FimH_{L} and FimH_{P} interact closely and FimH_{L} adopts a low-affinity mannose-binding state. FimH_{P} can allosterically decrease the ability of FimH_{L} to bind mannose through interactions with the base of FimH_{L}; while mannose binding to FimH_{L} induces FimH_{L} conformations that do not interact with FimH_{P}.

Previously, it has been reported the monoclonal antibodies against FimH_{L} in the low affinity conformation lead to a better inhibition of adhesion to the bladder compared to monoclonal antibodies against the mannose post-binding form (Tchesnokova et al., 2011, ‘Type 1 Fimbrial Adhesin FimH Elicits an Immune Response That Enhances Cell Adhesion of Escherichia coli’ Infect. Immun. 79(10): 3895-3904).

FimH with its non-complemented pilin domain is unstable and tends to aggregate. Of note, FimH has been typically used as antigen in complex with the periplasmic protein FimC. The FimC component did not directly contribute to reduction of bacterial colonization in mice, but rather in FimH stabilization, protecting it from degradation (Science 1997, 276, 607; FEMS Microbiol. Lett. 2000, 188, 147). To produce a stable FimH protein FimG donor strand peptide (FimG residues 1-14) has been added in vitro to displace the pilus assembly chaperone FimC from FimH. (Sauer M M, et al. Nat Commun. 2016 Mar. 7; 7:10738.). A low affinity conformation of FimH_{L} has also been obtained inserting a disulphide bridge, locking the mannose pocket (Kisiela D I, et al. Proc Natl Acad Sci USA. 2013 Nov. 19; 110(47):19089-94).

Use of FimHC complexes include significant production burdens—i.e., production of two polypeptides, which must then be complexed together, presenting an unwelcome complication and a significant storage problem since, for the antigens to be effective, stability of the complexes must be maintained during storage. The immunogenicity of FimH_{L} with disulphide bridge is variable due to low molecular weight of the portion, and full FimH with a disulphide bridge in the FimH_{L} domain proved difficult to express.

Accordingly, there remains an outstanding need for ExPEC antigens that are both immunologically effective and viable for production at scale.

### DESCRIPTION

Importantly, FimC stabilizes FimH in its extended post-binding-like form (Nat. Commun. 2016, 7, 10738). The present inventors have surprisingly found that, by a structure-guided design, it is possible to stabilize the pre-binding form of FimH in absence of FimC and/or improve the capacity of generated anti-FimH antibodies to inhibit bacterial adhesion to uroepithelial cells.

Accordingly, a first aspect of the invention provides a polypeptide having an amino acid sequence comprising or consisting of:
(a) FimH; or a variant, fragment and/or fusion of FimH, and
(b) a donor-strand complementing amino acid sequence,
wherein (b) is downstream of (a).

By “downstream” we mean or include an amino acid sequence that, within the primary amino acid sequence of a polypeptide, is located closer to the C-terminus of the polypeptide respective to a reference sequence.

Alternatively or additionally, the polypeptide of the invention comprises or consists of an amino acid sequence X-(a)-L-(b)-Y, wherein “(a)” is a FimH polypeptide; or a variant, fragment and/or fusion of FimH; “L” is an optional first linker; “(b)” is a donor-strand complementing amino acid sequence, “X” is an optional N-terminal amino acid sequence; “Y is an optional C-terminal amino acid sequence, wherein “Y” is not derived from FimC or FimH or a fragment thereof.

By ‘a donor-strand complementing amino acid sequence’ we mean an amino acid sequence capable of maintaining FimH in (a) the high-affinity conformation, relaxed (R) state, or (b) the low-affinity conformation, the tense (T) state. In one preferred embodiment, the donor strand complementing amino acid sequence is capable of maintaining FimH in the low affinity conformation, i.e. the tense (T) state.

By ‘the high-affinity conformation, relaxed (R) state’ we mean or include with mannose binding affinity closer to that of FimH in the high-affinity conformation than the low-affinity conformation (in particular, FimH from which the polypeptide of the invention was derived or principally derived, especially where complexed with FimC) e.g., at least 51%, 60%, 70%, 80%, 90%, 95%, 96%, 97%, 98%, 99% or 100% of the mannose binding affinity of FimH in the high-affinity conformation, for example, K_{d}<1.2 μM as disclosed in Kisiela D I, et al. Proc Natl Acad Sci USA. 2013 Nov. 19; 110(47):19089-94.

By ‘the low-affinity conformation, the tense (T) state’ we mean or include with mannose binding affinity closer to that of FimH in the low-affinity conformation than the high-affinity conformation (in particular, FimH from which the polypeptide of the invention was derived or principally derived, especially where complexed with FimC) e.g., less than 50%, 40%, 30%, 20%, 15%, 10%, 5%, 4%, 3%, 2% or 1% of the mannose binding affinity of FimH in the high-affinity conformation, for example, K_{d}˜ 300 μM or higher (i.e. has no detectable mannose binding affinity), as disclosed in Kisiela D I, et al. Proc Natl Acad Sci USA. 2013 Nov. 19; 110(47):19089-94. In one embodiment, the polypeptide of the invention is in the low-affinity conformation, for example has a mannose binding affinity of K_{d} of about, 100 μM, 200 μM, 300 μM, 400 μM, 500 μM, 600 μM, 700 μM, 800 μM, 900 μM, or 1 mM or has no detectable mannose binding affinity.

Mannose binding can be determined using any suitable means known in the art, for example, surface plasmon resonance (SPR) may be used tₒ verify binding, binding specificity and binding constants of FimH constructs with mannosylated bovine serum albumin (Man-BSA) and glucosylated bovine serum albumin (Glc-BSA) (negative control), see, for example Rabani et al., 2018, ‘Conformational switch of the bacterial adhesin FimH in the absence of the regulatory domain: Engineering a minimalistic allosteric system’ J. Biol. Chem., 293(5):1835-1849, and Bouckaert J, et al. Mol Microbiol. 2005 January; 55(2):441-55 which are incorporated by reference herein.

The conformation of FimH can also be assessed by measuring the binding of conformational antibodies, using any suitable means known in the art, for example, surface plasmon resonance and as described in the Examples. Exemplary antibodies are capable of recognising epitopes differently overlapping the manriose-binding pocket of FimH, for example antibodies binding to epitopes overlapping with the mannose binding pocket, for example epitopes limited to just one loop of the mannose-binding pocket. Exemplary antibodies are those disclosed in WO2016/183501, or in Kisiela D I, et al. Proc Natl Acad Sci USA. 2013 Nov. 19; 110(47):19089-94, Kisiela Di, et al. PLoS Pathog. 2015 May 14; 11(5):e1004857 and which are incorporated by reference herein. In one embodiment, the conformational antibody has a variable heavy chain (VH) sequence of SEQ ID NO: 125 and a variable light chain (VL) sequence of SEQ ID NO: 126. In one embodiment, the conformational antibody has a variable heavy chain (VH) sequence of SEQ ID NO: 127 and a variable light chain (VL) sequence of SEQ ID NO: 128.

VH of mAb 926
[SEQ ID NO: 125]
QVQLQQSGAELATPGASVKMSCKASGYTSTNYWIHWVKQRPGQGLEWIGY
INPTSGYTEYNQNFKDKATLTADKSSSTAYMQLTSLTSEDSAVYYCARGV
IRDFWGQGTTLTVSSAKTTAPSVYPLAPVCGDTTGSSVTLGCLVKGYFPE
PVTLTWNSGSLSSGVHTFPAVLQSDLYTLSSSVTVTSS
VL (kappa) of mAb 926
[SEQ ID NO: 126]
DVLMTQTPLSLPVSLGDQASISCRSSQNIVHNNGNTYLEWYLQSPGQSPK
LLIYKVSNRFSGVPDRFSGSGSGTDFTLKISRVEAEDLGVYYCFQGSHVP
FTFGSGTKLEIK
VH of mAb 475
[SEQ ID NO: 127]
QVQLQQSGAELVRPGSSVKISCKASGYAFSSYWMNWVKQRPGQGLEWIGQ
IYPRDGDTNYNGKFMDKVTLTADKSSNTAYMQLSSLTSEDSAVYFCEVGR
GFYGMDYWGQGTSVTVSSAKTTAPSVYPLAPVCGDTTGSSVTLGCLVKGY
FPEPVTLTWNSGSLSSGVHTFPAVLQSDLYTLSSSVTVTSS
VL (kappa) of mAb 475
[SEQ ID NO: 128]
DIVMTQSPKFMSTSVGDRVSVTCKASQNVSNVAWYQQKPGQSPKAMIYSA
SYRYSGVPGRFTGSGSGTDFTLTINNVQSEDLATYFCQQNSSFPFTFGGG
TKLEIK

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids (see below).

Thus, when an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

By ‘isolated’ we mean that the feature (e.g., the polypeptide) of the invention is provided in a context other than that in which it may be found naturally. One of skill in the art would understand that ‘isolated’ means altered ‘by the hand of man’ from its natural state, i.e., if it occurs in nature, it has been changed or removed from its original environment, or both. For example, a polynucleotide or a polypeptide naturally present in a living organism is not ‘isolated’ when in such living organism, but the same polynucleotide or polypeptide separated from the coexisting materials of its natural state is ‘isolated’ as the term is used in this disclosure. Further, a polynucleotide or polypeptide that is introduced into an organism by transformation, genetic manipulation or by any other recombinant method would be understood to be ‘isolated’ even if it is still present in said organism, which organism may be living or non-living, except where such transformation, genetic manipulation or other recombinant method produces an organism that is otherwise indistinguishable from the naturally-occurring organism.

By ‘polypeptide’ we mean or include polypeptides and proteins.

By ‘variant’ of the polypeptide we include insertions, deletions and/or substitutions, either conservative or non-conservative. In particular, the variant polypeptide may be a non-naturally occurring variant (i.e., does not, or is not known to, occur in nature). Variants may have at least 50% sequence identify with the/a reference sequence, for example, at least 60%, 70%, 80%, 90%, 95%, 96%, 97%, 98%, 99% or 99.5%.

‘Sequence identity’ or ‘identity’ can be determined by the Smith Waterman homology search algorithm as implemented in the MPSRCH program (Oxford Molecular), using an affine gap search with parameters gap open penalty=12 and gap extension penalty=1, or by the Needleman-Wunsch global alignment algorithm (see e.g. Rubin (2000) Pediatric. Clin. North Am. 47:269-285), using default parameters (e.g. with Gap opening penalty=10.0, and with Gap extension penalty=0.5, using the EBLOSUM62 scoring matrix). This algorithm is conveniently implemented in the needle tool in the EMBOSS package. Unless specified otherwise, where the application refers to sequence identity to a particular reference sequence, the identity is intended to be calculated over the entire length of that reference sequence. Alternatively, percent identity can be determined by methods well known in the art, for example using the LALIGN program (Huang and Miller, Adv. Appl. Math. (1991) 12:337-357, the disclosures of which are incorporated herein by reference) at the ExPASy facility website www.ch.embnet.org/software/LALIGN_form.html using as parameters the global alignment option, scoring matrix BLOSUM62, opening gap penalty −14, extending gap penalty −4. Alternatively, the percent sequence identity between two polypeptides may be determined using suitable computer programs, for example AlignX, Vector NTI Advance 10 (from Invitrogen Corporation) or the GAP program (from the University of Wisconsin Genetic Computing Group).

It will be appreciated that percent identity is calculated in relation to polymers (e.g., polypeptide or polynucleotide) whose sequence has been aligned.

Fragments and variants may be made using the methods of protein engineering and site-directed mutagenesis well known in the art (for example, see Molecular Cloning: a Laboratory Manual, 3rd edition, Sambrook & Russell, 2001, Cold Spring Harbor Laboratory Press, the disclosures of which are incorporated herein by reference).

It will be appreciated by skilled persons that the polypeptide of the invention, or fragment, variant or fusion thereof, may comprise one or more amino acids that are modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which exhibit endolysin activity. The term ‘peptidomimetic’ refers to a compound that mimics the conformation and desirable features of a particular polypeptide as a therapeutic agent.

For example, the polypeptides described herein include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, the disclosures of which are incorporated herein by reference. Such retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -y(CH2NH)— bond in place of the conventional amide linkage.

It will be appreciated that the polypeptide may conveniently be blocked at its N- or C-terminus so as to help reduce susceptibility to exoproteolytic digestion, e.g., by amidation.

As discussed herein, a variety of uncoded or modified amino acids such as D-amino acids and N-methyl amino acids may be used to modify polypeptides of the invention. In addition, a presumed bioactive conformation may be stabilised by a covalent modification, such as cyclisation or by incorporation of lactam, disulphide or other types of bridges. Methods of synthesis of cyclic homodetic peptides and cyclic heterodetic peptides, including disulphide, sulphide and alkylene bridges, are disclosed in U.S. Pat. No. 5,643,872. Other examples of cyclisation methods are discussed and disclosed in U.S. Pat. No. 6,008,058, the relevant disclosures in which documents are hereby incorporated by reference. A further approach to the synthesis of cyclic stabilised peptidomimetic compounds is ring-closing metathesis (RCM).

By ‘fusion’ of a polypeptide we include a polypeptide which is fused to any other polypeptide. For example, the polypeptide may comprise one or more additional amino acids, inserted internally and/or at the N- and/or C-termini of the amino acid sequence the polypeptides of the invention.

Thus, as described herein, in one embodiment the polypeptide of the first aspect of the invention comprises a polypeptide of the invention to which is fused an enzymatic domain from a different source (e.g., from a source other than the polypeptide of the first aspect of the invention). Examples of suitable enzymatic domains include: L-alanoyl-D-glutamate endopeptidase; D-glutamyl-m-DAP endopeptidase; interpeptide bridge-specific endopeptidase; N-acetyl-@-D-glucosaminidase (=muramoylhydrolase); N-acetyl-3-D-muramidase (=lysozyme); lytic transglycosylase. Also, N-acetylmuramoyl-L-alanine amidase from other sources could be utilised (see Loessner, 2005, Current Opinion in Microbiology 8: 480-487, the disclosures of which are incorporated herein by reference).

For example, the said polypeptide may be fused to a polypeptide such as glutathione-S-transferase (GST) or protein A in order to facilitate purification of said polypeptide. Examples of such GST fusions are well known to those skilled in the art. Similarly, the said polypeptide may be fused to an oligo-histidine tag such as His6 or to an epitope recognised by an antibody such as the well-known Myc tag epitope. Fusions to any fragment, variant or derivative of said polypeptide are also included in the scope of the invention. It will be appreciated that fusions (or variants or derivatives thereof) which retain desirable properties, e.g., antigenic activity, are preferred. It is also particularly preferred if the fusions are ones which are suitable for use in the methods described herein.

For example, the fusion may comprise a further portion which confers a desirable feature on the said polypeptide of the invention; for example, the portion may be useful in detecting or isolating the polypeptide, promoting cellular uptake of the polypeptide, or directing secretion of the protein from a cell. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

It will be appreciated by persons skilled in the art that the polypeptides of the invention also include pharmaceutically acceptable acid or base addition salts of the herein described polypeptides. The acids which are used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the polypeptides. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The polypeptide, or fragment, variant, fusion or derivative thereof, may also be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of activity loss and that use levels may have to be adjusted upward to compensate. Preferably, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

Polypeptides of the invention are preferably provided in purified or substantially purified form i.e., substantially free from other polypeptides (e.g. free from naturally-occurring polypeptides), particularly from other E. coli or host cell polypeptides, and are generally at least about 50% pure (by weight), for example at least 70%, 80%, 90%, 95%, 96%, 97%, 98% 99%, 99.5%, 99.5% or 100% pure by weight (i.e., less than 50% of a composition is made up of other expressed polypeptides). Thus, the antigens in the compositions are separated from the whole organism with which the antigen molecule is expressed.

The FimH of (a) may be of any Escherichia coli or Klebsiella pneumoniae species (or a variant, fragment and/or fusion thereof) but, alternatively or additionally, (a) comprises or consists of:
(A) the amino acid sequence of SEQ ID NO: 1 (Genbank Accession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (Genbank Accession no: AAN83822.1 (FimH of CFT073)), SEQ ID NO: 103, SEQ ID NO: 104 (Genbank Accession no: AJE58925.1 (FimH of E. coli 789)), SEQ ID NO: 105, SEQ ID NO: 106 (Genbank Accession No. AAC35864.1, corresponding to nucleic acid sequence AF089840.1 (FimH of IHE3034), or SEQ ID NO: 107,
(B) an amino acid sequence comprising from 1 to 10 single amino acid alterations compared to SEQ ID NO: 1 (Genbank Accession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (Genbank Accession no: AAN83822.1 (FimH of CFT073)), SEQ ID NO: 103, SEQ ID NO: 104 (Genbank Accession no: AJE58925.1 (FimH of E. coli 789)), SEQ ID NO: 105, SEQ ID NO: 106 (Genbank Accession No. AAC35864.1, corresponding to nucleic acid sequence AF089840.1 (FimH of IHE3034), or SEQ ID NO: 107, for example, 1, 2, 3, 4, 5, 6, 7, 8, 9 or 10 single amino acid alterations,
(C) an amino acid sequence with at least 70% sequence identity with SEQ ID NO: 1 (Genbank Accession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (Genbank Accession no: AAN83822.1 (FimH of CFT073)), SEQ ID NO: 103, SEQ ID NO: 104 (Genbank Accession no: AJE58925.1 (FimH of E. coli 789)), SEQ ID NO: 105, SEQ ID NO: 106 (Genbank Accession No. AAC35864.1, corresponding to nucleic acid sequence AF089840.1 (FimH of IHE3034), or SEQ ID NO: 107, for example, 80%, 85%, 90%, 91%. 92%, 93%, 94%, 95%, 96%, 97%, 98% or 99% sequence identity, and/or
(D) a fragment of at least 10 consecutive amino acids from SEQ ID NO: 1 (Genbank Accession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (Genbank Accession no: AAN83822.1 (FimH of CFT073)), SEQ ID NO: 103, SEQ ID NO: 104 (Genbank Accession no: AJE58925.1 (FimH of E. coli 789)), SEQ ID NO: 105, SEQ ID NO: 106 (Genbank Accession No. AAC35864.1, corresponding to nucleic acid sequence AF089840.1 (FimH of IHE3034), or SEQ ID NO: 107, for example, at least 20, 30, 40, 50, 60, 70, 80, 90, 100, 125, 150, 175, 200, 250, 275, 280, 290 or 300 consecutive amino acids.

GenBank: ELL41155.1 (signal peptide underlined)
[SEQ ID NO: 1]
MKRVITLFAVLLMGWSVNAWSFACKTANGTAIPIGGGSANVYVNLAPVVN
VGQNLVVDLSTQIFCHNDYPETITDYVTLQRGSAYGGVLSNFSGTVKYSG
SSYPFPTTSETPRVVYNSRTDKPWPVALYLTPVSSAGGVAIKAGSLIAVL
ILRQTNNYNSDDFQFVWNIYANNDVVVPTGGCDVSARDVTVTLPDYPGSV
PIPLTVYCAKSQNLGYYLSGTTADAGNSIFTNTASFSPAQGVGVQLTRNG
TIIPANNTVSLGAVGTSAVSLGLTANYARTGGQVTAGNVQSIIGVTFVYQ
GenBank: ELL41155.1 minus 21aa signal peptide
[SEQ ID NO: 2]
FACKTANGTAIPIGGGSANVYVNLAPVVNVGQNLVVDLSTQIFCHNDYPE
TITDYVTLQRGSAYGGVLSNFSGTVKYSGSSYPFPTTSETPRVVYNSRTD
KPWPVALYLTPVSSAGGVAIKAGSLIAVLILRQTNNYNSDDFQFVWNIYA
NNDVVVPTGGCDVSARDVTVTLPDYPGSVPIPLTVYCAKSQNLGYYLSGT
TADAGNSIFTNTASFSPAQGVGVQLTRNGTIIPANNTVSLGAVGTSAVSL
GLTANYARTGGQVTAGNVQSIIGVTFVYQ
Genbank Accession no: ABG72591.1
(FimH of UPEC 536)) (signal peptide underlined)
[SEQ ID NO: 100]
MIVMKRVITLFAVLLMGWSVNAWSFACKTANGTAIPIGGGSANVYVNLAP
AVNVGQNLVVDLSTQIFCHNDYPETITDYVTLQRGSAYGGVLSSFSGTVK
YNGSSYPFPTTSETPRVVYNSRTDKPWPVALYLTPVSSAGGVAIKAGSLI
AVLILRQTNNYNSDDFQFVWNIYANNDVVVPTGGCDVSARDVTVTLPDYP
GSVPIPLTVYCAKSQNLGYYLSGTTADAGNSIFTNTASFSPAQGVGVQLT
RNGTIIPANNTVSLGAVGTSAVSLGLTANYARTGGQVTAGNVQSIIGVTF
VYQ
Genbank Accession no: ABG72591.1
(FimH of UPEC 536) minus signal peptide
[SEQ ID NO: 101]
FACKTANGTAIPIGGGSANVYVNLAPAVNVGQNLVVDLSTQIFCHNDYPE
TITDYVTLQRGSAYGGVLSSFSGTVKYNGSSYPFPTTSETPRVVYNSRTD
KPWPVALYLTPVSSAGGVAIKAGSLIAVLILRQTNNYNSDDFQFVWNIYA
NNDVVVPTGGCDVSARDVTVTLPDYPGSVPIPLTVYCAKSQNLGYYLSGT
TADAGNSIFTNTASFSPAQGVGVQLTRNGTIIPANNTVSLGAVGTSAVSL
GLTANYARTGGQVTAGNVQSIIGVTFVYQ
Genbank Accession no: AAN83822.1
(FimH of CFT073) (signal peptide underlined)
[SEQ ID NO: 102]
MIVMKRVITLFAVLLMGWSVNAWSFACKTANGTAIPIGGGSANVYVNLAP
AVNVGQNLVVDLSTQIFCHNDYPETITDYVTLQRGSAYGGVLSSFSGTVK
YNGSSYPFPTTSETPRVVYNSRTDKPWPVALYLTPVSSAGGVAIKAGSLI
AVLILRQTNNYNSDDFQFVWNIYANNDVVVPTGGCDASARDVTVTLPDYP
GSVPIPLTVYCAKSQNLGYYLSGTTADAGNSIFTNTASFSPAQGVGVQLT
RNGTIIPANNTVSLGAVGTSAVSLGLTANYARTGGQVTAGNVQSIIGVTF
VYQ
Genbank Accession no: AAN83822.1
(FimH of CFT073) minus signal peptide
[SEQ ID NO: 103]
FACKTANGTAIPIGGGSANVYVNLAPAVNVGQNLVVDLSTQIFCHNDYPE
TITDYVTLQRGSAYGGVLSSFSGTVKYNGSSYPFPTTSETPRVVYNSRTD
KPWPVALYLTPVSSAGGVAIKAGSLIAVLILRQTNNYNSDDFQFVWNIYA
NNDVVVPTGGCDASARDVTVTLPDYPGSVPIPLTVYCAKSQNLGYYLSGT
TADAGNSIFTNTASFSPAQGVGVQLTRNGTIIPANNTVSLGAVGTSAVSL
GLTANYARTGGQVTAGNVQSIIGVTFVYQ
Genbank Accession no: AJE58925.1
(FimH of E. coli 789) (signal peptide underlined)
[SEQ ID NO: 104]
MIVMKRVITLFAVLLMGWSVNAWSFACKTANGTAIPIGGGSANVYVNLAP
VVNVGQNLVVDLSTQIFCHNDYPETITDYVTLQRGSAYGGVLSNFSGTVK
YSGSSYPFPTTSETPRVVYNSRTDKPWPVALYLTPVSSAGGVAIKAGSLI
AVLILRQTNNYNSDDFQFVWNIYANNDVVVPTGGCDVSARDVTVTLPDYP
GSVPIPLTVYCAKSQNLGYYLSGTTADAGNSIFTNTASFSPAQGVGVQLT
RNGTIIPANNTVSLGAVGTSAVSLGLTANYARTGGQVTAGNVQSIIGVTF
VYQ
Genbank Accession no: AJE58925.1
(FimH of E. coli 789) minus signal peptide
[SEQ ID NO: 105]
FACKTANGTAIPIGGGSANVYVNLAPVVNVGQNLVVDLSTQIFCHNDYPE
TITDYVTLQRGSAYGGVLSNFSGTVKYSGSSYPFPTTSETPRVVYNSRTD
KPWPVALYLTPVSSAGGVAIKAGSLIAVLILRQTNNYNSDDFQFVWNIYA
NNDVVVPTGGCDVSARDVTVTLPDYPGSVPIPLTVYCAKSQNLGYYLSGT
TADAGNSIFTNTASFSPAQGVGVQLTRNGTIIPANNTVSLGAVGTSAVSL
GLTANYARTGGQVTAGNVQSIIGVTFVYQ
Genbank Accession No. AAC35864.1,
(FimH of IHE3034), (signal peptide underlined)
[SEQ ID NO: 106]
MKRVITLFAVLLMGWSVNAWSFACKTANGTAIPIGGGSANVYVNLAPAVN
VGQNLVVDLSTQIFCHNDYPETITDYVTLQRGAAYGGVLSSFSGTVKYNG
SSYPFPTTSETPRVVYNSRTDKPWPVALYLTPVSSAGGVAIKAGSLIAVL
ILRQTNNYNSDDFQFVWNIYANNDVVVPTGGCDVSARDVTVTLPDYPGSV
PIPLTVYCAKSQNLGYYLSGTTADAGNSIFTNTASFSPAQGVGVQLTRNG
TIIPANNTVSLGAVGTSAVSLGLTANYARTGGQVTAGNVQSIIGVTFVYQ
Genbank Accession No. AAC35864.1,
(FimH of IHE3034), minus signal peptide
[SEQ ID NO: 107]
FACKTANGTAIPIGGGSANVYVNLAPAVNVGQNLVVDLSTQIFCHNDYPE
TITDYVTLQRGAAYGGVLSSFSGTVKYNGSSYPFPTTSETPRVVYNSRTD
KPWPVALYLTPVSSAGGVAIKAGSLIAVLILRQTNNYNSDDFQFVWNIYA
NNDVVVPTGGCDVSARDVTVTLPDYPGSVPIPLTVYCAKSQNLGYYLSGT
TADAGNSIFTNTASESPAQGVGVQLTRNGTIIPANNTVSLGAVGTSAVSL
GLTANYARTGGQVTAGNVQSIIGVTFVYQ

Alternatively or additionally, the polypeptide is a fragment, variant, fusion and/or derivative capable of inducing a specific immune response to a polypeptide selected from the group consisting of SEQ ID NO: 1, (Genbank Accession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (Genbank Accession no: AAN83822.1 (FimH of CFT073)), SEQ ID NO: 103, SEQ ID NO: 104 (Genbank Accession no: AJE58925.1 (FimH of E. coli 789)), SEQ ID NO: 105, or SEQ ID NO: 106 (Genbank Accession No. AAC35864.1, corresponding to nucleic acid sequence AF089840.1 (FimH of IHE3034), SEQ ID NO: 107

By “specific immune response” we mean or include the capability to induce an immune response in a subject that generates (e.g., stimulates the release of) antibody capable of binding to an amino acid sequence specified. It is preferred that the antibody is capable of binding in vivo, i.e., under the physiological conditions in which the amino acid sequence or polypeptide exists on or inside of a subject's body. Such binding specificity may be determined by methods well known in the art, such as e.g. ELISA, immunohistochemistry, immunoprecipitation, Western blots and flow cytometry using transfected cells expressing the/a polypeptide of the invention.

Alternatively, or additionally, the immune response is an immune-activating response, for example, a protective immune response. The polypeptide may be capable of eliciting an in vitro protective immune response and/or an in vivo protective immune response when administered to a subject.

In the presence of co-stimulatory signals, T cells differentiate into specific phenotypic subtypes. Several of these subtypes are involved in suppressing or terminating natural inflammatory signals. By “immune-activating response” we mean and/or include that polypeptide induces or is capable of inducing an immune response in a subject that does not result in suppressing or terminating inflammation or inflammatory signals and, preferably, results in the activation or enhancement of inflammation or inflammatory signals (e.g., cytokines).

The in vivo protective immune response may be elicited in a mammal. Alternatively or additionally, the mammal is selected from the group consisting of armadillo (dasypus novemcinctus), baboon (papio anubis; papio cynocephalus), camel (Camelus bactrianus, Camelus dromedarius, Camelus ferus), cat (felis catus), dog (Canis lupus familiaris), horse (Equus ferus caballus), ferret (Mustela putorius furo), goat (Capra aegagrus hircus), guinea pig (Cavia porcellus), golden hamster (Mesocricetus auratus), kangeroo (Macropus rufus), llama (Lama glama), mouse (Mus musculus), pig (Sus scrofa domesticus), rabbit (Oryctolagus cuniculus), rat (Rattus norvegicus), rhesus macaque (Macaca mulatta), sheep (Ovis aries), non-human primates, and human (Homo sapiens).

Alternatively or additionally, two glycine residues of the linker connecting FimH_{L} to FimH_{P} can be deleted to reduce the flexibility of FimH_{L} and reduce mannose binding. For example, glycine residues 196 and 197 of polypeptide portion (a), relative to SEQ ID NO: 1, glycine residues 180 and 181 of polypeptide portion (a), relative to SEQ ID NO: 1, glycine residues 183 and 184 of polypeptide portion (a), relative to SEQ ID NO: 100, glycine residues 183 and 184 of polypeptide portion (a), relative to SEQ ID NO: 102, glycine residues 183 and 184 of polypeptide portion (a), relative to SEQ ID NO: 104, are:
(i) present; or
(ii) deleted.

Alternatively or additionally, one or more amino acids of the polypeptide known or predicted to be N-glycosylated or O-glycosylated are substituted with amino acids unsusceptible or less susceptible to glycosylation, e.g., serine (S), aspartic acid (D), alanine (A) or glutamine (Q). Alternatively or additionally, only polypeptide portion (a) includes amino acid substitutions to reduce or abolish N- and/or O-glycosylation.

N- and/or O-glycosylation can be determined using any suitable means known in the art, for example, using the NetNGlyc 1.0 and NetOGlyc 4.0 Server (accessible at http://www.cbs.dtu.dk/-ervices/NetOGlyc/and http.//www.cbs.dtu.dk/services/NetOGlyc/) using default settings.

Alternatively or additionally, polypeptide portion (a) includes one or more of the following amino acid substitutions relative to SEQ ID NO: 2: N28S, N91D, N249D, N256D, or at the positions of SEQ ID NO: 101, 103 and 105 corresponding those positions of SEQ ID NO:2, for example, one, two, three or four of the amino acid substitutions.

Alternatively or additionally, the donor-strand complementing amino acid sequence (b) comprises or consists of:
(i) 6-28 amino acids of SEQ ID NO: 3; or a fragment and/or variant thereof, or
(ii) 8-36 amino acids of SEQ ID NO: 4; or a fragment and/or variant thereof,

FimG donor strand and flanking region
(donor strand underlined)
[SEQ ID NO: 3]
ASATIQAADVTITVNGKVVAKPCTVSTT
FimC donor strand and flanking region
(donor strand underlined)
[SEQ ID NO: 4]
PSMDKSKLTENTLQLAIISRIKLYYRPAKLALPPDQ

Alternatively or additionally, portion (b) comprises or consists of 6-28 amino acids of SEQ ID NO: 3 (or a fragment and/or variant thereof), which amino acids are selected from the group consisting of:
(i) amino acids 1-28 of SEQ ID NO: 3; or a fragment and/or variant thereof,
(ii) amino acids 2-27 of SEQ ID NO: 3; or a fragment and/or variant thereof,
(iii) amino acids 3-26 of SEQ ID NO: 3; or a fragment and/or variant thereof,
(iv) amino acids 4-25 of SEQ ID NO: 3; or a fragment and/or variant thereof,
(v) amino acids 5-24 of SEQ ID NO: 3; or a fragment and/or variant thereof,
(vi) amino acids 6-23 of SEQ ID NO: 3; or a fragment and/or variant thereof,
(vii) amino acids 7-22 of SEQ ID NO: 3; or a fragment and/or variant thereof,
(viii) amino acids 8-21 of SEQ ID NO: 3; or a fragment and/or variant thereof,
(ix) amino acids 9-20 of SEQ ID NO: 3; or a fragment and/or variant thereof,
(x) amino acids 10-19 of SEQ ID NO: 3; or a fragment and/or variant thereof,
(xi) amino acids 11-18 of SEQ ID NO: 3; or a fragment and/or variant thereof, and
(xii) amino acids 12-17 of SEQ ID NO: 3; or a fragment and/or variant thereof.

Alternatively or additionally, portion (b) comprises or consists of 8-36 amino acids of SEQ ID NO: 4 (or a fragment and/or variant thereof), which amino acids are selected from the group consisting of:
(i) amino acids 1-36 of SEQ ID NO: 4; or a fragment and/or variant thereof,
(ii) amino acids 2-35 of SEQ ID NO: 4; or a fragment and/or variant thereof,
(iii) amino acids 3-34 of SEQ ID NO: 4; or a fragment and/or variant thereof,
(iv) amino acids 4-33 of SEQ ID NO: 4; or a fragment and/or variant thereof,
(v) amino acids 5-32 of SEQ ID NO: 4; or a fragment and/or variant thereof,
(vi) amino acids 6-31 of SEQ ID NO: 4; or a fragment and/or variant thereof,
(vii) amino acids 7-30 of SEQ ID NO: 4; or a fragment and/or variant thereof,
(viii) amino acids 8-29 of SEQ ID NO: 4; or a fragment and/or variant thereof,
(ix) amino acids 9-28 of SEQ ID NO: 4; or a fragment and/or variant thereof,
(x) amino acids 10-27 of SEQ ID NO: 4; or a fragment and/or variant thereof,
(xi) amino acids 11-26 of SEQ ID NO: 4; or a fragment and/or variant thereof,
(xii) amino acids 12-25 of SEQ ID NO: 4; or a fragment and/or variant thereof,
(xiii) amino acids 13-24 of SEQ ID NO: 4; or a fragment and/or variant thereof,
(xiv) amino acids 14-23 of SEQ ID NO: 4; or a fragment and/or variant thereof,
(xv) amino acids 15-24 of SEQ ID NO: 4; or a fragment and/or variant thereof, and
(xvi) amino acids 16-23 of SEQ ID NO: 4; or a fragment and/or variant thereof.

Alternatively or additionally, the donor-strand complementing amino acid sequence (b) comprises or consists of:
(A) the amino acid sequence of SEQ ID NO: 5 or SEQ ID NO: 6,
(B) an amino acid sequence comprising from 1 to 10 single amino acid alterations compared to SEQ ID NO: 5 or SEQ ID NO: 6, for example, 1, 2, 3, 4, 5, 6, 7, 8, 9 or 10 single amino acid alterations,
(C) a fragment of at least 7 consecutive amino acids from SEQ ID NO: 5, for example, at least 8, 9, 10, 11, 12, or 13 consecutive amino acids from SEQ ID NO: 5, and/or
(D) a fragment of at least 7 consecutive amino acids from SEQ ID NO: 6, for example, at least 8, 9, 10, 11, 12, 13, 14, 15, 16, 17, or 18 consecutive amino acids from SEQ ID NO: 6.

FimG donor strand
[SEQ ID NO: 5]
ADVTITVNGKVVAK
FimC donor strand
[SEQ ID NO: 6]
ENTLQLAIISRIKLYYRP

In one preferred embodiment, the donor-strand complementing amino acid sequence (b) comprises or consists of SEQ ID NO: 5. Alternatively or additionally, the donor-strand complementing amino acid sequence (b) comprises or consists of SEQ ID NO: 6.

Alternatively or additionally, the donor-strand complementing amino acid sequence (b) is:
(i) directly joined to the C-terminus of (a), or
(ii) joined to the C-terminus of (a) via a first linker.

Alternatively or additionally, the first linker (or “L”) comprises or consists of 2-20 amino acids, for example, 3, 4, 5, 6, 7, 8, 9, 10, 11, 12, 13, 14, 15, 16, 17, 18, 19 or 20 amino acids. Alternatively or additionally, the first linker begins with proline. In a preferred embodiment, the first linker begins with proline. Alternatively or additionally, the first linker comprises or consists of polar amino acids, for example, wherein the first linker is entirely comprised of polar amino acids or, if the first linker begins with proline, the remainder of the amino acids are polar. Alternatively or additionally, the first linker comprises or consists of:
(i) PGDGN [SEQ ID NO: 7], or a variant or fusion thereof, or
(ii) DNKQ [SEQ ID NO: 8], or a variant or fusion thereof.

In one preferred embodiment the first linker (or “L”) comprises or consists of SEQ ID NO: 7.

Alternatively or additionally, the polypeptide comprises a protein purification affinity tag at the N-terminus, C-terminus and/or internally, for example, 6, 7, 8, 9 or 10 consecutive histidines.

Alternatively or additionally, “X” comprises a cell secretion leader sequence. Alternatively or additionally, the polypeptide comprises a cell secretion leader sequence:
(i) upstream of (a), or
(ii) at the N-terminus of the polypeptide.

Alternatively or additionally, the cell secretion leader sequence is selected from the group consisting of:
(i) METDTLLLWVLLLWVPGSTGD [SEQ ID NO: 9], or a variant or fusion thereof,
(ii) METDTLLLWVLLLWVPGSTGDAAQPARRARRTKLAL [SEQ ID NO: 10], or a variant or fusion thereof,
(iii) MRLLAKIICLMLWAICVA [SEQ ID NO: 11], or a variant or fusion thereof,
(iv) MGWSCIILFLVATATGVHS [SEQ ID NO: 12], or a variant or fusion thereof,
(v) METPAELLFLLLLWLPDTTG [SEQ ID NO: 13], or a variant or fusion thereof,
(vi) METDTLLLWVLLLWVPGSTG [SEQ ID NO: 108], or a variant or fusion thereof or
(vii) MEFGLSWVFLVAILEGVHC [SEQ ID NO: 14], or a variant or fusion thereof.

Alternatively, or additionally, “X” is a methionine (M) residue, particularly when the polypeptide is expressed in E. coli host cells.

Alternatively or additionally, the polypeptide comprises a nanoparticle domain at the N-terminus or C-terminus. Thus, in one embodiment “X” comprises a nanoparticle domain or “Y” comprises a nanoparticle domain. By ‘nanoparticle domain’ we mean or include amino acid sequences capable of self-assembly to form protein complexes, in particular, globular protein complexes. By ‘self-assembly’ we mean or include assembly with nanoparticle domains of the same type (e.g., if the nanoparticle domain is a ferritin domain, capable of assembling with other ferritin domains to form protein complexes, such as globular protein complexes). In particular, the nanoparticle domains of the invention are capable of self-assembly when they form a portion of the/a polypeptide of the invention.

Alternatively or additionally, the nanoparticle domain is selected from the group consisting of:
(a) ferritin (for example, [SEQ ID NO: 15] or [SEQ ID NO: 109] (Helicobacter pylori), [SEQ ID NO: 16](Escherichia coli)), or any one of [SEQ ID NO: 149]-[SEQ ID NO: 152] (stabilized Escherichia coli)), or a variant and/or fragment thereof,
(b) iMX313 (for example [SEQ ID NO: 17]), or a variant and/or fragment thereof,
(c) mI3 (for example [SEQ ID NO: 18]), or a variant and/or fragment thereof,
(d) encapsulin (for example [SEQ ID NO: 19]), or a variant and/or fragment thereof, or
(e) Self-assembling viral coat proteins, such as Acinetobacter phage AP205 coat protein (NCBI Reference Sequence: NP_085472.1), Hepatitis B virus core protein (HBc) [SEQ ID NO: 110], or bacteriophage Q3 [SEQ ID NO: 111], or a variant and/or fragment thereof.

H. pylori ferritin
[SEQ ID NO: 15]
DIIKLLNEQVNKEMNSSNLYMSMSSWCYTHSLDGAGLFLFDHAAEEYEH
AKKLIIFLNENNVPVQLTSISAPEHKFEGLTQIFQKAYEHEQHISESIN
NIVDHAIKSKDHATFNFLQWYVAEQHEEEVLFKDILDKIELIGNENHGL
YLADQYVKGIAKSRK
H. pylori ferritin (with terminal S)
[SEQ ID NO: 109]
DIIKLLNEQVNKEMNSSNLYMSMSSWCYTHSLDGAGLFLFDHAAEEYEH
AKKLIIFLNENNVPVQLTSISAPEHKFEGLTQIFQKAYEHEQHISESIN
NIVDHAIKSKDHATFNFLQWYVAEQHEEEVLFKDILDKIELIGNENHGL
YLADQYVKGIAKSRKS
E. coli ferritin
[SEQ ID NO: 16]
LKPEMIEKLNEQMNLELYSSLLYQQMSAWCSYHTFEGAAAFLRRHAQEE
MTHMQRLFDYLTDTGNLPRINTVESPFAEYSSLDELFQETYKHEQLITQ
KINELAHAAMTNQDYPTFNFLQWYVSEQHEEEKLFKSIIDKLSLAGKSG
EGLYFIDKELSTLDTQN
iMX313
[SEQ ID NO: 17]
KKQGDADVCGEVAYIQSVVSDCHVPTAELRTLLEIRKLFLEIQKLKVEL
QGLSKEG
mi3
[SEQ ID NO: 18]
MKMEELFKKHKIVAVLRANSVEEAKKKALAVFLGGVHLIEITFTVPDAD
TVIKELSFLKEMGAIIGAGTVTSVEQARKAVESGAEFIVSPHLDEEISQ
FAKEKGVFYMPGVMTPTELVKAMKLGHTILKLFPGEVVGPQFVKAMKGP
FPNVKFVPTGGVNLDNVCEWFKAGVLAVGVGSALVKGTPVEVAEKAKAF
VEKIRGCTE
encapsulin
[SEQ ID NO: 19]
MEFLKRSFAPLTEKQWQEIDNRAREIFKTQLYGRKFVDVEGPYGWEYAA
HPLGEVEVLSDENEVVKWGLRKSLPLIELRATFTLDLWELDNLERGKPN
VDLSSLEETVRKVAEFEDEVIFRGCEKSGVKGLLSFEERKIECGSTPKD
LLEAIVRALSIFSKDGIEGPYTLVINTDRWINFLKEEAGHYPLEKRVEE
CLRGGKIITTPRIEDALVVSERGGDFKLILGQDLSIGYEDREKDAVRLF
ITETFTFQVVNPEALILLKF
HBC
[SEQ ID NO: 110]
MDIDPYKEFGATVELLSFLPSDFFPSVRDLLDTASALYREALESPEHCS
PHHTALRQAILCWGELMTLATWVGNNLEDASRDLVVNYVNTNMGLKIRQ
LLWFHISCLTFGRETVLEYLVSFGVWIRTPPAYRPPNAPILSTLPETTV
V
Qbeta
[SEQ ID NO: 111]
MAKLETVTLGNIGKDGKQTLVLNPRGVNPTNGVASLSQAGAVPALEKRV
TVSVSQPSRNRKNYKVQVKIQNPTACTANGSCDPSVTRQAYADVTFSFT
QYSTDEERAFVRTELAALLASPLLIDAIDQLNPAY
1EUM_0_5-stabilized E. coli ferritin
[SEQ ID NO: 149]
LKPEMIEKLNEQMNLELYSSLLYQQMSAWCSYHGFEGAAAFLRRHAQEE
MTHMQRLFDYLTDTGNLPRIDTIPSPFAEYSSLDELFQETYKHEQLITQ
KINELAHAAMTNQDYPTFNFLQWYVAEQHEEEKLFKSIIDKLSLAGKSG
EGLYFIDKELSTLDTQN
1EUM_2-stabilized E. coli ferritin
[SEQ ID NO: 150]
LKPEMIEKLNEQMNLELYSSLLYQQMSAWCSYHGFEGAAAFLRRHAQEE
MTHMQRLFDYLTDTGNLPRINTIPSPFAEYSSLDELFQETYKHEQLITQ
KINELAHAAMTNQDYPTFNFLQWYVAEQHEEEKLFKSIIDKLSLAGKSG
EGLYFIDKELSTLDTQN
1EUM_2_5-stabilized E. coli ferritin
[SEQ ID NO: 151]
LKPEMIEKLNEQMNLELYSSLLYQQMSAWCSYHGFEGAAAFLRRHAQEE
MTHMQRLFDYLTDTGNLPRINTVPSPFAEYSSLDELFQETYKHEQLITQ
KINELAHAAMTNQDYPTFNFLQWYVAEQHEEEKLFKSIIDKLSLAGKSG
EGLYFIDKELSTLDTQN
1EUM_6-stabilized E. coli ferritin
[SEQ ID NO: 152]
LKPEMIEKLNEQMNLELYSSLLYQQMSAWCSYHGFEGAAAFLRRHAQEE
MTHMQRLFDYLTDTGNLPRINTVESPFAEYSSLDELFQETYKHEQLITQ
KINELAHAAMTNQDYPTFNFLQWYVSEQHEEEKLFKSIIDKLSLAGKSG
EGLYFIDKELSTLDTQN

Alternatively or additionally, the nanoparticle domain is:
(i) directly joined to the polypeptide, or
(ii) joined to the polypeptide via a second linker.

Alternatively or additionally, the second linker comprises or consists of between 2-20 amino acids, for example, 3, 4, 5, 6, 7, 8, 9, 10, 11, 12, 13, 14, 15, 16, 17, 18, 19 or 20 amino acids. Alternatively or additionally, the second linker comprises or consists glycines (G) and/or serines (S), or comprises at least 50% glycines (G) and/or serines (S), for example, at least 60%, 70%, 80&, 90% or 95% glycines (G) and/or serines (S).

Alternatively or additionally, the second linker is selected from the group consisting of:
(a) GSSGSGSGS [SEQ ID NO: 112] or a variant or fusion thereof,
(b) GGSGS [SEQ ID NO: 113] or a variant or fusion thereof,
(c) GGS or a variant or fusion thereof,
(d) SGSHHHHHHHHGGS [SEQ ID NO: 114], or a variant or fusion thereof,
(e) AKFVAAWTLKAAA [SEQ ID NO: 115] or a variant or a fusion thereof,
(f) GGGGSLVPRGSGGGGS [SEQ ID NO: 116], or a variant or a fusion thereof,
(g) EAAAKEAAAKEAAAKA [SEQ ID NO: 117], or a variant or a fusion thereof,
(h) SGSFVAAWTLKAAAGGS [SEQ ID NO: 118] or a variant or a fusion thereof, and
(i) SGSGSGGGGGGS [SEQ ID NO: 119] or a variant or a fusion thereof.

The linker AKFVAAWTLKAAA [SEQ ID NO: 115], also known as Pan HLA DR-binding epitope (PADRE) is a peptide that activates antigen specific-CD4+ T cells, which has been proposed as a carrier epitope suitable for use in the development of synthetic and recombinant vaccines, as disclosed in “Linear PADRE T Helper Epitope and Carbohydrate B Cell Epitope Conjugates Induce Specific High Titer IgG Antibody Responses” 10.4049/jimmunol.164.3.1625 whose disclosure is incorporated by reference herein. The linkers GGGGSLVPRGSGGGGS [SEQ ID NO: 116] and EAAAKEAAAKEAAAKA [SEQ ID NO: 117] are rigid linkers which are not capable of folding into an alpha helix.

Alternatively or additionally, the nanoparticle domain is:
(a) upstream of (a),
(b) at the N-terminus of the polypeptide,
(c) downstream of (b), or
(d) at the C-terminus of the polypeptide.

In a further aspect, it is provided a designed and de novo polypeptide monomer (and the nucleic acid molecules encoding them) capable of self assembling into nanoparticles (i.e., protein nanoparticles). Host cells, vectors or constructs, and method for making or using such polypeptide monomers and protein nanoparticles are also provided. The present invention further relates to nanoparticles (NPs) that have a surface structure comprising, or consisting of, at least one such polypeptide monomer and that, optionally, carries one or more antigen molecule.

The polypeptide monomer of the invention is mutated as compared to its wild type counterpart monomer (i.e., the E. coli bacterial ferritin [SEQ ID NO: 16]), and may thereby have an increased stability, such as an improved thermal stability or folding stability in kcal/mol as compared to its wild type counterpart monomer, which may thereby form a self-assembled nanoparticle with an improved thermal stability or folding stability in kcal/mol as compared to its wild type counterpart nanoparticle.

“Increased stability” means the molecule has a lower rate of unfolding, decreased misfolding, reduced protein domain movements, reduced protein domain rearrangements, increased half-life (in-vitro or in-vivo), increased shelf life, increased melting temperature (Tm) (meaning an increase in at least one melting temperature, if the molecule has two or more), lower folding free energy value (kcal/mol), lower binding free energy value (as in the case of a subunit that binds other subunits to form a macromolecule), or a combination thereof; as compared to a control molecule or its wild type counterpart under comparable or the same conditions (e.g., temperature and/or pH). For clarity of the example, if the stability of a molecule is increased via one or more mutations (“stabilizing mutations” such as one or more amino acid mutations), a “control molecule” or its “wild type counterpart” means a molecule that does not comprise the one or more stabilizing mutations. With respect to the present invention, a monomer or nanoparticle may be described as having an increased stability (e.g., increased thermostability and/or increased folding stability and/or increased binding stability) as compared to its wildtype counterpart molecule under comparable (or the same) conditions. “Conditions” as used herein includes experimental and physiological conditions. See, e.g., U.S. Pub. No. 2011/0229507; Clapp et al., 2011 J. Pharm. Sci. 100(2): 388-401, discussing increased stability via adjuvants and assessing antigen stability in altered pH, hydration, and temperature conditions; and Rossi et al., 2016 Infect. Immun. 84(6): 1735-1742. For clarity, “stability” may be specified as “thermostability” which means the molecule's resistance to unfolding at a particular temperature and which is usually conveyed in the field by the molecule's melting temperature(s), specifically an increase in the molecule's melting temperature (of which there may be more than one melting temperature for oligomeric proteins such as dimers or trimers), see Kumar et al. 2000 Prot. Eng. Des. Sel. “Factors enhancing protein thermostability” 13(3): 179-191; and Miotto et al. 2018 bioRxiv doi: 10.1101/354266 “Insights on protein thermal stability: a graph representation of molecule interactions”). As the context requires, the thermostability of two or more molecules (such as two or more modified molecules that each comprise one or more stabilizing mutation) may be compared and one may be said to be more thermostable than the other (i.e., have an enhanced or increased thermostability as compared to the other). Stability, especially thermostability, herein may be provided by the delta stability (dStability or dS) scoring method, which is the computationally-determined difference between the relative thermostability of an in-silico mutant protein and that of a control or its wild type counterpart (i.e., non-stabilized-mutant) protein. Methods of determining dStability are known (WO 2020/079586 (PCT/IB2019/058777), MALITO et al.) and may include the use of tools such as Molecular Operating Environment (MOE) software (REF: Molecular Operating Environment (MOE) software; Chemical Computing Group Inc., available at WorldWideWeb(www).chemcomp.com). dS is measured by kcal/mol. Lower dS values indicate higher protein stability, while higher dS values indicate lower protein stability. It may be specified that the mutant polypeptides of the present invention have a higher relative thermostability (in kcal/mol) as compared to a non-mutant polypeptide under the same or comparable experimental conditions. It may be further specified that the mutant polypeptides of the present invention have a lower dS value than a non-mutant polypeptide under the same or comparable experimental conditions. It will be understood from the present invention that a mutant polypeptide having a lower dS value as compared to a non-mutant polypeptide under the same or comparable experimental conditions is more stable than the non-mutant polypeptide. The stability enhancement can be assessed using differential scanning calorimetry (DSC) as discussed in Bruylants et al. 2005 Curr. Med. Chem. 12: 2011-2020 and Calorimetry Sciences Corporation's “Characterizing Protein stability by DSC” (Life Sciences Application Note, Doc. No. 2021102136 February 2006) or by differential scanning fluorimetry (DSF). An increase in (thermo) stability may be characterized as an at least about 2° C. increase in thermal transition midpoint (Tₘ), as assessed by DSC or DSF. See, for example, Thomas et al., 2013 Hum. Vaccin. Immunother. 9(4): 744-752. A “significant” increase in, or enhancement of, thermostability is defined as an increase of at least 5° C. in the calculated Tm of a complex (calculated by, for example, the protocol provided at Example 4.7 of WO 2020/079586 (PCT/IB2019/058777), MALITO et al.). For clarity, “stability” herein may be specified as “folding stability” which refers to the molecule's folding free energy (reported in kilocalories per mole (kcal/mol)) and which may be determined using a variety of known techniques (see the Examples section herein as well as, e.g., Zhang et al. 2012 Bioinformatics 28(5): 664-671). As the context requires, the folding stability of two or more molecules may be compared and one may be said to be more stable than the other because it has a lower folding free energy value (in kcal/mol). It may be specified that a monomer or nanoparticle of the present invention has a higher/increased folding stability as compared to a control molecule or its wild type counterpart under the same or comparable conditions (e.g., experimental conditions). A “significant” increase in, or enhancement of, folding stability is defined as a folding free energy change value that is at least 100 kcal/mol lower than the folding free energy change value (in kcal/mol) of the comparison molecule in comparable or the same conditions.

In one embodiment, the polypeptide monomer of the invention comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 16 and has one or more mutations from the group consisting of: glycine (G) at the position that aligns to residue 34 of SEQ ID NO: 16 (T34G mutation), aspartic acid (D) at the position that aligns to residue 70 of SEQ ID NO: 16 (N70D mutation), isoleucine (I) at the position that aligns to residue 72 of SEQ ID NO: 16 (V72I mutation) and alanine (A) at the position that aligns to residue 124 of SEQ ID NO: 16 (S124A mutation).

In one preferred embodiment, the polypeptide monomer comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 16 and has glycine (G) at the position that aligns to residue 34 of SEQ ID NO: 16 (T34G mutation), aspartic acid (D) at the position that aligns to residue 70 of SEQ ID NO: 16 (N70D mutation), isoleucine (I) at the position that aligns to residue 72 of SEQ ID NO: 16 (V72I mutation) and alanine (A) at the position that aligns to residue 124 of SEQ ID NO: 16 (S124A mutation). In one embodiment, the polypeptide monomer comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 149. In one embodiment, the polypeptide monomer comprises the amino acid sequence of SEQ ID NO: 149.

In one embodiment, the polypeptide monomer comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 16 and has glycine (G) at the position that aligns to residue 34 of SEQ ID NO: 16 (T34G mutation), isoleucine (I) at the position that aligns to residue 72 of SEQ ID NO: 16 (V72I mutation) and alanine (A) at the position that aligns to residue 124 of SEQ ID NO: 16 (S124A mutation). In one embodiment, the polypeptide monomer comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 150. In one embodiment, the polypeptide monomer comprises the amino acid sequence of SEQ ID NO: 150.

In one embodiment, the polypeptide monomer comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 16 and has glycine (G) at the position that aligns to residue 34 of SEQ ID NO: 16 (T34G mutation), and alanine (A) at the position that aligns to residue 124 of SEQ ID NO: 16 (S124A mutation). In one embodiment, the polypeptide monomer comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 151. In one embodiment, the polypeptide monomer comprises the amino acid sequence of SEQ ID NO: 151.

In one embodiment, the polypeptide monomer comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 16 and has glycine (G) at the position that aligns to residue 34 of SEQ ID NO: 16 (T34G mutation). In one embodiment, the polypeptide monomer comprises an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 152. In one embodiment, the polypeptide monomer comprises the amino acid sequence of SEQ ID NO: 152.

The designed and de novo polypeptide monomers of the present invention are capable of self-assembly into approximately spherical nanoparticles (e.g., with an exterior surface structure diameter of about 5 nm to about 30 nm, preferably of about 15 to 20 nm). The polypeptide monomers of the present invention may therefore be used for providing self-assembled protein nanoparticles and, optionally, wherein the self-assembled protein nanoparticle carries (e.g., displays) at least one antigen molecule, at least one immunostimulant molecule, or at least one antigen molecule and at least one immunostimulant molecule. In one embodiment, the nanoparticles of the present invention (e.g., approximately spherical nanoparticles of the present invention) consist of 24 monomer subunits (e.g., wherein at least one monomer subunit is a polypeptide monomer of the present invention) and have an underlying geometry that is octahedral symmetry.

Nanoparticles (naturally occurring and recombinant nanoparticles, e.g., computationally-designed nanoparticles), methods of making them, and their use as, for example, scaffolds (or “carriers”) of one or more antigens or immunostimulants (i.e., “pharmaceutically acceptable nanoparticles”) are known in the art.

As would be recognized by the art (see, e.g., Ueda et al. 2020 elife 9: e57659; Pan et al. 2020 Adv. Mater. 32:2002940), protein nanoparticle of the present invention may be used as a “scaffold” by which it carries (through conjugation, i.e., connection, attachment, linkage, fusion, bond or ligation to the exterior surface structure of the nanoparticle) an antigen, an immunostimulant, multiple copies of the same antigen, multiple copies of the same immunostimulant, a mixture of two or more antigens (e.g., two, three, four, or five antigens; i.e., antigen bi-, tri-, quadra-, or pentavalent), a mixture of two or more immunostimulants (e.g., two, three, four, or five immunostimulants; i.e., immunostimulant bi-, tri-, quadra-, or pentavalent), or a mixture of one or more antigen(s) with one or more immunostimulant(s).

In certain embodiments, the self-assembly of polypeptide monomers places their N-termini at the outer/external surface of the nanoparticle and their C-termini at the inner/core/interior surface of the nanoparticle. In this way, an antigen or immunostimulant that is linked to the N-terminus of a polypeptide monomer is displayed at the exterior surface of the assembled nanoparticle. In other embodiments, the self-assembly of polypeptide monomers places their C-termini at the outer/external surface of the nanoparticle and their N-termini at the inner/core/interior surface of the nanoparticle. In this way, an antigen or immunostimulant that is linked to the C-terminus of a polypeptide monomer is displayed at the exterior surface of the assembled nanoparticle. In certain other embodiments, an antigen or immunostimulant is linked to the N-terminus of a polypeptide monomer and an antigen or immunostimulant is linked to the C-terminus of that polypeptide monomer (antigen(s) and/or immunostimulant(s) being the same or different) such that an antigen or immunostimulant is displayed on the exterior surface and carried on the interior surface of the assembled nanoparticle.

So an embodiment of the present invention provides a nanoparticle carrying one or more molecule(s) (e.g., wherein the molecule(s) is/are heterologous as compared to one or more (e.g., all) of the nanoparticle monomers) and optionally wherein the one or more molecule(s) is/are displayed on the exterior surface of the nanoparticle. Where said one or more displayed molecules (e.g., antigen(s) and/or immunostimulant(s)) are proteins (e.g., are all proteins), they may be expressed as part of the polypeptide monomers (i.e., as fusion protein monomers), such that self-assembly of the nanoparticle results in display of the proteins on the nanoparticle exterior surface. Alternatively, a protein display molecule may be attached to the assembled nanoparticle, for example, by chemical or biological conjugation as discussed herein and as known in the art.

In a further embodiment of the present invention, the display molecule is a poly- or oligo-saccharide (such as a bacterial capsular polysaccharide); the saccharide may be linked to a nanoparticle to provide a “glycoconjugate”. see Polonskaya et al. 2017 J. Clin. Invest. 127(4):1492-1504; Pan et al. 2020 Adv. Mater. 32:2002940.

In one embodiment, the antigen is a polypeptide having an amino acid sequence comprising or consisting of (a) FimH; or a variant, fragment and/or fusion of FimH, and (b) a donor-strand complementing amino acid sequence, wherein (b) is downstream of (a), or as otherwise described herein. In one embodiment, the antigen comprises or consists of an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 124. In one embodiment, the antigen comprises or consists of the amino acid sequence of SEQ ID NO: 124.

In one embodiment, the nanoparticle of an amino acid sequence that has at least 80% sequence identity, for example at least 85%, 90%, 91%, 92%, 93%, 94%, 95%, 96%, 97%, 98%, or 99% sequence identity to the amino acid sequence SEQ ID NO: 130 or 153. In one embodiment, the nanoparticle comprises or consists of the amino acid sequence of SEQ ID NO: 130 or 153.

Therefore, certain embodiments of the present invention provide polypeptides that are capable of self-assembling into a nanoparticle (i.e., polypeptide monomers) as well as the nucleic acid molecules that encode such polypeptides. An amino acid sequence herein may comprise, or further comprise, a tag (e.g., a purification tag such as a histidine (e.g., 6×His tag), enterokinase tag, or myc tag), and a linker between the polypeptide monomer and the one or more molecule (e.g. antigen) being carried by the nanoparticle. Further, a nucleic acid sequence herein may encode an amino acid sequence that comprises a tag and/or a linker.

Alternatively, or additionally, the polypeptide includes a phenylalanine (Phe, F) residue at the N-terminus of the FimH polypeptide. Alternatively or additionally, when the polypeptide comprises a nanoparticle domain at the C-terminus or at the N-terminus, the polypeptide includes a phenylalanine (Phe, F) or an aspartic acid (Asp, D) residue at the N-terminus of the mature polypeptide, i.e., after cleavage or removal of a leader sequence, if present. The presence of an aspartic acid (Asp, D) residue at the N-terminus of the mature polypeptide, which comprises a nanoparticle domain at the C-terminus or at the N-terminus, is associated with an improved secretion of the polypeptide when expressed by a mammalian host cell.

Alternatively or additionally, the polypeptide comprises or consists of an amino acid sequence corresponding to:
(a) SEQ ID NO: 20, or a variant and/or fragment thereof,
(b) SEQ ID NO: 21, or a variant and/or fragment thereof,
(c) SEQ ID NO: 22, or a variant and/or fragment thereof,
(d) SEQ ID NO: 23, or a variant and/or fragment thereof,
(e) SEQ ID NO: 24, or a variant and/or fragment thereof,
(f) SEQ ID NO: 25, or a variant and/or fragment thereof,
(g) SEQ ID NO: 26, or a variant and/or fragment thereof,
(h) SEQ ID NO: 27, or a variant and/or fragment thereof,
(i) SEQ ID NO: 28, or a variant and/or fragment thereof,
(j) SEQ ID NO: 29, or a variant and/or fragment thereof,
(k) SEQ ID NO: 30, or a variant and/or fragment thereof,
(l) SEQ ID NO: 31, or a variant and/or fragment thereof,
(m) SEQ ID NO: 32, or a variant and/or fragment thereof,
(n) SEQ ID NO: 33, or a variant and/or fragment thereof,
(o) SEQ ID NO: 34, or a variant and/or fragment thereof,
(p) SEQ ID NO: 35, or a variant and/or fragment thereof,
(q) SEQ ID NO: 36, or a variant and/or fragment thereof,
(r) SEQ ID NO: 37, or a variant and/or fragment thereof,
(s) SEQ ID NO: 38, or a variant and/or fragment thereof,
(t) SEQ ID NO: 39, or a variant and/or fragment thereof,
(u) SEQ ID NO: 40, or a variant and/or fragment thereof,
(v) SEQ ID NO: 41, or a variant and/or fragment thereof,
(w) SEQ ID NO: 42, or a variant and/or fragment thereof,
(x) SEQ ID NO: 43, or a variant and/or fragment thereof,
(y) SEQ ID NO: 44, or a variant and/or fragment thereof,
(z) SEQ ID NO: 79, or a variant and/or fragment thereof,
(aa) SEQ ID NO: 80, or a variant and/or fragment thereof,
(bb) SEQ ID NO: 81, or a variant and/or fragment thereof,
(cc) SEQ ID NO: 82, or a variant and/or fragment thereof,
(dd) SEQ ID NO: 83, or a variant and/or fragment thereof,
(ee) SEQ ID NO: 84, or a variant and/or fragment thereof,
(ff) SEQ ID NO: 85, or a variant and/or fragment thereof,
(gg) SEQ ID NO: 86, or a variant and/or fragment thereof,
(hh) SEQ ID NO: 87, or a variant and/or fragment thereof,
(ii) SEQ ID NO: 88, or a variant and/or fragment thereof,
(jj) SEQ ID NO: 89, or a variant and/or fragment thereof,
(kk) any one of SEQ ID NO: 120-124, SEQ ID NO: 129-143 and 153 or a variant and/or a fragment thereof.

In one preferred embodiment, the polypeptide comprises or consists of an amino acid sequence corresponding to SEQ ID NO: 123 or SEQ ID NO:124. In one embodiment, the polypeptide comprises or consists of an amino acid sequence with at least 70% sequence identity to SEQ ID NO: 123 or SEQ ID NO:124, for example, 80%, 85%, 90%, 91%, 92%, 93%, 94%, 95%, 96%, 97%, 98% or 99% sequence identity to SEQ ID NO: 123 or SEQ ID NO:124.

Alternatively or additionally, the mannose binding of the polypeptide is at least 20% lower than that of native FimH complexed with native FimC (FimHC complex), e.g., at least 30%, 40%, 50%, 60%, 70%, 80%, 90%, 100% lower.

Mannose binding can be determined using any suitable means known in the art, for example, surface plasmon resonance may be used to verify binding, binding specificity and binding constants of FimH constructs with Man-BSA and Glc-BSA (negative control), see, for example Rabani et al., 2018, ‘Conformational switch of the bacterial adhesin FimH in the absence of the regulatory domain: Engineering a minimalistic allosteric system’ J. Biol. Chem., 293(5):1835-1849, which is incorporated by reference herein.

By ‘native FimH’ we mean or include wild-type FimH, in particular, wild-type FimH from which domain (a) of the polypeptide of the invention was derived (optionally, with the native N-terminal secretory sequence removed). Alternatively or additionally, we mean or include E. coli J96 FimH (e.g., SEQ ID NO: 1 or SEQ ID NO: 2), FimH of E. coli UPEC 536 (e.g., SEQ ID NO: 100 or SEQ ID NO: 101), FimH of E. coli CFT073 (e.g., SEQ ID NO: 102 or SEQ ID NO: 103), FimH of E. coli 789 (e.g., SEQ ID NO: 104 or SEQ ID NO: 105), FimH of E. coli IHE3034 (e.g., SEQ ID NO: 106 or SEQ ID NO: 107). In particular, we include FimH in the high-affinity conformation, relaxed (R) state (see above).

By ‘native FimC’ we mean or include wild-type FimC (optionally, with the native N-terminal secretory sequence removed). Alternatively or additionally, we mean or include E. coli J96 FimC, FimC of UPEC 536, FimC of E. coli CFT073, FimC of E. coli 789, FimC of E. coli IHE3034.

By ‘FimH complexed with native FimC’ and ‘FimHC complex’ we mean or include FimH bound to FimC as seen in the periplasm of bacteria naturally expressing FimH and FimC, in the manner and/or conditions taught in the present examples section, in the manner and/or conditions taught in (a) D. Choudhury, X-ray structure of the FimC-FimH chaperone-adhesin complex from uropathogenic Escherichia coli. Science 285, 1061-1066 (1999), (b) C.-S. Hung, Structural basis of tropism of Escherichia coli to the bladder during urinary tract infection. Mol. Microbiol. 44, 903-915 (2002), (c) I. Le Trong, Structural basis for mechanical force regulation of the adhesin FimH via finger trap-like 3 sheet twisting. Cell 141, 645-655 (2010), (d) G. Phan, Crystal structure of the FimD usher bound to its cognate FimC-FimH substrate. Nature 474, 49-53 (2011), or (e) S. Geibel, Structural and energetic basis of folded-protein transport by the FimD usher. Nature 496, 243-246 (2013).

Alternatively or additionally, the anti-FimH immunogenicity of the polypeptide is at least 20% higher than that of native FimH complexed with native FimC (in particular, we include FimH in the high-affinity conformation, relaxed (R) state (see above).), e.g., at least 30%, 40%, 50%, 60%, 70%, 80%, 90%, 100%, 150%, 200%, 300%, 400% or 500% higher. Immunogenicity can be determined by any suitable means known in the art for example, ELISA or Luminex (see Examples section).

Alternatively or additionally, the auto-aggregation induced by the polypeptide is at least 20% lower than that of native FimH, e.g., at least 30%, 40%, 50%, 60%, 70%, 80%, 90%, 100% lower. By ‘the auto-aggregation induced by the polypeptide is at least X % lower than that of native FimH’ (wherein ‘X’ is a number between 20 and 100) we mean or include that the polypeptide, when expressed by bacteria instead of native FimH, induces at least X % less bacterial auto-aggregation than otherwise equivalent bacteria expressing the equivalent native FimH. By ‘equivalent native FimH’ we mean or include the FimH native to the bacteria being used in the test, the native FimH from which the polypeptide of the invention was derived, and/or the native FimH with which the polypeptide of the invention has the highest sequence identity with. Any suitable means known in the art for determining auto-aggregation may be used but in one embodiment, the method used is that described in Schembri, Christiansen and Klemm, 2001, ‘FimH-mediated autoaggregation of Escherichia coli’ Molecular Microbiology, 41(6), 1419-1430, which is incorporated by reference herein; or Thomas et al., 2002, ‘Bacterial adhesion to target cells enhanced by shear force’ Cell, 109(7):913-23, which is incorporated by reference herein; or Hartman et al., 2012, ‘Inhibition of bacterial adhesion to live human cells: Activity and cytotoxicity of synthetic mannosides’ FEBS Letters, 586(10): 1459-1465, which is incorporated by reference herein; or Falk et al., 1995, ‘Chapter 9: Bacterial Adhesion and Colonization Assays’ Meth. Cell, Biol., 45:165-192, which is incorporated by reference herein; or Zanaboni et al., 2016, ‘A novel high-throughput assay to quantify the vaccine-induced inhibition of Bordetella pertussis adhesion to airway epithelia’ BMC Microbiol., 16:a215, which is incorporated by reference herein. Alternatively or additionally, bacterial adhesion is (in brief) measured with the BAI assay as follows: UPEC strains engineered to express the mCherry fluorescent marker, are incubated for 30 minutes with monolayers of SV-HUC-1 in 96 well plates in the presence of specific sera against FimH derivatives or positive/negative controls. After adhesion, cells are washed extensively to remove unbound bacteria and fixed with formaldehyde. Finally, the specific fluorescent signal associated with the adhered bacteria is recorded by the use of an automated high content screening microscope (Opera Phenix) and quantified with the Harmony software.

Alternatively or additionally, the polypeptide is capable of inhibiting bacterial adhesion by at least 20%, e.g., by at least 30%, 40%, 50%, 60%, 70%, 80%, 90%, or by 100%.

By ‘inhibiting bacterial adhesion’ we mean or include adhesion measured by proxy via bacterial motility or via the bacterial adhesion assay(s) described above (for example with the BAI assay) and/or in the present Examples section.

Alternatively or additionally, the polypeptide is capable of inhibiting hemagglutination of guinea pig red blood cells by at least 2-fold, e.g. by at least 3-fold, 4-fold, 5-fold, 10-fold, 15-fold, 20-fold, 25-fold, 30-fold, 40-fold, 50-fold, 60-fold, 70-fold, 80-fold, 90-fold, or 100-fold.

By ‘inhibiting hemagglutination’ we mean or include inhibition of hemagglutination as measured by the hemagglutination inhibition assay (HAI) described in Hultgren et al, Infect Immun 1986, 54, 613-620 and Jarvis C et al, ChemMedChem 2016, 11, 367-373 and/or in the Examples section.

Alternatively or additionally, the polypeptide is soluble by which we mean or include that at least 50% of the polypeptide w/w (e.g., present in a mixture and/or expressed by the/a cell) is in soluble form, for example at least 60%, 70%, 80&, 90%, 95% or 100% of the polypeptide is in soluble form.

A second aspect of the invention provides a nucleic acid encoding a polypeptide according to the first aspect, for example, DNA or RNA.

Alternatively or additionally, the nucleic acid has been codon optimised for expression in a selected prokaryotic or eukaryotic cell, for example, a yeast cell (e.g., Saccharomyces cerevisiae, Pichia pastoris), an insect cell (e.g., Spodopterafrugiperda Sf21 cells, or Sf9 cells), or a mammalian cell (Expi293, Expi293GNTI (Life Technologies), Chinese hamster ovary (CHO) cell, and Human embryonic kidney 293 cells (HEK 293)). By “codon optimized” is intended modification with respect to codon usage that may increase translation efficacy and/or half-life of the nucleic acid. Codon usage/optimization tables for many organisms are well known and publicly available (as provided by, e.g., Athey et al. 2017 BMC Bioinf. 18:391). Codon optimisation can be performed using any suitable means known in the art, for example, the method operated by GeneArt.

Alternatively or additionally, the nucleic comprises or consists of a nucleic acid sequence corresponding to:
(1) SEQ ID NO: 45, or a variant and/or fragment thereof,
(2) SEQ ID NO: 46, or a variant and/or fragment thereof,
(3) SEQ ID NO: 47, or a variant and/or fragment thereof,
(4) SEQ ID NO: 48, or a variant and/or fragment thereof,
(5) SEQ ID NO: 49, or a variant and/or fragment thereof,
(6) SEQ ID NO: 50, or a variant and/or fragment thereof,
(7) SEQ ID NO: 51, or a variant and/or fragment thereof,
(8) SEQ ID NO: 52, or a variant and/or fragment thereof,
(9) SEQ ID NO: 53, or a variant and/or fragment thereof,
(10) SEQ ID NO: 54, or a variant and/or fragment thereof,
(11) SEQ ID NO: 55, or a variant and/or fragment thereof,
(12) SEQ ID NO: 56, or a variant and/or fragment thereof,
(13) SEQ ID NO: 57, or a variant and/or fragment thereof,
(14) SEQ ID NO: 58, or a variant and/or fragment thereof,
(15) SEQ ID NO: 59, or a variant and/or fragment thereof,
(16) SEQ ID NO: 60, or a variant and/or fragment thereof,
(17) SEQ ID NO: 61, or a variant and/or fragment thereof,
(18) SEQ ID NO: 62, or a variant and/or fragment thereof,
(19) SEQ ID NO: 63, or a variant and/or fragment thereof,
(20) SEQ ID NO: 64, or a variant and/or fragment thereof,
(21) SEQ ID NO: 65, or a variant and/or fragment thereof,
(22) SEQ ID NO: 66, or a variant and/or fragment thereof,
(23) SEQ ID NO: 67, or a variant and/or fragment thereof,
(24) SEQ ID NO: 68, or a variant and/or fragment thereof,
(25) SEQ ID NO: 69, or a variant and/or fragment thereof,
(26) SEQ ID NO: 70, or a variant and/or fragment thereof,
(27) SEQ ID NO: 71, or a variant and/or fragment thereof,
(28) SEQ ID NO: 72, or a variant and/or fragment thereof,
(29) SEQ ID NO: 73, or a variant and/or fragment thereof,
(30) SEQ ID NO: 74, or a variant and/or fragment thereof,
(31) SEQ ID NO: 75, or a variant and/or fragment thereof,
(32) SEQ ID NO: 76, or a variant and/or fragment thereof,
(33) SEQ ID NO: 77, or a variant and/or fragment thereof,
(34) SEQ ID NO: 90, or a variant and/or fragment thereof,
(35) SEQ ID NO: 91, or a variant and/or fragment thereof,
(36) SEQ ID NO: 92, or a variant and/or fragment thereof,
(37) SEQ ID NO: 93, or a variant and/or fragment thereof,
(38) SEQ ID NO: 94, or a variant and/or fragment thereof,
(39) SEQ ID NO: 95, or a variant and/or fragment thereof,
(40) SEQ ID NO: 96, or a variant and/or fragment thereof,
(41) SEQ ID NO: 97, or a variant and/or fragment thereof,
(42) SEQ ID NO: 98, or a variant and/or fragment thereof, and
(43) SEQ ID NO: 99, or a variant and/or fragment thereof.

The skilled person will immediately appreciate that, where the nucleic acid of the invention is an RNA, T is replaced with U in the nucleic acid sequences of the invention (e.g., SEQ ID NOs: 45-99).

A third aspect of the invention provides a vector comprising the nucleic acid of the second aspect. Alternatively or additionally, the vector is a plasmid, for example, an expression plasmid. Alternatively or additionally, the plasmid is selected from the group consisting of pCDNA3.1 (Life Technologies), pCDNA3.4 (Life Technologies), pFUSE, pBROAD, pSEC, pCMV, pDSG-IBA, and pHEK293 Ultra, and the like. Alternatively or additionally, the plasmid is suitable for expression in bacterial host cells and in selected from the group consisting of pACYCDuet-1, pTrcHis2A, pET21, pET15TEV, pET22b+, pET303/CT-HIS, PET303/CT, pBAD/Myc-His A, pET303, pET24b(+), and the like.

Alternatively or additionally, the vector is a viral vector, for example, an RNA viral vector. Alternatively or additionally, the viral vector is selected from the group consisting of Adenoviral vectors, and CHAD.

A fourth aspect of the invention provides a cell, for example a host cell, comprising a nucleic acid of the second or a vector of the fourth aspect.

Suitable mammalian host cells are known in the art. Alternatively or additionally, the cell does not have N-acetylglucosaminyltransferase I (GnTI) activity. Alternatively or additionally, the cell is selected from the group consisting of Expi293, Expi293GNTI (Life Technologies), Chinese hamster ovary (CHO) cell, NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, Human embryonic kidney 293 cells (HEK 293), and the like.

Suitable bacterial host cells are known in the art. Exemplary bacterial host cells include any of the following and derivatives thereof: Escherichia coli from strains BL21(DE3), HMS174 (DE3), Origami 2 (DE3), BL21DE3T1r or T7shuffle express.

A fifth aspect of the invention provides a method of producing a polypeptide defined in the first aspect by expressing the protein in a cell as defined in the fourth aspect.

A sixth aspect of the invention provides a vaccine comprising the polypeptide defined in the first aspect, a nucleic acid defined in the second aspect, and/or a vector defined in the third aspect. Alternatively or additionally, the vaccine comprises an adjuvant.

In one embodiment, the vaccine of the invention comprises the polypeptide defined in the first aspect and an adjuvant comprising any one of: 3D-MPL, QS21 and liposomes, for example liposomes comprising cholesterol. In one embodiment, the vaccine of the invention comprises the polypeptide defined in the first aspect and an adjuvant comprising 3D-MPL, QS21 and liposomes comprising cholesterol.

The inventors have surprisingly found that vaccines comprising an adjuvant comprising 3D-MPL, QS21 and liposomes comprising cholesterol, such as the AS01 adjuvant, may elicit an improved immune response. By “improved immune response” we mean or include an increased level of immunoglobulin G (IgG) in the serum and/or in the urine of an animal, such as a mice, immunized with said vaccine respective to the level of IgG in the serum and/or in the urine of an animal, such as a mice, immunized with a reference or vaccine. For “increased level of IgG in the serum and/or in the urine” we mean or include cells by at least 2-fold, e.g. by at least 3-fold, 4-fold, 5-fold, 10-fold, 15-fold, 20-fold, 25-fold, 30-fold, 40-fold, or 50-fold. Said reference or control vaccine does not comprise an adjuvant comprising 3D-MPL, QS21 and liposomes comprising cholesterol; for example, said reference or control vaccine comprises the PHAD adjuvant.

The inventors have also surprisingly found that vaccines comprising the polypeptide defined in the first aspect and an adjuvant comprising 3D-MPL, QS21 and liposomes comprising cholesterol, such as the AS01 adjuvant, are capable of eliciting a protective immune response after one or two doses.

Immunogenic compositions (e.g., vaccines) will be pharmaceutically acceptable. They will usually include components in addition to the antigens e.g. they typically include one or more pharmaceutical carrier(s), excipient(s) and/or adjuvant(s). A thorough discussion of carriers and excipients is available in Current Protocols in Molecular Biology (F. M. Ausubel et al., eds., 1987) Supplement 30, which is incorporated by reference herein. Thorough discussions of vaccine adjuvants are available in Vaccine Design: The Subunit and Adjuvant Approach (eds. Powell & Newman) Plenum Press 1995 (ISBN 0-306-44867-X); and Vaccine Adjuvants: Preparation Methods and Research Protocols (Volume 42 of Methods in Molecular Medicine series), ISBN: 1-59259-083-7. Ed. O'Hagan which are incorporated by reference herein.

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilized during manufacture and are reconstituted into an aqueous form at the time of use. Thus, a composition of the invention may be dried, such as a lyophilized formulation. The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred. To improve thermal stability, a composition may include a temperature protective agent.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminium hydroxide adjuvant); or a citrate buffer. Buffers will typically be included in the 5-20 mM range.

The pH of a composition will generally be between 5.0 and 8.1, and more typically between 6.0 and 8.0 e.g., 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunizations (i.e. a ‘multidose’ kit). The inclusion of a preservative is preferred in multidose arrangements.

As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants.

Adjuvants

Vaccines and immunogenic compositions of the invention may also comprise an adjuvant in addition to the antigen. Adjuvants are used in vaccines in order to enhance and modulate the immune response to the antigen. The adjuvants described herein may be combined with any of the antigen(s) herein described.

The adjuvant may be any adjuvant known to the skilled person, but adjuvants include (but are not limited to) oil-in-water emulsions (for example MF59 or AS03), liposomes, saponins, TLR2 agonists, TLR3 agonists, TLR4 agonists, TLR5 agonists, TLR6 agonists, TLR7 agonists, TLR8 agonists, TLR9 agonists, aluminium salts, nanoparticles, microparticles, Immune stimulating complexes (ISCOMS), calcium fluoride and organic compound composites or combinations thereof.

Oil-In-Water Emulsions

In an embodiment of the present invention, there is provided a vaccine or immunogenic composition for use in the invention comprising an oil-in-water emulsion. Oil-in-water emulsions of the present invention comprise a metabolisable oil and an emulsifying agent. In order for any oil-in-water composition to be suitable for human administration, the oil phase of the emulsion system has to comprise a metabolisable oil. The meaning of the term metabolisable oil is well known in the art. Metabolisable can be defined as “being capable of being transformed by metabolism” (Dorland's Illustrated Medical Dictionary, W. B. Sanders Company, 25th edition, 1974). A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in an oil-in-water emulsion of the invention. Squalene is a metabolisable oil by virtue of the fact that it is an intermediate in the biosynthesis of cholesterol (Merck index, 10th Edition, entry no. 8619). In some embodiments, wherein the vaccines or immunogenic compositions of the invention comprise an oil-in-water emulsion, the metabolisable oil is present in the vaccine or in the immunogenic composition in an amount of 0.5% to 10% (v/v) of the total volume of the composition. The oil-in-water emulsion further comprises an emulsifying agent. The emulsifying agent may suitably be polyoxyethylene sorbitan monooleate (POLYSORBATE 80). Further, said emulsifying agent is suitably present in the vaccine or immunogenic composition in an amount of 0.125 to 4% (v/v) of the total volume of the composition. The oil-in-water emulsion may optionally comprise a tocol. Tocols are well known in the art and are described in EP0382271 B1. Suitably, the tocol may be alpha-tocopherol or a derivative thereof such as alpha-tocopherol succinate (also known as vitamin E succinate). Said tocol is suitably present in the adjuvant composition in an amount of 0.25% to 10% (v/v) of the total volume of the immunogenic composition. The oil-in-water emulsion may also optionally comprise sorbitan trioleate (SPAN 85).

The method of producing oil-in-water emulsions is well known to the person skilled in the art. Commonly, the method comprises mixing the oil phase (optionally comprising a tocol) with a surfactant such as a PBS/TWEEN80 solution, followed by homogenisation using a homogenizer; it would be clear to a person skilled in the art that a method comprising passing the mixture twice through a syringe needle is suitable for homogenising small volumes of liquid. Equally, the emulsification process in microfluidiser (e.g., M110S Microfluidics machine, maximum of 50 passes, for a period of 2 minutes at maximum pressure input of 6 bar (output pressure of about 850 bar)) could be adapted by the person skilled in the art to produce smaller or larger volumes of emulsion. The adaptation could be achieved by routine experimentation comprising the measurement of the resultant emulsion until a preparation was achieved with oil droplets of the required diameter.

In an oil-in-water emulsion, the oil and emulsifier should be in an aqueous carrier. The aqueous carrier may be, for example, phosphate buffered saline or citrate. In particular, the oil-in-water emulsion systems used in the present invention have a small oil droplet size in the sub-micron range. Suitably the droplet sizes will be in the range 120 to 750 nm, more particularly sizes from 120 to 600 nm in diameter. Even more particularly, the oil-in water emulsion contains oil droplets of which at least 70% by intensity are less than 500 nm in diameter, more particular at least 80% by intensity are less than 300 nm in diameter, more particular at least 90% by intensity are in the range of 120 to 200 nm in diameter.

The oil droplet size, i.e. diameter, according to the present invention is given by intensity. There are several ways of determining the diameter of the oil droplet size by intensity. Intensity is measured by use of a sizing instrument, suitably by dynamic light scattering such as the Malvern Zetasizer 4000 or preferably the Malvern Zetasizer 3000HS. A first possibility is to determine the z average diameter ZAD by dynamic light scattering (PCS-Photon correlation spectroscopy); this method additionally gives the polydispersity index (PDI), and both the ZAD and PDI are calculated with the cumulants algorithm. These values do not require the knowledge of the particle refractive index. A second means is to calculate the diameter of the oil droplet by determining the whole particle size distribution by another algorithm, either the Contin, or NNLS, or the automatic “Malvern” one (the default algorithm provided for by the sizing instrument). Most of the time, as the particle refractive index of a complex composition is unknown, only the intensity distribution is taken into consideration, and if necessary the intensity mean originating from this distribution.

ISCOMs

In some embodiments of the present invention, there are provided vaccines or immunogenic compositions of the invention comprising ISCOMs. ISCOMs are well known in the art (see Kersten & Crommelin, 1995, Biochimica et Biophysica Acta 1241: 117-138). ISCOMs comprise a saponin, cholesterol and phospholipids and form an open-cage-like structure of typically about 40 nm in size. ISCOMs result from the interaction of saponins, cholesterol and further phospholipids. A typical reaction mixture for the preparation of ISCOM is 5 mg/ml saponin and 1 mg/ml each for cholesterol and phospholipid. Phospholipids suitable for use in ISCOMs include, but are not limited, to phosphocholine (didecanoyl-L-α-phosphatidylcholine [DDPC], dilauroylphosphatidylcholine [DLPC], dimyristoylphosphatidylcholine [DMPC], dipalmitoyl phosphatidylcholine [DPPC], Distearoyl phosphatidylcholine [DSPC], Dioleoyl phosphatidylcholine [DOPC], 1-palmitoyl, 2-oleoylphosphatidylcholine [POPC], Dielaidoyl phosphatidylcholine [DEPC]), phosphoglycerol (1,2-Dimyristoyl-sn-glycero-3-phosphoglycerol [DMPG], 1,2-dipalmitoyl-sn-glycero-3-phosphoglycerol [DPPG], 1,2-distearoyl-sn-glycero-3-phosphoglycerol [DSPG], 1-palmitoyl-2-oleoyl-sn-glycero-3-phosphoglycerol [POPG]), phosphatidic acid (1,2-dimyristoyl-sn-glycero-3-phosphatidic acid [DMPA], dipalmitoyl phosphatidic acid [DPPA], distearoyl-phosphatidic acid [DSPA]), phosphoethanolamine (1,2-dimyristoyl-sn-glycero-3-phosphoethanolamine [DMPE], 1,2-Dipalmitoyl-sn-glycero-3-phosphoethanolamine [DPPE], 1,2-distearoyl-sn-glycero-3-phosphoethanolamine DSPE 1,2-Dioleoyl-sn-Glycero-3-Phosphoethanolamine [DOPE]), phoshoserine, polyethylene glycol [PEG] phospholipid (mPEG-phospholipid, polyglycerin-phospholipid, functionalized-phospholipid, terminal activated-phosholipid). In particular embodiments, ISCOMs comprise 1-palmitoyl-2-oleoyl-glycero-3-phosphoethanolamine. In further particular embodiments, highly purified phosphatidylcholine is used and can be selected from the group consisting of: Phosphatidylcholine (from egg), Phosphatidylcholine Hydrogenated (from egg) Phosphatidylcholine (from soy), Phosphatidylcholine Hydrogenated (from soy). In further particular embodiments, ISCOMs comprise phosphatidylethanolamine [POPE] or a derivative thereof. A number of saponins are suitable for use in ISCOMs. The adjuvant and haemolytic activity of individual saponins has been extensively studied in the art. For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit. Rev. Ther. Drug. Carrier Syst., 1996, 12 (1-2): 1-55; and EP0362279 B1. ISCOMs comprising fractions of Quil A have been used in the manufacture of vaccines (EP0109942 B1). These structures have been reported to have adjuvant activity (EP0109942 B1; WO 96/11711). Fractions of QuilA, derivatives of QuilA and/or combinations thereof are suitable saponin preparations for use in ISCOMs. The haemolytic saponins QS21 and QS17 (HPLC purified fractions of Quil A) have been described as potent adjuvants, and the method of their production is disclosed in U.S. Pat. No. 5,057,540 and EP0362279 B1. Also described in these references is the use of QS7 (a non-haemolytic fraction of Quil-A) which acts as a potent adjuvant for systemic vaccines. Use of QS21 is further described in Kensil et al. (1991. J. Immunology vol 146, 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711 and these are incorporated herein. Other particular QuilA fractions designated QH-A, QH-B, QH-C and a mixture of QH-A and QH-C designated QH-703 are disclosed in WO 96/011711 in the form of ISCOMs and are incorporated herein.

Microparticles

In some embodiments of the present invention, there is provided a vaccine or immunogenic composition of the invention comprising microparticles. Microparticles, compositions comprising microparticles, and methods of producing microparticles are well known in the art (see Singh et al. [2007 Expert Rev. Vaccines 6(5): 797-808] and WO 98/033487). The term “microparticle” as used herein, refers to a particle of about 10 nm to about 10,000 μm in diameter or length, derived from polymeric materials which have a variety of molecular weights and, in the case of the copolymers such as PLG, a variety of lactide:glycolide ratios. In particular, the microparticles will be of a diameter that permits parenteral administration to a subject without occluding the administrating device and/or the subject's capillaries. Microparticles are also known as microspheres. Microparticle size is readily determined by techniques well known in the art, such as photon correlation spectroscopy, laser diffractometry and/or scanning electron microscopy. Microparticles for use herein will be formed from materials that are sterilizable, non-toxic and biodegradable. Such materials include, without limitation, poly(a-hydroxy acid), polyhydroxybutyric acid, polycaprolactone, polyorthoester, polyanhydride.

Liposomes

In some embodiments of the present invention, there is provided a vaccine or immunogenic composition of the invention comprising liposomes. The term “liposomes” generally refers to uni- or multilamellar (particularly 2, 3, 4, 5, 6, 7, 8, 9, or 10 lamellar depending on the number of lipid membranes formed) lipid structures enclosing an aqueous interior. Liposomes and liposome formulations are well known in the art. Lipids, which are capable of forming liposomes, include all substances having fatty or fat-like properties. Lipids which can make up the lipids in the liposomes can be selected from the group comprising of glycerides, glycerophospholipides, glycerophosphinolipids, glycerophosphonolipids, sulfolipids, sphingolipids, phospholipids, isoprenolides, steroids, stearines, sterols, archeolipids, synthetic cationic lipids and carbohydrate containing lipids. Liposome size may vary from 30 nm to several μm depending on the phospholipid composition and the method used for their preparation. In particular embodiments of the invention, the liposome size will be in the range of 50 nm to 500 nm, and in further embodiments, 50 nm to 200 nm. Dynamic laser light scattering is a method used to measure the size of liposomes well known to those skilled in the art. The liposomes suitably contain a neutral lipid, for example phosphatidylcholine, which is suitably non-crystalline at room temperature, for example egg yolk phosphatidylcholine, dioleoyl phosphatidylcholine (DOPC) or dilauryl phosphatidylcholine. In a particular embodiment, the liposomes of the present invention contain DOPC. The liposomes may also contain a charged lipid which increases the stability of the liposome-saponin structure for liposomes composed of saturated lipids. In these cases, the amount of charged lipid is suitably 1 to 20% (w/w), preferably 5 to 10%. The ratio of sterol to phospholipid is 1 to 50% (mol/mol), suitably 20 to 25% (mol/mol).

Saponins

In some embodiments of the invention, the vaccine or immunogenic composition of the invention comprises a saponin. A particularly suitable saponin for use in the present invention is Quil A and its derivatives. Quil A is a saponin preparation isolated from the South American tree Quillaja Saponaria molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fragments of Quil A have been isolated by HPLC which retain adjuvant activity without the toxicity associated with Quil A (EP0362278), for example QS7 and QS21 (also known as QA7 and QA21). QS-21 is a natural saponin derived from the bark of Quillaja saponaria Molina, which induces CD8+ cytotoxic T cells (CTLs), Th1 cells and a predominant IgG2a antibody response and is a particular saponin in the context of the present invention. The saponin adjuvant within the immunogenic compositions of the invention in particular are immunologically active fractions of Quil A, such as QS-7 or QS-21, suitably QS-21. In particular embodiments, the vaccines and/or immunogenic compositions of the invention contain the immunologically active saponin fraction in substantially pure form. In particular, the vaccines or immunogenic compositions of the invention contain QS21 in substantially pure form, that is to say, the QS21 is at least 75%, 80%, 85%, 90% pure, for example at least 95% pure, or at least 98% pure.

In a particular embodiment, QS21 is provided with an exogenous sterol, such as cholesterol for example. Suitable sterols include β-sitosterol, stigmasterol, ergosterol, ergocalciferol and cholesterol. In a further particular embodiment, the adjuvant composition comprises cholesterol as sterol. These sterols are well known in the art, for example cholesterol is disclosed in the Merck Index, 11th Edition, page 341, as a naturally occurring sterol found in animal fat.

In one embodiment, the liposomes of the invention that comprise a saponin suitably contain a neutral lipid, for example phosphatidylcholine, which is suitably non-crystalline at room temperature, for example egg yolk phosphatidylcholine, dioleoyl phosphatidylcholine (DOPC) or dilauryl phosphatidylcholine. The liposomes may also contain a charged lipid which increases the stability of the liposome-QS21 structure for liposomes composed of saturated lipids. In these cases the amount of charged lipid is suitably 1 to 20% (w/w), particularly 5 to 10% (w/w). The ratio of sterol to phospholipid is 1 to 50% (mol/mol), suitably 20 to 25% (mol/mol).

Where the active saponin fraction is QS21, the ratio of QS21:sterol will typically be in the order of 1:100 to 1:1 (w/w), suitably between 1:10 to 1:1 (w/w), and preferably 1:5 to 1:1 (w/w). Suitably, excess sterol is present, the ratio of QS21:sterol being at least 1:2 (w/w). In one embodiment, the ratio of QS21:sterol is 1:5 (w/w). The sterol is suitably cholesterol.

Other useful saponins are derived from the plants Aesculus hippocastanum or Gyophilla struthium. Other saponins which have been described in the literature include Escin, which has been described in the Merck index (12^{th} Edition: entry 3737) as a mixture of saponins occurring in the seed of the horse chestnut tree, Lat: Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of Escin have been purified and shown to be biologically active (Yoshikawa et al., 1996, Chem Pharm Bull (Tokyo), 44(8): 1454-1464). Sapoalbin from Gypsophilla struthium (R. Vochten et al., 1968, J. Pharm. Belg. 42: p 213-226) has also been described in relation to ISCOM production for example.

A saponin, such as QS21, can be used at amounts between 1 and 100 μg per human dose of the adjuvant composition. QS21 may be used at a level of about 50 μg, for example between 40 to 60 μg, suitably between 45 to 55 μg or between 49 and 51 μg or 50 μg. In a further embodiment, the human dose of the adjuvant composition comprises QS21 at a level of about 25 μg, for example between 20 to 30 μg, suitably between 21 to 29 μg or between 22 to 28 μg or between 28 and 27 μg or between 24 and 26 μg, or 25 μg.

TLR4 Agonist

In some embodiments, the vaccine or immunogenic composition of the invention comprises a TLR4 agonist. By “TLR agonist” it is meant a component which is capable of causing a signaling response through a TLR signaling pathway, either as a direct ligand or indirectly through generation of endogenous or exogenous ligand (Sabroe et al, 2003, JI p 1630-5). A TLR4 agonist is capable of causing a signaling response through a TLR-4 signaling pathway. A suitable example of a TLR-4 agonist is a lipopolysaccharide, suitably a non-toxic derivative of lipid A, particularly monophosphoryl lipid A or more particularly 3-Deacylated monophoshoryl lipid A (3D-MPL).

3D-MPL is sold under the name MPL by GlaxoSmithKline Biologicals and is referred throughout the document as MPL or 3D-MPL. See, for example, U.S. Pat. Nos. 4,436,727; 4,877,611; 4,866,034 and 4,912,094. 3D-MPL primarily promotes CD4+ T cell responses with an IFN-gamma (Th1) phenotype. 3D-MPL can be produced according to the methods disclosed in GB 2 220 211 A. Chemically, it is a mixture of 3-deacylated monophosphoryl lipid A with 4, 5 or 6 acylated chains. In the compositions of the present invention, small particle 3D-MPL may be used to prepare the aqueous adjuvant composition. Small particle 3D-MPL has a particle size such that it may be sterile-filtered through a 0.22 m filter. Such preparations are described in WO 94/21292. Preferably, powdered 3D-MPL is used to prepare the aqueous adjuvant compositions of the present invention.

Other TLR-4 agonists which can be used are alkyl glucosaminide phosphates (AGPs) such as those disclosed in WO 98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840.

Other suitable TLR-4 agonists are as described in WO 03/011223 and in WO 03/099195, such as compound I, compound II and compound III disclosed on pages 4-5 of WO 03/011223 or on pages 3 to 4 of WO 03/099195 and in particular those compounds disclosed in WO 03/011223, as ER803022, ER803058, ER803732, ER804053, ER804057m ER804058, ER804059, ER804442, ER804680 and ER804764. For example, one suitable TLR-4 agonist is ER804057.

A TLR-4 agonist, such as a lipopolysaccharide, such as 3D-MPL, can be used at amounts between 1 and 100 μg per human dose of the adjuvant composition. 3D-MPL may be used at a level of about 50 μg, for example between 40 to 60 μg, suitably between 45 to 55 μg or between 49 to 51 μg or 50 μg per human dose. In a further embodiment, the human dose of the adjuvant composition comprises 3D-MPL at a level of about 25 μg, for example between 20 to 30 μg, suitably between 21 to 29 μg or between 22 to 28 μg or between 28 to 27 μg or between 24 to 26 μg, or 25 μg.

Synthetic derivatives of lipid A are known and thought to be TLR 4 agonists including, but not limited to:
OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)
OM294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO 99/64301 and WO 00/0462)
OM197 MP-Ac DP (3S-, 9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127).
PHAD (phosphorylated hexa-acyl disaccharide).

Other suitable TLR-4 ligands, capable of causing a signalling response through TLR-4 (Sabroe et al, JI 2003 p 1630-5) are, for example, lipopolysaccharide from gram-negative bacteria and its derivatives, or fragments thereof, in particular a non-toxic derivative of LPS (such as 3D-MPL). Other suitable TLR agonist are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, muramyl dipeptide (MDP) or F protein of respiratory syncytial virus (RSV). In one embodiment, the TLR agonist is HSP 60, 70 or 90.

TLR Agonists

Rather than a TLR4 agonist, other natural or synthetic agonists of TLR molecules may be used in vaccines or immunogenic composition of the invention. These include, but are not limited to, agonists for TLR2, TLR3, TLR5, TLR6, TLR7, TLR8 and TLR9.

In one embodiment of the present invention, a TLR agonist is used that is capable of causing a signalling response through TLR-1 (Sabroe et al, JI 2003 p 1630-5). Suitably, the TLR agonist capable of causing a signalling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam3Cys) LP which mimics the acetylated amino terminus of a bacterial lipoprotein and OspA LP from Borrelia burgdorferi.

In a further embodiment, a TLR agonist is used that is capable of causing a signalling response through TLR-2 (Sabroe et al, JI 2003 p 1630-5). Suitably, the TLR agonist capable of causing a signalling response through TLR-2 is one or more of a lipoprotein, a peptidoglycan, a bacterial lipopeptide from M. tuberculosis, B. burgdorferi, T. pallidum, peptidoglycans from species including Staphylococcus aureus, lipoteichoic acids, mannuronic acids, Neisseria porins, bacterial fimbriae, Yersinia virulence factors, CMV virions, measles haemagglutinin, and zymosan from yeast.

In a further embodiment, a TLR agonist is used that is capable of causing a signalling response through TLR-3 (Sabroe et al, JI 2003 p 1630-5). Suitably, the TLR agonist capable of causing a signalling response through TLR-3 is double stranded RNA (dsRNA), or polyinosinic-polycytidylic acid (Poly IC), a molecular nucleic acid pattern associated with viral infection.

In an alternative embodiment, a TLR agonist is used that is capable of causing a signalling response through TLR-5 (Sabroe et al, JI 2003 p 1630-5). Suitably, the TLR agonist capable of causing a signalling response through TLR-5 is bacterial flagellin. Said TLR-5 agonist may be flagellin or may be a fragment of flagellin which retains TLR-5 agonist activity. The flagellin can include a polypeptide selected from the group consisting of H. pylori, S. typhimurium, V. cholera, S. marcescens, S. flexneri, T. pallidum, L. pneumophilia, B. burgdorferi; C. difficile, R. meliloti, A. tumefaciens; R. lupine; B. clarridgeiae, P. mirabilis, B. subtilis, L. moncytogenes, P. aeruginosa and E. coli.

In a particular embodiment, the flagellin is selected from the group consisting of S. typhimurium flagellin B (Genbank Accession number AF045151), a fragment of S. typhimurium flagellin B, E. coli FliC. (Genbank Accession number AB028476); fragment of E. coli FIiC; S. typhimurium flagellin FliC (ATCC14028) and a fragment of S. typhimurium flagellin FliC In a further particular embodiment, said TLR-5 agonist is a truncated flagellin, as described in WO 09/156405 i.e. one in which the hypervariable domain has been deleted. In one aspect of this embodiment, said TLR-5 agonist is selected from the group consisting of: FliC_{Δ174-400}; FliC_{Δ161-405} and FliC_{Δ138-405}.

In a further particular embodiment, said TLR-5 agonist is a flagellin, as described in WO 09/128950. In a further embodiment, a TLR agonist is used that is capable of causing a signalling response through TLR-6 (Sabroe et al, JI 2003 p 1630-5). Suitably, the TLR agonist capable of causing a signalling response through TLR-6 is mycobacterial lipoprotein, di-acylated LP, and phenol-soluble modulin. Further TLR6 agonists are described in WO 03/043572.

In a further embodiment, a TLR agonist is used that is capable of causing a signalling response through TLR-7 (Sabroe et al, JI 2003 p 1630-5). Suitably, the TLR agonist capable of causing a signalling response through TLR-7 is a single stranded RNA (ssRNA), loxoribine, a guanosine analogue at positions N7 and C8, or an imidazoquinoline compound, or derivative thereof. In a particular embodiment, the TLR agonist is imiquimod. Further TLR7 agonists are described in WO 02/085905.

In a further embodiment, a TLR agonist is used that is capable of causing a signalling response through TLR-8 (Sabroe et al, JI 2003 p 1630-5). Suitably, the TLR agonist capable of causing a signalling response through TLR-8 is a single stranded RNA (ssRNA), an imidazoquinoline molecule with anti-viral activity, for example resiquimod (R848); resiquimod is also capable of recognition by TLR-7. Other TLR-8 agonists which may be used include those described in WO 04/071459.

In a further embodiment, a TLR agonist is used that is capable of causing a signalling response, such as one that comprises a CpG motif. The term “immunostimulatory oligonucleotide” is used herein to mean an oligonucleotide that is capable of activating a component of the immune system. In one embodiment, the immunostimulatory oligonucleotide comprises one or more unmethylated cytosine-guanosine (CpG) motifs. In a further embodiment, the immunostimulatory oligonucleotide comprises one or more unmethylated thymidine-guanosine (TG) motif or may be T-rich. By T-rich, it is meant that the nucleotide composition of the oligonucleotide comprises greater than 50, 60, 70 or 80% thymidine. In one embodiment, the oligonucleotide is not an immunostimulatory oligonucleotide and does not comprise an unmethylated CpG motif. In a further embodiment the immunostimulatory oligonucleotide is not T-rich and/or does not comprise an unmethylated TG motif.

The oligonucleotide may be modified in order to improve in vitro and/or in vivo stability. For example, in one embodiment, the oligonucleotides are modified so as to comprise a phosphorothioate backbone, i.e. internucleotide linkages. Other suitable modifications including diphosphorothioate, phosphoroamidate and methylphosphonate modifications as well as alternative internucleotide linkages to oligonucleotides are well known to those skilled in the art and are encompassed by the invention.

In another embodiment, the vaccines or immunogenic compositions of the invention further comprise an immunostimulant selected from the group consisting of: a TLR-1 agonist, a TLR-2 agonist, TLR-3 agonist, a TLR-4 agonist, a TLR-5 agonist, a TLR-6 agonist, a TLR-7 agonist, a TLR-8 agonist, TLR-9 agonist, or a combination thereof.

Calcium Composites

In some embodiments, the vaccine or immunogenic composition of the invention comprises a calcium fluoride composite, the composite comprising Ca, F, and Z. “Z” as used herein refers to an organic molecule. As used herein, a “composite” is a material that exists as a solid when dry, and that is insoluble, or poorly soluble, in pure water. In some aspects, Z comprises a functional group that forms an anion when ionized.

Such functional groups include without limitation one or more functional groups selected from the group consisting of: hydroxyl, hydroxylate, hydroxo, oxo, N-hydroxylate, hydroaxamate, N-oxide, bicarbonate, carbonate, carboxylate, fatty acid, thiolate, organic phosphate, dihydrogenophosphate, monohydrogenophosphate, monoesters of phosphoric acid, diesters of phosphoric acid, esters of phospholipid, phosphorothioate, sulphates, hydrogen sulphates, enolate, ascorbate, phosphoascorbate, phenolate, and imine-olates.

In some aspects, the calcium fluoride composites herein described comprise Z, where Z is an anionic organic molecule possessing an affinity for calcium and forming a water insoluble composite with calcium and fluoride. In further aspects, the calcium fluoride composites herein described comprise Z, where Z may be categorized as comprising a member of a chemical category selected from the group consisting of: hydroxyl, hydroxylates, hydroxo, oxo, N-hydroxylate, hydroaxamate, N-oxide, bicarbonates, carbonates, carboxylates and dicarboxylate, salts of carboxylic-acids, salts of QS21, extract of bark of Quillaja saponaria, extract of immunological active saponin, salts of saturated or unsaturated fatty acid, salts of oleic acid, salts of amino-acids, thiolates, thiolactate, salt of thiol-compounds, salts of cysteine, salts of N-acetyl-cysteine, L-2-Oxo-4-thiazolidinecarboxylate, phosphates, dihydrogenophosphates, monohydrogenophosphate, salts of phosphoric-acids, monoesters of phosphoric acids and their salts, diesters of phosphoric acids and their salts, esters of 3-O-desacyl-4′-monophophoryl lipid A, esters of 3D-MLA, MPL, esters of phospholipids, DOPC, dioleolyphosphatidic derivatives, phosphates from CpG motifs, phosphorothioates from CpG family, sulphates, hydrogen sulphates, salts of sulphuric acids, enolates, ascorbates, phosphoascorbate, phenolate, α-tocopherol, imine-olates, cytosine, methyl-cytosine, uracyl, thymine, barbituric acid, hypoxanthine, inosine, guanine, guanosine, 8-oxo-adenine, xanthine, uric acid, pteroic acid, pteroylglutamic acid, folic acid, riboflavin, and lumiflavin. In further aspects, the calcium fluoride composites herein described comprise Z, where Z is selected from the group consisting of: N-acetyl cysteine; thiolactate; adipate; carbonate; folic acid; glutathione; and uric acid. In some aspects, the calcium fluoride composites herein comprise Z, where Z is selected from the group consisting of: N-acetyl cysteine; adipate; carbonate; and folic acid. In further aspects, the calcium fluoride composites herein comprise Z, where Z is N-acetyl cysteine, and the composite comprises between 51% Ca, 48% F, no more than 1% N-acetyl cysteine (w/w) and 37% Ca, 26% F, and 37% N-acetyl cysteine (w/w). In further aspects, the calcium fluoride composites herein comprise Z, where Z is thiolactate, and the composite comprises between 51% Ca, 48% F, no more than 1% thiolactate (w/w) and 42% Ca, 30% F, 28% thiolactate (w/w). In further aspects, the calcium fluoride composites herein comprise Z, where Z is adipate, and the composite comprises between 51% Ca, 48% F, no more than 1% adipate (w/w) and 38% Ca, 27% F, 35% adipate (w/w). In further aspects, the calcium fluoride composites herein comprise Z, where Z is carbonate, and the composite comprises between 51% Ca, 48% F, no more than 1% carbonate (w/w) and 48% Ca, 34% F, 18% carbonate (w/w). In further aspects, the calcium fluoride composites herein comprise Z, where Z is folic acid, and the composite comprises between 51% Ca, 48% F, no more than 1% folic acid (w/w) and 22% Ca, 16% F, 62% folic acid (w/w). In further aspects, the calcium fluoride composites herein comprise Z, where Z is glutathione, and the composite comprises between 51% Ca, 48% F, no more than 1% glutathione (w/w) and 28% Ca, 20% F, 52% glutathione (w/w). In further aspects, the calcium fluoride composites herein comprise Z, where Z is uric acid, and the composite comprises between 51% Ca, 48% F, and no more than 1% uric acid (w/w) and 36% Ca, 26% F, and 38% uric acid (w/w).

Aluminium Salts

In one embodiment, the vaccine or immunogenic composition of the invention comprises an aluminium salt. Suitable aluminium salt adjuvants are well known to the skilled person and include but are not limited to aluminium phosphate, aluminium hydroxide or a combination thereof. Suitable aluminium salt adjuvants include but are not limited to REHYDRAGEL HS, ALHYDROGEL 85, REHYDRAGEL PM, REHYDRAGEL AB, REHYDRAGEL HPA, REHYDRAGEL LV, ALHYDROGEL or a combination thereof.

In particular, the aluminium salts may have a protein adsorption capacity of between 2.5 and 3.5, 2.6 and 3.4, 2.7 and 3.3 or 2.9 and 3.2, 2.5 and 3.7, 2.6 and 3.6, 2.7 and 3.5, or 2.8 and 3.4 protein (BSA)/ml aluminium salt. In a particular embodiment of the invention, the aluminium salt has a protein adsorption capacity of between 2.9 and 3.2 mg BSA/mg aluminium salt. Protein adsorption capacity of the aluminium salt can be measured by any means known to the skilled person. The protein adsorption capacity of the aluminium salt may be measured using the method as described in Example 1 of WO 12/136823 (which utilises BSA) or variations thereof.

Aluminium salts described herein (i.e. having the protein adsorption capacity described herein) may have a crystal size of between 2.8 and 5.7 nm as measured by X-ray diffraction, for example 2.9 to 5.6 nm, 2.8 to 3.5 nm, 2.9 to 3.4 nm or 3.4 to 5.6 nm or 3.3 and 5.7 nm as measured by X-ray diffraction. X-ray diffraction is well known to the skilled person. In a particular embodiment of the invention the crystal size is measured using the method described in Example 1 of WO 12/136823 or variations thereof.

The polypeptide(s) and/or nucleic acid(s) described herein may be administered to a subject by any route of administration, for example, orally, nasally, sublingually, intravenously, intramuscularly, intradermally (e.g. a skin patch with microprojections) or transdermally (e.g. an ointment or cream).

A seventh aspect of the invention provides a polypeptide defined in the first aspect, a nucleic acid defined in the second aspect, a vector defined in the third aspect, and/or a vaccine of the sixth aspect for use in medicine.

An eighth aspect provides a polypeptide defined in the first aspect, a nucleic acid defined in the second aspect, a vector defined in the third aspect, and/or a vaccine of the sixth aspect for use in raising an immune response in a mammal, for example, for treating and/or preventing one or more disease.

A ninth aspect provides the use of a polypeptide defined in the first aspect, a nucleic acid defined in the second aspect, a vector defined in the third aspect, and/or a vaccine of the sixth aspect for raising an immune response in a mammal, for example, for treating and/or preventing one or more disease.

A tenth aspect provides the use of a polypeptide defined in the first aspect, a nucleic acid defined in the second aspect, a vector defined in the third aspect, and/or a vaccine of the sixth aspect for the manufacture of a medicament for raising an immune response in a mammal, for example, for treating and/or preventing one or more disease.

An eleventh aspect provides a method of raising an immune response in a mammal, the method comprising or consisting of administering the mammal with an effective amount of a polypeptide defined in the first aspect, a nucleic acid defined in the second aspect, a vector defined in the third aspect, and/or a vaccine of the sixth aspect.

The use or method of any one of the seventh to eleventh aspects wherein the one or more disease is urinary tract infection (UTI). Alternatively or additionally, the UTI is caused by one or more bacterium of a genus selected from the group consisting of Escherichia and Klebsiella. Alternatively or additionally, the one or more bacterium is selected from the group consisting of Escherichia coli and Klebsiella pneumoniae. Alternatively or additionally, the Escherichia coli is a UroPathogenic Escherichia coli (UPEC). Alternatively or additionally, the one or more bacterium is selected from the group consisting of E. coli J96, E. coli UPEC 536, E. coli CFT073, E. coli UMN026, E. coli CLONE Dil4, E. coli CLONE Di2, E. coli CFT073; E. coli IA139, E. coli 536, E. coli NA114, and E. coli UTI89. Alternatively or additionally, the one or more bacterium is selected from the group consisting of the following K. pneumoniae strains: C3091, 3824, 3857, 3858, 3859, 3860, 3861, 3928, 3950, 3951, 4041, 4121, 4133, sp3, sp7, sp10, sp13, sp14, sp15, sp19, sp20, sp22, sp25, sp28, sp29, sp30, sp31, sp32, sp33, sp34, sp37, sp39, sp41, cas119, cas120, cas121, cas122, cas123, cas124, cas125, cas126, cas127, cas128, cas663, cas664, cas665, cas666, cas667, cas668, cas669, cas670, cas671, cas672, cas673, cas674, cas675, cas676, cas677, cas678, cas679, cas680, cas681, cas682, Kp342 and MGH78578.

Preferred, non-limiting examples which embody certain aspects of the invention will now be described, with reference to the following tables and figures.

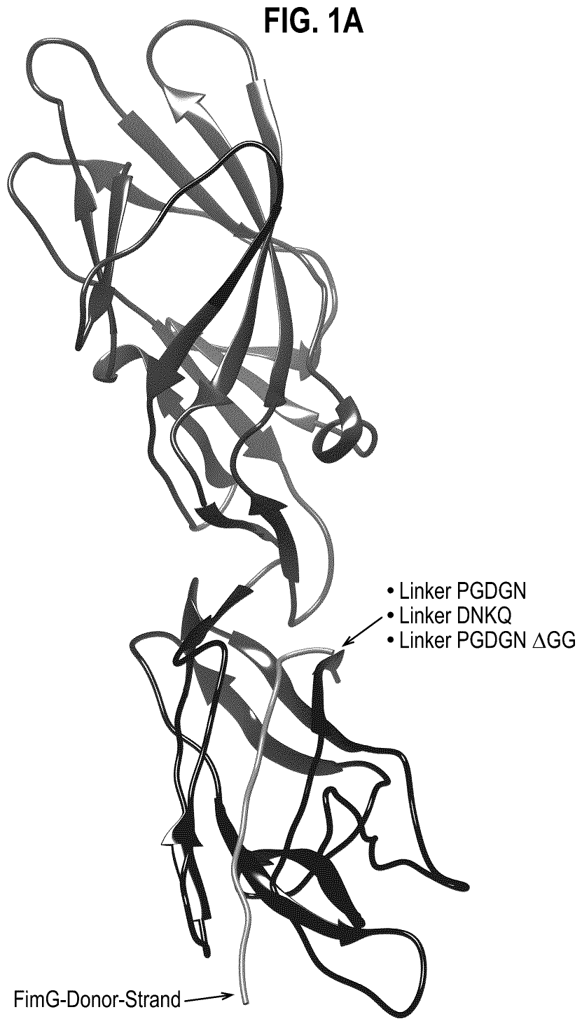

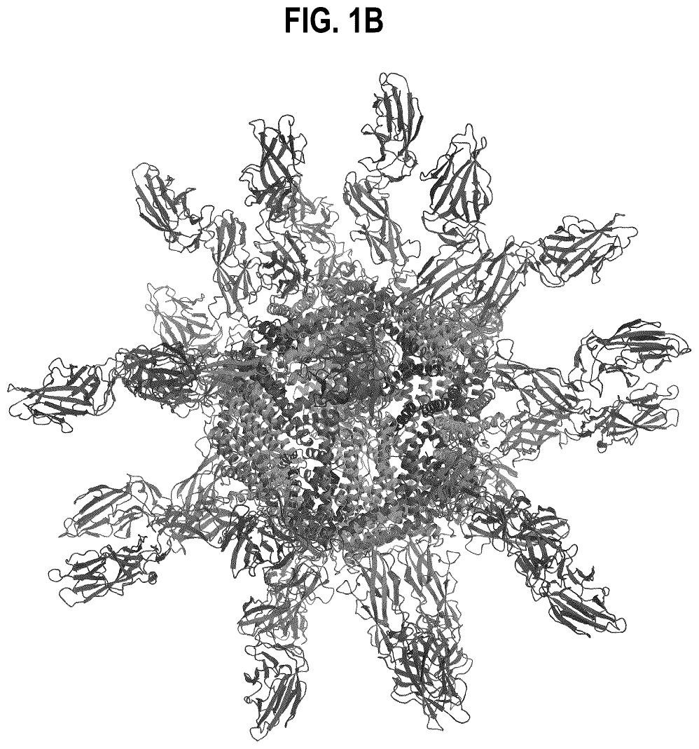

FIG. 1A and FIG. 1B. Schematic representation of FimH constructs.
A) FIG. 1A. Structure of stabilized FimH (PDB: 4XO9). Cartoon representation of FimH stabilized by FimGFimG donor strand (in blue—indicated by the arrows). Domain FimH_{L} is in yellow (top portion) while FimH_{P} in Red (bottom portion). Glycines natural linker between domains is represented in green sticks.
B) FIG. 1B Structure of FimH_DG_PGDGN_Ferritin. Aminoacidic sequence of FimH_DG_PGDGN (light blue) fused to ferritin (red). A linker composed by SGS-8H-GSG- is connecting FimH to ferritin molecule. IgK leader sequence for expression in mammalian cells and secretion into medium is in yellow followed by the extra N-terminal charged residues. Model of the 3D structure obtained with Rosetta common software. Cartoon representation of FimH_DG displayed on Ferritin surface. 24 FimH subunits are present and coloured in yellow\blue while ferritin in red.

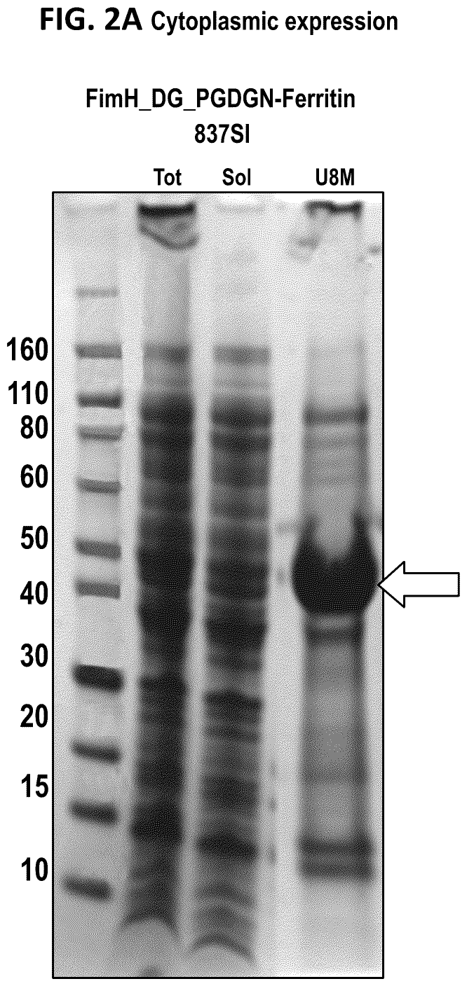

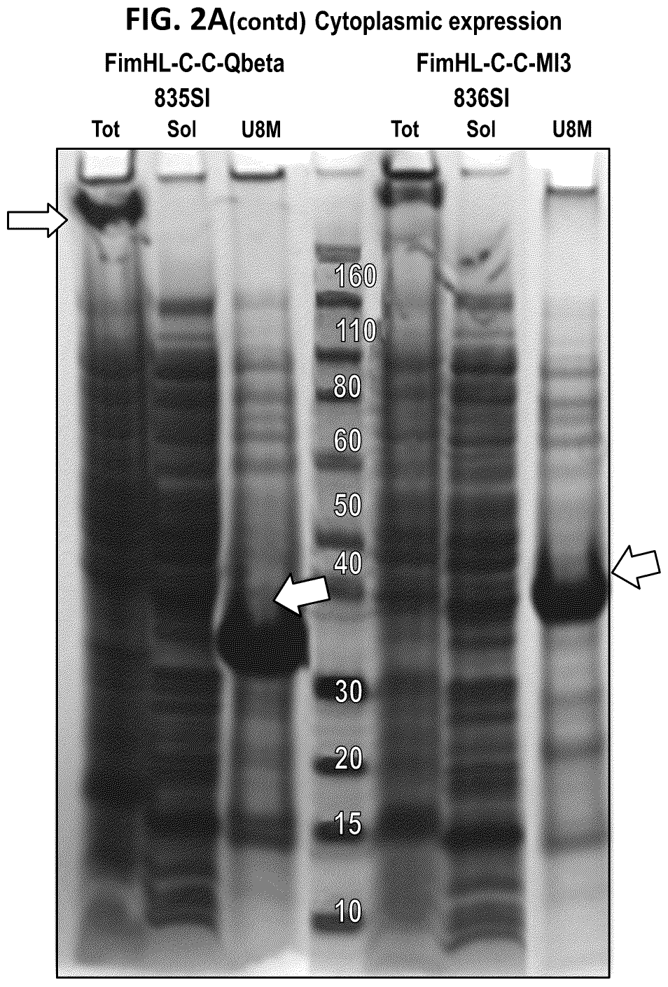

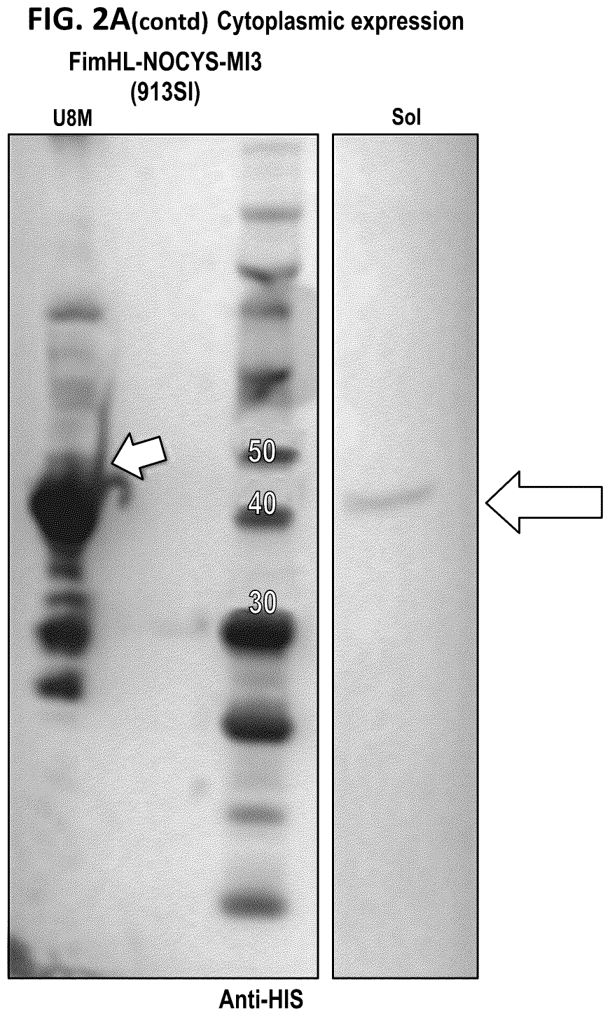

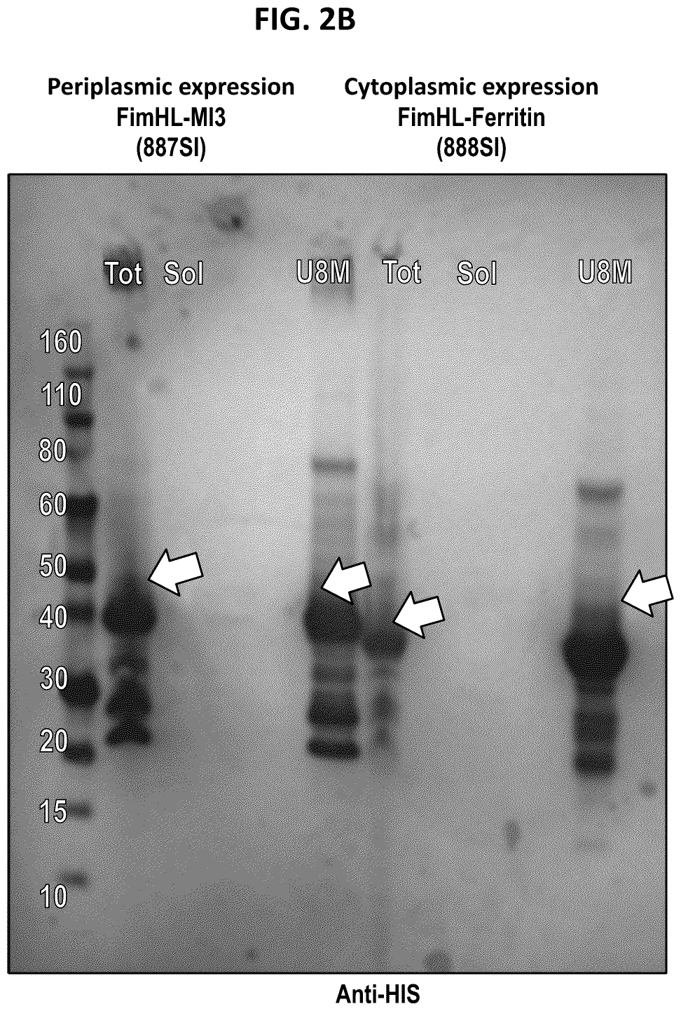

FIG. 2A and FIG. 2B. E. coli expression of FimH Nanoparticles results in inclusion Bodies formation:
A) FIG. 2A. SDS-PAGE analysis of E. coli cytoplasmic expression boiled and reduced samples of FimH_DG_(GSG4)-Ferritin, FimH_{L} cys-cys_QBeta, FimH_{L} cys-cys_ml3 and FimH_{L}-NOcys-M13. Constructs are expressed but can be detected only in the insoluble fraction (Urea 8M, U8M) and not in the soluble fraction (sol). The proteins cannot be detected in the total lysate fraction (Tot), due to insolubility; an accumulation of insoluble material can be detected in the upper part of the gel. Anti-His western blotting of E. coli cytoplasmic expression boiled and reduced samples of FimH_{L}-Nocys-MI3. The mutation of the internal disulphide bridge in FimH_{L} domain did not improve solubility as in the soluble fraction only a faint band can be detected.
B) SDS-PAGE analysis of E. coli periplasmic expression of FimH_{L}-M13 and cytoplasmic FimH_{L}-ferritin. Bands corresponding to FimH_{L}-M13 and Ferritin fusions were detected in the Total lysate and in the Insoluble fraction (U8M).

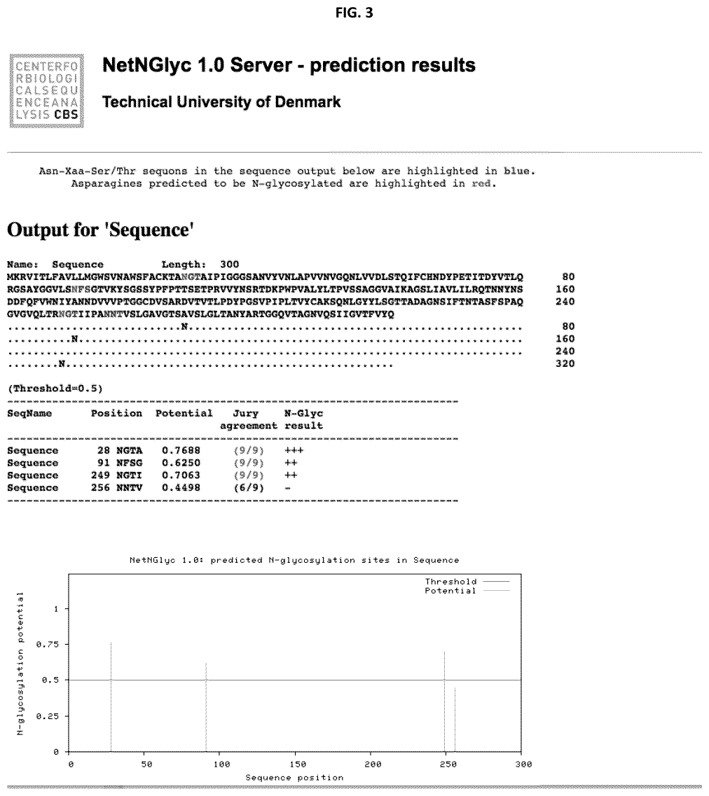

FIG. 3. Prediction of N-Glycosylation FimH sites using NetNGly prediction software.

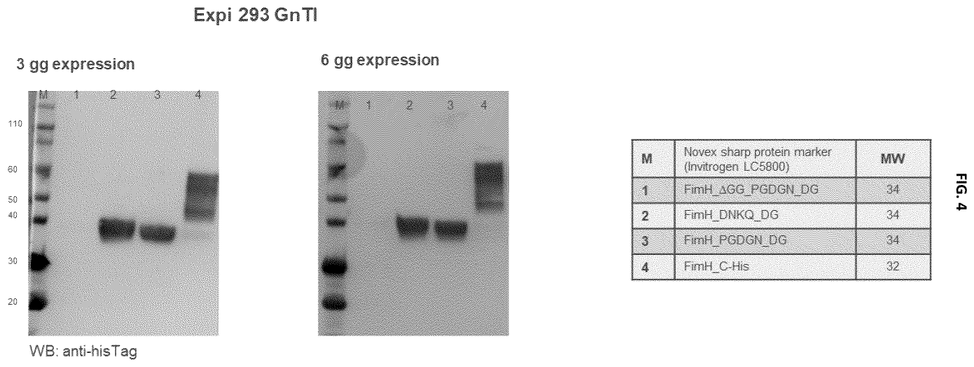

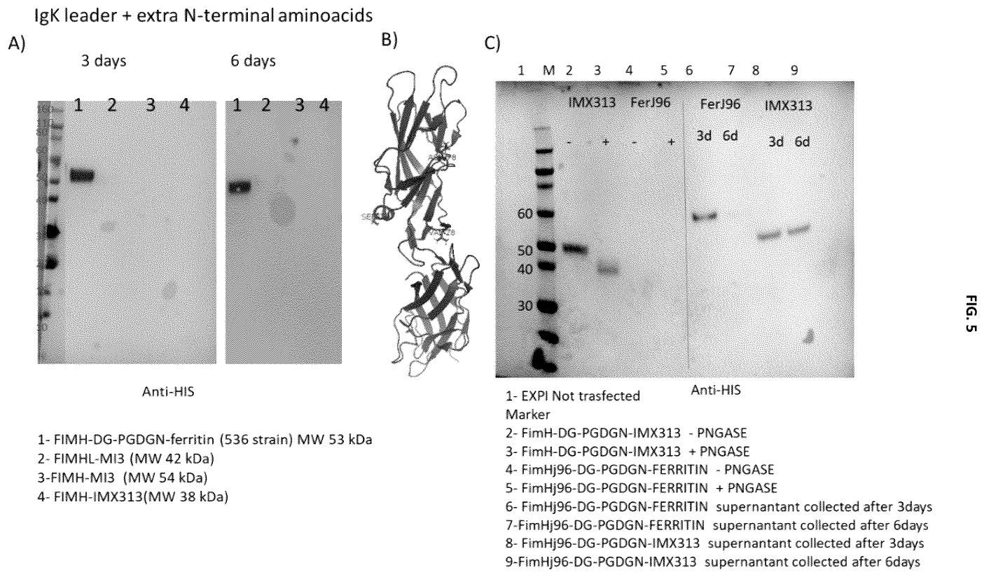

FIG. 4. Expression of Stabilized FimH constructs (FimH_ΔGG_PGDGN_DG: 930S1; FimH_DNKQ_DG: 931SI; FimH PGDGN_-DG: 932SI) and FimHC complex in mammalian cells.
FIG. 5. Western blot analysis of mammalian expressed constructs containing N-terminal extra amino acids.
(A) FIG. 5A: A band corresponding to FIMH nanoparticle was detected only for FIMH_DG_PGDGN-ferritin(995SI) after 3 days and 6 days post transfection.
(B) FIG. 5B: Cartoon representation of FimH from strain 536, the three different residues compared to J96 are highlighted and represented in sticks.
(C) FIG. 5C: PNGase treatment of FIMH_DG_PGDGN_IMX313 and FIMH_DG_PGDGN_ferritin from strain J96. After treatment a shift of the FIMH_DG_PGDGN_IMX313 at the correct MW was obtained, suggesting that the protein is glycosylated in mammalian cells. FIMH_DG_PGDGN_ferritin from strain J96, was not detected in both untreated and treated PNGase samples, suggesting that this protein degrades.

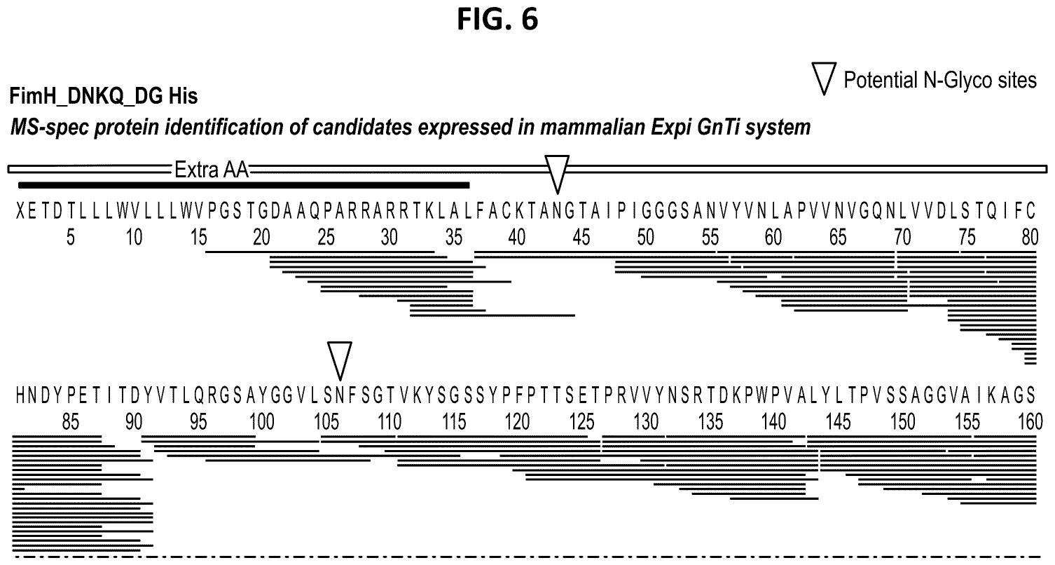

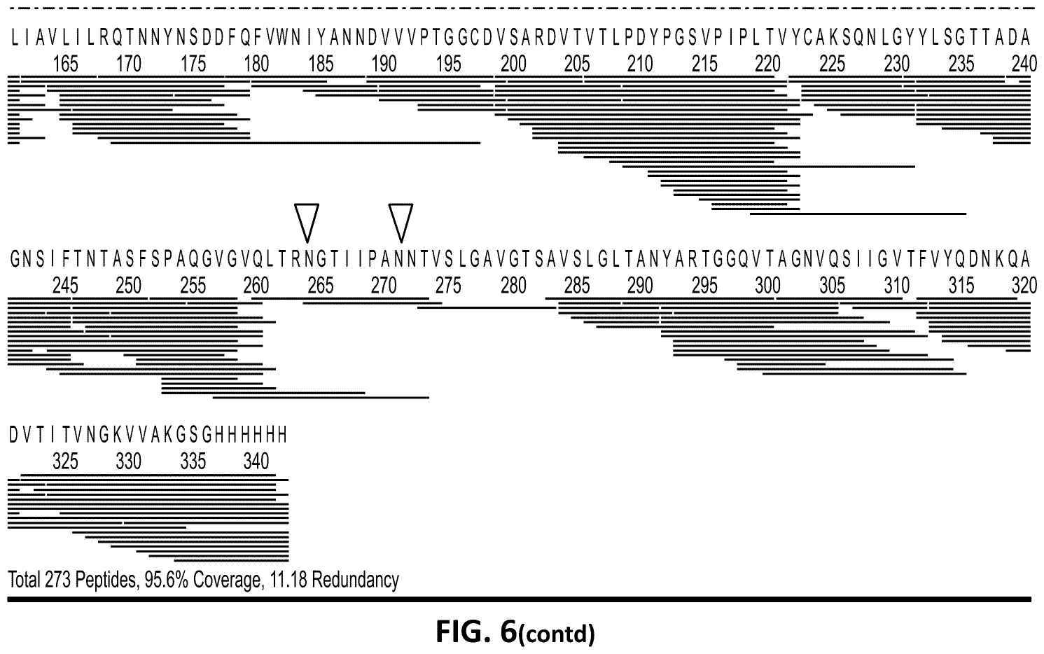

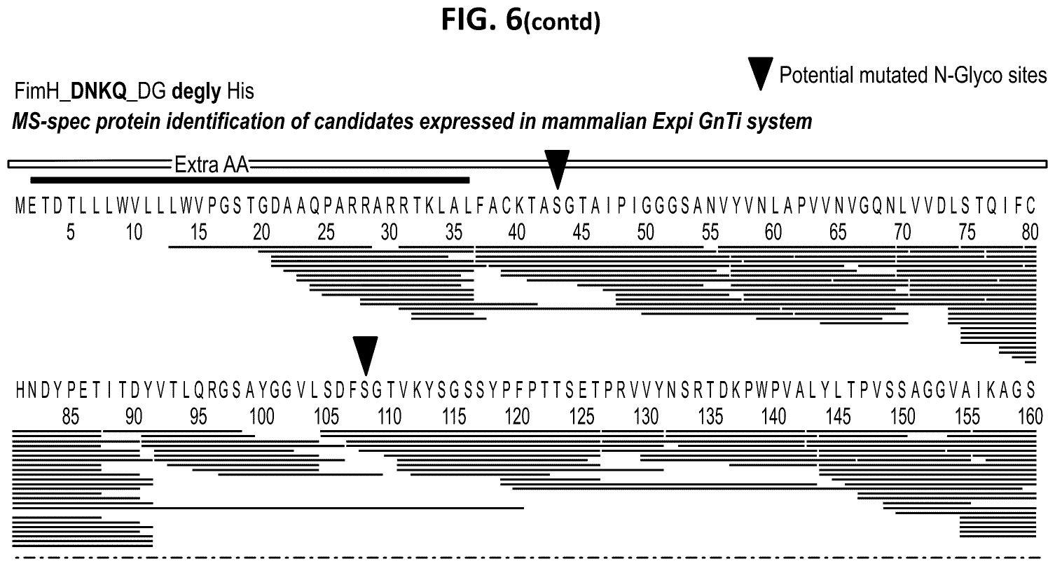

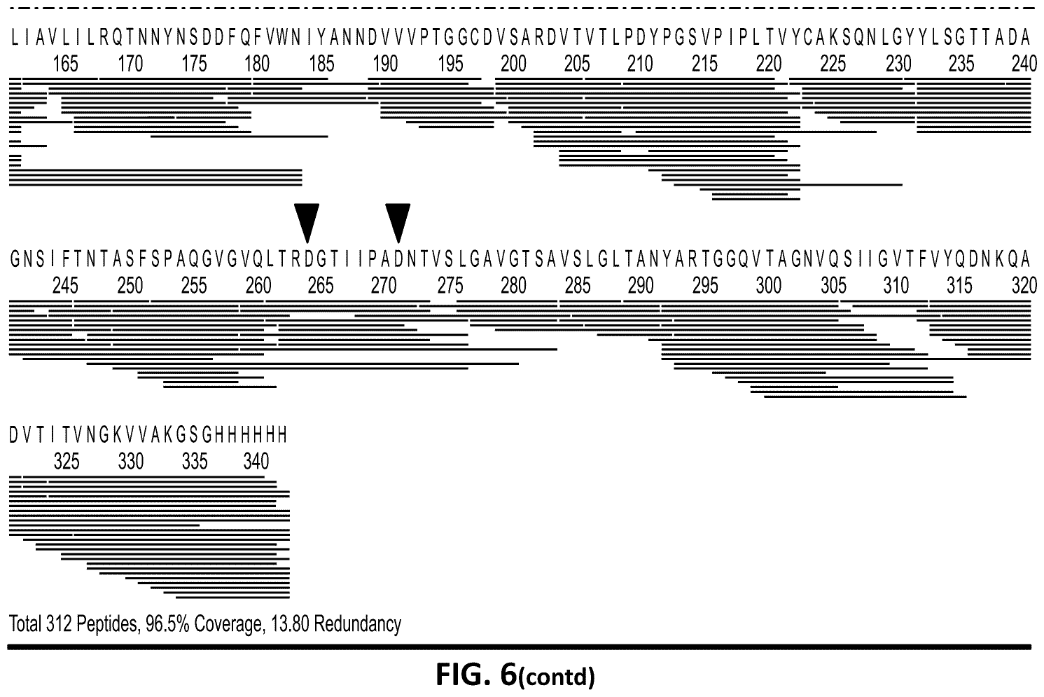

FIG. 6. MS-Spec peptide mapping.

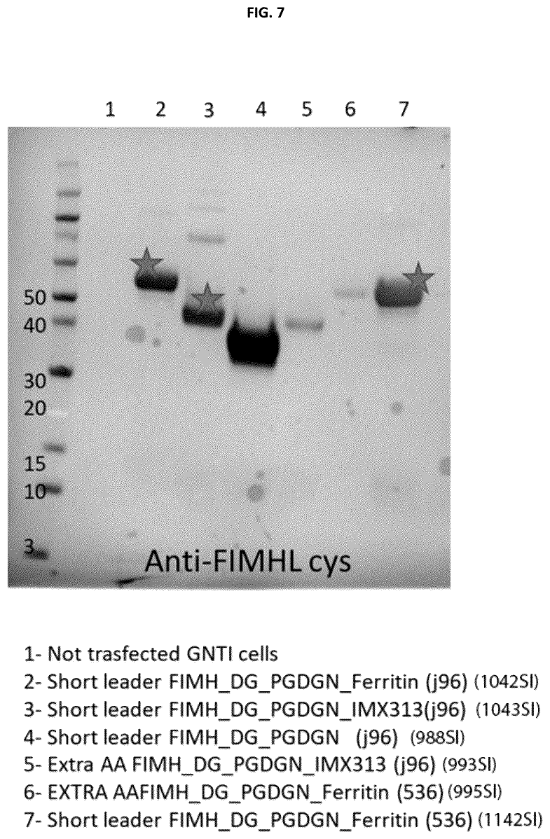

FIG. 7. Expression of candidates not containing extra N-terminal amino acids by Western blot.

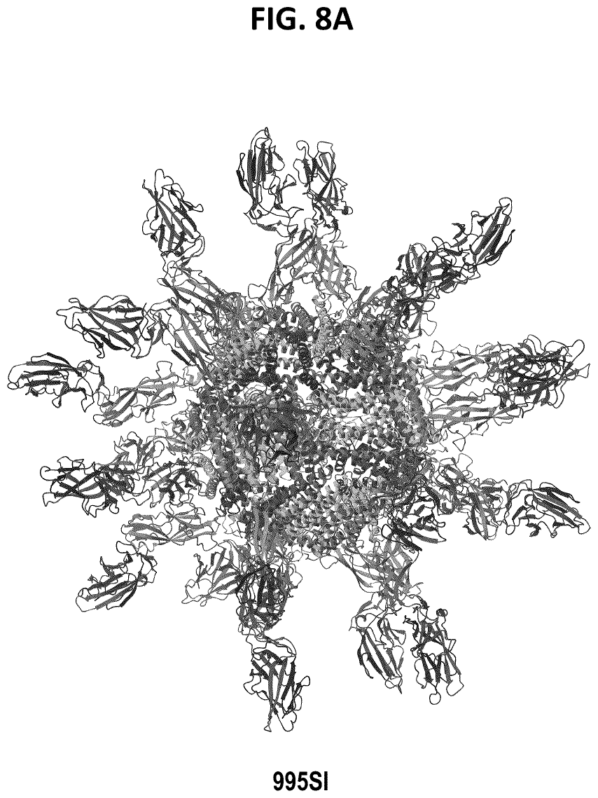

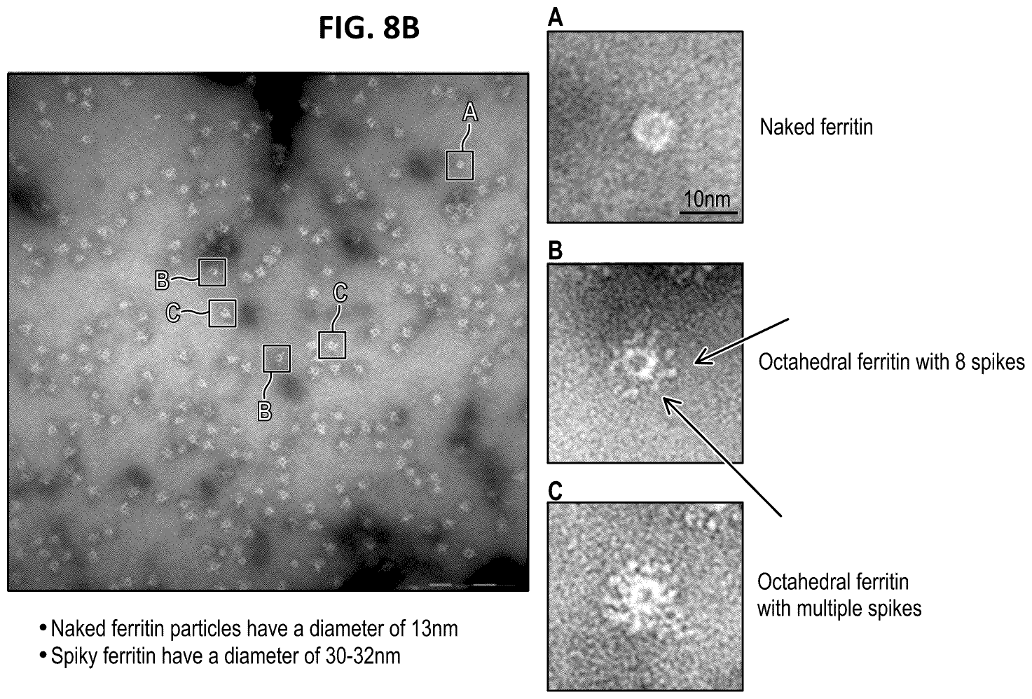

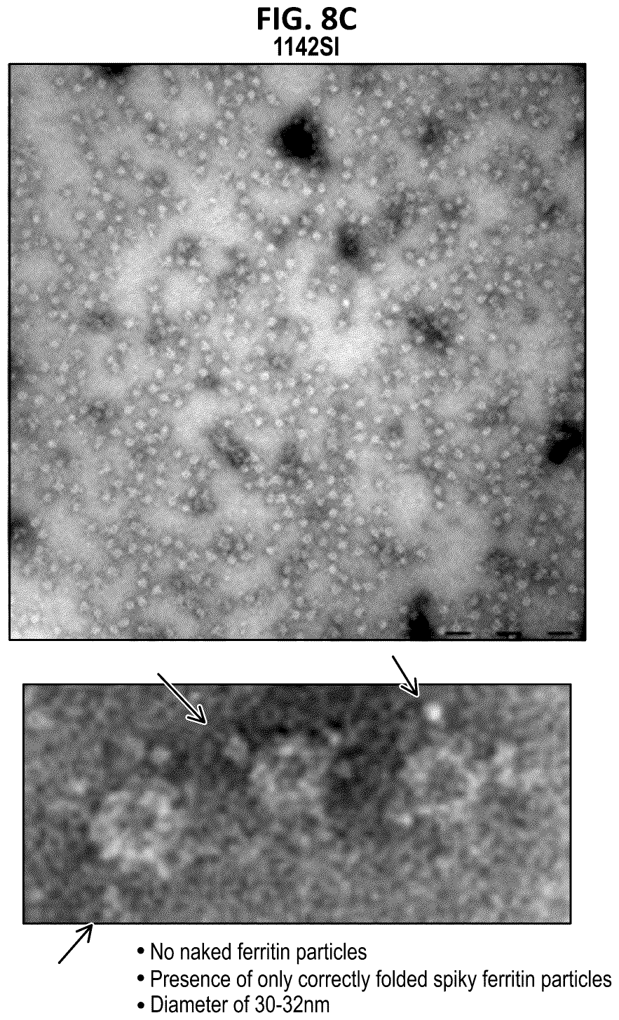

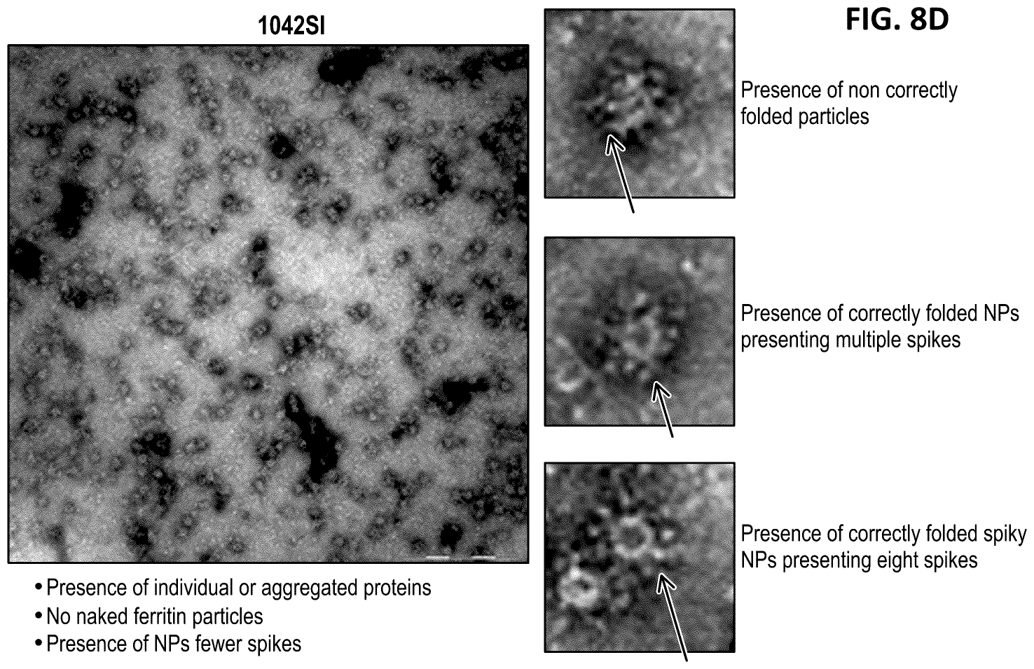

FIG. 8. Cryo-EM NS-EM (negative stain) of candidates with extra or without extra AA at N-Term.

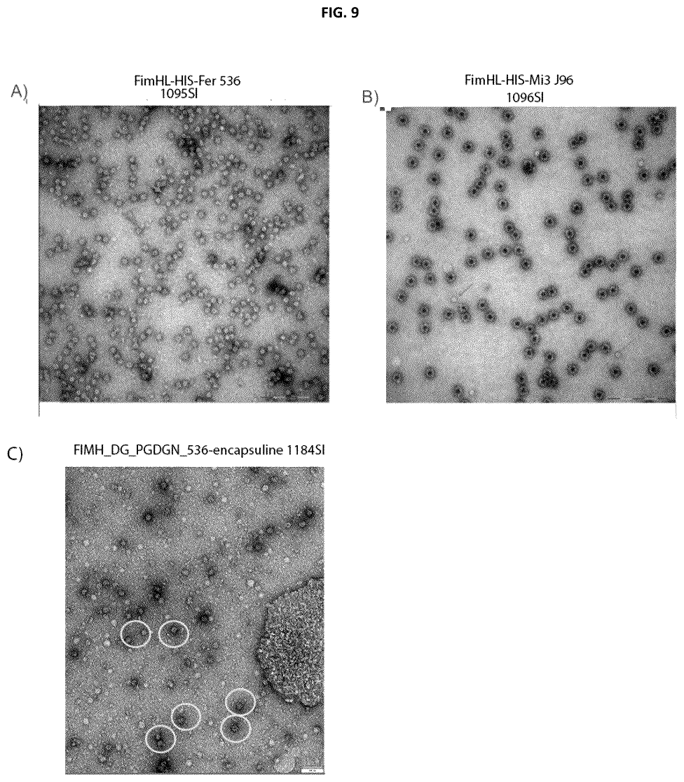

FIG. 9. Cryo-EM NS-EM (negative stain) of candidates without extra AA at N-Term.
A) FIG. 9A: Negative staining microscopy images of 109SSI FIMHL-ferritin (strain 536), NO extra amino acids.
B) FIG. 9B: Negative staining microscopy images of FIMHL-MI3 (strain J96) NO extra amino acids.
C) FIG. 9C: Negative staining microscopy images of 1184SI FIMH_DG_PGDGN_536-encapsuline, NO extra amino acids.

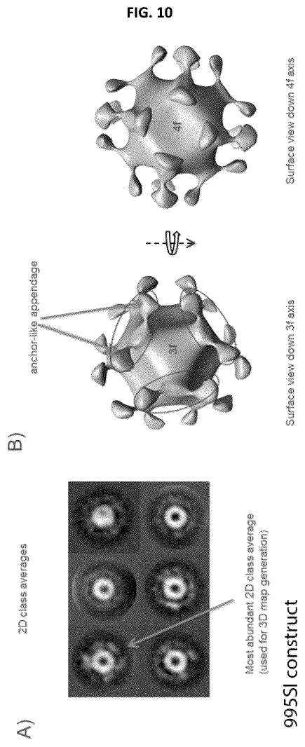

FIG. 10. 3D map shows the presence of three “anchor-like” appendages on the 3-fold axis.

FIG. 11. IgG titers measure by ELISA assay. Mice sera were tested at 21 (Post I, green), 35 (post II, blue), and 45 (post III, red) days post-vaccination. FimH_{L} produced from E. coli was used as ELISA plate coating.

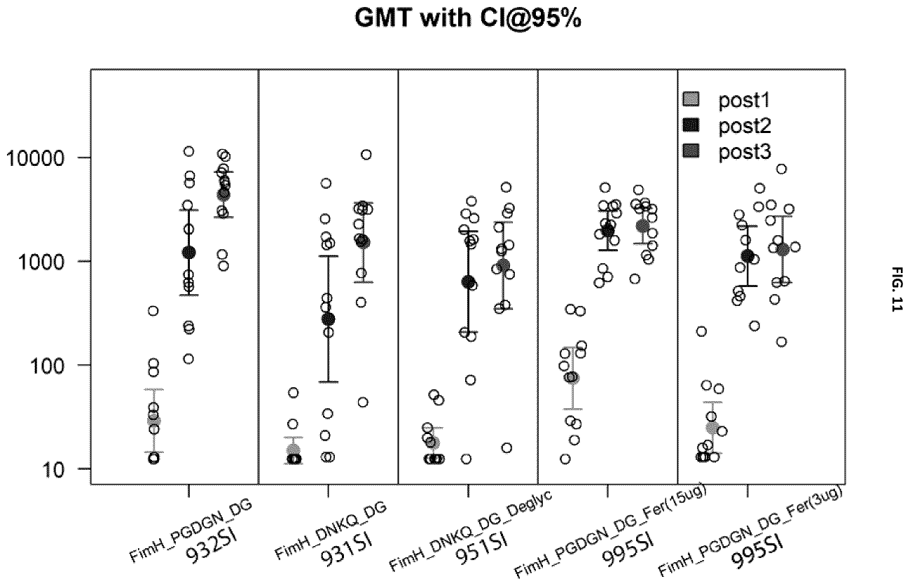

FIG. 12. Bacterial inhibition assay (BAI) on SV-HUC cells. Bacterial adhesion measured by microscopy analysis (OPERA Phenix) and SV-HUC (ATCC) cells were used. The Fluorescence Volume or Area of adherent bacteria (μm3 or 2) was used as readout. Pool of sera raised against recombinant protein FimHC, FimH_{L}-cys (purified from E. coli) were used as control. Pool of sera raised against recombinant protein purified from ExPIGnti expression mammalian system FimH_PGDGN_DG(932S1), FimH_DNKQ_DG(931S1), FimH_DNKQ_DG_Deglyc(951S1) and FimH_PGDGN_DG-Ferritin (995S1 were used to measure their ability to inhibit the bacterial binding to the SV-HUC cells. Pool of sera raised against AS01 were used as negative control.

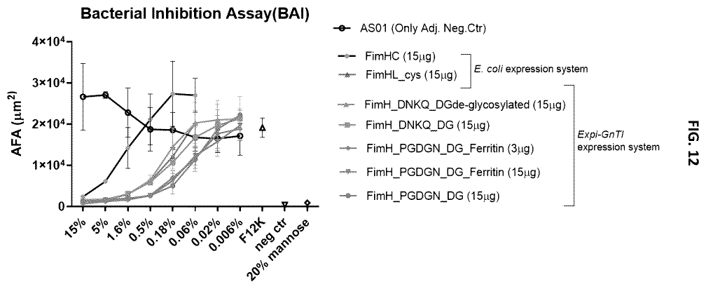

FIG. 13. Biochemical characterization of purified FimH_PGDGN_DG by SDS-PAGE, SE-UPLC and RP-UPLC.

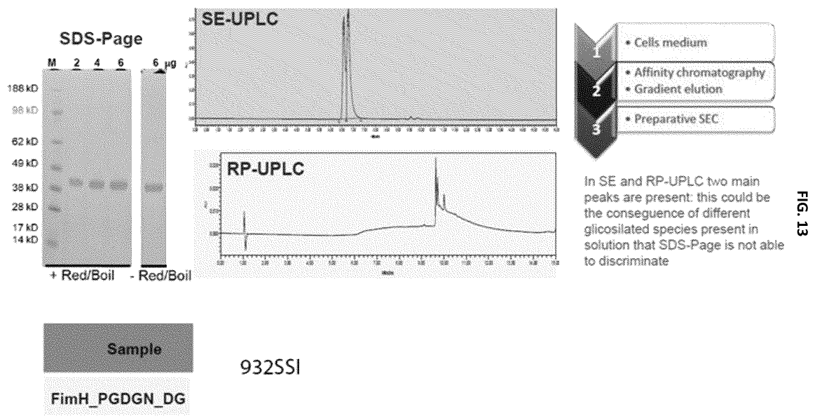

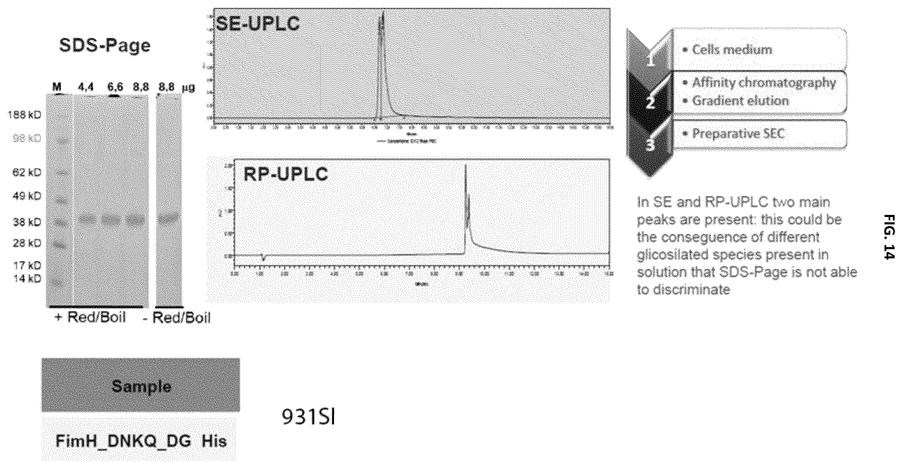

FIG. 14. Biochemical characterization of purified FimH_DNKQ_DG by SDS-PAGE, SE-UPLC and RP-UPLC.

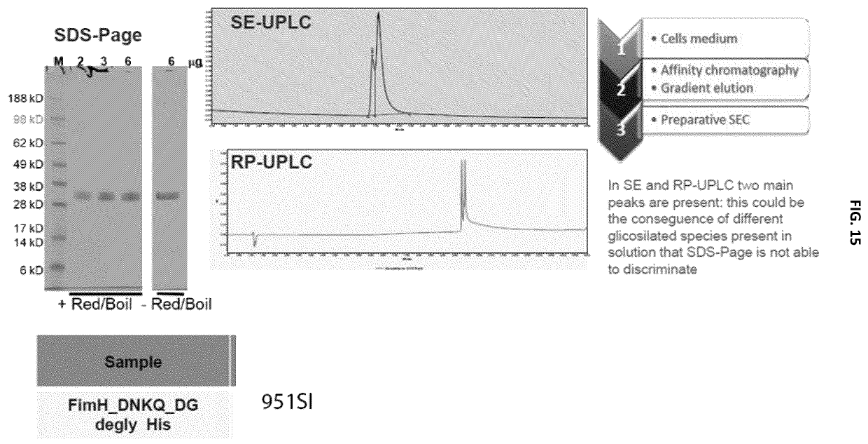

FIG. 15. Biochemical characterization of purified FimH_DNKQ_DG_deglycosylated by SDS-PAGE, SE-UPLC and RP-UPLC.

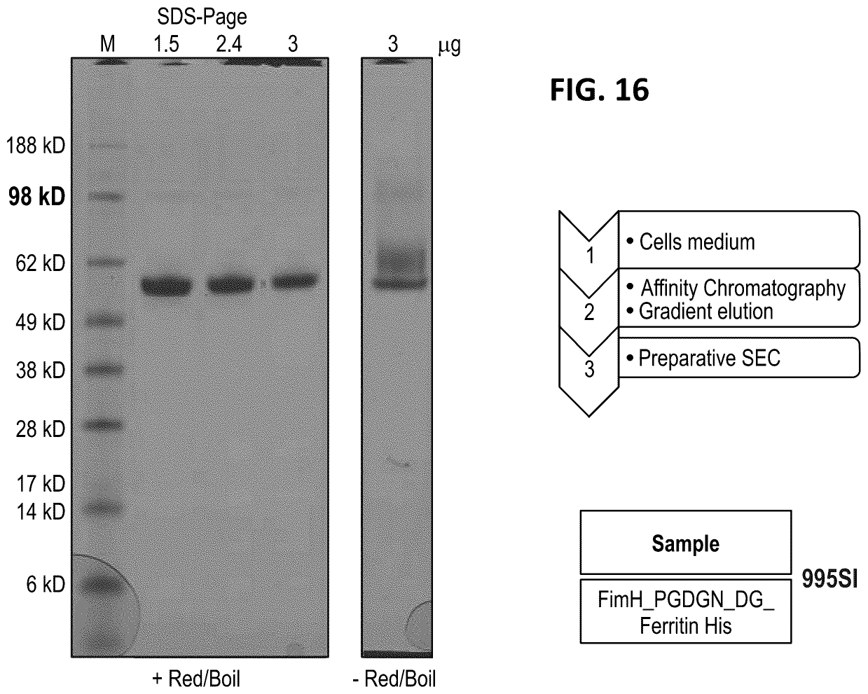

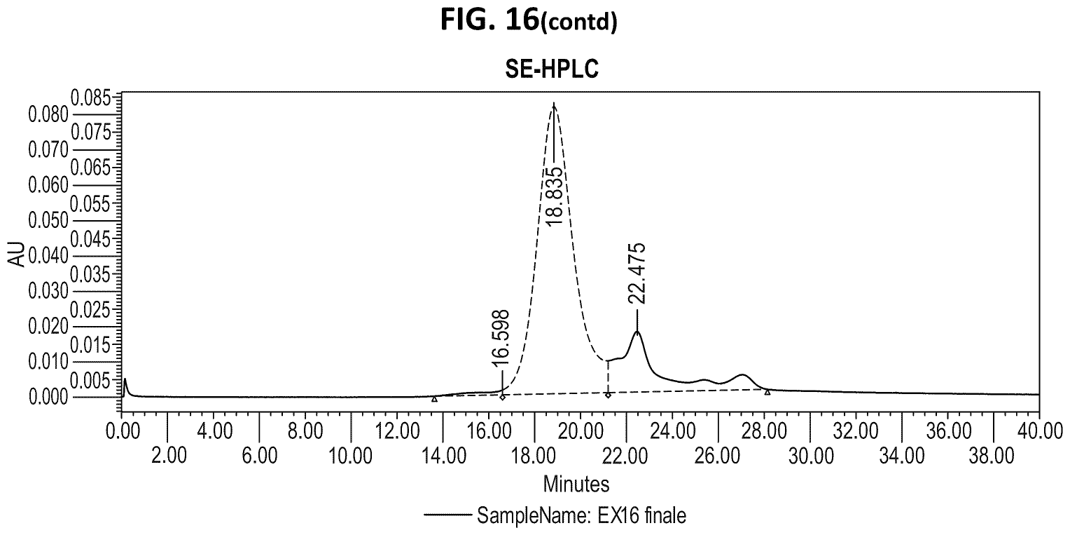

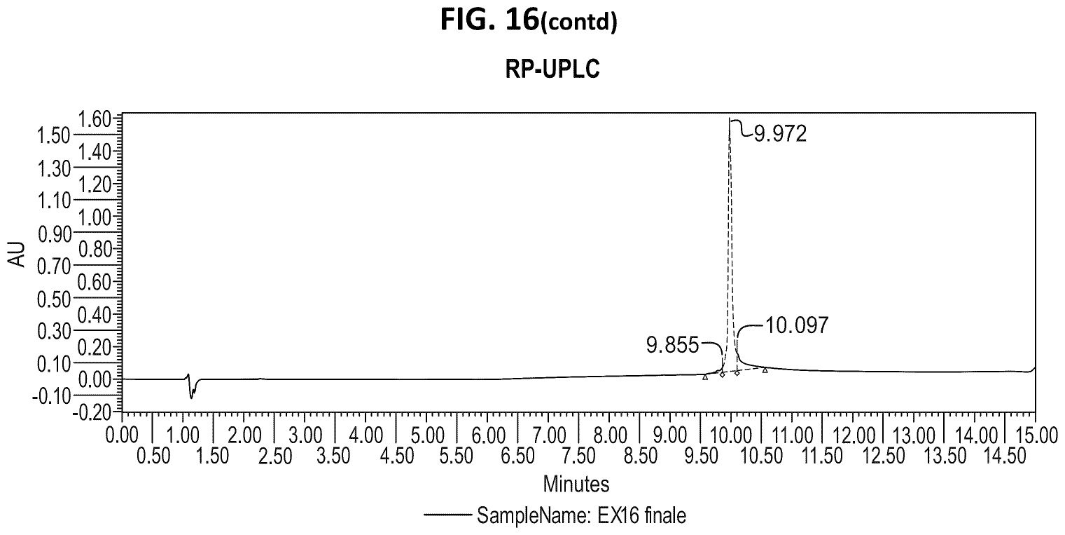

FIG. 16. Biochemical characterization of purified FIMH_DG_PGDGN_ferritin (sequence from UPEC 536 strain) with extra AA at N-Term.

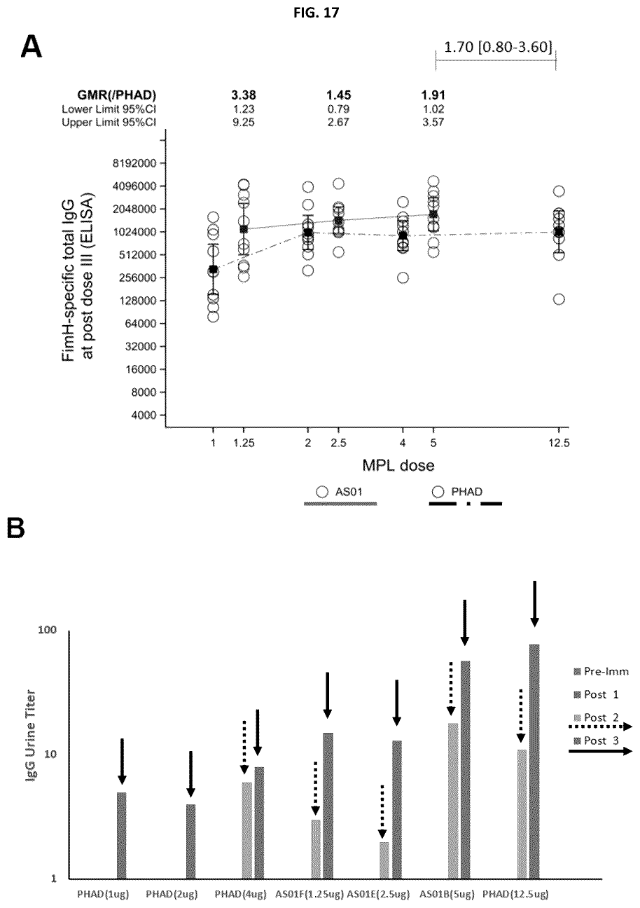

FIG. 17: FimH-specific total IgG (ELISA). FIG. 17 A) Anti-FimH IgG titers in mice sera at post 3 plotted as a function of MPL dose. FIG. 17B) Anti-FimH IgG titers in mice urine measured after 1st, 2nd and 3^{rd} vaccine dose. Pre-immune serum was used as negative control. FimHC was immunized in combination with the adjuvants using 1.6 μg of protein content.

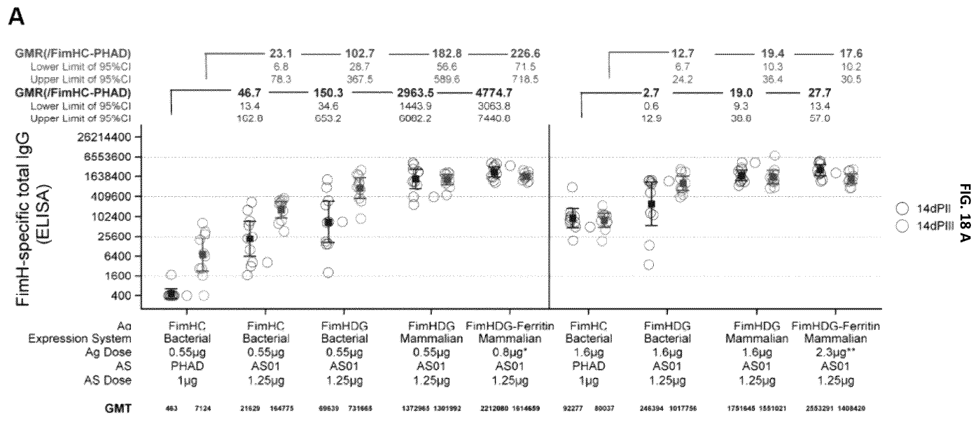

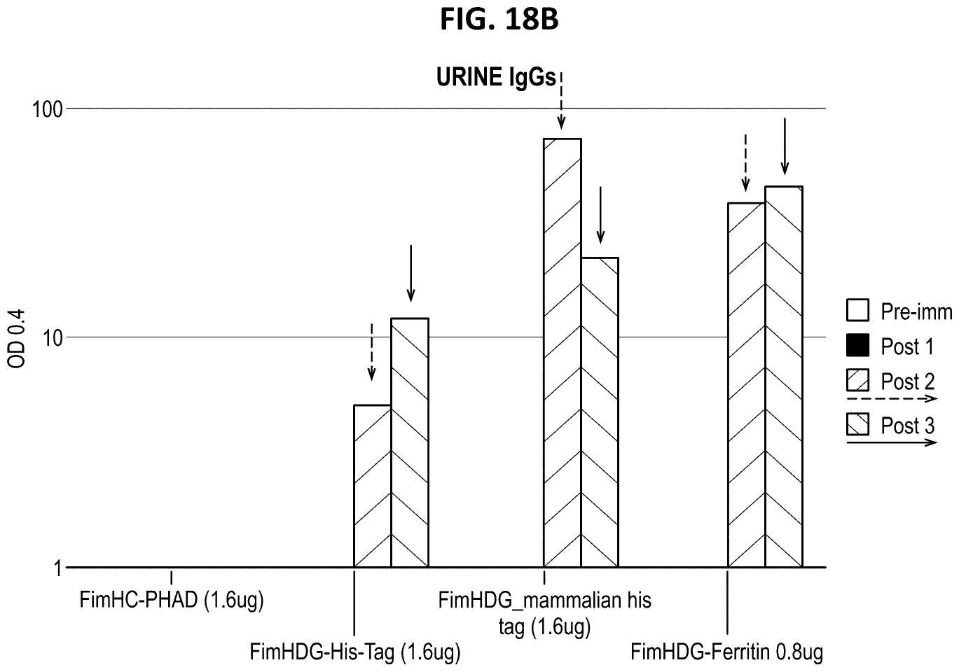

FIG. 18. FimH-specific total IgG (ELISA): comparison of bacterial and mammalian expression systems in sera and urine. FIG. 18 A) The antibody titers were assumed to be lognormally distributed and geometric mean titers (GMTs) and their two-sided 95% CIs were computed. For comparison of groups, an ANOVA model was fitted on log 10 titers with groups, timepoints and their interaction as fixed factors and a repeated statement for timepoints. Heterogeneity of variances was considered between groups. Geometric mean ratios and their 95% CIs were derived from this model. Antibodies response to each formulation was evaluated against FimHDG used for ELISA plate coating. All statistical analyses were performed using SAS 9.4. FIG. 18 B) FimH-specific total urine IgGs.

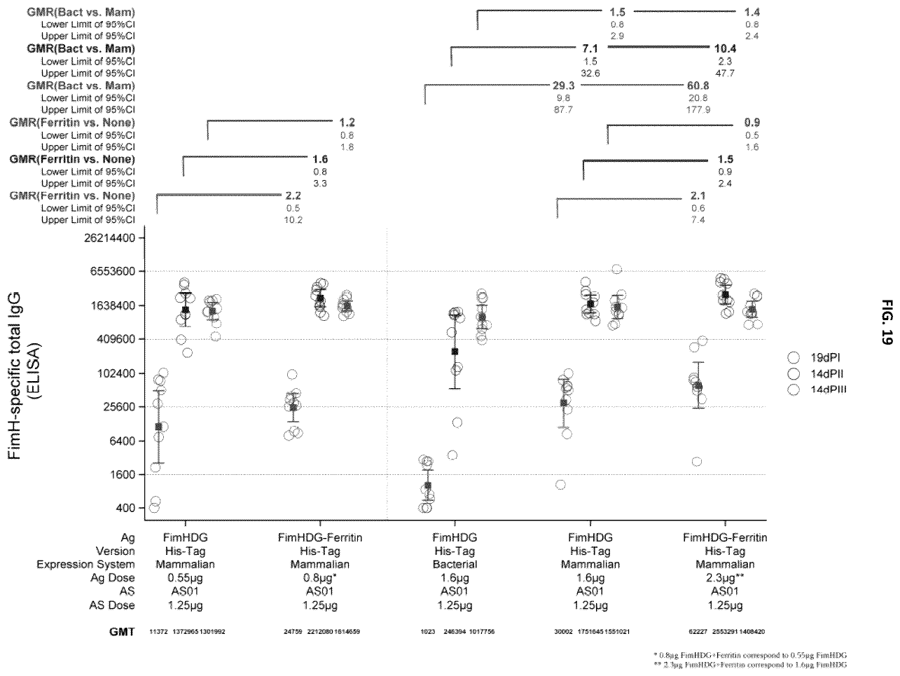

FIG. 19. FimH-specific total IgGs. ELISA post dose I results: The antibody titers were assumed to be lognormally distributed and geometric mean titers (GMTs) and their two-sided 95% CIs were computed. For comparison of groups, an ANOVA model was fitted on log 10 titers with groups, timepoints and their interaction as fixed factors and a repeated statement for timepoints. Heterogeneity of variances was considered between groups. Geometric mean ratios and their 95% CIs were derived from this model. Antibodies response to each formulation was evaluated against FimHDG used for ELISA plate coating. All statistical analyses were performed using SAS 9.4.

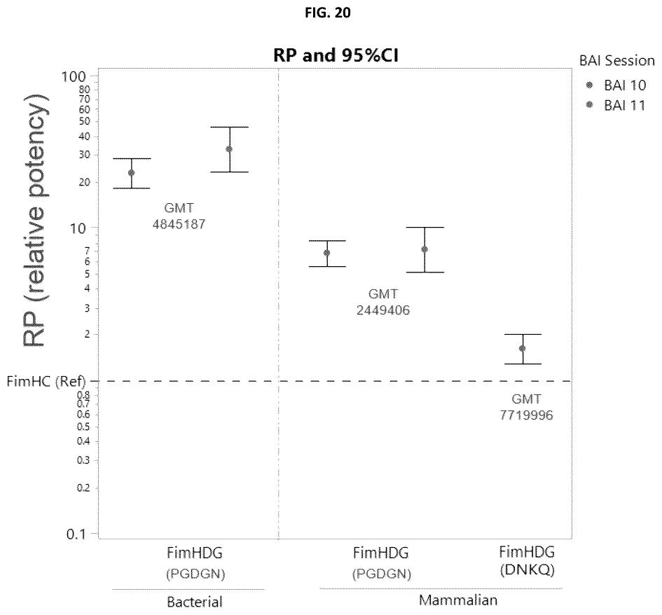

FIG. 20 FimH-DG elicits a functional immune response. Bacterial inhibition assay of selected constructs in comparison to FimHC. Relative potency is calculated as reported in the examples.

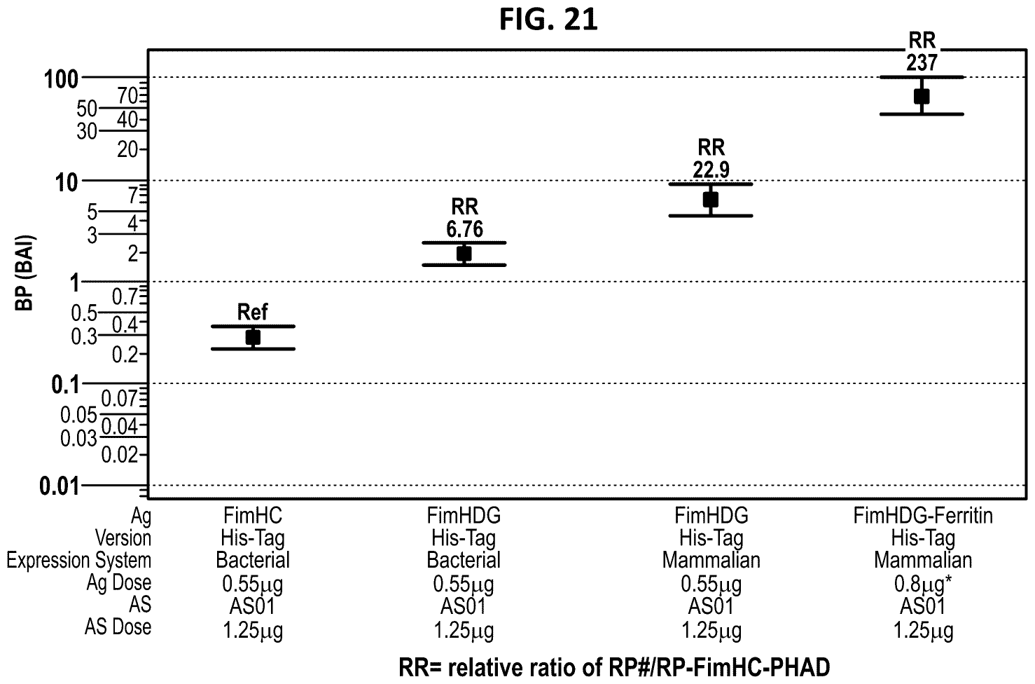

FIG. 21. Antibody ability of FimHDG antibodies to inhibit ExPEC adhesion using a bacterial inhibition assay (BAI). All candidates were formulated with AS01.

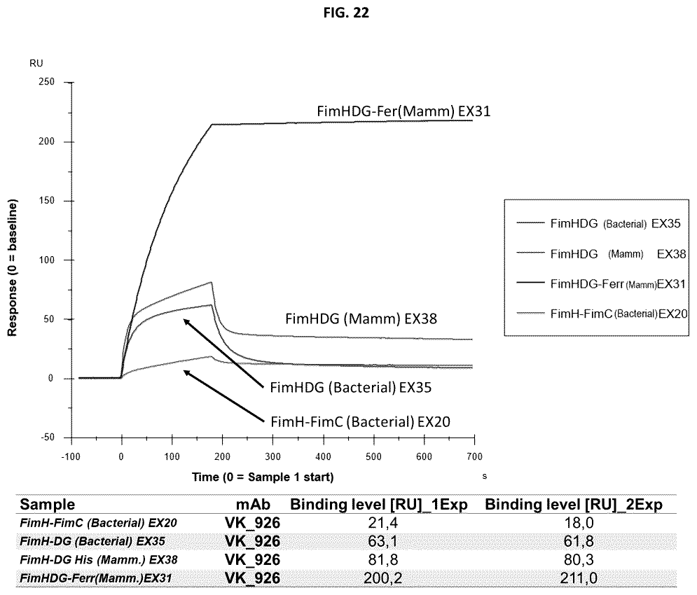

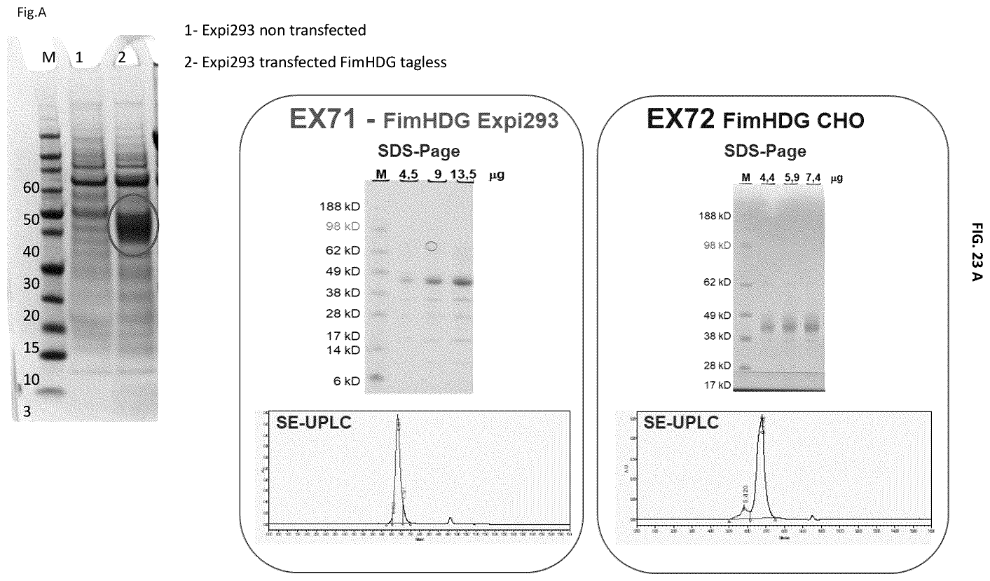

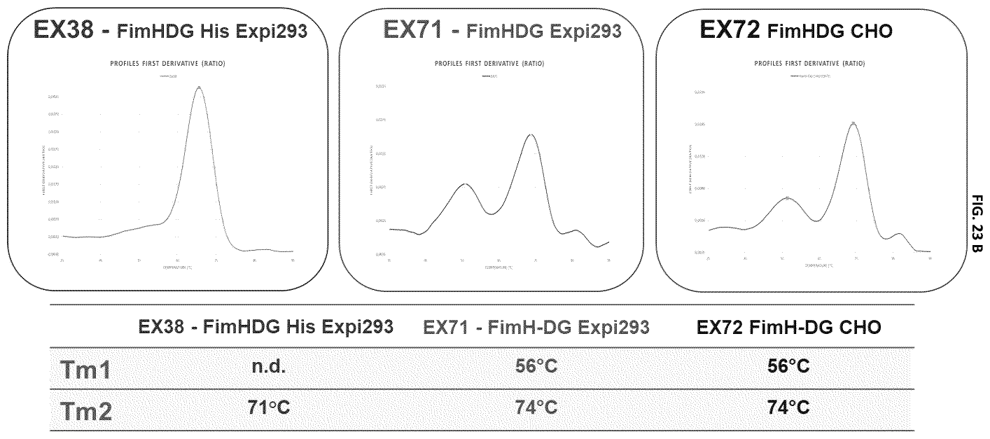

FIG. 22. SPR analysis of FimH samples and mAb926 interaction (Sensorgrams).

To study the interaction of FimH candidates and mAb926 a SPR analysis was performed resulting in a sensorgram representing a plot of response (ordinates) against time (abscissae) showing the progress of the interaction. Response was measured in Resonance Units (RU) which is directly proportional to the concentration of the molecules on the sensor chip surface. Each sensorgram is composed of two parts, corresponding to the association and dissociation phases of an interaction. The association is the first phase in a biomolecular interaction, during which the binding occurs, when analyte and ligand collide due to diffusion and when the collision has the correct orientation and enough energy. The dissociation is the phase in which the ligand-analyte complex dissociates; the profile of the dissociation can give information about the complex stability: the slower the dissociation, the higher the complex stability and vice versa.

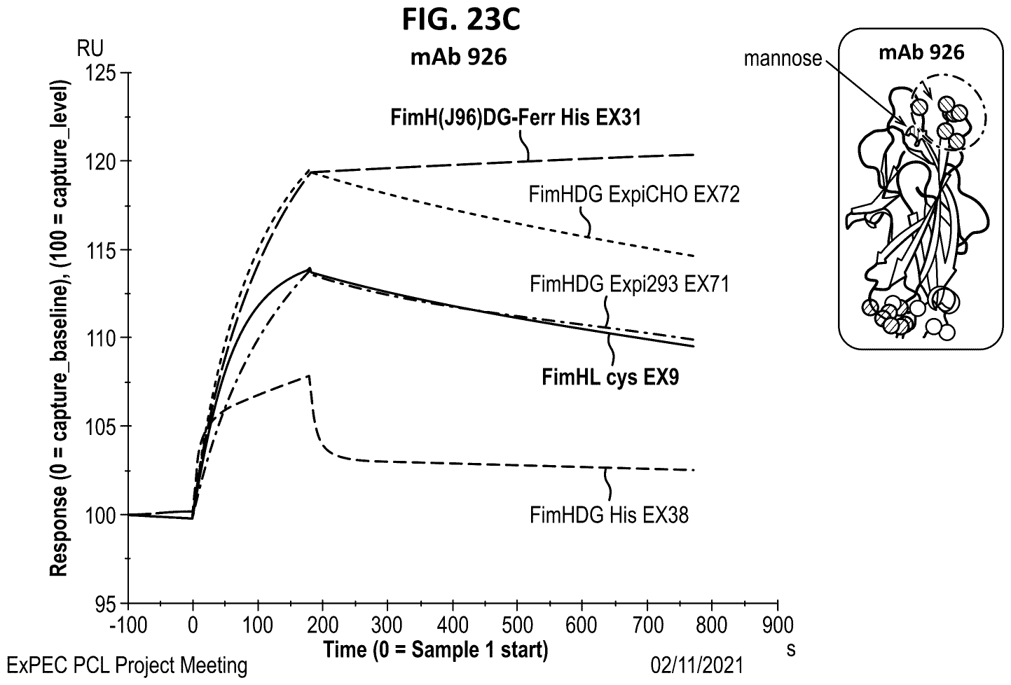

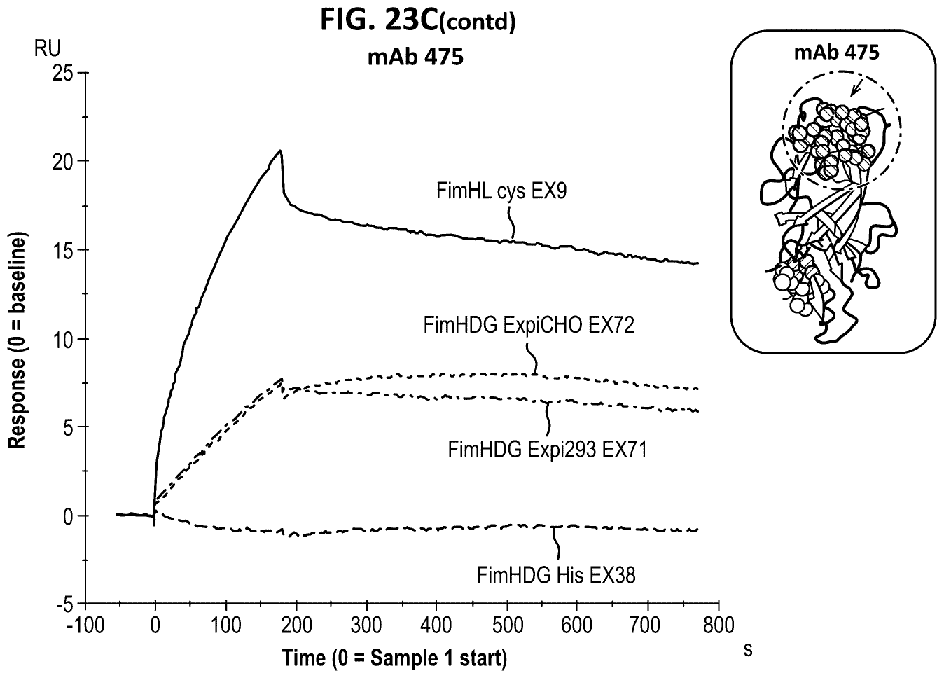

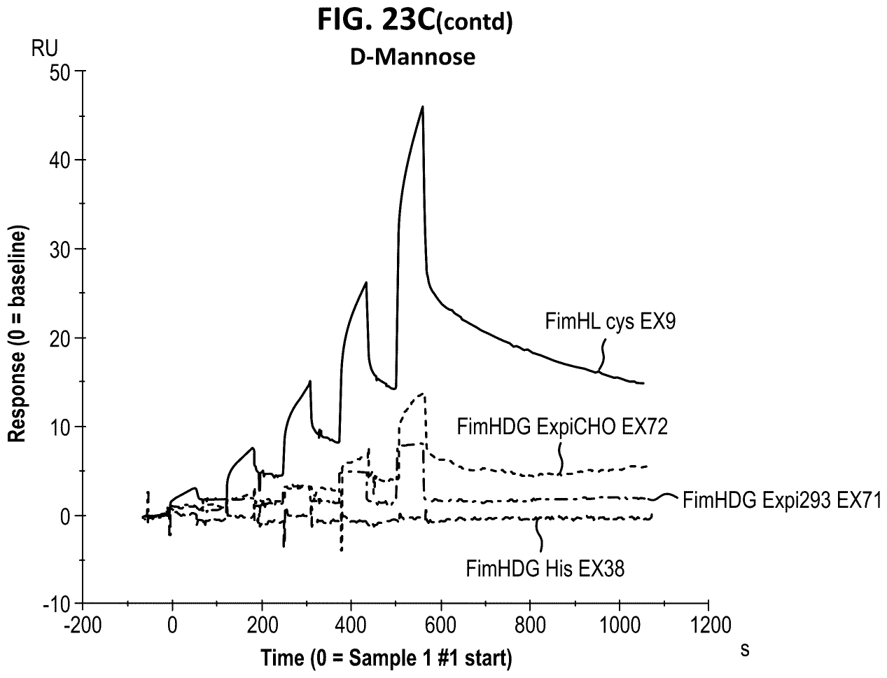

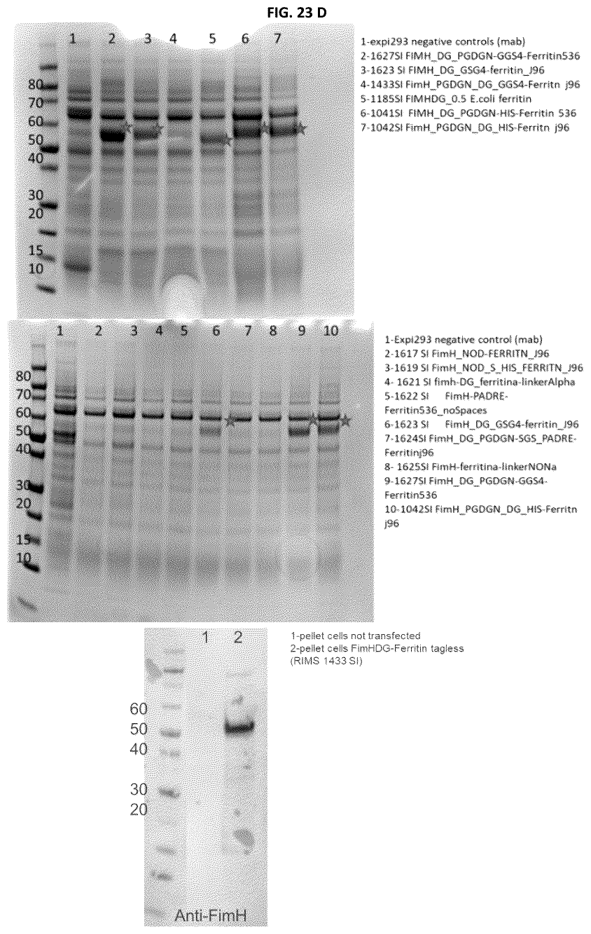

FIG. 23. FIG. 23 A: SDS page analysis of culture supernatant expressing FimHDG tagless in mammalian cells. SDS_Page analysis and SEC-UPLC analysis of purified FimHDG tagless from Expi293 cells and ExpiCHO cells. FIG. 23B: Nano-DSF profiles and melting temperatures values obtained for FimHDG tagless purified from Expi293 and ExpiCHO cells compared to the FimHDG containing the C-terminal His tag. FIG. 23 C: SPR binding analysis of mAbs 926 and 475 to FimHDG tagless compared to FimHDG His. SPR analysis of mannose binding to FimHDG tagless compared to FimHDG His. FIG. 23 D: SDS-Page analysis of supernatants of FimHDG-ferritin constructs containing different linkers and containing or not the initial Asp residue. Western blotting analysis of pellet from mammalian cells using anti-FimH specific mice serum.

FIG. 24. PROSS-based calculations of a symmetric monomer (relative to other 23 chains) in the octahedral E. coli nanoparticle (PDB 1EUM) to introduce stabilizing mutations with increased affinity or stability (bottom left of chart).

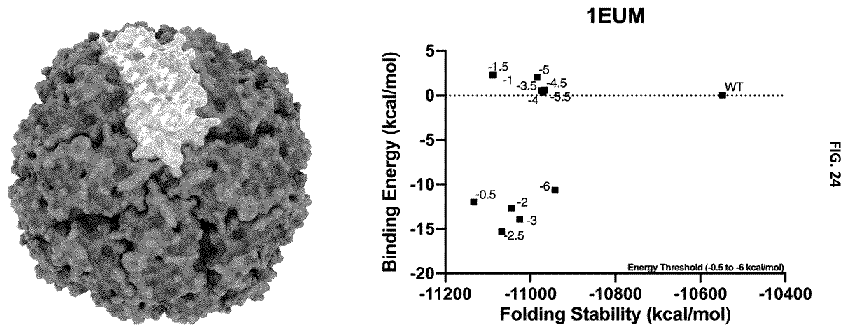

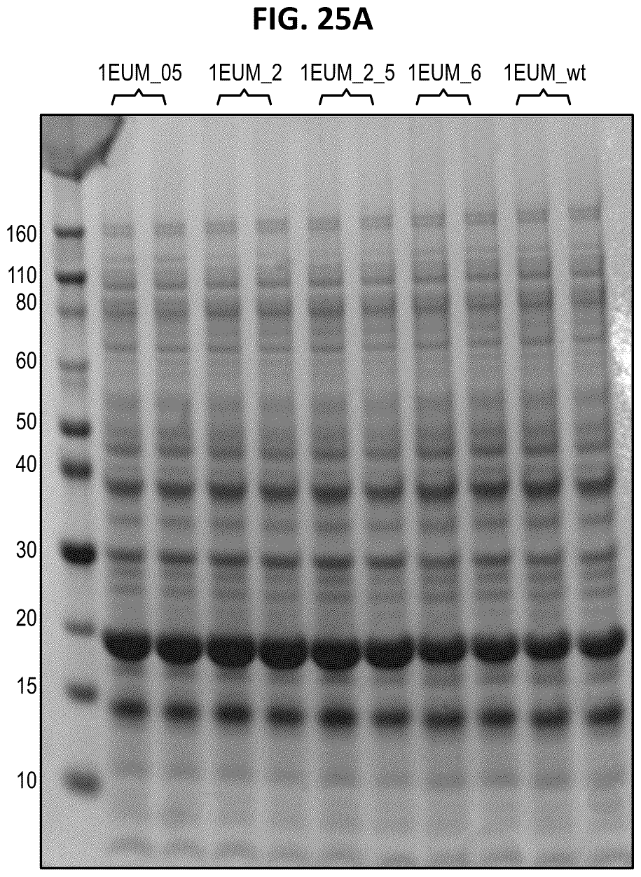

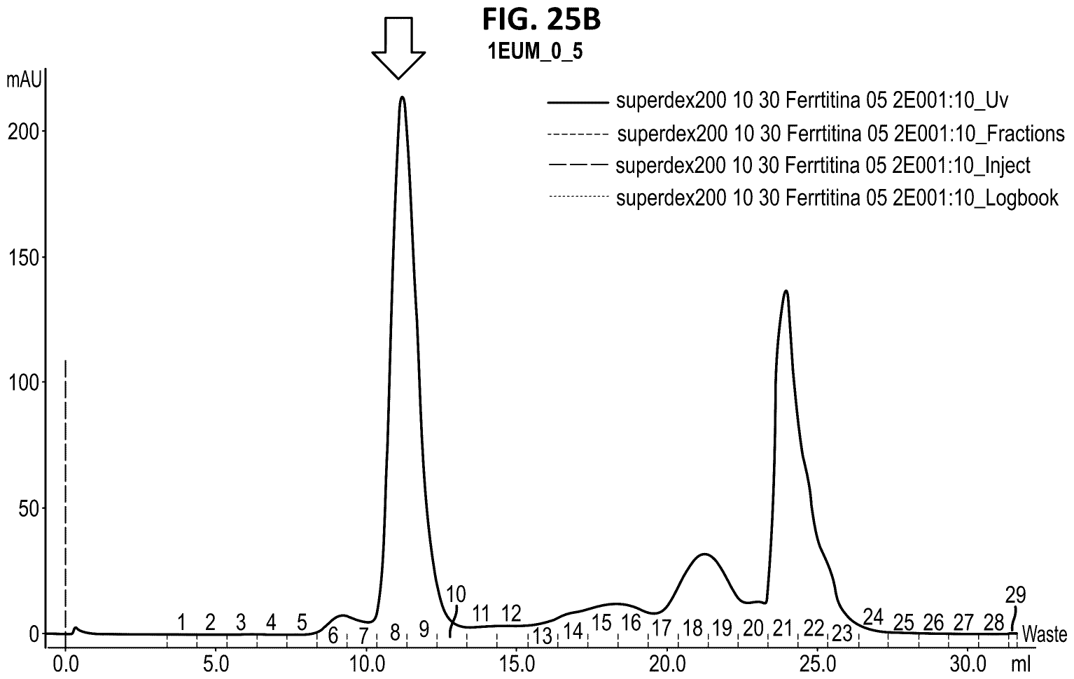

FIG. 25. FIG. 25 A: SDS page analysis of total (T) and soluble (S) extracts of WT E. coli ferritin and different mutants. FIG. 25 B SEC profile of mutant 0.5. All constructs had a profile with a strong peak (arrow) in the dead volume, which is compatible with the formation of a nanoparticle.

FIG. 26. NS-EM (negative stain) analysis of E. coli ferritin WT and different mutants (0.5, 2, 2.5, 6).

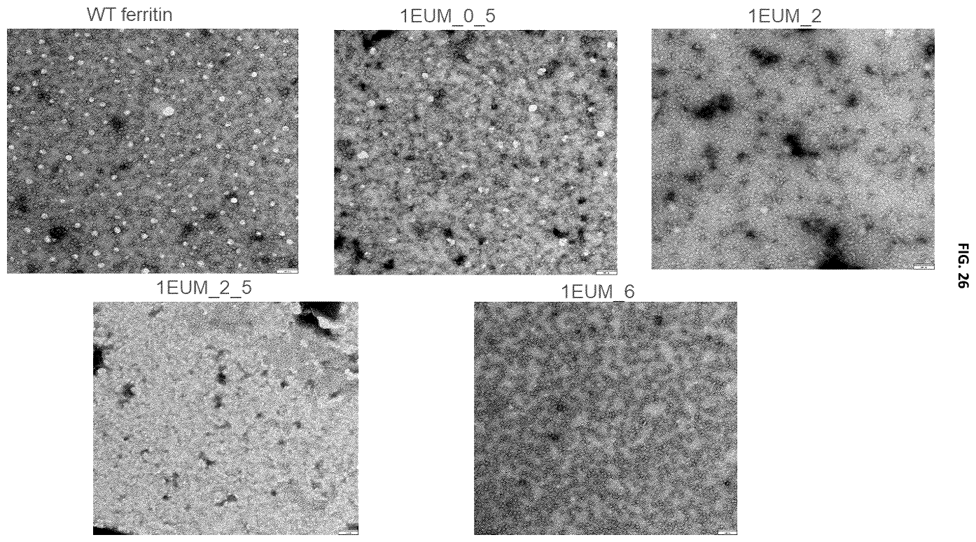

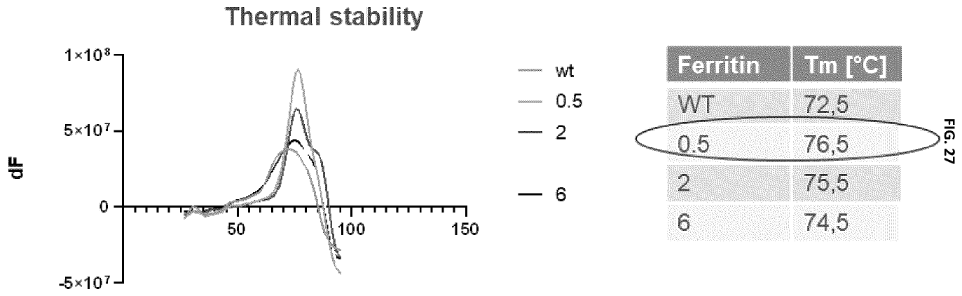

FIG. 27. Differential Scanning Fluorimetry analysis of ferritin constructs with thermal profiles. Graph on the left shows the derivate of fluorescence intensity vs. temperature. The circle, on the table on the right, indicates the mutant (0.5) with the highest Tₘ.

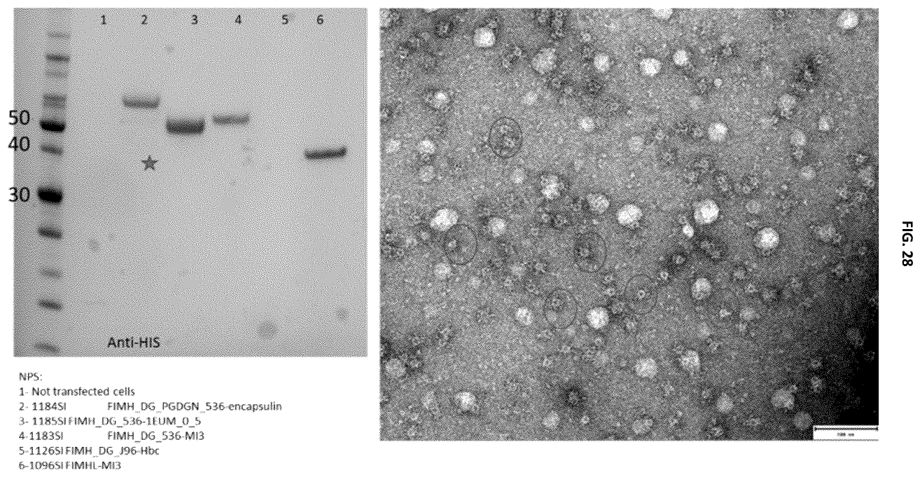

FIG. 28. On the left, Western Blotting analysis using anti-His antibody of supernatant expressing different nanoparticles constructs of FimH. The star indicates the E. coli nanoparticles FimHDG-ferritin (mutant 0.5).

On the right, TEM analysis show the presence of correctly formed ferritin nanoparticles.

### Examples

The inventors designed a stable un-complexed (in absence of FimC) variant of full-length FimH in which FimG donor strand peptide [SEQ ID NO: 5] was genetically fused through a linker of 4 or 5 residues (DNKQ [SEQ ID NO: 8] or PGDGN [SEQ ID NO: 7]) to the C-terminus of FimH_{P}, obtaining a “FimH_DG” protein with structural and functional properties of FimH in the assembled pilus. Linkers were designed by choosing highly polar charged residues (DNKQ) or inserting a Proline residue (PGDGN linker) as first residue of the linker that is predicted to support the turn in the secondary structure and to promote the correct protein architecture. In addition, a construct in which two glycines present in the linker that connects FimH_{L} to FimH_{P} were deleted, to further reduce the flexibility of FimH_{L} and reduce mannose binding (FIG. 1A).

Moreover, a nanoparticle design for FimH can be utilized to expose multiple copies of stabilized FimH and further increase its immunogenicity as enabler for a 1-2 dose vaccine.

Virus-like particle (VLP) and protein Nanoparticles (NPs) are display platforms for other antigens with potential to induce effective B- and T-cell responses. They have intrinsic ability to self-assemble into highly symmetric stable and organized structures. Several chimeric VLPs/NPs are under investigation in preclinical and clinical research worldwide. Particularly, ferritin scaffold has been genetically fused with viral hemagglutinin to obtain particles that were more immunogenic, in presence of adjuvant, at one dose compared to a seasonal flu vaccine (Nature 2013, 49, 104). The same approach has been used in preclinical research for many other antigens (Chen Y, et al. Vaccine. 2020 Jul. 31; 38(35):5647-5652). The challenge is not only to engineer a correctly assembled particle presenting the antigens of interest, but also to obtain it manufacturable and scalable. To explore the potential of self-assembling NPs and VLPs to display FimH candidates, different chimeras have been designed through genetic fusions and tested.

Helicobacter pylori ferritin nanoparticle is composed of 24 subunits, a total of eight trimers of the desired antigen can be display in the highly symmetrical octahedral cage structure of ferritin nanoparticles (FIG. 1B). Recently, protein i301 nanocage, a 60-mer NP based on the Thermotoga maritima 2-keto-3-deoxy-phosphogluconate (KDPG) aldolase have been computationally designed (Hsia Y, et al. Nature. 2016 Jul. 7; 535(7610):136-9.). 301 stability has been further improved by mutating two cysteines (m13) (Bruun TUJ, et al. ACS Nano. 2018 Sep. 25; 12(9):8855-8866) and by fusing SpyCatcher to the N-terminus of the protein.

We constructed recombinant plasmids to genetically fuse ferritin, m13 or encapsulin to FimH_DG_PGDGN stabilized antigen or FIMHL and FIMHLCys antigens. In order to separate the displayed antigen and the NP, a linker was added between the two sequences.

The linkers tested contain repetition of Gly and Ser residues but could also contain internal 8×His tag in order to allow protein purification. In order to increase protein expression and solubility in the E. coli cytoplasmic space of FimH NPs, FIMHL constructs mutated of the internal S_S bridge (C24SC65S) were also fused to Ferritin and mI3 and tested for expression and solubility.

Materials and Methods

Cloning and E. coli Expression

The FimH-NP bacterial constructs were synthesized by Geneart as DNA Strings and cloned directly into the pET15-tev, pET21 or pET22 (see table 1) with the Takara infusion cloning kit. Other constructs were purchased as synthetic genes from Geneart, with the protein of interest directly cloned into the expression vector (pTRC-HIS2A from Life Technologies). All synthetic genes were optimized for E. coli expression and contained N terminal, C-terminal or internal HIS tag to allow protein affinity purification. Proteins were expressed in BL21DE3T1r (NEB) or in T7shuffle express using HTMC medium and IPTG induction at 20° C. for 24 h.

After pellet recovery, it was resuspended in the lysis buffer cell lytic express (Merk) or B-Per solution (Pierce) for 1 h at 25° C. After centrifugation a visible inclusion bodies (IB) pellet was present, and it was resolubilized in Urea 8M (U8M). Protein expression and solubility was assessed by SDS-page of the samples collected from soluble fraction (S) and insoluble fraction (IB).

Recombinant Proteins Production in Mammalian Cells.

The FimH-NP mammalian constructs (See table 2) were synthesized by Geneart as synthetic genes in pCDNA3.1 or pCDNA3.4 (Life Technologies) vectors. All sequences were codon optimized for expression in mammalian cells and contained an N-terminal leader sequence for secretion into the cells medium. This sequence is the IgK murine leader sequence METDTLLLWVLLLWVPGSTGD [SEQ ID NO: 9], or the IgK murine leader sequence followed by 15 additional charged residues AAQPARRARRTKLAL [SEQ ID NO: 78]. (FIG. 1B) To produce recombinant FimH-NPs, the expression vectors were transfected into Expi293GNTI cells according to the manufacturer instructions (Life Technologies). The Expi293F GnTI- cell line is derived from engineered Expi293F cells that do not have N-acetylglucosaminyltransferase I (GnTI) activity and therefore lack complex N-glycans leading to homogeneously glycosylated recombinant proteins.

Briefly, 30 μg of pCDNA-FimH-NPs-expressing vectors were transfected into 30 ml culture containing 75×106 Expi293F cells using ExpiFectamine 293 Reagent. Cells were incubated at 37° C., 120 rpm, 8% CO2 and after 24 h, ExpiFectamine 293 Transfection enhancer 1 and 2 were added. Cells were further incubated at 37° C. for 144 h. Aliquots of cultures were harvested every 24 h and analyzed for NA expression by SDS-PAGE and Western Blot (WB). Seventy-two and 144 h after transfection, cell cultures were centrifuged at 1000 rpm for 7 min and the supernatants were harvested, pooled, clarified by centrifugation, filtered through a 0.22 μm filter, and stored at −20° C. until purification.

PNGase F Proteomics Grade, (P7367, sigma) was used to check glycosylation of mammalian expressed antigens according to manufacturer protocol.

Western blotting was performed using a standard protocol with anti-his-HRP antibodies by sigma diluted 1:1000 or with anti FimH_{L}-cys antibodies raised in mice using the bacterial FimH_{L}-cys purified protein and secondary anti-mouse-HRP antibodies.

Affinity chromatography with Ni2+ was used to purify NPs from culture supernatants. Fractions of interest were pooled and were concentrated by using 100 kDa cut-off spin concentrator (Millipore Amicon Ultra); sodium dodecyl sulphate-poly-acrylamide gel electrophoresis (SDS-PAGE) was performed to check protein purity. Recombinant FimH-NPs and FimH-DG antigens were purified by preparative size exclusion chromatography (SEC) equilibrate with PBS buffer.

All the collected fractions were checked for FimH-NPs or FimH-DG protein content by SDS-PAGE and interested fractions were pooled, filtered at 22 μm, aliquoted and stored at −20° C.

To assess protein size and purity, analytical SEC-HPLC and reverse phase RP-UPLC were performed. Moreover FimH-NPs were analysed by Dynamic light scattering in order to further determine the molecular weight and nanoparticle assembly and the proteins sequence identity was assessed by LC-MS.

Immunisation

Twelve CD1 mice (female) per groups were immunised with 15 micrograms of candidates expressed in mammalian or bacterial systems were adjuvanted with ASO1. All the mice were inoculated by subcutaneous injection (SC) with 200 μl (PBS dilution) of antigen mixture or adjuvant alone for three times. Blood was collected through the tail vein at 0 (preimmune), 21 (Post I), 35 (post II), and 45 or 49 (post III) days post-vaccination.

Analysis of FimH-Specific Antibody

Serum FimH-specific IgG were measured by enzyme linked immunosorbent assay (ELISA). Briefly, 96-well microtiter plates were coated with 100 μl antigen (1 μg/ml) to each well of a 96 well Nunc Maxsorp plate and incubated overnight at 4° C. 250 μl of (PVP) saturation buffer was added to each well and the plates incubated for 2 hours at 37° C. Wells were washed three times with PBT. Next, 100 μl of diluted sera were added to each well and the plates incubated for 2 hours at 37° C. Wells were washed three times with PBT. 100 μl of Alkaline phosphatase-conjugated secondary antibody serum diluted 1:2000 in dilution buffer were added to each well and the plates were incubated for 90 minutes at 37° C.

Wells were washed three times with PBT buffer. 100 μl of substrate p-nitrophenyl phosphate were added to each well and the plates were left at room temperature for 30 minutes. 100 μl 4N NaOH was added to each well and OD 405/620-630 nm was followed. The antibody titres were quantified as the dilution of serum that gives an absorbance of 0.4 OD using a multimode microplate reader.

BAI Assay

Bacteria (UT189 wt_mCherry clone2) cultivated in 3 passages of static liquid culture: the growth condition for inducing FimH expression. BAI assay performed with selected conditions: bacterial density of 0.012 OD/ml and incubation time of 30 min. Bacterial adhesion measured by microscopy analysis (OPERA Phenix). SV-HUC (ATCC) cells were cultivated in SV-HUC complete medium: F12K (Thermo Scientific) supplemented with 10% FBS and antibiotics. Pre-infection medium: complete media w/o antibiotics.

Tested sera (Heat Inactivated):
Serum ID

Anti-FimHL-cys
Anti AS01
FimH_PGDGN_DG
(mammalian)
FimH_DNKQ_DG
(mammalian)
FimH_DNKQ_DG Deglyc
(mammalian)
FimH_PGDGN_DG Fer, 15 ug
(mammalian)
FimH_PGDGN_DG Fer, 3 ug
(mammalian)

3×T75 flasks of SV-HUC cells (3×106 cells/ml, 95% vitality) were trypsinized (×5 min, at 37° C.). Cells were seeded in 96-well plates, seed 60 wells/plate with 3.5×104 cells/well (VF=200 ul/well) and incubated at 37° C., 5% CO2. Bacteria preparation consists in three passages of static liquid culture: UTI89 strains are inoculated in 20 ml LB (125-ml flask) from plate and are incubate at 37° C., O/N, in static condition. This dilution/incubation passage was repeated three times.

The medium of SV-HUC cells was exchanged with pre-infection medium w/o antibiotics (200 ul/well).

2× solutions of sera were prepared in U-bottom 96-well plate with F12K medium or F12K+ 10% FBS, as indicated below and further diluted with serial dilutions.

1 ml of Passage3 Bacterial culture UTI 89 mcherry Clone2 were transferred into single tubes and centrifuged at 4500×g for 5 min at room temperature. Bacteria were washed with PBS and pelleted. Finally, the bacterial pellet was resuspended at 0.5 OD600/ml with infection medium.

Infection was performed as follows: in each plate medium was sucked off and 50 ul/sample of 2× serum/mannose (20% D-(+)-Mannose) solutions or infection medium (ctrl positive & negative) were added followed by 50 ul/sample of 2× inoculum or infection medium (ctrl negative). Plates were incubated for 30 min and serum dilution from 15% to 0.06% was added. Plates were incubated at 37° C., 5% CO₂, for 30 min and the medium was removed and the plates wells were washed with PBS for three times. Bacteria were fixed using 4% formaldehyde (200 ul/well) solution. After incubation for 20 min, fixation solution was removed, and samples were washed 3 times with PBS (200 ul/well). DAPI (62248, ThermoScientific) solution was diluted 1:5000 in PBS and 100 ul were added to each well. Samples were incubated for 10 min at room temperature (in the dark). DAPI solution was removed, and PBS was added in each well (200 ul/well). Samples were stored at 4° C. in the dark and 3 h at RT before imaging with OPERA Phenix. Whole well area was acquired with a 10× air objective using the Alexafluor488 setting. For each field a Z-stack (4 planes) was acquired. Data were analysed with Harmony software. Total bacterial fluorescence area (single object ≤100 μm2) was calculated as a value of adherence.

Results

FimH Stabilized as Monomeric Antigens as Well as FimH-Stabilized Nanoparticles are Secreted as Soluble Proteins in Mammalian Expression System and can be Easily Purified by IMAC

As a first attempt, several FimH NPs constructs have been generated and tested in different conditions. T7 and pTac promoter, of pETvectors and pTrcHIs2A vector respectively have been used to test and solubility of the candidate antigens in E. coli. Moreover, both cytoplasmic and periplasmatic expression have been tested, as well as different E. coli strains optimized for disulphide bridges formation into the cytoplasmic space as the T7 Shuffle express. In order to increase protein expression and solubility in the E. coli cytoplasmic space of FimH NPs, FimHL constructs mutated of the internal S_S bridge were also fused to Ferritin and mI3 and tested for expression and solubility.

However, none of the constructs resulted in soluble protein expression suggesting that the E. coli expression system could be not optimal for obtaining FimH nanoparticles. The mutation of the internal disulphide bridge in FimHL domain did not improve significantly solubility as in the soluble fraction only a faint band was detected by western blotting analysis.

E. coli is a prokaryotic expression system that is strongly preferred for low-cost fermentation and easy process. However, the production of proteins by E. coli could results in recombinant proteins mainly expressed as inclusion bodies, which are insoluble and inactive, and may require complex refolding process in vitro (FIG. 2).

To overcome the problem of insolubility in E. coli, the inventors decided to switch to the mammalian EXPI293F expression system. First, the FimH sequence was analysed for N- and O-Glyco sites possibly responsible of glycosylation. FIG. 3 reports the position of putative N-glycosylation sites. O-Glyco site were not detected (data not shown).

In order to express bacterial protein in mammalian cells, reducing as much as possible the glycosylation which occurs in this system compared to the E. coli system, the inventors used a genetic mutated EXPI293F cell line called Expi293F GnTI (Thermofisher). This cell line is derived from engineered Expi293F cells but does not have N-acetylglucosaminyltransferase I (GnTI) activity and therefore lacks complex N-glycans leading to homogeneously glycosylated recombinant proteins.

The full length FimH proteins stabilized with the FimG donor strand (FimH-DG) from E. coli from strain 536 and/or J96 containing a secretion murine Ig-K chain leader sequence (plus extra amino acids at the N-terminus of the FimH_{L} domain (in some of the constructs; Table 1) alone or fused to protein NPs (ferritin, m13, IMX313, encapsulin and HBc) were used to transfect EXPI293 GNTI cells. The accumulation of secreted recombinant protein was characterized by measuring their expression in culture supernatants at 72 h and 144 h post-transfection by WB and SDS-PAGE. Both analyses revealed that FimH soluble expression could be obtained at high level for several constructs, while others could not be obtained. Expressed and soluble FimH-DG stabilized proteins and FimH-NPs containing the C-term 6×His tag or internal 8×His tag were purified from 72 h and 144 h pooled culture media using ion metal immobilized chromatography and preparative SEC chromatography. SDS-page analysis of proteins produced in mammalian expression system revealed that they run at a higher MW compared to the corresponding bacterial proteins, suggesting that they were glycosylated. Consequently, two constructs lacking the putative residues involved in N-glycosylation have been mutated, FimH_DNKQ_DG_deglyc and FimH_PGDGN_DG_deglyc, containing the extra amino acids N-Term and the following mutations N28S, N91D, N249D, N256D, were produced (Table 1).

Western blot analysis of supernatants of mammalian expressed constructs containing N-terminal extra amino acids revealed an expression band corresponding to FimH_DNKQ_DG, FimH_PGDGN_DG and FimHC complex. On the contrary, the FimH_ΔGG_PGDGN_DG (deletion of Gly resides connecting FimHL and FimHP) was not detected after 3 days and 6 days post-transfection (FIG. 4). Protein characterization of the purified products are reported in FIG. 13-16.

The constructs FimH_PGDGN_DG_Ferritin (strain 536; 995S1), containing N-terminal extra AA were successfully expressed and purified. On the contrary, all FimH non-FimG donor stand stabilized constructs (936Si)-FimH-IMX313 J96; (935Si)-FimH_mI3 j96; (929SI)-FimH_{L}-HIS-mI3 j96, all containing N-terminal extra amino acids, were not detected in culture supernatants.

PNGase treatment of FimH_DG_PGDGN_IMX313 and FimH_DG_PGDGN_ferritin from strain J96 revealed a shift of the FimH_DG_PGDGN_IMX313 at the correct MW in the treated sample, suggesting that the protein was glycosylated in mammalian cells. FIMH_DG_PGDGN_ferritin from strain J96, was not detected in both untreated and treated PNGase samples, suggesting that this protein was degraded. FimH_PGDGN_DG_Ferritin (strain J96) (1000S1), was not purified from collected supernatants from 3 days and 6 days, even if the protein was detected immediately after sample collection, due to degradation and this construct was not obtained (FIG. 5A-C).

In addition, the predicted N-glycosites reported in FIG. 3 were muted in serine or aspartic acid. The resulting FimH_DNKQ_DGDeglyc candidate showed a higher peptide mapping coverage in comparison to the WT sequence. This result indicates that a possible glycosylation might occur in correspondence of these specific mutated amino acids (FIG. 6)

Moreover, representative constructs reported in FIG. 7 were expressed removing the extra N-terminal amino acids (short leader). The FimH-DG_PDGDN_ferritin (strain 536, extra N-terminal AA) was obtained with a purity of 88% by RP-UPLC. (998SI) FinH_PGDGN_DG-HIS-IMX313 j96 was also well expressed and was successfully purified.

All these constructs were expressed as secreted soluble proteins in cells medium and were further purified as previously described. Western blotting analysis of supernatants with anti-FimHL-cys antibodies raised with bacterial stabilized protein recognized all mammalian expressed tested NPs (FIG. 7).

To confirm that FimH-Nps were correctly assembled, the purified proteins were examined by analytical SE-HPLC and DLS analysis. In SE-HPLC they eluted in a single large not-sharp peak. Based on the comparisons of the elution volumes (Ev) of ferritin NPs with the Ev of molecular weight (MW) standards run in the same conditions, the calculated MW of the FimH-DG-PGDGN-ferritin NPs is consistent with NPs composed by 24 subunits, as confirmed by DLS analysis.

The construct FimH-DG_PDGDN_ferritin SL (sequence from strain 536 or J96, lacking the extra N-terminal AA) resulted in highly expressed with final purity estimated by RP-HPLC.

FimHL-NPs constructs were also successfully purified, and the biochemical characterization confirmed the formation of NPs composed by 24 subunits, for (109551) FimHL-HIS-Fer 536 and by 60 subunits for (1096S1) FimHL-HIS-Mi3 J96.

Visualization of Generated FIMH-DG NPs

An additional confirmation that recombinant FimH-DG_PDGDN_ferritin extra AA (FIG. 9A-B) fusion protein FIMH_DG_PGDGN-HIS-Ferritin 536 short leader and FimH_PGDGN_DG_HIS-Ferritn j96 produced in mammalian expression system, form stable correctly assembled NPs was obtained by visualizing the purified proteins using negative stain electron microscopy TEM. As shown in FIG. 8B, (995S1) FimH_PGDGN_DG_Ferritin 536, containing N-terminal extra AA sample appeared as differentially oriented homogeneous population of octahedral particles decorated by spikes. Naked ferritin particles showed a diameter of 13 nm while spiky ferritin presented a diameter of 30-32 nm. The difference in diameter (8.5 nm) corresponds to the length of the FimH (calculated on the FimH model). Also, (1142S1) FimH_DG_PGDGN-HIS-Ferritin 536 is correctly folded and decorated by eight spikes of FimH trimers. No naked ferritin particles were present in the sample. Particles showed a diameter of 30-32 nm. (1042S) FimH_PGDGN_DG_HIS-Ferritn j96 sample presented a mixed population of NPs, with individual or aggregated proteins, correctly folded spiky NPs presenting eight spikes, presence of folded NPs with multiple spikes and NPs non correctly folded. No naked ferritin particles were detected (FIG. 8D).

Cryo-EM NS-EM (negative stain) of (1095S1) FimHL-HIS-Fer 536 and (1096Si) FimHL (J96)-mI3-his showed that that NPs expressed in mammalian system were fully assembled. FimHL-HIS-Mi3 J96(1096SI) presented correctly folded nanoparticles with an icosahedral shape, highly symmetrical of 40 nm and decorated by spikes, with few aggregates (FIG. 9A and FIG. 9B). In addition, 1185SI and 1184SI FIMH_DG_PGDGN_536-encapsuline both short leader at the N-term were correctly assembled (FIGS. 9 C and D). The constructs containing the stabilized FimH fused to IMX313 were also successfully purified (1043S1) FimH_DG_PGDGN_IMX313_HIS J96 and (998SI) FimH_PGDGN_DG-HIS-IMX313 j96 and the biochemical characterization confirmed the formation of high molecular weight (HMW) species. However, TEM analysis of these constructs showed the presence of only aggregated protein (data not shown).

Structural Features in 3D Reconstructions of Recombinant FimfH-DG-Ferritin NPs

Single particle reconstruction method was applied to TEM images in order to generate the three-dimensional structure of the assembled octahedral particles of (995SI) FimH_PGDGN_DG_Ferritin (FimH sequence from strain 536). Single boxed FimH-DG_PDGDN_ferritin nanoparticle (box size 64×64 pixel) were firstly band pass filtered in order to increase the signal-to noise ratio, then rotationally and translationally aligned, and finally centred before undergoing MSA for classification. FIG. 10A shows a selection of FimH-DG_PDGDN_ferritin most abundant 2D class averages, representative of the different orientations of the particle on the carbon film support. The 3D-EM structure (FIG. 10B) of the soluble FimH-DG_PDGDN_ferritin generated confirmed this structure to be composed of a highly symmetrical octahedral cage structure with the presence of three “anchor-like” appendages on the 3 fold.

FIH-DG Stabilized Proteins and FIMH-DG_PGDGN-Eerritin NPs are Highly Immunogenic in Mice

To assess the immunogenicity of candidates expressed in the mammalian system (FimH_PGDGN_DG, FimH_DNKQ_DG, FimH_DNKQ_DGDeglyc and FimH_PGDGN_DG_Ferritin), single sera from immunised mice were analysed by ELISA assay using the FimH lectin domain (FimHL) expressed in E. coli as coating. Overall, all the candidates elicited an IgG response. FimH_PGDGN_DG and FimH_PGDGN_DG_Ferritin showed similar IgG tites, however the NP candidate showed a more homogeneous and compact response at post II. This result suggested that NP generated an earlier efficacious response in comparison to the candidate expressed as recombinant protein. The FimH_PGDGN_DG_Ferritin immunised at two different doses (15 ug and 3 ug) showed that the lower dose (3 ug) was comparable to the higher (15 ug) dose in terms of total IgG response indicating that that the ferritin nanoparticles carrying recombinant FimH_DG_PGDGN protein had good immunogenicity even at the lower tested dose of 3 ug. In addition, the ferritin form led to a less scattered immune response at the second dose as compared to the other candidates, including the otherwise corresponding FimH construct lacking a nanoparticle domain (FIG. 11).

FimH-DG Stabilised Candidates (Produced in Mammalian System) Indicate a Stronger Capability to Inhibit Bacterial Adhesion with Respect to Recombinant (Bacterial Produced) Form

The ability of sera raised against FimH stabilised candidates to impede bacterial adhesion of human bladder cells was tested using an in vitro bacterial inhibition assay. Antibodies against vaccine candidates FimH_PGDGN_DG and FimH_PGDGN_DG_Ferritin were more efficacious than FimH_DNKQ_DG or bacterial-produced FimHL-cys candidate in inhibiting the bacterial adhesion to the urothelial cells. These results indicate that the FimH-based stabilised vaccine candidates expressed into a mammalian system have a great potential for further vaccine development. Furthermore, the linker used for FimH stabilisation played a crucial role for the functionality of the generated antibodies (FIG. 12), with constructs having the PGDGN linker being associated with improved results in terms of inhibition of bacterial adhesion.

Conclusions

Our study investigated novel FimH candidates stabilised with the donor stand strategy. Vaccine candidates were produced as single recombinant proteins or assembled into nanoparticles carrying FimH subunits. As expression in E. coli resulted in insoluble products, soluble antigen expression has been achieved by using a mammalian expression system, through transient transfection of EXP1293-GNTI cells. To our knowledge, the usage of this expression system has never been used before to produce bacterial proteins. In this case, mammalian expression system improved protein solubility, since FimH expressed from in E. coli was insoluble in all tested conditions. This expression system has allowed to produce stabilized FimH_DG antigens all in soluble form, as well as different FIMH nanoparticles (FimHL-mI3, FimHL-Ferritin and FimHH_DG_PGDGN-ferritin). On the contrary, when unstabilised FimH was fused to NP, no expression was detected, demonstrating that stabilisation through FimG complementing strand is necessary to produce full length isolated FimH protein in mammalian cells and to display the antigen on ferritin NPs. The deletion of the two glycine residues, which are the natural linker between FimHL and FimHP resulted in no expression of FimH_ΔGG_PGDGN_DG with extra N-terminal AA, suggesting that this deletion was detrimental for protein stability.

SDS-PAGE comparison of MW of bacterial insoluble proteins and the corresponding mammalian expressed protein show that they have different molecular weights, suggesting that the mammalian proteins are glycosylated, as confirmed by PNGase treatment. All constructs with the leader sequence (the IgK murine leader sequence alone or with extra amino acids) were successful in secreting FimH constructs into the expression medium. However, none of the constructs with extra amino acids resulted in more homogenous nanoparticles (FIG. 7 and FIG. 9), and no naked ferritin NPs were observed.

Structural data confirmed that all nanoparticles were correctly assembled, and FimH spikes were detected on the surface of ferritin (24 spikes) and m13 Nanoparticles (60 spikes).

Our data suggested that FimH stabilised candidates expressed into a mammalian system were immunogenic and the raised antibody were able to inhibit the bacterial adhesion to urothelial cells.

Effect of A501 Adjuvant: Improvement Over FimHC and FimH-DG

To evaluate the contribution of PHAD and AS01 adjuvants systems to the humoral response, FimHC protein complex was used as model antigen and was expressed as described in Langermann S, et al. Science. 1997 Apr. 25; 276(5312):607-11. IgGs antibodies raised after vaccination were determined, and relative titers were plotted as a function of MPL amount contained in the PHAD and AS01 formulations. Overall, AS01 induced a higher total IgG response than PHAD in mice sera (post-3) and urine (post-2 and -3). Moreover, AS01B used at 5 μg-MPL showed the same IgG level in comparison of PHAD containing 12.5 μg-MPL (FIG. 17 A and FIG. 17 B)

Improved Antigen Design and Adjuvanted Formulation Elicit Functional Immune Response after 2 Doses (Instead of 3)

To evaluate the immune response of FimHC complex and FimH-stabilized his-tagged forms (FimHDG, i.e. FimH-PGDGN-DG, wherein DG stands for donor strand complementing peptide from FimG, FimHDG-Ferritin, i.e. FimH-PGDGN-DG-linker (with His-tag)-Ferritin (from H. pylori)), different antigen doses (0.55 μg or 1.6 μg) adjuvanted with PHAD or AS01 were used for mice immunization. The FimHC complex was expressed as described in Langermann S, et al. Science. 1997 Apr. 25; 276(5312):607-11 Protein were expressed in bacterial or mammalian systems. FimH-specific total IgG titers (measured by ELISA) in the sera and urine of immunized mice, measured after second and third vaccine injection. IgG titers of post-2 and post-3 sera raised against different forms of FimHDG candidate, formulated with AS01, were determined (FIG. 18 A). IgG values were compared with those induced by vaccination with FimHC used in combination with the same AS01 adjuvant and PHAD (with MPL amounts comparable to those present in the AS01). As shown in FIG. 18 A, at 0.55 μg antigen dose it there was a clear enhancement of the antibody response with AS01 over PHAD for FimHC after the second and third administration. Also, a better immune response of FimHDG-HisTag, expressed and purified from a bacterial system, was observed in comparison to FimHC benchmark adjuvanted with PHAD.

Finally, both stabilized FimH candidates purified in mammalian cell (FimHDG-HisTag mammalian and FimHDG-His Tag Ferritin) showed higher response than FimHDG expressed in E. coli. Both 1.6 and 0.55 μg of mammalian FimHDG constructs induced IgG levels that plateaued after the 2nd and 3rd immunization. Furthermore, FimHDG at the second administration raised a higher response in compared to 3 doses of FimHC-PHAD at both the tested protein doses (Geometric mean ratio of 9.7 and 3 respectively) (FIG. 18 A). The antibody response against FimHDG was evaluated in urine collected by immunized groups with higher protein dose after 1^{st}, 2^{nd} and 3^{rd} dose. As observed in tested sera, higher IgG titers were measured for mice vaccinated with mammalian FimHDG formulations (FIG. 18 B).

For selected immunized groups the total IgG response at post-I was also determined. At post dose I, the FimHDG-Ferritin nanoparticle induced twice higher GMTs than FimHDG without Ferritin (at any Ag dose) although variability was higher than in the post-2 and post-3 responses (lead to big 95% CI including 1). As compared to bacterial derived antigen, the mammalian form adjuvanted with AS01 induced higher IgG responses at post dose I and II (observed GMRs ranged from 7.1 to 60.8 with all lower limits of 95% CI above 1), while the response was similar after the 3^{rd} dose (observed GMRs around 1.5-fold) (FIG. 19)

Comparison of Different Linkers of Constructs (Mammalian/Bacterial) in Terms of Relative Potency

To investigate the effect of different linkers FimHDG candidates expressed both in a bacterial and mammalian systems were compared to FimHC in terms of bacterial inhibition of the adhesion to uroepithelial cells (BAI). FIG. 20 showed that all FimHDG constructs were more functional than FimHC independently from the expression system used for the expression (bacterial or mammalian). Interestingly, FimHDG constructs harboring PGDGN linker were more effective in comparison to the DNKQ constructs. These data suggest the likers can stabilize FimH in different conformation consequently raising a different functional antibody response. The BAI assay has been conceived as a multiple dilution assay, where the tested samples together with a reference pool of sera are plated at different concentrations to estimate the dose-response curves. The signal is normalized between 0% and 100% before titer computation. The titer is express as Relative Potency (RP) of the tested sample against the reference pool, comparing the corresponding dose-response curve. In details, the RP is computed considering the dilution in logarithmic scale and fitting a 4 parameters logistic (4PL) constrained model (described in the Eur.Ph. chapter 5.3) where the standard and tested samples slope-factor, upper asymptote and lower asymptote are constrained to be equal. The RP is computed as the ratio between the Reference and the sample EC₅₀. The EC₅₀ are calculated from the 4PL constrained inflection point and back transformed (antilog). The model requires that the curves of the reference and the samples have the same slope-factor (parallelism) and the same maximum and minimum response level at the extreme parts (linearity). The suitability of the assumption of parallelism and linearity is assessed for each session evaluating the P-value to test deviations from parallelism, the P-value to test deviations from linearity and the slope ration between reference and sample.

FimH-DG Elicits a Functional Immune Response in Term of BAI, HAI and Conformational mAb Binding

Further, the antibody ability of anti-FimHDG antibodies to inhibit ExPEC adhesion using a bacterial inhibition assay (BAI) was assessed. Data on antibodies functionality revealed that sera raised against both bacterial and mammalian FimHDG constructs showed higher ability than FimHC benchmark sera to inhibit bacterial adhesion. Among the candidate tested, FimHDG-ferritin showed at least 10-fold higher functionality compared to the congener FimHDG construct (FIG. 21). Similar results were obtained performing an HAI assay. The FimHC complex was expressed as described in Langermann S, et al. Science. 1997 Apr. 25; 276(5312):607-11. “FimHDG” refers to FimH-PGDGN-DG, wherein DG stands for donor strand complementing peptide from FimG, “FimHDG-Ferritin” refers to FimH-PGDGN-DG-linker (with His-tag)-Ferritin (from H. pylori). The BAI assay and relative potency calculation were performed as described in the previous example.

FimHDG and mAb962 Binding

To study the interaction of FimHDG (i.e., FimH-PGDGN-DG, wherein DG stands for donor strand complementing peptide from FimG) and mAb926 (Dagmara I. Kisiela et al (2015) a SPR analysis was performed. FimHDG monomeric forms (i.e., FimH-PGDGN-DG, wherein DG stands for donor strand complementing peptide from FimG) obtained from bacterial or mammalian system showed similar binding to the mAb with slightly differences in the association and dissociation profiles. FimHDG-Ferritin (FimH-PGDGN-DG-linker (with His-tag)-Ferritin (from H. pylori)) resulted in a more stable interaction compared to the monomeric forms possibly due to the multimerization effect with increased avidity. By contrast, the lower interaction of mAb926 with FimHC in comparison with FimHDG suggested that the latter was stabilised in a pre-binding conformation as expected. In fact, mAb926 was generated against a FimH stabilized lectin domain with significantly reduced mannose binding capability (pre-binding conformation) (Dagmara I. Kisiela et al., (2013) while FimC stabilizes FimH in its extended post-binding-like form (Sauer et al., (2016), Nature Communications volume 7, Article number: 10738) (FIG. 22). The FimHC complex was expressed as described in Langermann S, et al. Science. 1997 Apr. 25; 276(5312):607-11.

Evaluation of New Linkers

Recombinant proteins production in mammalian cells.

In order to produce FimHDG (i.e., FimH-PGDGN-DG, wherein DG stands for donor strand complementing peptide from FimG) as well as FimHDG-nanoparticles not containing internal or C-terminal repeated His residues, new constructs have been designed inserting different linkers spacing the FimH_DG gene and the nanoparticle (NP) monomer. The FimH-NP mammalian constructs were synthesized by Geneart or Twist as synthetic genes in pCDNA3.4 (LifeTechnologies) vector. All sequences were codon optimized for expression in mammalian cells and contained an N-terminal leader sequence for secretion into the cells medium. This sequence is the IgK murine leader sequence METDTLLLWVLLLWVPGSTG, or the IgK murine leader sequence followed by and aspartic residue METDTLLLWVLLLWVPGSTGD in order to evaluate the contribute of this residue to efficient protein secretion. To produce recombinant FimH-NPs, the expression vectors were transfected into Expi293 cells and\or ExpiCHO cells according to the manufacturer instructions (Life Technologies) and culture supernatants were collected after 5 days of transfection. Protein purification was achieved by an ion exchange chromatography followed by a preparative SEC purification step.

NanoDSF Analysis

To assess the fluorescence-monitored unfolding of the FimHDG constructs a nano-DSF analysis was performed. Samples were manually loaded into nano-DSF grade standard capillaries in triplicates and transferred to a Prometheus NT.48 nano-DSF device. For intrinsic tryptophan fluorescence measurements, the excitation wavelength of 280 nm was used, and the emission of tryptophan fluorescence was measured at 330 nm, 350 nm, and their ratios (350 nm/330 nm). Data were analyzed with Prometheus PR. Control software (NanoTemper Technologies) and plotted using the fluorescence ratio against the temperature.

SPR Analysis

The FimHDG constructs were diluted with running buffer HBS-EP+ (0.01 M HEPES, 0.15 M NaCl, 0.003 M EDTA and 0.05% v/v Surfactant P20) and captured on the surface of a sensor chip NTA that was previously activated by injecting a 0.5 mM solution of Ni2+ ions and washed with 3 mM of EDTA. mAbs were captured at concentration of 20 ug/ml on the surface of a CM5 sensor coated with secondary anti-mouse IgG Fc. A 50 nM fixed concentration of each sample was injected on the surface of the sensor chip for 180 sec. The dissociation followed for 600 sec. Finally, the sensor chip was regenerated using 10 mM Glycine-HCl pH 1.7. The experiments were performed using a Biacore T200 Instrument (GE Healthcare) and analysed with Biacore T200 Evaluation software 3.0 (GE Healthcare).

Results:

The full length FimH-DG stabilized tagless protein containing a secretion murine Ig-K chain leader sequence alone and fused to protein NPs (ferritin) were used to transfect EXPI293 and ExpiCHO cells. The accumulation of secreted recombinant protein was characterized by measuring their expression in culture supernatants 5 days post-transfection by SDS-PAGE. The analysis revealed that FimHDG soluble expression could be obtained at high level for the tagless construct (figure A) in Expi293 cells and ExpiCHO. The proteins were further purified from the culture supernatants and biochemically characterized in comparison with the previously purified His-Tagged FimHDG and bacterial refolded FimHDG. FimHDG tagless was obtained with good purity level in SDS-Page and SE-UPLC from both EXpi293 and ExpiCHO cells. The proteins run with a higher molecular weight in SDS-Page (around 42 kD) vs the theoretical one (31 kD) due to glycosylation occurring in mammalian cells, compared to bacterial cells (FIG. 23 A).

The folding of the Tagless purified FimDG was analysed by nano-DSF and melting temperature were obtained and compared with the one obtained for FimHDG-HIS. FimH-DG showed a good thermal stability in Nano-DSF with 2 thermal transitions, relative to lectin (Tm1) and pilin (Tm2) domains, while the His-tag FimHDF molecule shows only one transition, probably due to a different folding. Moreover, tag-less proteins showed higher stability (higher melting temperatures values) of pilin domain transition respect the His-tag molecules. FIG. 23 B. This different folding in the His tagged construct compared to the tagless FimH DG is probably due to the absence of both N-terminal aspartic residue and C-terminal His-Tag.

SPR analysis (FIG. 23 C) of mammalian produced FimHDG tagless constructs show that mAb 926 can bind to the constructs with differences in the binding profile compared to the his-tag FimHDG protein. Moreover, the tagless FimHDG proteins show weak interactions with mAb VH_475 and mannose, on the contrary of the His-tagged FimHDG, in agreement with the different folding observed for the tagless construct in comparison with the His-tagged protein.

For the production of tagless FimHDG-ferritin NPs, the His tag has been replaced by different linkers to separate the FimHDG molecule and the nanoparticle monomer sequence. The linkers designed and tested are made of flexible residues like glycine and serine so that the connected protein domains are free to move relative to one another. We tested different length of linkers, longer linkers can ensure that two adjacent domains do not sterically interfere with one another, but could be more susceptible to degradation. The linker AKFVAAWTLKAAA, also known as Pan HLA DR-binding epitope (PADRE) is a peptide that activates antigen specific-CD4+ T cells, which has been proposed as a carrier epitope suitable for use in the development of synthetic and recombinant vaccines. The linkers GGGGSLVPRGSGGGGS and EAAAKEAAAKEAAAKA are rigid linkers. The linker AEAAAKEAAAKEAAAKA stabilized by Glu-Lys salt bridges, forms an alfa helix structure (Marqusee & Baldwin, 1987). As the tagless FimHDG and His-tagged FimHDG differ also for the initial aspartic residue, some of the linkers were also tested in absence and in presence of N-terminal aspartic residue. The plasmids coding for the different constructs were used for Expi293 transfection. After 5 days of transfection only constructs starting with N-terminal Aspartic residue (D) (tagless or his-tagged) show a band of secreted protein in the supernatant visible by SDS-page (FIG. 23 D). Constructs FimHDG_HIS_Ferritin 1619SI and 1042SI have the same sequence except for initial aspartic residue, but only the construct 1042SI is secreted and present in the culture supernatants of EXPI, confirming the importance of this residue at the N-terminus of FimHDG to achieve efficient FimHDG-ferritin nanoparticles secretion. Among the different linkers tested, only the constructs FimHDG-ferritin tagless 1623SI and 1627S1, from E. coli strain J96 and 536, which have initial aspartic residue, resulted to be secreted. A wester blotting analysis was also performed in order to assess the expression of the tagless FimHDG-ferritin 1433SI in absence of initial Asp residue, confirming that the protein is expressed in the pellet fraction while is not present only in the culture supernatant.

E. Coli Ferritin in Silico Stability Studies

Material and Methods

Evolutionary Constraints for the Sequence and Structure-Based Design of E. coli Ferritin

The goal of this research was to perform the design of symmetric systems such as self-assembling protein nanoparticles. This approach introduced stabilizing mutations using a combination of computational physics-based algorithms and evolutionary bioinformatics. To achieve this aim, consensus sequence design was performed on the asymmetric unit or monomer of E. coli ferritin (PDB: 1EUM WorldWideWeb(www).rcsb.org/structure/1EUM), using the Rosetta suite (Alford R F, et al. J Chem Theory Comput. 2017 Jun. 13; 13(6):3031-3048) for thermodynamic design, and non-redundant evolutionary homologs (PSI-BLAST, Altschul S F, et al. Nucleic Acids Res. 1997 Sep. 1; 25(17):3389-402) to limit mutation space. The designed models were constrained within a symmetric framework (DiMaio F, et al PLoS One. 2011; 6(6):e20450), in order to optimize the energetics of protein subunits at geometric interfaces. This symmetry-based pipeline was then implemented in a modified version of the structural bioinformatics tool, PROSS (Goldenzweig A, et al. Mol Cell. 2016 Jul. 21; 63(2):337-346), yielding a list of in silico stabilized sequences (SEQ ID NO: 149-152 and FIG. 24).

Protein Expression and Purification

Genes coding for the different mutants of E. coli stabilized ferritin and wild type ferritin were cloned into the pET15TEV vector, which contained an N-terminal 6×His-tag and a TEV cleavage site. Plasmids encoding for the different constructs were transformed into E. coli BL21DE3t1r competent cells. For protein expression, the cells were grown at 20° C. in HTMC ON and induced at 20° C. with 1 mM IPTG for 24 hours. The soluble proteins were extracted by chemical lysis using CeLytic™ Express (Sigma Aldrich) and purified by a nickel chelating column, followed by preparative size exclusion chromatography using a Superdex 200© Increase 10/300 GL column (Cytiva), with purity confirmed by SDS-PAGE (FIG. 25).

Transmission Electron Microscopy (TEM) Analysis

For negative staining: 5 μl of samples (diluted in 20 ng/microliter) were loaded for 30 seconds onto a glow-discharged copper 300-square mesh grid. After blotting the excess, the grid was negatively stained using Nano-W stain (Ted Pella, Inc) for 30 seconds. The samples were analyzed using a Tecnai G2 spirit and images were acquired using a Veleta CCD (FIG. 26).

ThermoFluor Assay

The ThermoFluor assay is a quick, temperature-based assay to assess the stability of proteins. In this method, each sample is diluted to a final concentration of 0.2 mg/ml, with an additional 4 μl of SYPRO Orange dye 1000× (Molecular Probes) to reach a final volume of 40 μl using buffer solution. This mix was pipetted into the wells of a 96-well thin-wall PCR plate (Bio-Rad), with water added to control samples. Each sample was analyzed in triplicate. The melting point (Tₘ) of each protein was determined by ramping from 25° C. to 100° C. with a scan-rate increment of 1° C. per min, taking a fluorescence measurement at each 1° C. step. The unfolding profile and melting temperature were monitored by a quantitative PCR thermo cycler (Stratagene). All DSF experiments were performed in triplicate. The derivates of fluorescence intensities were plotted as a function of temperature and the reported Tₘ is the inflection point of the sigmoid curve determined using GraphPad Prism software (FIG. 27).

Results

Recombinant production of an in silico stabilized E. coli ferritin nanoparticle

To obtain a stabilized nanoparticle from E. coli that is presenting an E. coli stabilized specific antigen (FimHDG, i.e., FimH-PGDGN-DG, wherein DG stands for donor strand complementing peptide from FimG), a native ferritin scaffold for the repetitive display of FimH was selected and computationally optimized. The Rosetta-based design approach maintained octahedral symmetry and focused on the interface between the monomer and the 23 other chains in the symmetric system (FIG. 24). This strategy of having a stabilized ferritin from E. coli that is presenting an E. coli specific antigen (FimH), is a rational approach for maintaining species or genus-specific designs by using a native scaffold for the repetitive display of an antigen.

The E. coli WT ferritin and four of the mutants, representative of all the in silico stabilized sequences generated by PROSS (SEQ ID NO: 149-152), were highly expressed and soluble when produced as recombinant His-tagged proteins in an E. coli cell line (FIG. 25). The constructs were successfully purified with an affinity purification step, followed by preparative size exclusion chromatography. The peak corresponding to the high molecular weight fraction was collected for all the constructs and further analyzed with electron microscopy to assess the correct formation of homogeneous and well-structured nanoparticles. From the TEM analysis, all the samples resulted in correctly folded ferritin nanoparticles, except for mutant 2.5 which had a non-uniform morphology (FIG. 26).

To identify the most stable E. coli ferritin nanoparticle, the thermal stability of recombinant ferritin constructs (WT, 0.5, 2, 6) was assessed by differential scanning fluorimetry (DSF) using Sypro Orange, which binds to hydrophobic residues and detects their exposure during protein unfolding. The ferritin proteins showed very high thermal stability, as expected for a protein nanocage, with the first unfolding transition being detected around 74° C.-76° C. This DSF analysis demonstrated that the E. coli mutant (0.5) protein exhibited the highest shift in thermal unfolding, leading to its selection as the preferred construct to be fused with the FimHDG antigen, based on this increase in stability.

Mammalian Production of E. coli Stabilized Ferritin Displaying the FimHDG Antigen

To test if the stabilized and native ferritin nanoparticle could be used as a scaffold for the display of FimHDG antigen (i.e., FimH-PGDGN-DG, wherein DG stands for donor strand complementing peptide from FimG), and as an alternative to H. pylori ferritin, the sequence of FimHDG (containing the secretion sequence Igk) was genetically fused to the gene of the stabilized ferritin (mutant 0.5). The two molecules were separated by a linker containing a repeated histidine sequence to allow for affinity purification of the recombinant secreted nanoparticles in mammalian cell culture supernatant. This construct was used for transfection of Expi293 Gnti cells, and the accumulation of secreted recombinant protein was characterized by assessing the expression in culture supernatants 5 days post-transfection by western blotting analysis, using anti-His antibody. The analysis revealed that FimHDG-ferritin (mutant 0.5) nanoparticles were successfully secreted in the cell supernatant. The purified FimHDG-ferritin (mutant 0.5) nanoparticles were visualized by transmission electron microscopy, confirming the correct morphology of the ferritin stabilized nanoparticles and the surface display of the FimHDG antigen with a size of around 20 nm (FIG. 28).

This data indicates that a stabilized E. coli ferritin nanoparticle displaying FimHDG can be successfully produced in mammalian cells, indicating that it is possible to design nanoparticles with antigens and scaffolds that are both native to the target pathogen.

TABLE 1
(A): Bacterial tested FimH-NP

| RIMS | Protein | Vector | | |
|---|---|---|---|---|
| Code | Name | Name | Expected AA sequence | Tag |
| 1097SI | FIMH_DG_ | pTrcHi | MFACKTANGTAIPIGGGSANVYVNLAPAVNVGQNLVVDLSTQIFCHND | internal- |
| | PGDGN_ | s2A | YPETITDYVTLQRGSAYGGVLSSFSGTVKYNGSSYPFPTTSETPRVVYNSR | His |
| | HIS- | | TDKPWPVALYLTPVSSAGGVAIKAGSLIAVLILRQTNNYNSDDFQFVWNI | |
| | Ferritin | | YANNDVVVPTGGCDVSARDVTVTLPDYPGSVPIPLTVYCAKSQNLGYYL | |
| | 536 | | SGTTADAGNSIFTNTASFSPAQGVGVQLTRNGTIIPANNTVSLGAVGTSA | |
| | | | VSLGLTANYARTGGQVTAGNVQSIIGVTFVYQPGDGNADVTITVNGKV | |
| | | | VAKSGSHHHHHHHHGGSDIIKLLNEQVNKEMNSSNLYMSMSSWCYTH | |
| | | | SLDGAGLFLFDHAAEEYEHAKKLIIFLNENNVPVQLTSISAPEHKFEGLTQI | |
| | | | FQKAYEHEQHISESINNIVDHAIKSKDHATFNFLQWYVAEQHEEEVLFKD | |
| | | | ILDKIELIGNENHGLYLADQYVKGIAKSRK [SEQ ID NO: 20] | |
| 1064SI | LS- | pET21 | MKYLLPTAAAGLLLLAAQPAMAFacktangtaipigggsanvyvnlapvvnvgq | C-His |
| | FIMHL- | | nlvvdlstqifchndypetitdyvtlqrgsayggvlsnfsgtvkysgssypfpttsetprvvy | |
| | IMX313- | | nsrtdkpwpvalyltpvssaggvaikagsliavlilrqtnnynsddfqfvwniyanndvvv | |
| | HIS | | ptggSSGSGSGSKKQGDADVCGEVAYIQSVVSDCHVPTAELRTLLEIRKLF | |
| | | | LEIQKLKVELQGLSKEGGGSGSHHHHHHHH [SEQ ID NO: 21] | |
| 955SI | FIMHL- | pTrcHis2A | MfaSktangtaipigggsanvyvnlapvvnvgqnlvvdlstqifShndypetitdyvtlqr | C-His |
| | S24S65- | | gsayggvlsnfsgtvkysgssypfpttsetprvvynsrtdkpwpvalyltpvssaggvaika | |
| | IMX313 | | gsliavlilrqtnnynsddfqfvwniyanndvvvptggSSGSGSGSKKQGDADVCG | |
| | | | EVAYIQSVVSDCHVPTAELRTLLEIRKLFLEIQKLKVELQGLSKEGGGSGSH | |
| | | | HHHHH [SEQ ID NO: 22] | |
| 954SI | FIMHL- | pTrcHi | MFASKTANGTAIPIGGGSANVYVNLAPAVNVGQNLVVDLSTQIFSHNDY | internal |
| | S24S65- | s2A | PETITDYVTLQRGSAYGGVLSSFSGTVKYNGSSYPFPTTSETPRVVYNSRT | |
| | foldon- | | DKPWPVALYLTPVSSAGGVAIKAGSLIAVLILRQTNNYNSDDFQFVWNIY | |
| | ferritin | | ANNDVVVPTGSGYIPEAPRDGQAYVRKDGEWVLLSTFLGSGHHHHHH | |
| | | | GSGDIIKLLNEQVNKEMNSSNLYMSMSSWCYTHSLDGAGLFLFDHAAE | |
| | | | EYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYL | |
| | | | ADQYVKGIAKSRK [SEQ ID NO: 23] | |
| 940SI | FIMHL- | pTrcHi | MFASKTANGTAIPIGGGSANVYVNLAPAVNVGQNLVVDLSTQIFSHNDY | C-His |
| | S24S65- | s2A | PETITDYVTLQRGSAYGGVLSSFSGTVKYNGSSYPFPTTSETPRVVYNSRT | |
| | Mi3 | | DKPWPVALYLTPVSSAGGVAIKAGSLIAVLILRQTNNYNSDDFQFVWNIY | |
| | | | ANNDVVVPTGGSGGSGGSMKMEELFKKHKIVAVLRANSVEEAKKKALA | |
| | | | VFLGGVHLIEITFTVPDADTVIKELSFLKEMGAIIGAGTVTSVEQARKAVES | |
| | | | GAEFIVSPHLDEEISQFAKEKGVFYMPGVMTPTELVKAMKLGHTILKLFP | |
| | | | GEVVGPQFVKAMKGPFPNVKFVPTGGVNLDNVCEWFKAGVLAVGVGS | |
| | | | ALVKGTPVEVAEKAKAFVEKIRGCTEGSGSGSGSGSHHHHHH [SEQ ID NO: 24] | |
| 939SI | FIMHL- | pTrcHi | MFACKTANGTAIPIGGGSANVYVNLAPAVNVGQNLVVDLSTQIFCHND | C-His |
| | mI3 | s2A | YPETITDYVTLQRGSAYGGVLSSFSGTVKYNGSSYPFPTTSETPRVVYNSR | |
| | | | TDKPWPVALYLTPVSSAGGVAIKAGSLIAVLILRQTNNYNSDDFQFVWNI | |
| | | | YANNDVVVPTGGSGGSGGSMKMEELFKKHKIVAVLRANSVEEAKKKAL | |
| | | | AVFLGGVHLIEITFTVPDADTVIKELSFLKEMGAIIGAGTVTSVEQARKAVE | |
| | | | SGAEFIVSPHLDEEISQFAKEKGVFYMPGVMTPTELVKAMKLGHTILKLF | |
| | | | PGEVVGPQFVKAMKGPFPNVKFVPTGGVNLDNVCEWFKAGVLAVGV | |
| | | | GSALVKGTPVEVAEKAKAFVEKIRGCTEGSGSGSGSGSHHHHHH | |
| | | | [SEQ ID NO: 25] | |
| 913SI | FimHL- | pET21 | MfaSktangtaipigggsanvyvnlapavnvgqnlvvdlstqifShndypetitdyvtlqr | C-His |
| | NOCYS- | | gsayggvlsnfsgtvkysgssypfpttsetprvvynsrtdkpwpvalyltpvssaggvaika | |
| | MI3 | | gsliavlilrqtnnynsddfqfvwniyanndvvvptggGGSGGSGGSGGSMKMEE | |
| | | | LFKKHKIVAVLRANSVEEAKKKALAVFLGGVHLIEITFTVPDADTVIKELSFL | |
| | | | KEMGAIIGAGTVTSVEQARKAVESGAEFIVSPHLDEEISQFAKEKGVFYM | |
| | | | PGVMTPTELVKAMKLGHTILKLFPGEVVGPQFVKAMKGPFPNVKFVPT | |
| | | | GGVNLDNVCEWFKAGVLAVGVGSALVKGTPVEVAEKAKAFVEKIRGCT | |
| | | | EGSGSGSGSGSHHHHHH [SEQ ID NO: 26] | |
| 904SI | FimHdel | pET21 | Mfacktangtaipigggsanvyvnlapvvnvgqnlvvdlstqifchndypetitdyvtlqr | C-His |
| | taGG_ | | gsayggvlsnfsgtvkysgssypfpttsetprvvynsrtdkpwpvalyltpvssaggvaika | |
| | PGDGNDG_ | | gsliavlilrqtnnynsddfqfvwniyanndvvvptcdvsardvtvtlpdypgsvpipltvy | |
| | mi3 | | caksqnlgyylsgttadagnsiftntasfspaqgvgvqltrngtiipanntvslgavgtsavsl | |
| | | | gltanyartggqvtagnvqsiigvtfvyqPGDGNADVTITVNGKVVAKGSGGGG | |
| | | | MKMEELFKKHKIVAVLRANSVEEAKKKALAVFLGGVHLIEITFTVPDADT | |
| | | | VIKELSFLKEMGAIIGAGTVTSVEQARKAVESGAEFIVSPHLDEEISQFAKE | |
| | | | KGVFYMPGVMTPTELVKAMKLGHTILKLFPGEVVGPQFVKAMKGPFPN | |
| | | | VKFVPTGGVNLDNVCEWFKAGVLAVGVGSALVKGTPVEVAEKAKAFVE | |
| | | | KIRGCTEGSGSGSGSGSHHHHHH [SEQ ID NO: 27] | |
| 888SI | FimHL- | pET15 | MGSSHHHHHHENLYFQGFACKTANGTAIPIGGGSANVYVNLAPAVNV | N-His |
| | GSG4- | TEV | GQNLVVDLSTQIFCHNDYPETITDYVTLQRGSAYGGVLSSFSGTVKYNGS | |
| | Ferritin | | SYPFPTTSETPRVVYNSRTDKPWPVALYLTPVSSAGGVAIKAGSLIAVLILR | |
| | | | QTNNYNSDDFQFVWNIYANNDVVVPTGSGGGGDIIKLLNEQVNKEMN | |
| | | | SSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRK | |
| | | | [SEQ ID NO: 28] | |
| 887SI | pelBLS- | pET22 | MKYLLPTAAAGLLLLAAQPAMAFacktangtaipigggsanvyvnlapvvnvgq | C-His |
| | FimHL- | | nlvvdlstqifchndypetitdyvtlqrgsayggvlsnfsgtvkysgssypfpttsetprvvy | |
| | mI3 | | nsrtdkpwpvalyltpvssaggvaikagsliavlilrqtnnynsddfqfvwniyanndvvv | |
| | | | ptggGSGMKMEELFKKHKIVAVLRANSVEEAKKKALAVFLGGVHLIEITFT | |
| | | | VPDADTVIKELSFLKEMGAIIGAGTVTSVEQARKAVESGAEFIVSPHLDEE | |
| | | | ISQFAKEKGVFYMPGVMTPTELVKAMKLGHTILKLFPGEVVGPQFVKA | |
| | | | MKGPFPNVKFVPTGGVNLDNVCEWFKAGVLAVGVGSALVKGTPVEVA | |
| | | | EKAKAFVEKIRGCTEGSGSGSGSHHHHHH [SEQ ID NO: 29] | |
| 837SI | FimH_DG_ | pET15 | MGSSHHHHHHENLYFQGDVVVPTGGCDVSARDVTVTLPDYPGSVPIPL | N-His |
| | Ferritin | TEV | TVYCAKSQNLGYYLSGTTADAGNSIFTNTASFSPAQGVGVQLTRNGTIIP | |
| | (GSGGGG) | | ANNTVSLGAVGTSAVSLGLTANYARTGGQVTAGNVQSIIGVTFVYQPGD | |
| | | | GNADVTITVNGKVVAKGSGGGGDIIKLLNEQVNKEMNSSNLYMSMSS | |
| | | | WCYTHSLDGAGLFLFDHAAEEYEHAKKLIIFLNENNVPVQLTSISAPEHKF | |
| | | | EGLTQIFQKAYEHEQHISESINNIVDHAIKSKDHATFNFLQWYVAEQHEE | |
| | | | EVLFKDILDKIELIGNENHGLYLADQYVKGIAKSRK [SEQ ID NO: 30] | |
| 836SI | FimHL- | pET21 | MfacktangtaipigggsanvyvnlapvCnvgqnCvvdlstqifchndypetitdyvtlq | C-His |
| | C-C-MI3 | | rgsayggvlsnfsgtvkysgssypfpttsetprvvynsrtdkpwpvalyltpvssaggvaik | |
| | | | agsliavlilrqtnnynsddfqfvwniyanndvvvptggGGSGGSGGSGGSMKME | |
| | | | ELFKKHKIVAVLRANSVEEAKKKALAVFLGGVHLIEITFTVPDADTVIKELSF | |
| | | | LKEMGAIIGAGTVTSVEQARKAVESGAEFIVSPHLDEEISQFAKEKGVFY | |
| | | | MPGVMTPTELVKAMKLGHTILKLFPGEVVGPQFVKAMKGPFPNVKFVP | |
| | | | TGGVNLDNVCEWFKAGVLAVGVGSALVKGTPVEVAEKAKAFVEKIRGC | |
| | | | TEGSGSGSGSGSHHHHHH [SEQ ID NO: 31] | |
| 835SI | FimHL- | pET21 | MfacktangtaipigggsanvyvnlapvCnvgqnCvvdlstqifchndypetitdyvtlq | Tagless |
| | C-C- | | rgsayggvlsnfsgtvkysgssypfpttsetprvvynsrtdkpwpvalyltpvssaggvaik | |
| | qBeta | | agsliavlilrqtnnynsddfqfvwniyanndvvvptggGGSGGSGGSGGSAKLET | |
| | | | VTLGNIGKDGKQTLVLNPRGVNPTNGVASLSQAGAVPALEKRVTVSVSQ | |
| | | | PSRNRKNYKVQVKIQNPTACTANGSCDPSVTRQAYADVTFSFTQYSTDE | |
| | | | ERAFVRTELAALLASPLLIDAIDQLNPAY [SEQ ID NO: 32] | |

TABLE 1
(B): FimH espressed as single recombinant protein in E. coli

| RIMS | Protein | Vector | | |
|---|---|---|---|---|
| Code | Name | Name | Expected AA sequence | Tag |
| 1023SI | FimH_ | pET22 | MFACKTANGTAIPIGGGSANVYVNLAPVVNVGQNLVVDLSTQIFCHND | C-His |
| | DNKQ_DG | b+ | YPETITDYVTLQRGSAYGGVLSNFSGTVKYSGSSYPFPTTSETPRVVYNSR | |
| | citopl | | TDKPWPVALYLTPVSSAGGVAIKAGSLIAVLILRQTNNYNSDDFQFVWNI | |
| | pET22b | | YANNDVVVPTGGCDVSARDVTVTLPDYPGSVPIPLTVYCAKSQNLGYYL | |
| | + | | SGTTADAGNSIFTNTASFSPAQGVGVQLTRNGTIIPANNTVSLGAVGTSA | |
| | | | VSLGLTANYARTGGQVTAGNVQSIIGVTFVYQDNKQADVTITVNGKVV | |
| | | | AKGSGHHHHHH [SEQ ID NO: 120] | |
| 1024SI | FimH_ | pET22 | MFACKTANGTAIPIGGGSANVYVNLAPVVNVGQNLVVDLSTQIFCHND | C-His |
| | PGDGN_ | b+ | YPETITDYVTLQRGSAYGGVLSNFSGTVKYSGSSYPFPTTSETPRVVYNSR | |
| | DG | | TDKPWPVALYLTPVSSAGGVAIKAGSLIAVLILRQTNNYNSDDFQFVWNI | |
| | citopl | | YANNDVVVPTGGCDVSARDVTVTLPDYPGSVPIPLTVYCAKSQNLGYYL | |
| | pET22b | | SGTTADAGNSIFTNTASFSPAQGVGVQLTRNGTIIPANNTVSLGAVGTSA | |
| | + | | VSLGLTANYARTGGQVTAGNVQSIIGVTFVYQPGDGNADVTITVNGKV | |
| | | | VAKGSGHHHHHH [SEQ ID NO: 121] | |
| 1025SI | FimH_ | pET22 | MFACKTANGTAIPIGGGSANVYVNLAPVVNVGQNLVVDLSTQIFCHND | C-His |
| | DGG_ | b+ | YPETITDYVTLQRGSAYGGVLSNFSGTVKYSGSSYPFPTTSETPRVVYNSR | |
| | PGDGN_DG | | TDKPWPVALYLTPVSSAGGVAIKAGSLIAVLILRQTNNYNSDDFQFVWNI | |
| | citopl | | YANNDVVVPTCDVSARDVTVTLPDYPGSVPIPLTVYCAKSQNLGYYLSGT | |
| | pET22b | | TADAGNSIFTNTASFSPAQGVGVQLTRNGTIIPANNTVSLGAVGTSAVSL | |
| | + | | GLTANYARTGGQVTAGNVQSIIGVTFVYQPGDGNADVTITVNGKVVAK | |
| | | | GSGHHHHHH [SEQ ID NO: 122] | |
| 1122SI | FimH_ | pET24 | MFACKTANGTAIPIGGGSANVYVNLAPVVNVGQNLVVDLSTQIFCHND | Tagless |
| | PGDGN_ | b(+) | YPETITDYVTLQRGSAYGGVLSNFSGTVKYSGSSYPFPTTSETPRVVYNSR | |
| | DG | | TDKPWPVALYLTPVSSAGGVAIKAGSLIAVLILRQTNNYNSDDFQFVWNI | |
| | citopl | | YANNDVVVPTGGCDVSARDVTVTLPDYPGSVPIPLTVYCAKSQNLGYYL | |
| | PET24 | | SGTTADAGNSIFTNTASFSPAQGVGVQLTRNGTIIPANNTVSLGAVGTSA | |
| | Tagless | | VSLGLTANYARTGGQVTAGNVQSIIGVTFVYQPGDGNADVTITVNGKV | |
| | | | VAK [SEQ ID NO: 123] | |
| | FimH_ | | FACKTANGTAIPIGGGSANVYVNLAPVVNVGQNLVVDLSTQIFCHNDYP | tagless |
| | PGDGN_ | | ETITDYVTLQRGSAYGGVLSNFSGTVKYSGSSYPFPTTSETPRVVYNSRTD | |
| | DG | | KPWPVALYLTPVSSAGGVAIKAGSLIAVLILRQTNNYNSDDFQFVWNIYA | |
| | citopl | | NNDVVVPTGGCDVSARDVTVTLPDYPGSVPIPLTVYCAKSQNLGYYLSG | |
| | tagless | | TTADAGNSIFTNTASFSPAQGVGVQLTRNGTIIPANNTVSLGAVGTSAVS | |
| | no Met | | LGLTANYARTGGQVTAGNVQSIIGVTFVYQPGDGNADVTITVNGKVVA | |
| | | | K [SEQ ID NO: 124] | |

TABLE 2
Mammalian-expressed FimH as single recombinant proteins and Nanoparticles:

| RIMS | Protein | | | Ex- | |
|---|---|---|---|---|---|
| Code | Name | | Expected AA sequence | pression | Tag |
| 1096SI | FIMHL- | short | METDTLLLWVLLLWVPGSTGDFACKTANGTAIPIGGGSANVYVNLA | yes | inter- |
| | HIS-Mi3 | leader | PVVNVGQNLVVDLSTQIFCHNDYPETITDYVTLQRGSAYGGVLSNFS | | nal |
| | J96 | | GTVKYSGSSYPFPTTSETPRVVYNSRTDKPWPVALYLTPVSSAGGVAI | | HIS |
| | | | KAGSLIAVLILRQTNNYNSDDFQFVWNIYANNDVVVPTGGGSGSHH | | |
| | | | HHHHHHGGSMKMEELFKKHKIVAVLRANSVEEAKKKALAVFLGGV | | |
| | | | HLIEITFTVPDADTVIKELSFLKEMGAIIGAGTVTSVEQARKAVESGAE | | |
| | | | FIVSPHLDEEISQFAKEKGVFYMPGVMTPTELVKAMKLGHTILKLFPG | | |
| | | | EVVGPQFVKAMKGPFPNVKFVPTGGVNLDNVCEWFKAGVLAVGV | | |
| | | | GSALVKGTPVEVAEKAKAFVEKIRGCTE [SEQ ID NO: 33] | | |
| 1095SI | FIMHL- | short | METDTLLLWVLLLWVPGSTGDFACKTANGTAIPIGGGSANVYVNLA | yes | inter- |
| | HIS-Fer | leader | PAVNVGQNLVVDLSTQIFCHNDYPETITDYVTLQRGSAYGGVLSSFS | | nal |
| | 536 | | GTVKYNGSSYPFPTTSETPRVVYNSRTDKPWPVALYLTPVSSAGGVAI | | HIS |
| | | | KAGSLIAVLILRQTNNYNSDDFQFVWNIYANNDVVVPTGGGSGSHH | | |
| | | | HHHHHHGGSDIIKLLNEQVNKEMNSSNLYMSMSSWCYTHSLDGAG | | |
| | | | LFLFDHAAEEYEHAKKLIIFLNENNVPVQLTSISAPEHKFEGLTQIFQKA | | |
| | | | YEHEQHISESINNIVDHAIKSKDHATFNFLQWYVAEQHEEEVLFKDIL | | |
| | | | DKIELIGNENHGLYLADQYVKGIAKSRK [SEQ ID NO: 34] | | |
| 1043SI | FIMH_ | short | METDTLLLWVLLLWVPGSTGDFACKTANGTAIPIGGGSANVYVNLA | yes | C- |
| | DG_ | leader | PVVNVGQNLVVDLSTQIFCHNDYPETITDYVTLQRGSAYGGVLSNFS | | His |
| | PGDGN_ | | GTVKYSGSSYPFPTTSETPRVVYNSRTDKPWPVALYLTPVSSAGGVAI | | |
| | IMX313_ | | KAGSLIAVLILRQTNNYNSDDFQFVWNIYANNDVVVPTGGCDVSAR | | |
| | HIS | | DVTVTLPDYPGSVPIPLTVYCAKSQNLGYYLSGTTADAGNSIFTNTASF | | |
| | J96 | | SPAQGVGVQLTRNGTIIPANNTVSLGAVGTSAVSLGLTANYARTGG | | |
| | | | QVTAGNVQSIIGVTFVYQPGDGNADVTITVNGKVVAKGSSGSGSGS | | |
| | | | KKQGDADVCGEVAYIQSVVSDCHVPTAELRTLLEIRKLFLEIQKLKVEL | | |
| | | | QGLSKEGGGSGSHHHHHH [SEQ ID NO: 35] | | |
| 1042SI | FimH_ | short | METDTLLLWVLLLWVPGSTGDFACKTANGTAIPIGGGSANVYVNLA | yes | inter- |
| | PGDGN_ | leader | PVVNVGQNLVVDLSTQIFCHNDYPETITDYVTLQRGSAYGGVLSNFS | | nal |
| | DG_HIS- | | GTVKYSGSSYPFPTTSETPRVVYNSRTDKPWPVALYLTPVSSAGGVAI | | HIS |
| | Ferritn | | KAGSLIAVLILRQTNNYNSDDFQFVWNIYANNDVVVPTGGCDVSAR | | |
| | j96 | | DVTVTLPDYPGSVPIPLTVYCAKSQNLGYYLSGTTADAGNSIFTNTASF | | |
| | | | SPAQGVGVQLTRNGTIIPANNTVSLGAVGTSAVSLGLTANYARTGG | | |
| | | | QVTAGNVQSIIGVTFVYQPGDGNADVTITVNGKVVAKSGSHHHHH | | |
| | | | HHHGGSDIIKLLNEQVNKEMNSSNLYMSMSSWCYTHSLDGAGLFLF | | |
| | | | DHAAEEYEHAKKLIIFLNENNVPVQLTSISAPEHKFEGLTQIFQKAYEH | | |
| | | | EQHISESINNIVDHAIKSKDHATFNFLQWYVAEQHEEEVLFKDILDKIE | | |
| | | | LIGNENHGLYLADQYVKGIAKSRK [SEQ ID NO: 36] | | |
| 1142SI | FIMH_ | short | METDTLLLWVLLLWVPGSTGDFACKTANGTAIPIGGGSANVYVNLA | yes | inter- |
| | DG_ | leader | PAVNVGQNLVVDLSTQIFCHNDYPETITDYVTLQRGSAYGGVLSSFS | | nal |
| | PGDGN- | | GTVKYNGSSYPFPTTSETPRVVYNSRTDKPWPVALYLTPVSSAGGVAI | | HIS |
| | HIS- | | KAGSLIAVLILRQTNNYNSDDFQFVWNIYANNDVVVPTGGCDVSAR | | |
| | Ferritin | | DVTVTLPDYPGSVPIPLTVYCAKSQNLGYYLSGTTADAGNSIFTNTASF | | |
| | 536 | | SPAQGVGVQLTRNGTIIPANNTVSLGAVGTSAVSLGLTANYARTGG | | |
| | | | QVTAGNVQSIIGVTFVYQPGDGNADVTITVNGKVVAKSGSHHHHH | | |
| | | | HHHGGSDIIKLLNEQVNKEMNSSNLYMSMSSWCYTHSLDGAGLFLF | | |
| | | | DHAAEEYEHAKKLIIFLNENNVPVQLTSISAPEHKFEGLTQIFQKAYEH | | |
| | | | EQHISESINNIVDHAIKSKDHATFNFLQWYVAEQHEEEVLFKDILDKIE | | |
| | | | LIGNENHGLYLADQYVKGIAKSRK [SEQ ID NO: 37] | | |
| 1000SI | FimH_ | EXTRA | METDTLLLWVLLLWVPGSTGDAAQPARRARRTKLALFACKTANGTA | NO | inter- |
| | PGDGN_ | AA | IPIGGGSANVYVNLAPVVNVGQNLVVDLSTQIFCHNDYPETITDYVTL | | nal |
| | DG-HIS- | | QRGSAYGGVLSNFSGTVKYSGSSYPFPTTSETPRVVYNSRTDKPWPV | | HIS |
| | Ferritin | | ALYLTPVSSAGGVAIKAGSLIAVLILRQTNNYNSDDFQFVWNIYANN | | |
| | J96 | | DVVVPTGGCDVSARDVTVTLPDYPGSVPIPLTVYCAKSQNLGYYLSG | | |
| | | | TTADAGNSIFTNTASFSPAQGVGVQLTRNGTIIPANNTVSLGAVGTS | | |
| | | | AVSLGLTANYARTGGQVTAGNVQSIIGVTFVYQPGDGNADVTITVN | | |
| | | | GKVVAKSGSHHHHHHHHGGSDIIKLLNEQVNKEMNSSNLYMSMSS | | |
| | | | WCYTHSLDGAGLFLFDHAAEEYEHAKKLIIFLNENNVPVQLTSISAPE | | |
| | | | HKFEGLTQIFQKAYEHEQHISESINNIVDHAIKSKDHATFNFLQWYVA | | |
| | | | EQHEEEVLFKDILDKIELIGNENHGLYLADQYVKGIAKSRK | | |
| | | | [SEQ ID NO: 38] | | |
| 999SI | FimH_ | EXTRA | METDTLLLWVLLLWVPGSTGDAAQPARRARRTKLALFACKTANGTA | NO | inter- |
| | PGDGN_ | AA | IPIGGGSANVYVNLAPVVNVGQNLVVDLSTQIFCHNDYPETITDYVTL | | nal |
| | DG-HIS- | | QRGSAYGGVLSNFSGTVKYSGSSYPFPTTSETPRVVYNSRTDKPWPV | | HIS |
| | MI3 j96 | | ALYLTPVSSAGGVAIKAGSLIAVLILRQTNNYNSDDFQFVWNIYANN | | |
| | | | DVVVPTGGCDVSARDVTVTLPDYPGSVPIPLTVYCAKSQNLGYYLSG | | |
| | | | TTADAGNSIFTNTASFSPAQGVGVQLTRNGTIIPANNTVSLGAVGTS | | |
| | | | AVSLGLTANYARTGGQVTAGNVQSIIGVTFVYQPGDGNADVTITVN | | |
| | | | GKVVAKSGSHHHHHHHHGGSMKMEELFKKHKIVAVLRANSVEEAK | | |
| | | | KKALAVFLGGVHLIEITFTVPDADTVIKELSFLKEMGAIIGAGTVTSVE | | |
| | | | QARKAVESGAEFIVSPHLDEEISQFAKEKGVFYMPGVMTPTELVKAM | | |
| | | | KLGHTILKLFPGEVVGPQFVKAMKGPFPNVKFVPTGGVNLDNVCEW | | |
| | | | FKAGVLAVGVGSALVKGTPVEVAEKAKAFVEKIRGCTE | | |
| | | | [SEQ ID NO: 39] | | |
| 998SI | FimH_ | EXTRA | METDTLLLWVLLLWVPGSTGDAAQPARRARRTKLALFACKTANGTA | yes | C_ |
| | PGDGN_ | AA | IPIGGGSANVYVNLAPVVNVGQNLVVDLSTQIFCHNDYPETITDYVTL | | his |
| | DG-HIS- | | QRGSAYGGVLSNFSGTVKYSGSSYPFPTTSETPRVVYNSRTDKPWPV | | |
| | IMX313 | | ALYLTPVSSAGGVAIKAGSLIAVLILRQTNNYNSDDFQFVWNIYANN | | |
| | j96 | | DVVVPTGGCDVSARDVTVTLPDYPGSVPIPLTVYCAKSQNLGYYLSG | | |
| | | | TTADAGNSIFTNTASFSPAQGVGVQLTRNGTIIPANNTVSLGAVGTS | | |
| | | | AVSLGLTANYARTGGQVTAGNVQSIIGVTFVYQPGDGNADVTITVN | | |
| | | | GKVVAKGSSGSGSGSKKQGDADVCGEVAYIQSVVSDCHVPTAELRT | | |
| | | | LLEIRKLFLEIQKLKVELQGLSKEGGGSGSHHHHHH [SEQ ID NO: 40] | | |
| 995SI | FimH_ | EXTRA | METDTLLLWVLLLWVPGSTGDAAQPARRARRTKLALFACKTANGTA | yes | inter- |
| | PGDGN_ | AA | IPIGGGSANVYVNLAPAVNVGQNLVVDLSTQIFCHNDYPETITDYVTL | | nal |
| | DG_Ferr | | QRGSAYGGVLSSFSGTVKYNGSSYPFPTTSETPRVVYNSRTDKPWPV | | HIS |
| | itin | | ALYLTPVSSAGGVAIKAGSLIAVLILRQTNNYNSDDFQFVWNIYANN | | |
| | (536) | | DVVVPTGGCDVSARDVTVTLPDYPGSVPIPLTVYCAKSQNLGYYLSG | | |
| | | | TTADAGNSIFTNTASFSPAQGVGVQLTRNGTIIPANNTVSLGAVGTS | | |
| | | | AVSLGLTANYARTGGQVTAGNVQSIIGVTFVYQPGDGNADVTITVN | | |
| | | | GKVVAKSGSHHHHHHHHGGSDIIKLLNEQVNKEMNSSNLYMSMSS | | |
| | | | WCYTHSLDGAGLFLFDHAAEEYEHAKKLIIFLNENNVPVQLTSISAPE | | |
| | | | HKFEGLTQIFQKAYEHEQHISESINNIVDHAIKSKDHATFNFLQWYVA | | |
| | | | EQHEEEVLFKDILDKIELIGNENHGLYLADQYVKGIAKSRK | | |
| | | | [SEQ ID NO: 41] | | |
| 936SI | FimH- | EXTRA | METDTLLLWVLLLWVPGSTGDAAQPARRARRTKLALfacktangtaipi | NO | C- |
| | IMX313 | AA | gggsanvyvnlapvvnvgqnlvvdlstqifchndypetitdyvtlqrgsa | | His |
| | j96 | | yggvlsnfsgtvkysgssypfpttsetprvvynsrtdkpwpvalyltp | | |
| | | | vssaggvaikagsliavlilrqtnnynsddfqfvwniyanndvvvpt | | |
| | | | cdvsardvtvtlpdypgsvpipltvycaksqnlgyylsgttadagns | | |
| | | | iftntasfspaqgvgvqltrngtiipanntvslgavgtsavslglta | | |
| | | | nyartggqvtagnvqsiigvtfvyqGSSGSGSGSKKQGDADVCGEVAYIQS | | |
| | | | VVSDCHVPTAELRTLLEIRKLFLEIQKLKVELQGLSKEGGGSGSHHHH | | |
| | | | HH [SEQ ID NO: 42] | | |
| 935SI | FimH_ | EXTRA | METDTLLLWVLLLWVPGSTGDAAQPARRARRTKLALfacktangtaipi | NO | C- |
| | mi3 | AA | gggsanvyvnlapvvnvgqnlvvdlstqifchndypetitdyvtlqr | | His |
| | j96 | | gsayggvlsnfsgtvkysgssypfpttsetprvvynsrtdkpwpvalyl | | |
| | | | tpvssaggvaikagsliavlilrqtnnynsddfqfvwniyanndvvvpt | | |
| | | | cdvsardvtvtlpdypgsvpipltvycaksqnlgyylsgttadagnsift | | |
| | | | ntasfspaqgvgvqltrngtiipanntvslgavgtsavslglta | | |
| | | | nyartggqvtagnvqsiigvtfvyqGSGGGGMKMEELFKKHKIVAVLRANS | | |
| | | | VEEAKKKALAVFLGGVHLIEITFTVPDADTVIKELSFLKEMGAIIGAGTV | | |
| | | | TSVEQARKAVESGAEFIVSPHLDEEISQFAKEKGVFYMPGVMTPTEL | | |
| | | | VKAMKLGHTILKLFPGEVVGPQFVKAMKGPFPNVKFVPTGGVNLDN | | |
| | | | VCEWFKAGVLAVGVGSALVKGTPVEVAEKAKAFVEKIRGCTEGSGS | | |
| | | | GSGSGSHHHHHHHH [SEQ ID NO: 43] | | |
| 929SI | FIMHL- | EXTRA | METDTLLLWVLLLWVPGSTGDAAQPARRARRTKLALFACKTANGTA | NO | inter- |
| | HIS-mI3 | AA | IPIGGGSANVYVNLAPVVNVGQNLVVDLSTQIFCHNDYPETITDYVTL | | nal |
| | j96 | | QRGSAYGGVLSNFSGTVKYSGSSYPFPTTSETPRVVYNSRTDKPWPV | | HIS |
| | | | ALYLTPVSSAGGVAIKAGSLIAVLILRQTNNYNSDDFQFVWNIYANN | | |
| | | | DVVVPTGSGGHHHHHHHHGSGSMKMEELFKKHKIVAVLRANSVEE | | |
| | | | AKKKALAVFLGGVHLIEITFTVPDADTVIKELSFLKEMGAIIGAGTVTSV | | |
| | | | EQARKAVESGAEFIVSPHLDEEISQFAKEKGVFYMPGVMTPTELVKA | | |
| | | | MKLGHTILKLFPGEVVGPQFVKAMKGPFPNVKFVPTGGVNLDNVCE | | |
| | | | WFKAGVLAVGVGSALVKGTPVEVAEKAKAFVEKIRGCTE | | |
| | | | [SEQ ID NO: 44] | | |
| 951SI | FimH_ | EXTRA | METDTLLLWVLLLWVPGSTGDAAQPARRARRTKLALFACKTASGTAI | yes | C- |
| | DNKQ_ | AA | PIGGGSANVYVNLAPVVNVGQNLVVDLSTQIFCHNDYPETITDYVTL | | His |
| | DG_ | | QRGSAYGGVLSDFSGTVKYSGSSYPFPTTSETPRVVYNSRTDKPWPV | | |
| | deglyc | | ALYLTPVSSAGGVAIKAGSLIAVLILRQTNNYNSDDFQFVWNIYANN | | |
| | | | DVVVPTGGCDVSARDVTVTLPDYPGSVPIPLTVYCAKSQNLGYYLSG | | |
| | | | TTADAGNSIFTNTASFSPAQGVGVQLTRDGTIIPADNTVSLGAVGTS | | |
| | | | AVSLGLTANYARTGGQVTAGNVQSIIGVTFVYQDNKQADVTITVNG | | |
| | | | KVVAKGSGHHHHHH* [SEQ ID NO: 79] | | |
| 932SI | FimH_ | EXTRA | METDTLLLWVLLLWVPGSTGDAAQPARRARRTKLALFACKTANGTA | yes | C- |
| | PGDGN_ | AA | IPIGGGSANVYVNLAPVVNVGQNLVVDLSTQIFCHNDYPETITDYVTL | | His |
| | DG | | QRGSAYGGVLSNFSGTVKYSGSSYPFPTTSETPRVVYNSRTDKPWPV | | |
| | | | ALYLTPVSSAGGVAIKAGSLIAVLILRQTNNYNSDDFQFVWNIYANN | | |
| | | | DVVVPTGGCDVSARDVTVTLPDYPGSVPIPLTVYCAKSQNLGYYLSG | | |
| | | | TTADAGNSIFTNTASFSPAQGVGVQLTRNGTIIPANNTVSLGAVGTS | | |
| | | | AVSLGLTANYARTGGQVTAGNVQSIIGVTFVYQPGDGNADVTITVN | | |
| | | | GKVVAKGSGHHHHHH* [SEQ ID NO: 80] | | |
| 931SI | FimH_ | EXTRA | METDTLLLWVLLLWVPGSTGDAAQPARRARRTKLALFACKTANGTA | yes | C- |
| | DNKQ_ | AA | IPIGGGSANVYVNLAPVVNVGQNLVVDLSTQIFCHNDYPETITDYVTL | | His |
| | DG | | QRGSAYGGVLSNFSGTVKYSGSSYPFPTTSETPRVVYNSRTDKPWPV | | |
| | | | ALYLTPVSSAGGVAIKAGSLIAVLILRQTNNYNSDDFQFVWNIYANN | | |
| | | | DVVVPTGGCDVSARDVTVTLPDYPGSVPIPLTVYCAKSQNLGYYLSG | | |
| | | | TTADAGNSIFTNTASFSPAQGVGVQLTRNGTIIPANNTVSLGAVGTS | | |
| | | | AVSLGLTANYARTGGQVTAGNVQSIIGVTFVYQDNKQADVTITVNG | | |
| | | | KVVAKGSGHHHHHH* [SEQ ID NO: 81] | | |
| 930SI | FimH_ | EXTRA | METDTLLLWVLLLWVPGSTGDAAQPARRARRTKLALFACKTANGTA | no | C- |
| | DeltaGG_ | AA | IPIGGGSANVYVNLAPVVNVGQNLVVDLSTQIFCHNDYPETITDYVTL | | His |
| | PGDGN_ | | QRGSAYGGVLSNFSGTVKYSGSSYPFPTTSETPRVVYNSRTDKPWPV | | |
| | DG | | ALYLTPVSSAGGVAIKAGSLIAVLILRQTNNYNSDDFQFVWNIYANN | | |
| | | | DVVVPTCDVSARDVTVTLPDYPGSVPIPLTVYCAKSQNLGYYLSGTTA | | |
| | | | DAGNSIFTNTASFSPAQGVGVQLTRNGTIIPANNTVSLGAVGTSAVS | | |
| | | | LGLTANYARTGGQVTAGNVQSIIGVTFVYQPGDGNADVTITVNGKV | | |
| | | | VAKGSGHHHHHH* [SEQ ID NO: 82] | | |
| 989SI | FimH_ | short | METDTLLLWVLLLWVPGSTGDFACKTANGTAIPIGGGSANVYVNLA | yes | C- |
| | DGG_sI | leader | PVVNVGQNLVVDLSTQIFCHNDYPETITDYVTLQRGSAYGGVLSNFS | | His |
| | | | GTVKYSGSSYPFPTTSETPRVVYNSRTDKPWPVALYLTPVSSAGGVAI | | |
| | | | KAGSLIAVLILRQTNNYNSDDFQFVWNIYANNDVVVPTCDVSARDV | | |
| | | | TVTLPDYPGSVPIPLTVYCAKSQNLGYYLSGTTADAGNSIFTNTASFSP | | |
| | | | AQGVGVQLTRNGTIIPANNTVSLGAVGTSAVSLGLTANYARTGGQV | | |
| | | | TAGNVQSIIGVTFVYQPGDGNADVTITVNGKVVAKGSGHHHHHH** | | |
| | | | [SEQ ID NO: 83] | | |
| 988SI | FimH_ | short | METDTLLLWVLLLWVPGSTGDFACKTANGTAIPIGGGSANVYVNLA | yes | C- |
| | PGDGN_sI | leader | PVVNVGQNLVVDLSTQIFCHNDYPETITDYVTLQRGSAYGGVLSNFS | | His |
| | | | GTVKYSGSSYPFPTTSETPRVVYNSRTDKPWPVALYLTPVSSAGGVAI | | |
| | | | KAGSLIAVLILRQTNNYNSDDFQFVWNIYANNDVVVPTGGCDVSAR | | |
| | | | DVTVTLPDYPGSVPIPLTVYCAKSQNLGYYLSGTTADAGNSIFTNTASF | | |
| | | | SPAQGVGVQLTRNGTIIPANNTVSLGAVGTSAVSLGLTANYARTGG | | |
| | | | QVTAGNVQSIIGVTFVYQPGDGNADVTITVNGKVVAKGSGHHHHH | | |
| | | | H* [SEQ ID NO: 84] | | |
| 987SI | FimH_ | short | METDTLLLWVLLLWVPGSTGDFACKTANGTAIPIGGGSANVYVNLA | yes | C- |
| | DNKQ_sI | leader | PVVNVGQNLVVDLSTQIFCHNDYPETITDYVTLQRGSAYGGVLSNFS | | His |
| | | | GTVKYSGSSYPFPTTSETPRVVYNSRTDKPWPVALYLTPVSSAGGVAI | | |
| | | | KAGSLIAVLILRQTNNYNSDDFQFVWNIYANNDVVVPTGGCDVSAR | | |
| | | | DVTVTLPDYPGSVPIPLTVYCAKSQNLGYYLSGTTADAGNSIFTNTASF | | |
| | | | SPAQGVGVQLTRNGTIIPANNTVSLGAVGTSAVSLGLTANYARTGG | | |
| | | | QVTAGNVQSIIGVTFVYQDNKQADVTITVNGKVVAKGSGHHHHHH* | | |
| | | | [SEQ ID NO: 85] | | |
| 1183SI | FIMH_ | short | METDTLLLWVLLLWVPGSTGDFACKTANGTAIPIGGGSANVYVNLA | yes | inter- |
| | DG_ | leader | PAVNVGQNLVVDLSTQIFCHNDYPETITDYVTLQRGSAYGGVLSSFS | | nal |
| | PGDGN_ | | GTVKYNGSSYPFPTTSETPRVVYNSRTDKPWPVALYLTPVSSAGGVAI | | HIS |
| | 536-MI3 | | KAGSLIAVLILRQTNNYNSDDFQFVWNIYANNDVVVPTGGCDVSAR | | |
| | | | DVTVTLPDYPGSVPIPLTVYCAKSQNLGYYLSGTTADAGNSIFTNTASF | | |
| | | | SPAQGVGVQLTRNGTIIPANNTVSLGAVGTSAVSLGLTANYARTGG | | |
| | | | QVTAGNVQSIIGVTFVYQPGDGNADVTITVNGKVVAKSGSHHHHH | | |
| | | | HHHGGSMKMEELFKKHKIVAVLRANSVEEAKKKALAVFLGGVHLIEI | | |
| | | | TFTVPDADTVIKELSFLKEMGAIIGAGTVTSVEQARKAVESGAEFIVSP | | |
| | | | HLDEEISQFAKEKGVFYMPGVMTPTELVKAMKLGHTILKLFPGEVVG | | |
| | | | PQFVKAMKGPFPNVKFVPTGGVNLDNVCEWFKAGVLAVGVGSALV | | |
| | | | KGTPVEVAEKAKAFVEKIRGCTE* [SEQ ID NO: 86] | | |
| 1184SI | FIMH_ | short | METDTLLLWVLLLWVPGSTGDFACKTANGTAIPIGGGSANVYVNLA | yes | inter- |
| SI | DG_ | leader | PAVNVGQNLVVDLSTQIFCHNDYPETITDYVTLQRGSAYGGVLSSFS | | nal |
| | PGDGN_ | | GTVKYNGSSYPFPTTSETPRVVYNSRTDKPWPVALYLTPVSSAGGVAI | | HIS |
| | 536- | | KAGSLIAVLILRQTNNYNSDDFQFVWNIYANNDVVVPTGGCDVSAR | | |
| | encap- | | DVTVTLPDYPGSVPIPLTVYCAKSQNLGYYLSGTTADAGNSIFTNTASF | | |
| | suline | | SPAQGVGVQLTRNGTIIPANNTVSLGAVGTSAVSLGLTANYARTGG | | |
| | | | QVTAGNVQSIIGVTFVYQPGDGNADVTITVNGKVVAKSGSHHHHH | | |
| | | | HHHGGSMEFLKRSFAPLTEKQWQEIDNRAREIFKTQLYGRKFVDVE | | |
| | | | GPYGWEYAAHPLGEVEVLSDENEVVKWGLRKSLPLIELRATFTLDLW | | |
| | | | ELDNLERGKPNVDLSSLEETVRKVAEFEDEVIFRGCEKSGVKGLLSFEE | | |
| | | | RKIECGSTPKDLLEAIVRALSIFSKDGIEGPYTLVINTDRWINFLKEEAG | | |
| | | | HYPLEKRVEECLRGGKIITTPRIEDALVVSERGGDFKLILGQDLSIGYED | | |
| | | | REKDAVRLFITETFTFQVVNPEALILLKF* [SEQ ID NO: 87] | | |
| 1127SI | HBcFIM | short | METDTLLLWVLLLWVPGSTGDDIDPYKEFGASVELLSFLPSDFFPSIR | no | C- |
| | HLJ96 | leader | DLLDTASALYREALESPEHCSPHHTALRQAILCWGELMNLATWVGS | | His |
| | | | NLEDPGSGGGGFACKTANGTAIPIGGGSANVYVNLAPVVNVGQNL | | |
| | | | VVDLSTQIFCHNDYPETITDYVTLQRGSAYGGVLSNFSGTVKYSGSSY | | |
| | | | PFPTTSETPRVVYNSRTDKPWPVALYLTPVSSAGGVAIKAGSLIAVLIL | | |
| | | | RQTNNYNSDDFQFVWNIYANNDVVVPTGGGSGGASRELVVSYVNV | | |
| | | | NMGLKIRQLLWFHISCLTFGRETVLEYLVSFGVWIRTPPAYRPPNAPIL | | |
| | | | STLPETTVVGSGGGGHHHHHH* [SEQ ID NO: 88] | | |
| 1126SI | HBcFIM | short | METDTLLLWVLLLWVPGSTGDDIDPYKEFGASVELLSFLPSDFFPSIR | no | C- |
| | HDGJ96 | leader | DLLDTASALYREALESPEHCSPHHTALRQAILCWGELMNLATWVGS | | His |
| | | | NLEDPGSGGGGFACKTANGTAIPIGGGSANVYVNLAPVVNVGQNL | | |
| | | | VVDLSTQIFCHNDYPETITDYVTLQRGSAYGGVLSNFSGTVKYSGSSY | | |
| | | | PFPTTSETPRVVYNSRTDKPWPVALYLTPVSSAGGVAIKAGSLIAVLIL | | |
| | | | RQTNNYNSDDFQFVWNIYANNDVVVPTGGCDVSARDVTVTLPDYP | | |
| | | | GSVPIPLTVYCAKSQNLGYYLSGTTADAGNSIFTNTASFSPAQGVGV | | |
| | | | QLTRNGTIIPANNTVSLGAVGTSAVSLGLTANYARTGGQVTAGNVQ | | |
| | | | SIIGVTFVYQPGDGNADVTITVNGKVVAKGSGGGGASRELVVSYVNV | | |
| | | | NMGLKIRQLLWFHISCLTFGRETVLEYLVSFGVWIRTPPAYRPPNAPIL | | |
| | | | STLPETTVVGSGGGGHHHHHH [SEQ ID NO: 89] | | |
| | D_ | GSGG | METDTLLLWVLLLWVPGSTGDFACKTANGTAIPIGGGSANVYVNLA | | |
| | FimHDG_ | GGG | PVVNVGQNLVVDLSTQIFCHNDYPETITDYVTLQRGSAYGGVLSNFS | | |
| | Fer_ | linker | GTVKYSGSSYPFPTTSETPRVVYNSRTDKPWPVALYLTPVSSAGGVAI | | |
| | GSG4 | | KAGSLIAVLILRQTNNYNSDDFQFVWNIYANNDVVVPTGGCDVSAR | | |
| | | | DVTVTLPDYPGSVPIPLTVYCAKSQNLGYYLSGTTADAGNSIFTNTASF | | |
| | | | SPAQGVGVQLTRNGTIIPANNTVSLGAVGTSAVSLGLTANYARTGG | | |
| | | | QVTAGNVQSIIGVTFVYQPGDGNADVTITVNGKVVAKGSGGGGDII | | |
| | | | KLLNEQVNKEMNSSNLYMSMSSWCYTHSLDGAGLFLFDHAAEEYE | | |
| | | | HAKKLIIFLNENNVPVQLTSISAPEHKFEGLTQIFQKAYEHEQHISESIN | | |
| | | | NIVDHAIKSKDHATFNFLQWYVAEQHEEEVLFKDILDKIELIGNENHG | | |
| | | | LYLADQYVKGIAKSRK--[SEQ ID NO: 129] | | |
| | D_ | E. coli | METDTLLLWVLLLWVPGSTGDFACKTAQGTAIPIGGGSANVYVNLA | | |
| | FimHDG_ | ferritin | PVVNVGQNLVVDLSTQIFCHNDYPETITDYVTLQRGSAYGGVLSQFS | | |
| | Fer0.5_ | 0.5 | GTVKYSGSSYPFPTTSETPRVVYNSRTDKPWPVALYLTPVSSAGGVAI | | |
| | deglyc_ | tagless | KAGSLIAVLILRQTNNYNSDDFQFVWNIYANNDVVVPTGGCDVSAR | | |
| | tagless | | DVTVTLPDYPGSVPIPLTVYCAKSQNLGYYLSGTTADAGNSIFTNTASF | | |
| | | | SPAQGVGVQLTRQGTIIPAQNTVSLGAVGTSAVSLGLTANYARTGG | | |
| | | | QVTAGNVQSIIGVTFVYQPGDGNADVTITVNGKVVAKGSGGGGGG | | |
| | | | MLKPEMIEKLNEQMNLELYSSLLYQQMSAWCSYHGFEGAAAFLRR | | |
| | | | HAQEEMTHMQRLFDYLTDTGNLPRIDTIPSPFAEYSSLDELFQETYKH | | |
| | | | EQLITQKINELAHAAMTNQDYPTFNFLQWYVAEQHEEEKLFKSIIDKL | | |
| | | | SLAGKSGEGLYFIDKELSTLDTQN----[SEQ ID NO: 130] | | |
| | FimHDG_ | N −−> Q | METDTLLLWVLLLWVPGSTGFACKTAQGTAIPIGGGSANVYVNLAP | | |
| | deglyc_ | mutation | VVNVGQNLVVDLSTQIFCHNDYPETITDYVTLQRGSAYGGVLSQFSG | | |
| | _tagless | for | TVKYSGSSYPFPTTSETPRVVYNSRTDKPWPVALYLTPVSSAGGVAIK | | |
| | | avoiding | AGSLIAVLILRQTNNYNSDDFQFVWNIYANNDVVVPTGGCDVSARD | | |
| | | glyco- | VTVTLPDYPGSVPIPLTVYCAKSQNLGYYLSGTTADAGNSIFTNTASFS | | |
| | | silation | PAQGVGVQLTRQGTIIPAQNTVSLGAVGTSAVSLGLTANYARTGGQ | | |
| | | | VTAGNVQSIIGVTFVYQPGDGNADVTITVNGKVVAK---- | | |
| | | | [SEQ ID NO: 131] | | |
| | D_ | Initial | METDTLLLWVLLLWVPGSTGDFACKTAQGTAIPIGGGSANVYVNLA | | |
| | FimHDG_ | D; | PVVNVGQNLVVDLSTQIFCHNDYPETITDYVTLQRGSAYGGVLSQFS | | |
| | deglyc_ | N −−> Q | GTVKYSGSSYPFPTTSETPRVVYNSRTDKPWPVALYLTPVSSAGGVAI | | |
| | tagless | mutation | KAGSLIAVLILRQTNNYNSDDFQFVWNIYANNDVVVPTGGCDVSAR | | |
| | | for | DVTVTLPDYPGSVPIPLTVYCAKSQNLGYYLSGTTADAGNSIFTNTASF | | |
| | | avoiding | SPAQGVGVQLTRQGTIIPAQNTVSLGAVGTSAVSLGLTANYARTGG | | |
| | | glyco- | QVTAGNVQSIIGVTFVYQPGDGNADVTITVNGKVVAK---- | | |
| | | silation | [SEQ ID NO: 132] | | |
| | FimHDG_ | | METDTLLLWVLLLWVPGSTGFACKTAQGTAIPIGGGSANVYVNLAP | | |
| | N7Q_ | | VVNVGQNLVVDLSTQIFCHNDYPETITDYVTLQRGSAYGGVLSNFSG | | |
| | tagless | | TVKYSGSSYPFPTTSETPRVVYNSRTDKPWPVALYLTPVSSAGGVAIK | | |
| | | | AGSLIAVLILRQTNNYNSDDFQFVWNIYANNDVVVPTGGCDVSARD | | |
| | | | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAK----- | | |
| | | | [SEQ ID NO: 133] | | |
| | D_ | N −−> Q | METDTLLLWVLLLWVPGSTGDFACKTAQGTAIPIGGGSANVYVNLA | | |
| | FimHDG_ | mutation | PVVNVGQNLVVDLSTQIFCHNDYPETITDYVTLQRGSAYGGVLSQFS | | |
| | Fer_ | for | GTVKYSGSSYPFPTTSETPRVVYNSRTDKPWPVALYLTPVSSAGGVAI | | |
| | deglyc_ | avoiding | KAGSLIAVLILRQTNNYNSDDFQFVWNIYANNDVVVPTGGCDVSAR | | |
| | tagless | glyco- | DVTVTLPDYPGSVPIPLTVYCAKSQNLGYYLSGTTADAGNSIFTNTASF | | |
| | | silation | SPAQGVGVQLTRQGTIIPAQNTVSLGAVGTSAVSLGLTANYARTGG | | |
| | | | QVTAGNVQSIIGVTFVYQPGDGNADVTITVNGKVVAKSGSGSGGGG | | |
| | | | GGSDIIKLLNEQVNKEMQSSNLYMSMSSWCYTHSLDGAGLFLFDHA | | |
| | | | AEEYEHAKKLIFLNENNVPVQLTSISAPEHKFEGLTQIFQKAYEHEQHI | | |
| | | | SESINNIVDHAIKSKDHATFNFLQWYVAEQHEEEVLFKDILDKIELIGN | | |
| | | | ENHGLYLADQYVKGIAKSRK----[SEQ ID NO: 134] | | |
| | FimH_ | Tagless | METDTLLLWVLLLWVPGSTGFACKTANGTAIPIGGGSANVYVNLAP | | |
| | PGDGN_ | FimHDG | VVNVGQNLVVDLSTQIFCHNDYPETITDYVTLQRGSAYGGVLSNFSG | | |
| | GGS | | TVKYSGSSYPFPTTSETPRVVYNSRTDKPWPVALYLTPVSSAGGVAIK | | |
| | 4- | | AGSLIAVLILRQTNNYNSDDFQFVWNIYANNDVVVPTGGCDVSARD | | |
| | Ferritn | | VTVTLPDYPGSVPIPLTVYCAKSQNLGYYLSGTTADAGNSIFTNTASFS | | |
| | j96 | | PAQGVGVQLTRNGTIIPANNTVSLGAVGTSAVSLGLTANYARTGGQ | | |
| | | | VTAGNVQSIIGVTFVYQPGDGNADVTITVNGKVVAKGGSGGSGGSG | | |
| | | | GSDIIKLLNEQVNKEMNSSNLYMSMSSWCYTHSLDGAGLFLFDHAA | | |
| | | | EEYEHAKKLIIFLNENNVPVQLTSISAPEHKFEGLTQIFQKAYEHEQHIS | | |
| | | | ESINNIVDHAIKSKDHATFNFLQWYVAEQHEEEVLFKDILDKIELIGNE | | |
| | | | NHGLYLADQYVKGIAKSRKS* [SEQ ID NO: 135] | | |
| 1397SI | FimH_ | Tagless | METDTLLLWVLLLWVPGSTGFACKTANGTAIPIGGGSANVYVNLAP | | |
| | PGDGN_ | FimHDG | VVNVGQNLVVDLSTQIFCHNDYPETITDYVTLQRGSAYGGVLSNFSG | | |
| | DG j96 | | TVKYSGSSYPFPTTSETPRVVYNSRTDKPWPVALYLTPVSSAGGVAIK | | |
| | | | AGSLIAVLILRQTNNYNSDDFQFVWNIYANNDVVVPTGGCDVSARD | | |
| | | | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAK | | |
| | | | [SEQ ID NO: 136] | | |
| | FIMHDG_ | linker | METDTLLLWVLLLWVPGSTGFACKTANGTAIPIGGGSANVYVNLAP | | |
| | PADRE | PADRE | AVNVGQNLVVDLSTQIFCHNDYPETITDYVTLQRGSAYGGVLSSFSG | | |
| | encap- | | TVKYNGSSYPFPTTSETPRVVYNSRTDKPWPVALYLTPVSSAGGVAIK | | |
| | suline536 | | AGSLIAVLILRQTNNYNSDDFQFVWNIYANNDVVVPTGGCDVSARD | | |
| | | | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAKFVAAWTLKAA | | |
| | | | AMEFLKRSFAPLTEKQWQEIDNRAREIFKTQLYGRKFVDVEGPYGW | | |
| | | | EYAAHPLGEVEVLSDENEVVKWGLRKSLPLIELRATFTLDLWELDNLE | | |
| | | | RGKPNVDLSSLEETVRKVAEFEDEVIFRGCEKSGVKGLLSFEERKIECG | | |
| | | | STPKDLLEAIVRALSIFSKDGIEGPYTLVINTDRWINFLKEEAGHYPLEK | | |
| | | | RVEECLRGGKIITTPRIEDALVVSERGGDFKLILGQDLSIGYEDREKDAV | | |
| | | | RLFITETFTFQVVNPEALILLKF-[SEQ ID NO: 137] | | |
| | FIMH_ | mixed | METDTLLLWVLLLWVPGSTGFACKTANGTAIPIGGGSANVYVNLAP | | |
| | NOD- | linker | VVNVGQNLVVDLSTQIFCHNDYPETITDYVTLQRGSAYGGVLSNFSG | | |
| | FERRITN | with G | TVKYSGSSYPFPTTSETPRVVYNSRTDKPWPVALYLTPVSSAGGVAIK | | |
| | J96 | S and | AGSLIAVLILRQTNNYNSDDFQFVWNIYANNDVVVPTGGCDVSARD | | |
| | | H | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAKSGSHHHGSGG | | |
| | | | GGGSDIIKLLNEQVNKEMNSSNLYMSMSSWCYTHSLDGAGLFLFDH | | |
| | | | AAEEYEHAKKLIIFLNENNVPVQLTSISAPEHKFEGLTQIFQKAYEHEQ | | |
| | | | HISESINNIVDHAIKSKDHATFNFLQWYVAEQHEEEVLFKDILDKIELIG | | |
| | | | NENHGLYLADQYVKGIAKSRK [SEQ ID NO: 138] | | |
| | FIMH_ | rigid | METDTLLLWVLLLWVPGSTGFACKTANGTAIPIGGGSANVYVNLAP | | |
| | DG_NOAL | linker | VVNVGQNLVVDLSTQIFCHNDYPETITDYVTLQRGSAYGGVLSNFSG | | |
| | FA- | | TVKYSGSSYPFPTTSETPRVVYNSRTDKPWPVALYLTPVSSAGGVAIK | | |
| | ferritin | | AGSLIAVLILRQTNNYNSDDFQFVWNIYANNDVVVPTGGCDVSARD | | |
| | J96 | | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAKGGGGSLVPRG | | |
| | | | SGGGGSDIIKLLNEQVNKEMNSSNLYMSMSSWCYTHSLDGAGLFLF | | |
| | | | DHAAEEYEHAKKLIIFLNENNVPVQLTSISAPEHKFEGLTQIFQKAYEH | | |
| | | | EQHISESINNIVDHAIKSKDHATFNFLQWYVAEQHEEEVLFKDILDKIE | | |
| | | | LIGNENHGLYLADQYVKGIAKSRKS--[SEQ ID NO: 139] | | |
| | FIMH_ | HIS | METDTLLLWVLLLWVPGSTGFACKTANGTAIPIGGGSANVYVNLAP | | |
| | NOD_S_ | linker- | VVNVGQNLVVDLSTQIFCHNDYPETITDYVTLQRGSAYGGVLSNFSG | | |
| | HIS_ | terminal | TVKYSGSSYPFPTTSETPRVVYNSRTDKPWPVALYLTPVSSAGGVAIK | | |
| | FERRITN_ | SRKS | AGSLIAVLILRQTNNYNSDDFQFVWNIYANNDVVVPTGGCDVSARD | | |
| | J96 | | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAKSGSHHHHHH | | |
| | | | HHGGSDIIKLLNEQVNKEMNSSNLYMSMSSWCYTHSLDGAGLFLFD | | |
| | | | HAAEEYEHAKKLIIFLNENNVPVQLTSISAPEHKFEGLTQIFQKAYEHE | | |
| | | | QHISESINNIVDHAIKSKDHATFNFLQWYVAEQHEEEVLFKDILDKIELI | | |
| | | | GNENHGLYLADQYVKGIAKSRKS--[SEQ ID NO: 140] | | |
| | FIMH_ | | METDTLLLWVLLLWVPGSTGFACKTANGTAIPIGGGSANVYVNLAP | | |
| | DG_ | | AVNVGQNLVVDLSTQIFCHNDYPETITDYVTLQRGSAYGGVLSSFSG | | |
| | PGDGN- | | TVKYNGSSYPFPTTSETPRVVYNSRTDKPWPVALYLTPVSSAGGVAIK | | |
| | PADRE- | | AGSLIAVLILRQTNNYNSDDFQFVWNIYANNDVVVPTGGCDVSARD | | |
| | Ferritin | | VTVTLPDYPGSVPIPLTVYCAKSQNLGYYLSGTTADAGNSIFTNTASFS | | |
| | 536 | | PAQGVGVQLTRNGTIIPANNTVSLGAVGTSAVSLGLTANYARTGGQ | | |
| | | | VTAGNVQSIIGVTFVYQPGDGNADVTITVNGKVVAKSGSFVAAWTL | | |
| | | | KAAAGGSDIIKLLNEQVNKEMNSSNLYMSMSSWCYTHSLDGAGLFL | | |
| | | | FDHAAEEYEHAKKLIIFLNENNVPVQLTSISAPEHKFEGLTQIFQKAYE | | |
| | | | HEQHISESINNIVDHAIKSKDHATFNFLQWYVAEQHEEEVLFKDILDKI | | |
| | | | ELIGNENHGLYLADQYVKGIAKSRKS-[SEQ ID NO: 141] | | |
| | fimh- | Rigid | METDTLLLWVLLLWVPGSTGFACKTANGTAIPIGGGSANVYVNLAP | | |
| | DG_ | linker | VVNVGQNLVVDLSTQIFCHNDYPETITDYVTLQRGSAYGGVLSNFSG | | |
| | ferri- | | TVKYSGSSYPFPTTSETPRVVYNSRTDKPWPVALYLTPVSSAGGVAIK | | |
| | tina- | | AGSLIAVLILRQTNNYNSDDFQFVWNIYANNDVVVPTGGCDVSARD | | |
| | linkerAl | | VTVTLPDYPGSVPIPLTVYCAKSQNLGYYLSGTTADAGNSIFTNTASFS | | |
| | pha | | PAQGVGVQLTRNGTIIPANNTVSLGAVGTSAVSLGLTANYARTGGQ | | |
| | | | VTAGNVQSIIGVTFVYQPGDGNADVTITVNGKVVAKAEAAAKEAAA | | |
| | | | KEAAAKADIIKLLNEQVNKEMNSSNLYMSMSSWCYTHSLDGAGLFL | | |
| | | | FDHAAEEYEHAKKLIIFLNENNVPVQLTSISAPEHKFEGLTQIFQKAYE | | |
| | | | HEQHISESINNIVDHAIKSKDHATFNFLQWYVAEQHEEEVLFKDILDKI | | |
| | | | ELIGNENHGLYLADQYVKGIAKSRKS--[SEQ ID NO: 142] | | |
| | FIMH- | Linker | METDTLLLWVLLLWVPGSTGFACKTANGTAIPIGGGSANVYVNLAP | | |
| | PADRE- | PADRE | AVNVGQNLVVDLSTQIFCHNDYPETITDYVTLQRGSAYGGVLSSFSG | | |
| | Ferritin | | TVKYNGSSYPFPTTSETPRVVYNSRTDKPWPVALYLTPVSSAGGVAIK | | |
| | 536_noS | | AGSLIAVLILRQTNNYNSDDFQFVWNIYANNDVVVPTGGCDVSARD | | |
| | paces | | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAKFVAAWTLKAA | | |
| | | | ADIIKLLNEQVNKEMNSSNLYMSMSSWCYTHSLDGAGLFLFDHAAE | | |
| | | | EYEHAKKLIIFLNENNVPVQLTSISAPEHKFEGLTQIFQKAYEHEQHISE | | |
| | | | SINNIVDHAIKSKDHATFNFLQWYVAEQHEEEVLFKDILDKIELIGNEN | | |
| | | | HGLYLADQYVKGIAKSRKS--[SEQ ID NO: 143] | | |
| | FIMH_ | | METDTLLLWVLLLWVPGSTGDFACKTANGTAIPIGGGSANVYVNLA | | |
| | DG_GSG4- | | PVVNVGQNLVVDLSTQIFCHNDYPETITDYVTLQRGSAYGGVLSNFS | | |
| | ferritin | | GTVKYSGSSYPFPTTSETPRVVYNSRTDKPWPVALYLTPVSSAGGVAI | | |
| | J96 | | KAGSLIAVLILRQTNNYNSDDFQFVWNIYANNDVVVPTGGCDVSAR | | |
| | | | DVTVTLPDYPGSVPIPLTVYCAKSQNLGYYLSGTTADAGNSIFTNTASF | | |
| | | | SPAQGVGVQLTRNGTIIPANNTVSLGAVGTSAVSLGLTANYARTGG | | |
| | | | QVTAGNVQSIIGVTFVYQPGDGNADVTITVNGKVVAKSGSGSGGGG | | |
| | | | GGSDIIKLLNEQVNKEMNSSNLYMSMSSWCYTHSLDGAGLFLFDHA | | |
| | | | AEEYEHAKKLIIFLNENNVPVQLTSISAPEHKFEGLTQIFQKAYEHEQHI | | |
| | | | SESINNIVDHAIKSKDHATFNFLQWYVAEQHEEEVLFKDILDKIELIGN | | |
| | | | ENHGLYLADQYVKGIAKSRK--[SEQ ID NO: 144] | | |
| | FIMH_ | | METDTLLLWVLLLWVPGSTGFACKTANGTAIPIGGGSANVYVNLAP | | |
| | DG_ | | VVNVGQNLVVDLSTQIFCHNDYPETITDYVTLQRGSAYGGVLSNFSG | | |
| | PGDGN- | | TVKYSGSSYPFPTTSETPRVVYNSRTDKPWPVALYLTPVSSAGGVAIK | | |
| | SGS_ | | AGSLIAVLILRQTNNYNSDDFQFVWNIYANNDVVVPTGGCDVSARD | | |
| | PADRE- | | VTVTLPDYPGSVPIPLTVYCAKSQNLGYYLSGTTADAGNSIFTNTASFS | | |
| | Ferritin | | PAQGVGVQLTRNGTIIPANNTVSLGAVGTSAVSLGLTANYARTGGQ | | |
| | j96 | | VTAGNVQSIIGVTFVYQPGDGNADVTITVNGKVVAKSGSFVAAWTL | | |
| | | | KAAAGGSDIIKLLNEQVNKEMNSSNLYMSMSSWCYTHSLDGAGLFL | | |
| | | | FDHAAEEYEHAKKLIIFLNENNVPVQLTSISAPEHKFEGLTQIFQKAYE | | |
| | | | HEQHISESINNIVDHAIKSKDHATFNFLQWYVAEQHEEEVLFKDILDKI | | |
| | | | ELIGNENHGLYLADQYVKGIAKSRKS-[SEQ ID NO: 145] | | |
| | fimh- | Rigid | METDTLLLWVLLLWVPGSTGFACKTANGTAIPIGGGSANVYVNLAP | | |
| | ferritina | linker | VVNVGQNLVVDLSTQIFCHNDYPETITDYVTLQRGSAYGGVLSNFSG | | |
| | linkerN | | TVKYSGSSYPFPTTSETPRVVYNSRTDKPWPVALYLTPVSSAGGVAIK | | |
| | ONa | | AGSLIAVLILRQTNNYNSDDFQFVWNIYANNDVVVPTGGCDVSARD | | |
| | | | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAKGGGGSLVPRG | | |
| | | | SGGGGSDIIKLLNEQVNKEMNSSNLYMSMSSWCYTHSLDGAGLFLF | | |
| | | | DHAAEEYEHAKKLIIFLNENNVPVQLTSISAPEHKFEGLTQIFQKAYEH | | |
| | | | EQHISESINNIVDHAIKSKDHATFNFLQWYVAEQHEEEVLFKDILDKIE | | |
| | | | LIGNENHGLYLADQYVKGIAKSRKS--[SEQ ID NO: 146] | | |
| | FIMH- | Linker | METDTLLLWVLLLWVPGSTGFACKTANGTAIPIGGGSANVYVNLAP | | |
| | Ferritin | PADRE | VVNVGQNLVVDLSTQIFCHNDYPETITDYVTLQRGSAYGGVLSNFSG | | |
| | J96_noS | | TVKYSGSSYPFPTTSETPRVVYNSRTDKPWPVALYLTPVSSAGGVAIK | | |
| | paces | | AGSLIAVLILRQTNNYNSDDFQFVWNIYANNDVVVPTGGCDVSARD | | |
| | | | VTVTLPDYPGSVPIPLTVYCAKSQNLGYYLSGTTADAGNSIFTNTASFS | | |
| | | | PAQGVGVQLTRNGTIIPANNTVSLGAVGTSAVSLGLTANYARTGGQ | | |
| | | | VTAGNVQSIIGVTFVYQPGDGNADVTITVNGKVVAKFVAAWTLKAA | | |
| | | | ADIIKLLNEQVNKEMNSSNLYMSMSSWCYTHSLDGAGLFLFDHAAE | | |
| | | | EYEHAKKLIIFLNENNVPVQLTSISAPEHKFEGLTQIFQKAYEHEQHISE | | |
| | | | SINNIVDHAIKSKDHATFNFLQWYVAEQHEEEVLFKDILDKIELIGNEN | | |
| | | | HGLYLADQYVKGIAKSRKS--[SEQ ID NO: 147] | | |
| | FIMH_ | With | METDTLLLWVLLLWVPGSTGDFACKTANGTAIPIGGGSANVYVNLA | | |
| | DG_ | N- | PAVNVGQNLVVDLSTQIFCHNDYPETITDYVTLQRGSAYGGVLSSFS | | |
| | PGDGN- | terminal | GTVKYNGSSYPFPTTSETPRVVYNSRTDKPWPVALYLTPVSSAGGVAI | | |
| | GGS4- | D | KAGSLIAVLILRQTNNYNSDDFQFVWNIYANNDVVVPTGGCDVSAR | | |
| | Ferritin | | DVTVTLPDYPGSVPIPLTVYCAKSQNLGYYLSGTTADAGNSIFTNTASF | | |
| | 536 | | SPAQGVGVQLTRNGTIIPANNTVSLGAVGTSAVSLGLTANYARTGG | | |
| | | | QVTAGNVQSIIGVTFVYQPGDGNADVTITVNGKVVAKGGSGGSGGS | | |
| | | | GGSDIIKLLNEQVNKEMNSSNLYMSMSSWCYTHSLDGAGLFLFDHA | | |
| | | | AEEYEHAKKLIIFLNENNVPVQLTSISAPEHKFEGLTQIFQKAYEHEQHI | | |
| | | | SESINNIVDHAIKSKDHATFNFLQWYVAEQHEEEVLFKDILDKIELIGN | | |
| | | | ENHGLYLADQYVKGIAKSRK-[SEQ ID NO: 148] | | |
| | Fusion | | METDTLLLWVLLLWVPGSTGDFACKTANGTAIPIGGGSANVYVNLA | | |
| | of | | PAVNVGQNLVVDLSTQIFCHNDYPETITDYVTLQRGSAYGGVLSSFS | | |
| | FimH_D | | GTVKYNGSSYPFPTTSETPRVVYNSRTDKPWPVALYLTPVSSAGGVAI | | |
| | G to | | KAGSLIAVLILRQTNNYNSDDFQFVWNIYANNDVVVPTGGCDVSAR | | |
| | E. Coli | | DVTVTLPDYPGSVPIPLTVYCAKSQNLGYYLSGTTADAGNSIFTNTASF | | |
| | Ferritin | | SPAQGVGVQLTRNGTIIPANNTVSLGAVGTSAVSLGLTANYARTGG | | |
| | stabilize | | QVTAGNVQSIIGVTFVYQPGDGNADVTITVNGKVVAKSGSHHHHH | | |
| | d 0.5 | | HHHGGSMLKPEMIEKLNEQMNLELYSSLLYQQMSAWCSYHGFEGA | | |
| | | | AAFLRRHAQEEMTHMQRLFDYLTDTGNLPRIDTIPSPFAEYSSLDELF | | |
| | | | QETYKHEQLITQKINELAHAAMTNQDYPTFNFLQWYVAEQHEEEKL | | |
| | | | FKSIIDKLSLAGKSGEGLYFIDKELSTLDTQN-[SEQ ID NO: 153] | | |

TABLE 3
construct nucleic acid sequences

| | |
|---|---|
| FIMH_DG_ | ATGGAAACCGATACACTGCTGCTGTGGGTGCTGTTGCTCTGGGTTCCAGGCTCTACAGGCGATTTTGC |
| 536- | CTGCAAGACCGCCAACGGCACAGCCATTCCTATTGGCGGAGGCAGCGCCAATGTGTACGTTAACCTGG |
| 1EUM_ | CTCCTGCCGTGAACGTGGGCCAGAATCTGGTGGTGGATCTGAGCACCCAGATCTTTTGCCACAACGAC |
| 0_5 | TACCCCGAGACAATCACCGACTACGTGACACTGCAGAGAGGCTCTGCTTACGGCGGCGTGCTGTCTAG |
| | CTTTAGCGGCACCGTGAAGTACAACGGCAGCAGCTACCCATTTCCTACCACCAGCGAGACACCCAGAG |
| | TGGTGTACAACAGCAGAACCGACAAGCCCTGGCCTGTGGCTCTGTACCTGACACCTGTTAGTTCTGCC |
| | GGCGGAGTGGCCATTAAGGCCGGATCTCTGATTGCCGTGCTGATCCTGCGGCAGACCAACAACTACA |
| | ACAGCGACGACTTCCAGTTCGTGTGGAACATCTACGCCAACAACGACGTGGTGGTGCCTACAGGCGG |
| | ATGTGATGTGTCCGCCAGAGATGTGACAGTGACCCTGCCTGATTACCCCGGCTCTGTGCCTATTCCTCT |
| | GACCGTGTACTGCGCCAAGAGCCAGAACCTGGGCTACTACCTGTCTGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTGTCTCTTGG |
| | CCTGACAGCCAACTATGCCAGAACAGGCGGACAAGTGACAGCCGGCAATGTGCAGTCTATCATCGGC |
| | GTGACCTTCGTGTATCAGCCTGGCGACGGAAACGCCGATGTGACCATCACAGTGAATGGCAAGGTGG |
| | TGGCCAAGAGCGGAAGCCACCACCATCATCACCATCACCACGGCGGCTCTATGCTGAAGCCCGAGATG |
| | ATCGAGAAGCTGAACGAGCAGATGAACCTGGAACTGTACAGCTCCCTGCTGTACCAGCAGATGAGCG |
| | CCTGGTGTAGCTATCACGGATTTGAGGGCGCTGCCGCCTTTCTGAGAAGGCACGCCCAAGAGGAAAT |
| | GACCCACATGCAGCGGCTGTTCGACTACCTGACCGATACCGGCAATCTGCCCAGAATCGACACAATCC |
| | CATCTCCATTCGCCGAGTACAGCAGCCTGGACGAGCTGTTCCAAGAAACCTACAAGCACGAGCAGCTG |
| | ATCACCCAGAAGATCAACGAACTGGCCCATGCCGCCATGACCAACCAGGACTACCCTACCTTCAACTTC |
| | CTGCAGTGGTACGTGGCCGAGCAGCACGAGGAAGAGAAGCTGTTCAAGAGCATCATCGACAAGCTGA |
| | GCCTGGCCGGAAAGTCTGGCGAGGGCCTGTACTTTATCGACAAAGAGCTGAGCACACTGGATACCCA |
| | GAACTGA [SEQ ID NO: 45] |
| FIMH_DG_ | ATGGAAACCGATACACTGCTGCTGTGGGTGCTGTTGCTCTGGGTTCCAGGCTCTACAGGCGATTTTGC |
| PGDGN_ | CTGCAAGACCGCCAACGGCACAGCCATTCCTATTGGCGGAGGCAGCGCCAATGTGTACGTTAACCTGG |
| 536-MI3 | CTCCTGCCGTGAACGTGGGCCAGAATCTGGTGGTGGATCTGAGCACCCAGATCTTTTGCCACAACGAC |
| | TACCCCGAGACAATCACCGACTACGTGACACTGCAGAGAGGCTCTGCTTACGGCGGCGTGCTGTCTAG |
| | CTTTAGCGGCACCGTGAAGTACAACGGCAGCAGCTACCCATTTCCTACCACCAGCGAGACACCCAGAG |
| | TGGTGTACAACAGCAGAACCGACAAGCCCTGGCCTGTGGCTCTGTACCTGACACCTGTTAGTTCTGCC |
| | GGCGGAGTGGCCATTAAGGCCGGATCTCTGATTGCCGTGCTGATCCTGCGGCAGACCAACAACTACA |
| | ACAGCGACGACTTCCAGTTCGTGTGGAACATCTACGCCAACAACGACGTGGTGGTGCCTACAGGCGG |
| | ATGTGATGTGTCCGCCAGAGATGTGACAGTGACCCTGCCTGATTACCCCGGCTCTGTGCCTATTCCTCT |
| | GACCGTGTACTGCGCCAAGAGCCAGAACCTGGGCTACTACCTGTCTGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTGTCTCTTGG |
| | CCTGACAGCCAACTATGCCAGAACAGGCGGACAAGTGACAGCCGGCAATGTGCAGTCTATCATCGGC |
| | GTGACCTTCGTGTATCAGCCTGGCGACGGAAACGCCGATGTGACCATCACAGTGAATGGCAAGGTGG |
| | TGGCCAAGAGCGGAAGCCACCACCATCATCACCATCACCACGGCGGCAGCATGAAGATGGAAGAACT |
| | GTTCAAGAAGCACAAGATCGTCGCCGTGCTGCGGGCCAATTCTGTGGAAGAGGCCAAAAAAAAGGCC |
| | CTGGCCGTGTTTCTTGGCGGAGTGCACCTGATCGAGATCACCTTTACCGTGCCTGACGCCGACACCGT |
| | GATCAAAGAGCTGAGCTTCCTGAAAGAGATGGGCGCCATCATCGGAGCCGGCACAGTGACATCTGTT |
| | GAGCAGGCCAGAAAGGCCGTGGAATCTGGCGCCGAGTTTATCGTGTCCCCTCACCTGGATGAGGAAA |
| | TCAGCCAGTTCGCCAAAGAAAAGGGCGTGTTCTACATGCCCGGCGTGATGACACCTACAGAGCTGGTC |
| | AAAGCCATGAAGCTGGGCCACACCATCCTGAAGCTGTTTCCAGGCGAAGTCGTGGGCCCTCAGTTCGT |
| | GAAAGCTATGAAGGGCCCATTTCCAAACGTGAAGTTCGTGCCCACTGGCGGCGTGAACCTGGATAAT |
| | GTGTGCGAGTGGTTCAAGGCTGGCGTGCTGGCTGTTGGAGTTGGCTCTGCTCTGGTCAAGGGCACAC |
| | CTGTGGAAGTGGCTGAGAAGGCCAAGGCCTTCGTGGAAAAGATCAGAGGCTGCACCGAGTGA |
| | [SEQ ID NO: 46] |
| FIMH_DG_ | ATGGAAACCGATACACTGCTGCTGTGGGTGCTGTTGCTCTGGGTTCCAGGCTCTACAGGCGATTTTGC |
| PGDGN_ | CTGCAAGACCGCCAACGGCACAGCCATTCCTATTGGCGGAGGCAGCGCCAATGTGTACGTTAACCTGG |
| 536- | CTCCTGCCGTGAACGTGGGCCAGAATCTGGTGGTGGATCTGAGCACCCAGATCTTTTGCCACAACGAC |
| encapsuline | TACCCCGAGACAATCACCGACTACGTGACACTGCAGAGAGGCTCTGCTTACGGCGGCGTGCTGTCTAG |
| | CTTTAGCGGCACCGTGAAGTACAACGGCAGCAGCTACCCATTTCCTACCACCAGCGAGACACCCAGAG |
| | TGGTGTACAACAGCAGAACCGACAAGCCCTGGCCTGTGGCTCTGTACCTGACACCTGTTAGTTCTGCC |
| | GGCGGAGTGGCCATTAAGGCCGGATCTCTGATTGCCGTGCTGATCCTGCGGCAGACCAACAACTACA |
| | ACAGCGACGACTTCCAGTTCGTGTGGAACATCTACGCCAACAACGACGTGGTGGTGCCTACAGGCGG |
| | ATGTGATGTGTCCGCCAGAGATGTGACAGTGACCCTGCCTGATTACCCCGGCTCTGTGCCTATTCCTCT |
| | GACCGTGTACTGCGCCAAGAGCCAGAACCTGGGCTACTACCTGTCTGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTGTCTCTTGG |
| | CCTGACAGCCAACTATGCCAGAACAGGCGGACAAGTGACAGCCGGCAATGTGCAGTCTATCATCGGC |
| | GTGACCTTCGTGTATCAGCCTGGCGACGGAAACGCCGATGTGACCATCACAGTGAATGGCAAGGTGG |
| | TGGCCAAGAGCGGAAGCCACCACCATCATCACCATCACCACGGCGGCAGCATGGAATTTCTGAAGAG |
| | AAGCTTCGCCCCACTGACCGAGAAGCAGTGGCAAGAGATCGACAACCGGGCCAGAGAGATCTTCAAG |
| | ACCCAGCTGTACGGCCGGAAGTTCGTGGATGTGGAAGGCCCTTATGGCTGGGAGTATGCCGCTCATC |
| | CTCTGGGCGAAGTGGAAGTGCTGAGCGACGAGAATGAGGTCGTGAAGTGGGGCCTGAGAAAGAGCC |
| | TGCCTCTGATCGAGCTGAGAGCCACCTTCACACTGGACCTGTGGGAACTCGACAACCTGGAAAGGGG |
| | CAAGCCCAATGTGGACCTGAGCAGCCTGGAAGAGACAGTGCGGAAGGTGGCCGAGTTCGAGGACGA |
| | AGTGATCTTCAGAGGCTGCGAGAAGTCTGGCGTGAAGGGCCTGCTGAGCTTCGAGGAACGGAAGATC |
| | GAGTGTGGCAGCACCCCTAAGGATCTGCTGGAAGCCATCGTGCGGGCCCTGAGCATCTTCTCTAAGGA |
| | TGGCATCGAGGGCCCCTACACACTGGTCATCAACACCGACCGGTGGATCAACTTCCTGAAAGAGGAA |
| | GCCGGCCACTATCCTCTGGAAAAGCGCGTGGAAGAGTGCCTGAGAGGCGGCAAGATCATCACAACCC |
| | CTAGAATCGAGGACGCCCTGGTGGTTTCTGAGAGAGGCGGAGACTTCAAGCTGATCCTTGGCCAGGA |
| | CCTGTCCATCGGCTACGAGGACAGAGAAAAAGACGCCGTGCGGCTGTTCATCACCGAAACCTTCACCT |
| | TCCAAGTGGTCAACCCCGAGGCTCTGATTCTGCTGAAGTTCTGA [SEQ ID NO: 47] |
| HBcFIM | ATGGAAACCGATACACTGCTGCTGTGGGTGCTGTTGCTCTGGGTTCCAGGATCTACCGGCGACGACAT |
| HLJ96 | CGACCCCTACAAAGAGTTTGGCGCCAGCGTCGAGCTGCTGAGCTTCCTGCCTAGCGACTTCTTCCCTTC |
| | CATCCGGGATCTGCTGGATACCGCTAGCGCCCTGTATAGAGAGGCCCTGGAAAGCCCTGAGCACTGCT |
| | CTCCACATCACACAGCCCTGAGACAGGCCATCCTGTGTTGGGGCGAACTGATGAATCTGGCCACCTGG |
| | GTCGGAAGCAACCTGGAAGATCCTGGTTCTGGCGGCGGAGGCTTTGCCTGTAAAACAGCCAATGGCA |
| | CCGCCATTCCTATCGGAGGCGGCAGCGCCAATGTGTACGTTAACCTGGCTCCTGTGGTCAACGTGGGC |
| | CAGAATCTGGTGGTGGACCTGAGCACCCAGATCTTTTGCCACAACGACTACCCCGAGACAATCACCGA |
| | CTACGTGACACTGCAGAGAGGCTCTGCTTACGGCGGCGTGCTGAGCAATTTTTCCGGCACAGTGAAGT |
| | ACAGCGGCAGCAGCTACCCATTTCCTACCACCAGCGAGACACCCAGAGTGGTGTACAACAGCAGAACC |
| | GACAAGCCCTGGCCTGTGGCTCTGTACCTGACACCTGTTAGTTCTGCTGGCGGAGTGGCCATCAAGGC |
| | CGGATCTCTGATTGCCGTGCTGATCCTGCGGCAGACCAACAACTACAACAGCGACGACTTCCAGTTCG |
| | TGTGGAACATCTACGCCAACAACGACGTGGTGGTGCCTACAGGCGGAGGATCTGGCGGAGCTTCTAG |
| | AGAACTGGTCGTGTCCTACGTGAACGTGAACATGGGCCTGAAGATCCGGCAGCTGCTCTGGTTTCACA |
| | TCAGCTGTCTGACCTTCGGCCGGGAAACCGTGCTGGAATACCTGGTGTCCTTCGGCGTGTGGATCAGA |
| | ACCCCTCCTGCCTATAGACCTCCTAACGCTCCCATCCTGAGCACACTGCCTGAGACAACAGTTGTTGGA |
| | AGCGGAGGCGGAGGCCACCACCATCACCATCAT [SEQ ID NO: 48] |
| HBcFIM | ATGGAGACCGACACCCTGCTGCTGTGGGTGCTGCTGCTGTGGGTGCCCGGCAGCACCGGCGACGACA |
| HDGJ96 | TCGACCCCTACAAGGAGTTCGGCGCCAGCGTGGAGCTGCTGAGCTTCCTGCCCAGCGACTTCTTCCCC |
| | AGCATCCGGGACCTGCTGGACACCGCCAGCGCCCTGTACCGGGAGGCCCTGGAGAGCCCCGAGCACT |
| | GCAGCCCCCACCACACCGCCCTGCGGCAGGCCATCCTGTGCTGGGGCGAGCTGATGAACCTGGCCAC |
| | CTGGGTGGGCAGCAACCTGGAGGACCCCGGCAGCGGCGGCGGCGGCTTCGCCTGCAAGACCGCCAA |
| | CGGCACCGCCATCCCCATCGGCGGCGGCAGCGCCAACGTGTACGTGAACCTGGCCCCCGTGGTGAAC |
| | GTGGGCCAGAACCTGGTGGTGGACCTGAGCACCCAGATCTTCTGCCACAACGACTACCCCGAGACCAT |
| | CACCGACTACGTGACCCTGCAGCGGGGCAGCGCCTACGGCGGCGTGCTGAGCAACTTCAGCGGCACC |
| | GTGAAGTACAGCGGCAGCAGCTACCCCTTCCCCACCACCAGCGAGACCCCCCGGGTGGTGTACAACA |
| | GCCGGACCGACAAGCCCTGGCCCGTGGCCCTGTACCTGACCCCCGTGAGCAGCGCCGGCGGCGTGGC |
| | CATCAAGGCCGGCAGCCTGATCGCCGTGCTGATCCTGCGGCAGACCAACAACTACAACAGCGACGACT |
| | TCCAGTTCGTGTGGAACATCTACGCCAACAACGACGTGGTGGTGCCCACCGGCGGCTGCGACGTGAG |
| | CGCCCGGGACGTGACCGTGACCCTGCCCGACTACCCCGGCAGCGTGCCCATCCCCCTGACCGTGTACT |
| | GCGCCAAGAGCCAGAACCTGGGCTACTACCTGAGCGGCACCACCGCCGACGCCGGCAACAGCATCTT |
| | CACCAACACCGCCAGCTTCAGCCCCGCCCAGGGCGTGGGCGTGCAGCTGACCCGGAACGGCACCATC |
| | ATCCCCGCCAACAACACCGTGAGCCTGGGCGCCGTGGGCACCAGCGCCGTGAGCCTGGGCCTGACCG |
| | CCAACTACGCCCGGACCGGCGGCCAGGTGACCGCCGGCAACGTGCAGAGCATCATCGGCGTGACCTT |
| | CGTGTACCAGCCCGGCGACGGCAACGCCGACGTGACCATCACCGTGAACGGCAAGGTGGTGGCCAA |
| | GGGCAGCGGCGGCGGCGGCGCCAGCCGGGAGCTGGTGGTGAGCTACGTGAACGTGAACATGGGCCT |
| | GAAGATCCGGCAGCTGCTGTGGTTCCACATCAGCTGCCTGACCTTCGGCCGGGAGACCGTGCTGGAG |
| | TACCTGGTGAGCTTCGGCGTGTGGATCCGGACCCCCCCCGCCTACCGGCCCCCCAACGCCCCCATCCTG |
| | AGCACCCTGCCCGAGACCACCGTGGTGGGCAGCGGCGGCGGCGGCCACCACCACCACCACCAC |
| | [SEQ ID NO: 49] |
| FIMHL- | ATGGAAACCGATACACTGCTGCTGTGGGTGCTGTTGCTCTGGGTTCCAGGCTCTACAGGCGATTTTGC |
| HIS-Mi3 | CTGCAAGACCGCCAACGGCACAGCCATTCCTATTGGCGGAGGCAGCGCCAATGTGTACGTGAACCTG |
| J96 | GCTCCTGTGGTCAACGTGGGCCAGAATCTGGTGGTGGACCTGAGCACCCAGATCTTTTGCCACAACGA |
| | CTACCCCGAGACAATCACCGACTACGTGACACTGCAGAGAGGCTCTGCTTACGGCGGCGTGCTGAGC |
| | AATTTTTCCGGCACAGTGAAGTACAGCGGCAGCAGCTACCCATTTCCTACCACCAGCGAGACACCCAG |
| | AGTGGTGTACAACAGCAGAACCGACAAGCCCTGGCCTGTGGCTCTGTACCTGACACCTGTTAGTTCTG |
| | CCGGCGGAGTGGCCATTAAGGCCGGATCTCTGATTGCCGTGCTGATCCTGCGGCAGACCAACAACTAC |
| | AACAGCGACGACTTCCAGTTCGTGTGGAACATCTACGCCAACAACGACGTGGTGGTGCCTACAAGCG |
| | GCAGCCACCACCATCATCACCATCACCACGGCGGCAGCATGAAGATGGAAGAACTGTTCAAGAAGCA |
| | CAAGATCGTCGCCGTGCTGCGGGCCAATTCTGTGGAAGAGGCCAAAAAAAAGGCCCTGGCCGTGTTT |
| | CTTGGCGGAGTGCACCTGATCGAGATCACCTTTACCGTGCCTGACGCCGACACCGTGATCAAAGAGCT |
| | GAGCTTCCTGAAAGAGATGGGCGCCATCATCGGAGCCGGAACCGTGACATCTGTTGAGCAGGCCAGA |
| | AAGGCCGTGGAATCTGGCGCCGAGTTTATCGTGTCCCCTCACCTGGATGAGGAAATCAGCCAGTTCGC |
| | CAAAGAAAAGGGCGTGTTCTACATGCCCGGCGTGATGACACCTACAGAGCTGGTCAAAGCCATGAAG |
| | CTGGGCCACACCATCCTGAAGCTGTTTCCAGGCGAAGTCGTGGGCCCTCAGTTCGTGAAAGCTATGAA |
| | GGGCCCATTTCCAAACGTGAAGTTCGTGCCCACTGGCGGCGTCAACCTGGATAATGTGTGCGAGTGGT |
| | TCAAGGCTGGCGTGCTGGCTGTTGGAGTGGGATCTGCTCTGGTCAAGGGCACACCTGTGGAAGTGGC |
| | TGAGAAGGCCAAGGCCTTCGTGGAAAAGATCAGAGGCTGCACCGAGTGA [SEQ ID NO: 50] |
| FIMHL- | ATGGAAACCGATACACTGCTGCTGTGGGTGCTGTTGCTCTGGGTTCCAGGCTCTACAGGCGATTTTGC |
| HIS-Fer | CTGCAAGACCGCCAACGGCACAGCCATTCCTATTGGCGGAGGCAGCGCCAATGTGTACGTTAACCTGG |
| 536 | CTCCTGCCGTGAACGTGGGCCAGAATCTGGTGGTGGATCTGAGCACCCAGATCTTTTGCCACAACGAC |
| | TACCCCGAGACAATCACCGACTACGTGACACTGCAGAGAGGCTCTGCTTACGGCGGCGTGCTGTCTAG |
| | CTTTAGCGGCACCGTGAAGTACAACGGCAGCAGCTACCCATTTCCTACCACCAGCGAGACACCCAGAG |
| | TGGTGTACAACAGCAGAACCGACAAGCCCTGGCCTGTGGCTCTGTACCTGACACCTGTTAGTTCTGCC |
| | GGCGGAGTGGCCATTAAGGCCGGATCTCTGATTGCCGTGCTGATCCTGCGGCAGACCAACAACTACA |
| | ACAGCGACGACTTCCAGTTCGTGTGGAACATCTACGCCAACAACGACGTGGTGGTGCCTACAAGCGG |
| | CAGCCACCACCATCATCACCATCACCACGGCGGCAGCGACATCATCAAGCTGCTGAACGAGCAAGTGA |
| | ACAAAGAGATGAACAGCAGCAACCTGTACATGAGCATGAGCAGCTGGTGCTACACACACAGCCTGGA |
| | TGGCGCCGGACTGTTCCTGTTTGATCATGCCGCCGAGGAATACGAGCACGCCAAGAAGCTGATCATCT |
| | TCCTGAACGAGAACAACGTGCCCGTGCAGCTGACATCTATCAGCGCCCCTGAGCACAAGTTCGAGGGC |
| | CTGACACAGATCTTCCAGAAGGCCTACGAACACGAGCAGCACATCAGCGAGAGCATCAACAACATCGT |
| | GGACCACGCCATCAAGAGCAAGGATCACGCCACCTTCAACTTTCTGCAGTGGTACGTGGCCGAACAGC |
| | ACGAGGAAGAGGTGCTGTTCAAGGACATCCTGGACAAGATCGAGCTGATCGGCAACGAGAACCACG |
| | GCCTGTACCTGGCCGATCAGTATGTGAAGGGAATCGCCAAGAGCCGGAAGTGA [SEQ ID NO: 51] |
| FIMH_DG_ | ATGGAAACCGATACACTGCTGCTGTGGGTGCTGTTGCTCTGGGTTCCAGGATCTACAGGGGATTTTGC |
| PGDGN_ | CTGCAAGACCGCCAATGGCACAGCCATTCCTATTGGCGGCGGAAGCGCCAATGTGTACGTGAACCTG |
| IMX313_ | GCTCCTGTGGTCAACGTGGGCCAGAATCTGGTGGTGGACCTGAGCACCCAGATCTTTTGCCACAACGA |
| HISj96 | CTACCCCGAGACAATCACCGACTACGTGACACTGCAGAGAGGCTCTGCTTACGGCGGCGTGCTGAGC |
| | AATTTTTCCGGCACAGTGAAGTACAGCGGCAGCAGCTACCCATTTCCTACCACCAGCGAGACACCCAG |
| | AGTGGTGTACAACAGCAGAACCGACAAGCCCTGGCCTGTGGCTCTGTACCTGACACCTGTTAGTTCTG |
| | CCGGCGGAGTGGCCATTAAGGCCGGATCTCTGATTGCCGTGCTGATCCTGCGGCAGACCAACAACTAC |
| | AACAGCGACGACTTCCAGTTCGTGTGGAACATCTACGCCAACAACGACGTGGTGGTGCCTACAGGCG |
| | GATGTGATGTGTCCGCCAGAGATGTGACAGTGACCCTGCCTGATTACCCCGGCTCTGTGCCTATTCCTC |
| | TGACCGTGTACTGCGCCAAGTCTCAGAACCTGGGCTACTACCTGAGCGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTGTCTCTTGG |
| | CCTGACAGCCAACTATGCCAGAACAGGCGGACAAGTGACAGCCGGCAATGTGCAGTCTATCATCGGC |
| | GTGACCTTCGTGTATCAGCCTGGCGACGGAAATGCCGACGTGACCATCACAGTGAATGGCAAGGTGG |
| | TGGCCAAGGGCAGCTCAGGCTCTGGCTCTGGATCTAAAAAACAGGGCGACGCCGATGTGTGTGGCGA |
| | GGTGGCATATATCCAGAGCGTGGTGTCCGATTGTCACGTGCCAACCGCCGAGCTGAGAACCCTGCTG |
| | GAAATCCGGAAGCTGTTCCTCGAAATTCAGAAGCTGAAGGTCGAGCTGCAGGGCCTGTCTAAAGAAG |
| | GCGGAGGAAGCGGATCTCACCACCACCATCACCACTGATGA [SEQ ID NO: 52] |
| FimH_ | ATGGAAACCGATACACTGCTGCTGTGGGTGCTGTTGCTCTGGGTTCCAGGATCTACAGGGGATTTTGC |
| PGDGNDG_ | CTGCAAGACCGCCAATGGCACAGCCATTCCTATTGGCGGCGGAAGCGCCAATGTGTACGTGAACCTG |
| HIS- | GCTCCTGTGGTCAACGTGGGCCAGAATCTGGTGGTGGACCTGAGCACCCAGATCTTTTGCCACAACGA |
| Ferritn | CTACCCCGAGACAATCACCGACTACGTGACACTGCAGAGAGGCTCTGCTTACGGCGGCGTGCTGAGC |
| j96 | AATTTTTCCGGCACAGTGAAGTACAGCGGCAGCAGCTACCCATTTCCTACCACCAGCGAGACACCCAG |
| | AGTGGTGTACAACAGCAGAACCGACAAGCCCTGGCCTGTGGCTCTGTACCTGACACCTGTTAGTTCTG |
| | CCGGCGGAGTGGCCATTAAGGCCGGATCTCTGATTGCCGTGCTGATCCTGCGGCAGACCAACAACTAC |
| | AACAGCGACGACTTCCAGTTCGTGTGGAACATCTACGCCAACAACGACGTGGTGGTGCCTACAGGCG |
| | GATGTGATGTGTCCGCCAGAGATGTGACAGTGACCCTGCCTGATTACCCCGGCTCTGTGCCTATTCCTC |
| | TGACCGTGTACTGCGCCAAGTCTCAGAACCTGGGCTACTACCTGAGCGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTGTCTCTTGG |
| | CCTGACAGCCAACTATGCCAGAACAGGCGGACAAGTGACAGCCGGCAATGTGCAGTCTATCATCGGC |
| | GTGACCTTCGTGTATCAGCCTGGCGACGGAAATGCCGACGTGACCATCACAGTGAATGGCAAGGTGG |
| | TGGCCAAGAGCGGAAGCCACCACCATCATCACCATCACCACGGCGGCAGCATGAAGATGGAAGAACT |
| | GTTCAAGAAGCACAAGATCGTCGCCGTGCTGCGGGCCAATTCTGTGGAAGAGGCCAAAAAAAAGGCC |
| | CTGGCCGTGTTTCTTGGCGGAGTGCACCTGATCGAGATCACCTTTACCGTGCCTGACGCCGACACCGT |
| | GATCAAAGAGCTGAGCTTCCTGAAAGAGATGGGCGCCATCATCGGAGCCGGAACCGTGACATCTGTT |
| | GAGCAGGCCAGAAAGGCCGTGGAATCTGGCGCCGAGTTTATCGTGTCCCCTCACCTGGATGAGGAAA |
| | TCAGCCAGTTCGCCAAAGAAAAGGGCGTGTTCTACATGCCCGGCGTGATGACACCTACAGAGCTGGTC |
| | AAAGCCATGAAGCTGGGCCACACCATCCTGAAGCTGTTTCCAGGCGAAGTCGTGGGCCCTCAGTTCGT |
| | GAAAGCTATGAAGGGCCCATTTCCAAACGTGAAGTTCGTGCCCACTGGCGGCGTCAACCTGGATAATG |
| | TGTGCGAGTGGTTCAAGGCTGGCGTGCTGGCTGTTGGAGTTGGCTCTGCTCTGGTCAAGGGCACACCT |
| | GTGGAAGTGGCTGAGAAGGCCAAGGCCTTCGTGGAAAAGATCAGAGGCTGCACCGAGTGATGA |
| | [SEQ ID NO: 53] |
| FIMH_DG_ | ATGGAAACCGATACACTGCTGCTGTGGGTGCTGTTGCTCTGGGTTCCAGGATCTACAGGGGATGTTTG |
| PGDGN- | CCTGCAAGACCGCCAATGGCACAGCCATTCCTATTGGCGGCGGAAGCGCCAATGTGTACGTTAACCTG |
| HIS- | GCTCCTGCCGTGAACGTGGGCCAGAATCTGGTGGTGGATCTGAGCACCCAGATCTTTTGCCACAACGA |
| Ferritin | CTACCCCGAGACAATCACCGACTACGTGACACTGCAGAGAGGCTCTGCTTACGGCGGCGTGCTGTCTA |
| 536 | GCTTTAGCGGCACCGTGAAGTACAACGGCAGCAGCTACCCATTTCCTACCACCAGCGAGACACCCAGA |
| | GTGGTGTACAACAGCAGAACCGACAAGCCCTGGCCTGTGGCTCTGTACCTGACACCTGTTAGTTCTGC |
| | CGGCGGAGTGGCCATTAAGGCCGGATCTCTGATTGCCGTGCTGATCCTGCGGCAGACCAACAACTACA |
| | ACAGCGACGACTTCCAGTTCGTGTGGAACATCTACGCCAACAACGACGTGGTGGTGCCTACAGGCGG |
| | ATGTGATGTGTCCGCCAGAGATGTGACAGTGACCCTGCCTGATTACCCCGGCTCTGTGCCTATTCCTCT |
| | GACCGTGTACTGCGCCAAGTCTCAGAACCTGGGCTACTACCTGAGCGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTGTCTCTTGG |
| | CCTGACAGCCAACTATGCCAGAACAGGCGGACAAGTGACAGCCGGCAATGTGCAGTCTATCATCGGC |
| | GTGACCTTCGTGTATCAGCCTGGCGACGGAAATGCCGACGTGACCATCACAGTGAATGGCAAGGTGG |
| | TGGCCAAGAGCGGAAGCCACCACCATCATCACCATCACCACGGCGGCAGCGACATCATCAAGCTGCTG |
| | AACGAGCAAGTGAACAAAGAGATGAACAGCAGCAACCTGTACATGAGCATGAGCAGCTGGTGCTACA |
| | CACACAGCCTGGATGGCGCCGGACTGTTCCTGTTTGATCATGCCGCCGAGGAATACGAGCACGCCAA |
| | GAAGCTGATCATCTTCCTGAACGAGAACAACGTGCCCGTCCAGCTGACATCTATCAGCGCCCCTGAGC |
| | ACAAGTTCGAGGGCCTGACACAGATCTTCCAGAAGGCCTACGAACACGAGCAGCACATCAGCGAGAG |
| | CATCAACAACATCGTGGACCACGCCATCAAGAGCAAGGATCACGCCACCTTCAACTTTCTGCAGTGGT |
| | ACGTGGCCGAACAGCACGAGGAAGAGGTGCTGTTCAAGGACATCCTGGACAAGATCGAGCTGATCG |
| | GCAACGAGAACCACGGCCTGTACCTGGCCGATCAGTATGTGAAGGGAATCGCCAAGAGCCGCAAGTG |
| | A [SEQ ID NO: 54] |
| FimH_ | ATGGAAACCGATACACTGCTGCTGTGGGTGCTGTTGCTCTGGGTTCCAGGATCTACAGGGGATGCCGC |
| PGDGN_ | TCAACCTGCTCGGAGAGCCAGAAGAACAAAGCTGGCCCTGTTTGCCTGCAAGACCGCCAATGGCACA |
| DG-HIS- | GCCATTCCTATTGGCGGCGGAAGCGCCAATGTGTACGTGAACCTGGCTCCTGTGGTCAACGTGGGCCA |
| Ferritin | GAATCTGGTGGTGGACCTGAGCACCCAGATCTTTTGCCACAACGACTACCCCGAGACAATCACCGACT |
| J96 | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCTACAGGCGGATGTGATGTGTCCGCCAGAGAT |
| | GTGACAGTGACCCTGCCTGATTACCCCGGCTCTGTGCCTATTCCTCTGACCGTGTACTGCGCCAAGTCT |
| | CAGAACCTGGGCTACTACCTGAGCGGCACAACAGCCGATGCCGGCAACAGCATCTTTACCAACACCGC |
| | CAGCTTCAGCCCTGCTCAAGGTGTTGGAGTGCAGCTGACCCGGAACGGAACAATCATCCCCGCCAACA |
| | ATACCGTGTCTCTGGGAGCTGTGGGCACCTCTGCTGTGTCTCTTGGCCTGACAGCCAACTATGCCAGA |
| | ACAGGCGGACAAGTGACAGCCGGCAATGTGCAGTCTATCATCGGCGTGACCTTCGTGTATCAGCCTG |
| | GCGACGGAAATGCCGACGTGACCATCACAGTGAATGGCAAGGTGGTGGCCAAGAGCGGAAGCCACC |
| | ACCATCATCACCATCACCACGGCGGCAGCGACATCATCAAGCTGCTGAACGAGCAAGTGAACAAAGA |
| | GATGAACAGCAGCAACCTGTACATGAGCATGAGCAGCTGGTGCTACACACACAGCCTGGATGGCGCC |
| | GGACTGTTCCTGTTTGATCATGCCGCCGAGGAATACGAGCACGCCAAGAAGCTGATCATCTTCCTGAA |
| | CGAGAACAACGTGCCCGTCCAGCTGACATCTATCAGCGCCCCTGAGCACAAGTTCGAGGGCCTGACAC |
| | AGATCTTCCAGAAGGCCTACGAACACGAGCAGCACATCAGCGAGAGCATCAACAACATCGTGGACCA |
| | CGCCATCAAGAGCAAGGATCACGCCACCTTCAACTTTCTGCAGTGGTACGTGGCCGAACAGCACGAG |
| | GAAGAGGTGCTGTTCAAGGACATCCTGGACAAGATCGAGCTGATCGGCAACGAGAACCACGGCCTGT |
| | ACCTGGCCGATCAGTATGTGAAGGGAATCGCCAAGAGCCGCAAGTGA [SEQ ID NO: 55] |
| FimH_ | ATGGAAACCGATACACTGCTGCTGTGGGTGCTGTTGCTCTGGGTTCCAGGATCTACAGGGGATGCCGC |
| PGDGN_ | TCAACCTGCTCGGAGAGCCAGAAGAACAAAGCTGGCCCTGTTTGCCTGCAAGACCGCCAATGGCACA |
| DG-HIS- | GCCATTCCTATTGGCGGCGGAAGCGCCAATGTGTACGTGAACCTGGCTCCTGTGGTCAACGTGGGCCA |
| MI3 j96 | GAATCTGGTGGTGGACCTGAGCACCCAGATCTTTTGCCACAACGACTACCCCGAGACAATCACCGACT |
| | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCTACAGGCGGATGTGATGTGTCCGCCAGAGAT |
| | GTGACAGTGACCCTGCCTGATTACCCCGGCTCTGTGCCTATTCCTCTGACCGTGTACTGCGCCAAGTCT |
| | CAGAACCTGGGCTACTACCTGAGCGGCACAACAGCCGATGCCGGCAACAGCATCTTTACCAACACCGC |
| | CAGCTTCAGCCCTGCTCAAGGTGTTGGAGTGCAGCTGACCCGGAACGGAACAATCATCCCCGCCAACA |
| | ATACCGTGTCTCTGGGAGCTGTGGGCACCTCTGCTGTGTCTCTTGGCCTGACAGCCAACTATGCCAGA |
| | ACAGGCGGACAAGTGACAGCCGGCAATGTGCAGTCTATCATCGGCGTGACCTTCGTGTATCAGCCTG |
| | GCGACGGAAATGCCGACGTGACCATCACAGTGAATGGCAAGGTGGTGGCCAAGAGCGGAAGCCACC |
| | ACCATCATCACCATCACCACGGCGGCAGCATGAAGATGGAAGAACTGTTCAAGAAGCACAAGATCGT |
| | CGCCGTGCTGCGGGCCAATTCTGTGGAAGAGGCCAAAAAAAAGGCCCTGGCCGTGTTTCTTGGCGGA |
| | GTGCACCTGATCGAGATCACCTTTACCGTGCCTGACGCCGACACCGTGATCAAAGAGCTGAGCTTCCT |
| | GAAAGAGATGGGCGCCATCATCGGAGCCGGAACCGTGACATCTGTTGAGCAGGCCAGAAAGGCCGT |
| | GGAATCTGGCGCCGAGTTTATCGTGTCCCCTCACCTGGATGAGGAAATCAGCCAGTTCGCCAAAGAAA |
| | AGGGCGTGTTCTACATGCCCGGCGTGATGACACCTACAGAGCTGGTCAAAGCCATGAAGCTGGGCCA |
| | CACCATCCTGAAGCTGTTTCCAGGCGAAGTCGTGGGCCCTCAGTTCGTGAAAGCTATGAAGGGCCCAT |
| | TTCCAAACGTGAAGTTCGTGCCCACTGGCGGCGTCAACCTGGATAATGTGTGCGAGTGGTTCAAGGCT |
| | GGCGTGCTGGCTGTTGGAGTTGGCTCTGCTCTGGTCAAGGGCACACCTGTGGAAGTGGCTGAGAAGG |
| | CCAAGGCCTTCGTGGAAAAGATCAGAGGCTGCACCGAGTGATGA [SEQ ID NO: 56] |
| FimH_ | ATGGAAACCGATACACTGCTGCTGTGGGTGCTGTTGCTCTGGGTTCCAGGATCTACAGGGGATGCCGC |
| PGDGN_ | TCAACCTGCTCGGAGAGCCAGAAGAACAAAGCTGGCCCTGTTTGCCTGCAAGACCGCCAATGGCACA |
| DG-HIS- | GCCATTCCTATTGGCGGCGGAAGCGCCAATGTGTACGTGAACCTGGCTCCTGTGGTCAACGTGGGCCA |
| IMX313 | GAATCTGGTGGTGGACCTGAGCACCCAGATCTTTTGCCACAACGACTACCCCGAGACAATCACCGACT |
| j96 | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCTACAGGCGGATGTGATGTGTCCGCCAGAGAT |
| | GTGACAGTGACCCTGCCTGATTACCCCGGCTCTGTGCCTATTCCTCTGACCGTGTACTGCGCCAAGTCT |
| | CAGAACCTGGGCTACTACCTGAGCGGCACAACAGCCGATGCCGGCAACAGCATCTTTACCAACACCGC |
| | CAGCTTCAGCCCTGCTCAAGGTGTTGGAGTGCAGCTGACCCGGAACGGAACAATCATCCCCGCCAACA |
| | ATACCGTGTCTCTGGGAGCTGTGGGCACCTCTGCTGTGTCTCTTGGCCTGACAGCCAACTATGCCAGA |
| | ACAGGCGGACAAGTGACAGCCGGCAATGTGCAGTCTATCATCGGCGTGACCTTCGTGTATCAGCCTG |
| | GCGACGGAAATGCCGACGTGACCATCACAGTGAATGGCAAGGTGGTGGCCAAGGGCAGCTCAGGCT |
| | CTGGCTCTGGATCTAAAAAACAGGGCGACGCCGATGTGTGTGGCGAGGTGGCATATATCCAGAGCGT |
| | GGTGTCCGATTGTCACGTGCCAACCGCCGAGCTGAGAACCCTGCTGGAAATCCGGAAGCTGTTCCTCG |
| | AAATTCAGAAGCTGAAGGTCGAGCTGCAGGGCCTGTCTAAAGAAGGCGGAGGAAGCGGATCTCACC |
| | ACCACCATCACCACTGATGAC [SEQ ID NO: 57] |
| FimH_ | ATGGAAACCGATACACTGCTGCTGTGGGTGCTGTTGCTCTGGGTTCCAGGATCTACAGGGGATGCCGC |
| PGDGN_ | TCAACCTGCTCGGAGAGCCAGAAGAACAAAGCTGGCCCTGTTTGCCTGCAAGACCGCCAATGGCACA |
| DG_Ferritin | GCCATTCCTATTGGCGGCGGAAGCGCCAATGTGTACGTTAACCTGGCTCCTGCCGTGAACGTGGGCCA |
| (536) | GAATCTGGTGGTGGATCTGAGCACCCAGATCTTTTGCCACAACGACTACCCCGAGACAATCACCGACT |
| | ACGTGACACTGCAGAGAGGCTCTGCTTACGGCGGCGTGCTGTCTAGCTTTAGCGGCACCGTGAAGTAC |
| | AACGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCTACAGGCGGATGTGATGTGTCCGCCAGAGAT |
| | GTGACAGTGACCCTGCCTGATTACCCCGGCTCTGTGCCTATTCCTCTGACCGTGTACTGCGCCAAGTCT |
| | CAGAACCTGGGCTACTACCTGAGCGGCACAACAGCCGATGCCGGCAACAGCATCTTTACCAACACCGC |
| | CAGCTTCAGCCCTGCTCAAGGTGTTGGAGTGCAGCTGACCCGGAACGGAACAATCATCCCCGCCAACA |
| | ATACCGTGTCTCTGGGAGCTGTGGGCACCTCTGCTGTGTCTCTTGGCCTGACAGCCAACTATGCCAGA |
| | ACAGGCGGACAAGTGACAGCCGGCAATGTGCAGTCTATCATCGGCGTGACCTTCGTGTATCAGCCTG |
| | GCGACGGAAATGCCGACGTGACCATCACAGTGAATGGCAAGGTGGTGGCCAAGAGCGGAAGCCACC |
| | ACCATCATCACCATCACCACGGCGGCAGCGACATCATCAAGCTGCTGAACGAGCAAGTGAACAAAGA |
| | GATGAACAGCAGCAACCTGTACATGAGCATGAGCAGCTGGTGCTACACACACAGCCTGGATGGCGCC |
| | GGACTGTTCCTGTTTGATCATGCCGCCGAGGAATACGAGCACGCCAAGAAGCTGATCATCTTCCTGAA |
| | CGAGAACAACGTGCCCGTCCAGCTGACATCTATCAGCGCCCCTGAGCACAAGTTCGAGGGCCTGACAC |
| | AGATCTTCCAGAAGGCCTACGAACACGAGCAGCACATCAGCGAGAGCATCAACAACATCGTGGACCA |
| | CGCCATCAAGAGCAAGGATCACGCCACCTTCAACTTTCTGCAGTGGTACGTGGCCGAACAGCACGAG |
| | GAAGAGGTGCTGTTCAAGGACATCCTGGACAAGATCGAGCTGATCGGCAACGAGAACCACGGCCTGT |
| | ACCTGGCCGATCAGTATGTGAAGGGAATCGCCAAGAGCCGCAAGTGA [SEQ ID NO: 58] |
| FimH- | ATGGAAACCGATACACTGCTGCTGTGGGTGCTGTTGCTCTGGGTTCCAGGATCTACAGGGGATGCCGC |
| IMX313 | TCAACCTGCTCGGAGAGCCAGAAGAACAAAGCTGGCCCTGTTTGCCTGCAAGACCGCCAATGGCACA |
| j96 | GCCATTCCTATTGGCGGCGGAAGCGCCAATGTGTACGTGAACCTGGCTCCTGTGGTCAACGTGGGCCA |
| | GAATCTGGTGGTGGACCTGAGCACCCAGATCTTTTGCCACAACGACTACCCCGAGACAATCACCGACT |
| | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCCACCTGTGATGTGTCCGCTAGAGATGTGACA |
| | GTGACCCTGCCTGATTACCCCGGCTCTGTGCCTATTCCTCTGACCGTGTACTGCGCCAAGTCTCAGAAC |
| | CTGGGCTACTACCTGAGCGGCACAACAGCCGATGCCGGCAACAGCATCTTTACCAACACCGCCAGCTT |
| | CAGCCCTGCTCAAGGTGTTGGAGTGCAGCTGACCCGGAACGGAACAATCATCCCCGCCAACAATACCG |
| | TGTCTCTGGGAGCTGTGGGCACATCTGCAGTTTCTCTGGGCCTGACAGCCAACTATGCCAGAACAGGC |
| | GGACAAGTGACAGCCGGCAATGTGCAGTCTATCATCGGCGTGACATTCGTGTATCAGGGCAGCTCTG |
| | GCAGCGGCTCTGGATCTAAAAAACAGGGCGACGCCGATGTGTGTGGCGAGGTGGCATATATCCAGAG |
| | CGTGGTGTCCGATTGTCACGTGCCAACCGCCGAGCTGAGAACCCTGCTGGAAATCCGGAAGCTGTTCC |
| | TCGAAATTCAGAAGCTGAAGGTCGAGCTGCAGGGCCTGTCTAAAGAAGGCGGAGGAAGCGGATCTC |
| | ACCACCACCATCACCACTGA [SEQ ID NO: 59] |
| FimH_ | ATGGAAACCGATACACTGCTGCTGTGGGTGCTGTTGCTCTGGGTTCCAGGATCTACAGGGGATGCCGC |
| mi3 j96 | TCAACCTGCTCGGAGAGCCAGAAGAACAAAGCTGGCCCTGTTTGCCTGCAAGACCGCCAATGGCACA |
| | GCCATTCCTATTGGCGGCGGAAGCGCCAATGTGTACGTGAACCTGGCTCCTGTGGTCAACGTGGGCCA |
| | GAATCTGGTGGTGGACCTGAGCACCCAGATCTTTTGCCACAACGACTACCCCGAGACAATCACCGACT |
| | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCCACCTGTGATGTGTCCGCTAGAGATGTGACA |
| | GTGACCCTGCCTGATTACCCCGGCTCTGTGCCTATTCCTCTGACCGTGTACTGCGCCAAGTCTCAGAAC |
| | CTGGGCTACTACCTGAGCGGCACAACAGCCGATGCCGGCAACAGCATCTTTACCAACACCGCCAGCTT |
| | CAGCCCTGCTCAAGGTGTTGGAGTGCAGCTGACCCGGAACGGAACAATCATCCCCGCCAACAATACCG |
| | TGTCTCTGGGAGCTGTGGGCACATCTGCAGTTTCTCTGGGCCTGACAGCCAACTATGCCAGAACAGGC |
| | GGACAAGTGACAGCCGGCAATGTGCAGTCTATCATCGGCGTGACCTTCGTGTACCAAGGATCTGGCG |
| | GAGGCGGCATGAAGATGGAAGAACTGTTCAAGAAACACAAGATCGTGGCCGTGCTGCGGGCCAATTC |
| | TGTGGAAGAGGCCAAAAAAAAGGCCCTGGCCGTGTTTCTCGGAGGCGTGCACCTGATCGAGATCACC |
| | TTTACCGTGCCTGACGCCGACACCGTGATCAAAGAGCTGAGCTTCCTGAAAGAGATGGGCGCCATCAT |
| | CGGAGCCGGAACCGTGACATCTGTTGAGCAGGCCAGAAAGGCCGTGGAATCTGGCGCCGAGTTTATC |
| | GTGTCCCCTCACCTGGATGAGGAAATCAGCCAGTTCGCCAAAGAAAAGGGCGTGTTCTACATGCCCGG |
| | CGTGATGACACCTACAGAGCTGGTCAAAGCCATGAAGCTGGGCCACACCATCCTGAAGCTGTTTCCAG |
| | GCGAAGTCGTGGGCCCTCAGTTCGTGAAAGCTATGAAGGGCCCATTTCCAAACGTGAAGTTCGTGCCC |
| | ACTGGCGGAGTGAATCTGGACAACGTGTGCGAGTGGTTCAAGGCTGGCGTGCTGGCTGTTGGAGTTG |
| | GCTCTGCTCTGGTCAAGGGCACACCTGTGGAAGTGGCTGAGAAGGCCAAGGCCTTCGTGGAAAAGAT |
| | CAGAGGCTGTACCGAAGGCAGCGGCTCTGGAAGCGGATCTGGATCTCACCACCATCATCACCATCACC |
| | ACTGA [SEQ ID NO: 60] |
| FIMHL- | ATGGAAACCGATACACTGCTGCTGTGGGTGCTGTTGCTCTGGGTTCCAGGATCTACAGGGGATGCCGC |
| HIS-mI3 | TCAACCTGCTCGGAGAGCCAGAAGAACAAAGCTGGCCCTGTTTGCCTGCAAGACCGCCAATGGCACA |
| j96 | GCCATTCCTATTGGCGGCGGAAGCGCCAATGTGTACGTGAACCTGGCTCCTGTGGTCAACGTGGGCCA |
| | GAATCTGGTGGTGGACCTGAGCACCCAGATCTTTTGCCACAACGACTACCCCGAGACAATCACCGACT |
| | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCTACAGGATCTGGCGGACACCACCATCATCACC |
| | ATCACCACGGCAGCGGCTCCATGAAGATGGAAGAACTGTTCAAGAAGCACAAGATCGTCGCCGTGCT |
| | GCGGGCCAATTCTGTGGAAGAGGCCAAAAAAAAGGCCCTGGCCGTGTTTCTTGGCGGAGTGCACCTG |
| | ATCGAGATCACCTTTACCGTGCCTGACGCCGACACCGTGATCAAAGAGCTGAGCTTCCTGAAAGAGAT |
| | GGGCGCCATCATCGGAGCCGGAACCGTGACATCTGTTGAGCAGGCCAGAAAGGCCGTGGAATCTGGC |
| | GCCGAGTTTATCGTGTCCCCTCACCTGGATGAGGAAATCAGCCAGTTCGCCAAAGAAAAGGGCGTGTT |
| | CTACATGCCCGGCGTGATGACACCTACAGAGCTGGTCAAAGCCATGAAGCTGGGCCACACCATCCTGA |
| | AGCTGTTTCCAGGCGAAGTCGTGGGCCCTCAGTTCGTGAAAGCTATGAAGGGCCCATTTCCAAACGTG |
| | AAGTTCGTGCCCACTGGCGGCGTCAACCTGGATAATGTGTGCGAGTGGTTCAAGGCTGGCGTGCTGG |
| | CTGTTGGAGTGGGATCTGCTCTGGTCAAGGGCACACCTGTGGAAGTGGCTGAGAAGGCCAAGGCCTT |
| | CGTGGAAAAGATCAGAGGCTGCACCGAGTGA [SEQ ID NO: 61] |
| FIMH_DG_ | ATGTTTGCATGTAAAACCGCAAATGGCACCGCAATTCCGATTGGTGGTGGTAGCGCAAATGTTTATGT |
| PGDGN_ | TAATCTGGCACCGGCAGTTAATGTTGGTCAGAATCTGGTTGTTGATCTGAGCACCCAGATTTTTTGCCA |
| HIS- | TAATGATTATCCGGAAACCATCACCGATTATGTTACCCTGCAGCGTGGTAGTGCATATGGTGGTGTTCT |
| Ferritin | GAGCAGCTTTAGCGGCACCGTGAAATATAACGGTAGCAGCTATCCGTTTCCGACCACCAGTGAAACAC |
| 536 | CGCGTGTTGTGTATAATAGCCGTACCGATAAACCGTGGCCTGTTGCACTGTATCTGACACCGGTTAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | GGTTGTGATGTTAGCGCACGTGATGTTACCGTTACACTGCCGGATTATCCTGGTAGCGTTCCGATTCCG |
| | CTGACCGTTTATTGTGCAAAAAGCCAGAACCTGGGTTATTATCTGAGCGGCACCACCGCAGATGCAGG |
| | TAATAGCATTTTTACCAATACCGCCAGCTTTAGTCCGGCACAAGGTGTTGGTGTTCAGCTGACCCGTAA |
| | TGGCACCATTATTCCGGCAAATAATACCGTTAGCCTGGGTGCAGTTGGCACCAGCGCAGTGAGCCTGG |
| | GTCTGACCGCCAATTATGCACGTACCGGTGGTCAGGTTACCGCAGGTAATGTTCAGAGCATTATTGGT |
| | GTTACCTTTGTGTATCAGCCTGGTGATGGTAATGCAGATGTGACCATTACCGTGAATGGTAAAGTTGTT |
| | GCCAAAAGCGGTAGTCATCATCACCACCATCATCATCACGGTGGTAGCGATATCATCAAACTGCTGAA |
| | TGAACAGGTGAACAAAGAAATGAATAGCAGCAACCTGTATATGAGCATGAGCAGCTGGTGTTATACC |
| | CATAGCCTGGATGGTGCAGGTCTGTTTCTGTTTGATCATGCAGCCGAAGAATATGAGCACGCAAAAAA |
| | ACTGATCATCTTCCTGAATGAAAATAATGTTCCGGTGCAGCTGACCAGCATTAGCGCTCCGGAACATA |
| | AATTTGAAGGTCTGACACAGATTTTTCAGAAAGCCTATGAACATGAACAGCACATTAGCGAAAGCATT |
| | AACAACATTGTGGATCACGCCATCAAAAGCAAAGATCATGCAACCTTTAACTTTCTGCAGTGGTATGTT |
| | GCAGAACAGCATGAAGAAGAAGTGCTGTTTAAAGACATCCTGGATAAAATTGAACTGATCGGCAACG |
| | AAAATCATGGTCTGTATCTGGCAGATCAGTATGTTAAAGGTATTGCGAAAAGCCGCAAATAA |
| | [SEQ ID NO: 62] |
| LS- | ATGAAGTATCTGCTGCCGACCGCAGCAGCGGGTCTGCTGCTGCTGGCAGCACAGCCTGCAATGGCATT |
| FIMHL- | TGCATGTAAAACCGCAAATGGCACCGCAATTCCGATTGGTGGTGGTAGCGCAAATGTTTATGTTAATC |
| IMX313- | TGGCACCGGTTGTTAATGTTGGTCAGAATCTGGTTGTTGATCTGAGCACCCAGATTTTTTGCCATAATG |
| HIS | ATTATCCGGAAACCATCACCGATTATGTTACCCTGCAGCGTGGTAGTGCATATGGTGGTGTTCTGAGC |
| | AATTTTAGCGGCACCGTGAAATATAGCGGTAGCAGCTATCCGTTTCCGACCACCAGTGAAACACCGCG |
| | TGTTGTGTATAATAGCCGTACCGATAAACCGTGGCCTGTTGCACTGTATCTGACACCGGTGAGCAGTG |
| | CCGGTGGTGTTGCAATTAAAGCAGGTAGCCTGATTGCAGTTCTGATTCTGCGTCAGACCAATAACTAT |
| | AACTCCGATGATTTTCAGTTTGTGTGGAACATCTATGCCAATAATGATGTTGTTGTTCCGACCGGTGGT |
| | AGCAGCGGTAGCGGTTCAGGTAGCAAAAAACAGGGTGATGCAGATGTTTGTGGTGAAGTTGCATATA |
| | TTCAGAGCGTTGTTAGCGATTGTCATGTTCCGACAGCAGAACTGCGTACCCTGCTGGAAATTCGTAAA |
| | CTGTTTCTGGAAATCCAGAAGCTGAAAGTTGAACTGCAGGGTCTGAGCAAAGAAGGTGGCGGAAGCG |
| | GTAGCCATCACCATCACCATCACTGA [SEQ ID NO: 63] |
| FIMHL- | ATGTTTGCAAGCAAAACCGCAAATGGCACCGCAATTCCGATTGGTGGTGGTAGCGCAAATGTTTATGT |
| S24S65- | TAATCTGGCACCGGTTGTTAATGTTGGTCAGAATCTGGTTGTTGATCTGAGCACCCAGATTTTTAGCCA |
| IMX313 | TAATGATTATCCGGAAACCATCACCGATTATGTTACCCTGCAGCGTGGTAGTGCATATGGTGGTGTTCT |
| | GAGCAATTTTAGCGGCACCGTGAAATATAGCGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTTAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | GGTAGTAGCGGTAGTGGTAGCGGTTCAAAAAAACAGGGTGATGCAGATGTTTGTGGTGAAGTTGCAT |
| | ATATTCAGAGCGTTGTTAGCGATTGTCATGTGCCGACCGCAGAACTGCGTACCCTGCTGGAAATTCGT |
| | AAACTGTTTCTGGAAATCCAGAAGCTGAAAGTTGAACTGCAGGGTCTGAGTAAAGAAGGTGGTGGTA |
| | GTGGTAGCCATCACCATCATCATCACTAATAA [SEQ ID NO: 64] |
| FIMHL- | ATGTTTGCAAGCAAAACCGCAAATGGCACCGCAATTCCGATTGGTGGTGGTAGCGCAAATGTTTATGT |
| S24S65- | TAATCTGGCACCGGCAGTTAATGTTGGTCAGAATCTGGTTGTTGATCTGAGCACCCAGATTTTTAGCCA |
| foldon- | TAATGATTATCCGGAAACCATCACCGATTATGTTACCCTGCAGCGTGGTAGTGCATATGGTGGTGTTCT |
| ferritin | GAGCAGCTTTAGCGGCACCGTGAAATATAACGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTTAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | TCAGGTTATATTCCGGAAGCACCGCGTGATGGTCAGGCATATGTTCGTAAAGATGGTGAATGGGTTCT |
| | GCTGAGCACCTTTTTAGGTAGCGGTCATCATCACCATCATCATGGTAGCGGTGATATCATTAAACTGCT |
| | GAATGAACAGGTGAACAAAGAGATGAATAGCAGCAATCTGTATATGAGCATGAGCAGCTGGTGTTAT |
| | ACCCATAGCCTGGATGGTGCAGGTCTGTTTCTGTTTGATCATGCAGCCGAAGAATATGAGCACGCAAA |
| | AAAACTGATCATCTTCCTGAATGAAAATAATGTTCCGGTTCAGCTGACCAGCATTAGCGCTCCGGAACA |
| | TAAATTTGAAGGTCTGACACAGATTTTTCAGAAAGCCTATGAACATGAACAGCACATTAGCGAAAGCA |
| | TTAACAACATTGTGGATCACGCCATCAAAAGCAAAGATCATGCAACCTTTAACTTTCTGCAGTGGTATG |
| | TTGCAGAACAGCATGAAGAAGAAGTGCTGTTTAAAGACATCCTGGATAAAATTGAACTGATCGGCAAC |
| | GAAAATCATGGTCTGTATCTGGCAGATCAGTATGTTAAAGGTATTGCCAAAAGCCGCAAGTAATAA |
| | [SEQ ID NO: 65] |
| FIMHL- | ATGTTTGCAAGCAAAACCGCAAATGGCACCGCAATTCCGATTGGTGGTGGTAGCGCAAATGTTTATGT |
| S24S65- | TAATCTGGCACCGGCAGTTAATGTTGGTCAGAATCTGGTTGTTGATCTGAGCACCCAGATTTTTAGCCA |
| Mi3 | TAATGATTATCCGGAAACCATCACCGATTATGTTACCCTGCAGCGTGGTAGTGCATATGGTGGTGTTCT |
| | GAGCAGCTTTAGCGGCACCGTGAAATATAACGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTTAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | GGTAGTGGTGGTTCAGGTGGTAGCATGAAAATGGAAGAACTGTTCAAAAAGCACAAAATTGTTGCCG |
| | TTCTGCGTGCAAATAGCGTTGAAGAAGCAAAAAAGAAAGCACTGGCCGTTTTTTTAGGTGGTGTGCAT |
| | CTGATTGAAATCACCTTTACCGTTCCGGATGCAGATACCGTTATTAAAGAACTGAGCTTTCTGAAAGAA |
| | ATGGGTGCAATTATTGGTGCAGGCACCGTTACCAGCGTTGAACAGGCACGTAAAGCAGTTGAAAGCG |
| | GTGCAGAATTTATTGTTAGTCCGCATCTGGATGAAGAAATTAGCCAGTTTGCAAAAGAAAAGGGCGTG |
| | TTTTATATGCCTGGTGTTATGACCCCGACCGAACTGGTTAAAGCAATGAAACTGGGTCATACCATCCTG |
| | AAACTGTTTCCGGGTGAAGTTGTTGGTCCGCAGTTTGTGAAAGCCATGAAAGGTCCTTTTCCGAACGTT |
| | AAATTTGTGCCGACAGGTGGCGTGAATCTGGATAATGTTTGTGAATGGTTTAAAGCCGGTGTTCTGGC |
| | CGTTGGTGTTGGTAGTGCCCTGGTGAAAGGTACACCGGTTGAAGTTGCAGAAAAAGCAAAAGCCTTT |
| | GTGGAAAAAATTCGTGGTTGTACCGAAGGTAGCGGTAGCGGTTCAGGTAGTGGTAGCCATCACCATC |
| | ATCATCACTAATAA [SEQ ID NO: 66] |
| FIMHL- | ATGTTTGCATGTAAAACCGCAAATGGCACCGCAATTCCGATTGGTGGTGGTAGCGCAAATGTTTATGT |
| mI3 | TAATCTGGCACCGGCAGTTAATGTTGGTCAGAATCTGGTTGTTGATCTGAGCACCCAGATTTTTTGCCA |
| | TAATGATTATCCGGAAACCATCACCGATTATGTTACCCTGCAGCGTGGTAGTGCATATGGTGGTGTTCT |
| | GAGCAGCTTTAGCGGCACCGTGAAATATAACGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTTAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | GGTAGTGGTGGTTCAGGTGGTAGCATGAAAATGGAAGAACTGTTCAAAAAGCACAAAATTGTTGCCG |
| | TTCTGCGTGCAAATAGCGTTGAAGAAGCAAAAAAGAAAGCACTGGCCGTTTTTTTAGGTGGTGTGCAT |
| | CTGATTGAAATCACCTTTACCGTTCCGGATGCAGATACCGTTATTAAAGAACTGAGCTTTCTGAAAGAA |
| | ATGGGTGCAATTATTGGTGCAGGCACCGTTACCAGCGTTGAACAGGCACGTAAAGCAGTTGAAAGCG |
| | GTGCAGAATTTATTGTTAGTCCGCATCTGGATGAAGAAATTAGCCAGTTTGCAAAAGAAAAGGGCGTG |
| | TTTTATATGCCTGGTGTTATGACCCCGACCGAACTGGTTAAAGCAATGAAACTGGGTCATACCATCCTG |
| | AAACTGTTTCCGGGTGAAGTTGTTGGTCCGCAGTTTGTGAAAGCCATGAAAGGTCCTTTTCCGAACGTT |
| | AAATTTGTGCCGACAGGTGGCGTGAATCTGGATAATGTTTGTGAATGGTTTAAAGCCGGTGTTCTGGC |
| | CGTTGGTGTTGGTAGTGCCCTGGTGAAAGGTACACCGGTTGAAGTTGCAGAAAAAGCAAAAGCCTTT |
| | GTGGAAAAAATTCGTGGTTGTACCGAAGGTAGCGGTAGCGGTTCAGGTAGTGGTAGCCATCACCATC |
| | ATCATCACTAATAA [SEQ ID NO: 67] |
| FimHL- | ATGTTCGCAAGCAAAACCGCAAATGGCACCGCAATTCCGATTGGTGGTGGTAGCGCAAATGTTTATGT |
| NOCYS- | TAATCTGGCACCGGCAGTTAATGTTGGTCAGAATCTGGTTGTTGATCTGAGCACCCAGATTTTTAGCCA |
| MI3 | TAATGATTATCCGGAAACCATCACCGATTATGTTACCCTGCAGCGTGGTAGTGCATATGGTGGTGTTCT |
| | GAGCAATTTTAGCGGCACCGTGAAATATAGCGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTTAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | GGTGGTGGCAGTGGTGGTTCAGGCGGTAGCGGTGGTAGCATGAAAATGGAAGAACTGTTCAAAAAG |
| | CACAAAATTGTTGCCGTTCTGCGTGCAAATAGCGTTGAAGAAGCAAAAAAGAAAGCACTGGCCGTTTT |
| | TTTAGGTGGTGTGCATCTGATTGAAATCACCTTTACCGTTCCGGATGCAGATACCGTTATTAAAGAACT |
| | GAGCTTTCTGAAAGAAATGGGTGCAATTATTGGTGCAGGCACCGTTACCAGCGTTGAACAGGCACGT |
| | AAAGCAGTTGAAAGCGGTGCAGAATTTATTGTTAGTCCGCATCTGGATGAAGAAATTAGCCAGTTTGC |
| | AAAAGAAAAGGGCGTGTTTTATATGCCTGGTGTTATGACCCCGACCGAACTGGTTAAAGCAATGAAAC |
| | TGGGTCATACCATCCTGAAACTGTTTCCGGGTGAAGTTGTTGGTCCGCAGTTTGTGAAAGCCATGAAA |
| | GGTCCTTTTCCGAACGTTAAATTTGTGCCGACAGGTGGCGTGAATCTGGATAATGTTTGTGAATGGTTT |
| | AAAGCCGGTGTTCTGGCAGTTGGTGTTGGTAGTGCCCTGGTGAAAGGTACACCGGTTGAAGTTGCAG |
| | AAAAAGCAAAAGCCTTTGTGGAAAAAATTCGTGGTTGTACCGAAGGTAGCGGTAGTGGTAGCGGTTC |
| | AGGTAGCCATCACCATCACCATCACTGA [SEQ ID NO: 68] |
| FimHdel | ATGTTCGCCTGCAAAACCGCAAATGGCACCGCAATTCCGATTGGTGGTGGTAGCGCAAATGTTTATGT |
| taGG_ | TAATCTGGCACCGGTTGTTAATGTTGGTCAGAATCTGGTTGTTGATCTGAGCACCCAGATTTTTTGCCA |
| PGDGNDG_ | TAATGATTATCCGGAAACCATCACCGATTATGTTACCCTGCAGCGTGGTAGTGCATATGGTGGTGTTCT |
| mi3 | GAGCAATTTTAGCGGCACCGTGAAATATAGCGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTGAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCTGT |
| | GATGTTAGCGCACGTGATGTTACCGTTACACTGCCGGATTATCCTGGTAGCGTTCCGATTCCGCTGACC |
| | GTTTATTGTGCAAAAAGCCAGAACCTGGGTTATTATCTGAGCGGCACCACCGCAGATGCAGGTAATAG |
| | CATTTTTACCAATACCGCAAGCTTTAGTCCGGCACAAGGTGTTGGTGTTCAGCTGACCCGTAATGGCAC |
| | CATTATTCCGGCAAATAATACCGTTAGCCTGGGTGCAGTTGGCACCAGCGCAGTGAGCCTGGGTCTGA |
| | CCGCCAATTATGCACGTACCGGTGGTCAGGTTACCGCAGGTAATGTTCAGAGCATTATTGGTGTTACCT |
| | TTGTGTATCAGCCTGGTGATGGTAATGCAGATGTGACCATTACCGTGAATGGTAAAGTTGTTGCAAAA |
| | GGTAGCGGTGGTGGTGGCATGAAAATGGAAGAACTGTTCAAAAAACACAAGATTGTTGCCGTTCTGC |
| | GTGCAAATAGCGTTGAAGAAGCAAAAAAGAAAGCACTGGCCGTTTTTTTAGGTGGTGTGCATCTGATT |
| | GAAATCACCTTTACCGTTCCGGATGCAGATACCGTTATTAAAGAACTGAGCTTTCTGAAAGAAATGGG |
| | TGCAATTATTGGCGCAGGCACCGTTACCAGCGTTGAACAGGCACGTAAAGCAGTTGAAAGCGGTGCA |
| | GAATTTATTGTTAGTCCGCATCTGGATGAAGAAATTAGCCAGTTTGCAAAAGAAAAGGGCGTGTTTTA |
| | TATGCCTGGTGTTATGACCCCGACCGAACTGGTTAAAGCAATGAAACTGGGTCATACCATCCTGAAAC |
| | TGTTTCCGGGTGAAGTTGTTGGTCCGCAGTTTGTGAAAGCCATGAAAGGTCCTTTTCCGAACGTTAAAT |
| | TTGTGCCGACCGGTGGCGTGAATCTGGATAATGTTTGTGAATGGTTTAAAGCCGGTGTTCTGGCAGTT |
| | GGTGTTGGTAGTGCCCTGGTGAAAGGTACACCGGTTGAAGTTGCAGAAAAAGCAAAAGCCTTTGTGG |
| | AAAAAATTCGTGGTTGTACCGAAGGTAGTGGTAGCGGCAGCGGTAGCGGTTCACATCACCATCACCAT |
| | CACTGA [SEQ ID NO: 69] |
| FimHL- | ATGGGCAGCAGCCATCATCATCATCATCACGAACTGTACTTCCAGGGCTTTGCATGTAAAACCGCAAAT |
| GSG4- | GGCACCGCAATTCCGATTGGTGGTGGTAGCGCAAATGTTTATGTTAATCTGGCACCGGCAGTTAATGT |
| Ferritin | TGGTCAGAATCTGGTTGTTGATCTGAGCACCCAGATTTTTTGCCATAATGATTATCCGGAAACCATCAC |
| | CGATTATGTTACCCTGCAGCGTGGTAGTGCATATGGTGGTGTTCTGAGCAGCTTTAGCGGCACCGTGA |
| | AATATAACGGTAGCAGCTATCCGTTTCCGACCACCAGTGAAACACCGCGTGTTGTGTATAATAGCCGT |
| | ACCGATAAACCGTGGCCTGTTGCACTGTATCTGACACCGGTTAGCAGTGCCGGTGGTGTTGCAATTAA |
| | AGCAGGTAGCCTGATTGCAGTTCTGATTCTGCGTCAGACCAATAACTATAACTCCGATGATTTTCAGTT |
| | TGTGTGGAACATCTATGCCAATAATGATGTTGTTGTTCCGACCGGTAGCGGTGGTGGTGGCGATATTA |
| | TCAAACTGCTGAATGAACAGGTGAACAAAGAAATGAATAGCAGCAACCTGTATATGAGCATGAGCAG |
| | CTGGTGTTATACCCATAGCCTGGATGGTGCAGGTCTGTTTCTGTTTGATCATGCAGCCGAAGAATATGA |
| | GCACGCAAAAAAACTGATCATCTTCCTGAATGAAAATAATGTTCCGGTGCAGCTGACCAGCATTAGCG |
| | CTCCGGAACATAAATTTGAAGGTCTGACACAGATTTTTCAGAAAGCCTATGAACATGAACAGCACATT |
| | AGCGAAAGCATTAACAACATTGTGGATCACGCCATCAAAAGCAAAGATCATGCAACCTTTAACTTTCTG |
| | CAGTGGTATGTTGCAGAACAGCATGAAGAAGAAGTGCTGTTTAAAGACATCCTGGATAAAATTGAACT |
| | GATCGGCAATGAAAATCACGGTCTGTATCTGGCAGATCAGTATGTTAAAGGTATTGCCAAAAGCCGCA |
| | AATAA [SEQ ID NO: 70] |
| pelBLS- | ATGAAATACCTGCTGCCGACCGCTGCTGCTGGTCTGCTGCTCCTCGCTGCCCAGCCGGCGATGGCCTTT |
| FimHL- | GCATGTAAAACCGCAAATGGCACCGCAATTCCGATTGGTGGTGGTAGCGCAAATGTTTATGTTAATCT |
| mI3 | GGCACCGGTTGTTAATGTTGGTCAGAATCTGGTTGTTGATCTGAGCACCCAGATTTTTTGCCATAATGA |
| | TTATCCGGAAACCATCACCGATTATGTTACCCTGCAGCGTGGTAGTGCATATGGTGGTGTTCTGAGCA |
| | ATTTTAGCGGCACCGTGAAATATAGCGGTAGCAGCTATCCGTTTCCGACCACCAGTGAAACACCGCGT |
| | GTTGTGTATAATAGCCGTACCGATAAACCGTGGCCTGTTGCACTGTATCTGACACCGGTGAGCAGTGC |
| | CGGTGGTGTTGCAATTAAAGCAGGTAGCCTGATTGCAGTTCTGATTCTGCGTCAGACCAATAACTATA |
| | ACTCCGATGATTTTCAGTTTGTGTGGAACATCTATGCCAATAATGATGTTGTTGTTCCGACCGGTGGTG |
| | GTTCAGGTATGAAAATGGAAGAACTGTTCAAAAAGCACAAGATTGTTGCCGTTCTGCGTGCAAATAGC |
| | GTTGAAGAAGCAAAAAAGAAAGCACTGGCCGTTTTTTTAGGTGGTGTGCATCTGATTGAAATCACCTT |
| | TACCGTTCCGGATGCAGATACCGTTATTAAAGAACTGAGCTTTCTGAAAGAAATGGGTGCAATTATTG |
| | GCGCAGGCACCGTTACCAGCGTTGAACAGGCACGTAAAGCAGTTGAAAGCGGTGCAGAATTTATTGT |
| | TAGTCCGCATCTGGATGAAGAAATTAGCCAGTTTGCAAAAGAAAAGGGCGTGTTTTATATGCCTGGTG |
| | TTATGACCCCGACCGAACTGGTTAAAGCAATGAAACTGGGTCATACCATCCTGAAACTGTTTCCGGGT |
| | GAAGTTGTTGGTCCGCAGTTTGTGAAAGCCATGAAAGGTCCTTTTCCGAACGTTAAATTTGTGCCGAC |
| | AGGTGGCGTGAATCTGGATAATGTTTGTGAATGGTTTAAAGCCGGTGTTCTGGCAGTTGGTGTTGGTA |
| | GTGCCCTGGTGAAAGGTACACCGGTTGAAGTTGCAGAAAAAGCAAAAGCCTTTGTGGAAAAAATTCG |
| | TGGTTGTACCGAAGGTAGTGGTAGCGGTTCAGGTAGCCACCACCACCACCACCACTGA |
| | [SEQ ID NO: 71] |
| FimH_DG_ | ATGGGCAGCAGCCATCATCATCATCATCACGAACTGTACTTCCAGGGCTTTGCATGTAAAACCGCAAAT |
| Ferritin | GGCACCGCAATTCCGATTGGTGGTGGTAGCGCAAATGTTTATGTTAATCTGGCACCGGCAGTTAATGT |
| (GSGGGG) | TGGTCAGAATCTGGTTGTTGATCTGAGCACCCAGATTTTTTGCCATAATGATTATCCGGAAACCATCAC |
| | CGATTATGTTACCCTGCAGCGTGGTAGTGCATATGGTGGTGTTCTGAGCAGCTTTAGCGGCACCGTGA |
| | AATATAACGGTAGCAGCTATCCGTTTCCGACCACCAGTGAAACACCGCGTGTTGTGTATAATAGCCGT |
| | ACCGATAAACCGTGGCCTGTTGCACTGTATCTGACACCGGTTAGCAGTGCCGGTGGTGTTGCAATTAA |
| | AGCAGGTAGCCTGATTGCAGTTCTGATTCTGCGTCAGACCAATAACTATAACTCCGATGATTTTCAGTT |
| | TGTGTGGAACATCTATGCCAATAATGATGTTGTTGTTCCGACCGGTGGTTGTGATGTTAGCGCACGTG |
| | ATGTTACCGTTACACTGCCGGATTATCCTGGTAGCGTTCCGATTCCGCTGACCGTTTATTGTGCAAAAA |
| | GCCAGAACCTGGGTTATTATCTGAGCGGCACCACCGCAGATGCAGGTAATAGCATTTTTACCAATACC |
| | GCAAGCTTTAGTCCGGCACAAGGTGTTGGTGTTCAGCTGACCCGTAATGGCACCATTATTCCGGCAAA |
| | TAATACCGTTAGCCTGGGTGCAGTTGGCACCAGCGCAGTGAGCCTGGGTCTGACCGCCAATTATGCAC |
| | GTACCGGTGGTCAGGTTACCGCAGGTAATGTTCAGAGCATTATTGGTGTTACCTTTGTGTATCAGCCTG |
| | GTGATGGTAATGCAGATGTGACCATTACCGTGAATGGTAAAGTTGTTGCAAAAGGTAGCGGTGGTGG |
| | TGGCGATATTATCAAACTGCTGAATGAACAGGTGAACAAAGAAATGAATAGCAGCAACCTGTATATGA |
| | GCATGAGCAGCTGGTGTTATACCCATAGCCTGGATGGTGCAGGTCTGTTTCTGTTTGATCATGCAGCC |
| | GAAGAATATGAGCACGCAAAAAAACTGATCATCTTCCTGAATGAAAATAATGTTCCGGTGCAGCTGAC |
| | CAGCATTAGCGCTCCGGAACATAAATTTGAAGGTCTGACACAGATTTTTCAGAAAGCCTATGAACATG |
| | AACAGCACATTAGCGAAAGCATTAACAACATTGTGGATCACGCCATCAAAAGCAAAGATCATGCAACC |
| | TTTAACTTTCTGCAGTGGTATGTTGCAGAACAGCATGAAGAAGAAGTGCTGTTTAAAGACATCCTGGA |
| | TAAAATTGAACTGATCGGCAATGAAAATCACGGTCTGTATCTGGCAGATCAGTATGTTAAAGGTATTG |
| | CCAAAAGCCGCAAATAA [SEQ ID NO: 72] |
| Fim HL- | ATGTTCGCCTGCAAAACCGCAAATGGCACCGCAATTCCGATTGGTGGTGGTAGCGCAAATGTTTATGT |
| C-C-MI3 | TAATCTGGCACCGGTTTGTAATGTTGGTCAGAATTGTGTTGTTGATCTGAGCACCCAGATTTTTTGCCA |
| | TAATGATTATCCGGAAACCATCACCGATTATGTTACCCTGCAGCGTGGTAGTGCATATGGTGGTGTTCT |
| | GAGCAATTTTAGCGGCACCGTGAAATATAGCGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTGAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | GGTGGTGGCAGTGGTGGTTCAGGCGGTAGCGGTGGTAGCATGAAAATGGAAGAACTGTTCAAAAAG |
| | CACAAAATTGTTGCCGTTCTGCGTGCAAATAGCGTTGAAGAAGCAAAAAAGAAAGCACTGGCCGTTTT |
| | TTTAGGTGGTGTGCATCTGATTGAAATCACCTTTACCGTTCCGGATGCAGATACCGTTATTAAAGAACT |
| | GAGCTTTCTGAAAGAAATGGGTGCAATTATTGGTGCAGGCACCGTTACCAGCGTTGAACAGGCACGT |
| | AAAGCAGTTGAAAGCGGTGCAGAATTTATTGTTAGTCCGCATCTGGATGAAGAAATTAGCCAGTTTGC |
| | AAAAGAAAAGGGCGTGTTTTATATGCCTGGTGTTATGACCCCGACCGAACTGGTTAAAGCAATGAAAC |
| | TGGGTCATACCATCCTGAAACTGTTTCCGGGTGAAGTTGTTGGTCCGCAGTTTGTGAAAGCCATGAAA |
| | GGTCCTTTTCCGAACGTTAAATTTGTGCCGACAGGTGGCGTGAATCTGGATAATGTTTGTGAATGGTTT |
| | AAAGCCGGTGTTCTGGCAGTTGGTGTTGGTAGTGCCCTGGTGAAAGGTACACCGGTTGAAGTTGCAG |
| | AAAAAGCAAAAGCCTTTGTGGAAAAAATTCGTGGTTGTACCGAAGGTAGCGGTAGTGGTAGCGGTTC |
| | AGGTAGCCATCACCATCACCATCACTGA [SEQ ID NO: 73] |
| Fim HL- | ATGTTCGCCTGCAAAACCGCAAATGGCACCGCAATTCCGATTGGTGGTGGTAGCGCAAATGTTTATGT |
| C-C- | TAATCTGGCACCGGTTTGTAATGTTGGTCAGAATTGTGTTGTTGATCTGAGCACCCAGATTTTTTGCCA |
| qBeta | TAATGATTATCCGGAAACCATCACCGATTATGTTACCCTGCAGCGTGGTAGTGCATATGGTGGTGTTCT |
| | GAGCAATTTTAGCGGCACCGTGAAATATAGCGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTGAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | GGTGGTGGCAGTGGTGGTTCAGGCGGTAGCGGTGGCAGCGCCAAACTGGAAACCGTTACACTGGGT |
| | AATATTGGTAAAGATGGTAAACAGACCCTGGTTCTGAATCCGCGTGGTGTTAATCCGACCAATGGTGT |
| | TGCCAGCCTGAGCCAGGCAGGCGCAGTTCCGGCACTGGAAAAACGTGTTACCGTTAGCGTTAGCCAG |
| | CCGAGCCGTAATCGTAAAAACTATAAAGTTCAGGTGAAAATCCAGAATCCGACCGCATGTACCGCCAA |
| | TGGTAGCTGTGATCCGAGCGTTACCCGTCAGGCATATGCAGATGTTACCTTTAGTTTTACCCAGTATAG |
| | CACCGATGAAGAACGTGCATTTGTTCGTACCGAACTGGCAGCACTGCTGGCAAGTCCGCTGCTGATTG |
| | ATGCAATTGATCAGCTGA [SEQ ID NO: 74] |
| HBcAgNC_ | ATGGATATCGATCCGTATAAAGAATTTGGTGCAAGCGTTGAACTGCTGAGCTTTCTGCCGAGCGATTTT |
| fimHL | TTTCCGAGCATTCGTGATCTGCTGGATACCGCAAGCGCACTGTATCGTGAAGCACTGGAAAGTCCGGA |
| splitted | ACATTGTAGTCCGCATCATACCGCACTGCGTCAGGCAATTCTGTGTTGGGGTGAACTGATGAATCTGG |
| | CAACCTGGGTTGGTAGCAATCTGGAAGATCCGTAGAAGGAGATATACATATGTTTGCATGTAAAACCG |
| | CAAATGGCACCGCAATTCCGATTGGTGGTGGTAGCGCAAATGTTTATGTTAATCTGGCACCGGTTGTT |
| | AATGTTGGTCAGAATCTGGTTGTTGATCTGAGCACCCAGATTTTTTGCCATAATGATTATCCGGAAACC |
| | ATCACCGATTATGTTACCCTGCAGCGTGGTAGTGCATATGGTGGTGTTCTGAGCAATTTTAGCGGCACC |
| | GTGAAATATAGCGGTAGCAGCTATCCGTTTCCGACCACCAGTGAAACACCGCGTGTTGTGTATAATAG |
| | CCGTACCGATAAACCGTGGCCTGTTGCGCTGTATCTGACACCGGTGAGCAGTGCCGGTGGTGTTGCAA |
| | TTAAAGCAGGTAGCCTGATTGCAGTTCTGATTCTGCGTCAGACCAATAACTATAACTCCGATGATTTTC |
| | AGTTTGTGTGGAACATCTATGCCAATAATGATGTTGTTGTTCCGACCGGTGGTGGTTCAGGTGCCAGC |
| | CGTGAACTGGTTGTTAGCTATGTTAATGTGAATATGGGCCTGAAAATTCGTCAGCTGCTGTGGTTTCAT |
| | ATTTCATGTCTGACCTTTGGTCGTGAAACCGTTCTGGAATATCTGGTTAGCTTTGGTGTTTGGATTCGTA |
| | CCCCTCCGGCATATCGTCCGCCTAATGCACCGATTCTGAGTACCCTGCCGGAAACAACCGTTGTTTGAG |
| | GATCC [SEQ ID NO: 75] |
| HBcAgNC_ | ATGAAATATCTGCTGCCGACCGCAGCAGCGGGTCTGCTGCTGCTGGCAGCACAGCCTGCAATGGCAG |
| fimHL- | GTCATCATCACCATCATCATAGCGGTGGTATGGATATTGATCCGTATAAAGAATTTGGTGCCAGCGTTG |
| LS | AACTGCTGAGCTTTCTGCCGAGCGATTTTTTTCCGAGCATTCGTGATCTGCTGGATACCGCAAGCGCAC |
| | TGTATCGTGAAGCACTGGAAAGTCCGGAACATTGTAGTCCGCATCATACCGCACTGCGTCAGGCAATT |
| | CTGTGTTGGGGTGAACTGATGAATCTGGCAACCTGGGTTGGTAGCAATCTGGAAGATCCGTAGAAGG |
| | AGATATACATATGAAATACCTGTTACCGACAGCCGCAGCAGGCCTGTTACTGTTAGCAGCCCAGCCAG |
| | CCATGGCATTTGCATGTAAAACCGCAAATGGCACCGCAATTCCGATTGGTGGTGGTAGCGCAAATGTT |
| | TATGTTAATCTGGCACCGGTTGTTAATGTTGGTCAGAATCTGGTTGTTGATCTGAGCACCCAGATTTTT |
| | TGCCATAATGATTATCCGGAAACCATCACCGATTATGTTACCCTGCAGCGTGGTAGTGCATATGGTGGT |
| | GTTCTGAGCAATTTTAGCGGCACCGTGAAATATAGCGGTAGCAGCTATCCGTTTCCGACCACCAGTGA |
| | AACACCGCGTGTTGTGTATAATAGCCGTACCGATAAACCGTGGCCTGTTGCGCTGTATCTGACACCGG |
| | TGAGCAGTGCCGGTGGTGTTGCAATTAAAGCAGGTAGCCTGATTGCAGTTCTGATTCTGCGTCAGACC |
| | AATAACTATAACTCCGATGATTTTCAGTTTGTGTGGAACATCTATGCCAATAATGATGTTGTTGTTCCGA |
| | CCGGTGGTGGTTCAGGTGCAAGCCGTGAACTGGTTGTTAGCTATGTTAATGTGAATATGGGCCTGAAA |
| | ATTCGTCAGCTGCTGTGGTTTCATATTTCATGTCTGACCTTTGGTCGTGAAACCGTTCTGGAATATCTGG |
| | TTAGCTTTGGTGTTTGGATTCGTACCCCTCCGGCATATCGTCCGCCTAATGCACCGATTCTGAGTACCCT |
| | GCCGGAAACAACCGTTGTTTGACTCGAG [SEQ ID NO: 76] |
| FIMHL- | ATGTTCGCCTGCAAAACCGCAAATGGCACCGCAATTCCGATTGGTGGTGGTAGCGCAAATGTTTATGT |
| MI3 | TAATCTGGCACCGGTTGTTAATGTTGGTCAGAATCTGGTTGTTGATCTGAGCACCCAGATTTTTTGCCA |
| | TAATGATTATCCGGAAACCATCACCGATTATGTTACCCTGCAGCGTGGTAGTGCATATGGTGGTGTTCT |
| | GAGCAATTTTAGCGGCACCGTGAAATATAGCGGTAGCAGCTATCCGTTTCCGACCACCAGTGAAACAC |
| | CGCGTGTTGTGTATAATAGCCGTACCGATAAACCGTGGCCTGTTGCACTGTATCTGACACCGGTGAGC |
| | AGTGCCGGTGGTGTTGCAATTAAAGCAGGTAGCCTGATTGCAGTTCTGATTCTGCGTCAGACCAATAA |
| | CTATAACTCCGATGATTTTCAGTTTGTGTGGAACATCTATGCCAATAATGATGTTGTTGTTCCGACCGGT |
| | AGCGGTGGTGGTGGCATGAAAATGGAAGAACTGTTCAAAAAACACAAGATTGTTGCCGTTCTGCGTG |
| | CAAATAGCGTTGAAGAAGCAAAAAAGAAAGCACTGGCCGTTTTTTTAGGTGGTGTGCATCTGATTGAA |
| | ATCACCTTTACCGTTCCGGATGCAGATACCGTTATTAAAGAACTGAGCTTTCTGAAAGAAATGGGTGC |
| | AATTATTGGCGCAGGCACCGTTACCAGCGTTGAACAGGCACGTAAAGCAGTTGAAAGCGGTGCAGAA |
| | TTTATTGTTAGTCCGCATCTGGATGAAGAAATTAGCCAGTTTGCAAAAGAAAAGGGCGTGTTTTATAT |
| | GCCTGGTGTTATGACCCCGACCGAACTGGTTAAAGCAATGAAACTGGGTCATACCATCCTGAAACTGT |
| | TTCCGGGTGAAGTTGTTGGTCCGCAGTTTGTGAAAGCCATGAAAGGTCCTTTTCCGAACGTTAAATTTG |
| | TGCCGACCGGTGGCGTGAATCTGGATAATGTTTGTGAATGGTTTAAAGCCGGTGTTCTGGCAGTTGGT |
| | GTTGGTAGTGCCCTGGTGAAAGGTACACCGGTTGAAGTTGCAGAAAAAGCAAAAGCCTTTGTGGAAA |
| | AAATTCGTGGTTGTACCGAAGGTAGTGGTAGCGGCAGCGGTAGCGGTTCACATCACCATCACCATCAC |
| | TGA [SEQ ID NO: 77] |
| FimH_DNKQ_ | ATGGAAACCGATACACTGCTGCTGTGGGTGCTGTTGCTCTGGGTTCCAGGATCTACAGGGGATGCCGC |
| DG_deglyc | TCAACCTGCTCGGAGAGCCAGAAGAACAAAGCTGGCCCTGTTTGCCTGTAAAACCGCCAGCGGCACA |
| | GCCATTCCTATTGGCGGAGGCAGCGCCAATGTGTACGTGAACCTGGCTCCTGTGGTCAACGTGGGCCA |
| | GAATCTGGTGGTGGACCTGAGCACCCAGATCTTTTGCCACAACGACTACCCCGAGACAATCACCGACT |
| | ACGTGACACTGCAGAGAGGCTCTGCTTACGGCGGCGTGCTGAGCGATTTTTCCGGCACAGTGAAGTA |
| | CAGCGGCAGCAGCTACCCATTTCCTACCACCAGCGAGACACCCAGAGTGGTGTACAACAGCAGAACC |
| | GACAAGCCCTGGCCTGTGGCTCTGTACCTGACACCTGTTAGTTCTGCCGGCGGAGTGGCCATTAAGGC |
| | CGGATCTCTGATTGCCGTGCTGATCCTGCGGCAGACCAACAACTACAACAGCGACGACTTCCAGTTCG |
| | TGTGGAACATCTACGCCAACAACGACGTGGTGGTGCCTACAGGCGGATGTGATGTGTCCGCCAGAGA |
| | TGTGACAGTGACCCTGCCTGATTACCCCGGCTCTGTGCCTATTCCTCTGACCGTGTACTGCGCCAAGTC |
| | TCAGAACCTGGGCTACTACCTGAGCGGCACAACAGCCGATGCCGGCAACAGCATCTTTACCAACACCG |
| | CCAGCTTCAGCCCTGCTCAAGGTGTTGGAGTGCAGCTGACCAGAGATGGCACAATCATCCCCGCCGAC |
| | AATACCGTGTCTCTGGGCGCTGTTGGCACATCTGCAGTTTCTCTGGGCCTGACCGCCAACTATGCCAGA |
| | ACAGGTGGACAAGTGACCGCCGGCAATGTGCAGTCTATCATCGGCGTGACATTCGTGTATCAGGACA |
| | ACAAGCAGGCCGACGTGACCATCACCGTGAATGGCAAAGTGGTGGCCAAAGGCTCTGGCCATCACCA |
| | CCACCATCACTG [SEQ ID NO: 90] |
| FimH_ | ATGGAAACCGACACACTGCTGCTGTGGGTGCTGCTTTTGTGGGTGCCAGGATCTACAGGGGATGCCG |
| PGDGN_ | CTCAACCTGCTCGGAGAGCCAGAAGAACAAAGCTGGCCCTGTTTGCCTGCAAGACCGCCAATGGCACA |
| DG | GCCATTCCTATTGGCGGCGGAAGCGCCAATGTGTACGTGAACCTGGCTCCTGTGGTCAACGTGGGCCA |
| | GAATCTGGTGGTGGACCTGAGCACCCAGATCTTTTGCCACAACGACTACCCCGAGACAATCACCGACT |
| | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCTACAGGCGGATGTGATGTGTCCGCCAGAGAT |
| | GTGACAGTGACCCTGCCTGATTACCCCGGCTCTGTGCCTATTCCTCTGACCGTGTACTGCGCCAAGTCT |
| | CAGAACCTGGGCTACTACCTGAGCGGCACAACAGCCGATGCCGGCAACAGCATCTTTACCAACACCGC |
| | CAGCTTCAGCCCTGCTCAAGGTGTTGGAGTGCAGCTGACCCGGAACGGAACAATCATCCCCGCCAACA |
| | ATACCGTGTCTCTGGGAGCTGTGGGCACCTCTGCTGTTTCTCTGGGCCTGACAGCCAACTATGCCAGA |
| | ACAGGCGGACAAGTGACAGCCGGCAATGTGCAGTCTATCATCGGCGTGACATTCGTGTATCAGGACA |
| | ACAAGCAGGCCGACGTGACCATCACCGTGAATGGCAAAGTGGTGGCCAAAGGCTCTGGCCAT |
| | [SEQ ID NO: 91] |
| FimH_ | ATGGAAACCGACACACTGCTGCTGTGGGTGCTGCTTTTGTGGGTGCCAGGATCTACAGGGGATGCCG |
| DNKQ_DG | CTCAACCTGCTCGGAGAGCCAGAAGAACAAAGCTGGCCCTGTTTGCCTGCAAGACCGCCAATGGCACA |
| | GCCATTCCTATTGGCGGCGGAAGCGCCAATGTGTACGTGAACCTGGCTCCTGTGGTCAACGTGGGCCA |
| | GAATCTGGTGGTGGACCTGAGCACCCAGATCTTTTGCCACAACGACTACCCCGAGACAATCACCGACT |
| | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCTACAGGCGGATGTGATGTGTCCGCCAGAGAT |
| | GTGACAGTGACCCTGCCTGATTACCCCGGCTCTGTGCCTATTCCTCTGACCGTGTACTGCGCCAAGTCT |
| | CAGAACCTGGGCTACTACCTGAGCGGCACAACAGCCGATGCCGGCAACAGCATCTTTACCAACACCGC |
| | CAGCTTCAGCCCTGCTCAAGGTGTTGGAGTGCAGCTGACCCGGAACGGAACAATCATCCCCGCCAACA |
| | ATACCGTGTCTCTGGGAGCTGTGGGCACCTCTGCTGTTTCTCTGGGCCTGACAGCCAACTATGCCAGA |
| | ACAGGCGGACAAGTGACAGCCGGCAATGTGCAGTCTATCATCGGCGTGACCTTCGTGTATCAGCCTG |
| | GCGACGGAAATGCCGACGTGACCATCACAGTGAATGGCAAGGTGGTGGCCAAAGGCTCTGGACACCA |
| | CCACCATCACCACTG [SEQ ID NO: 92] |
| FimH_ | ATGGAAACCGACACACTGCTGCTGTGGGTGCTGCTTTTGTGGGTGCCAGGATCTACAGGGGATGCCG |
| DeltaGG_ | CTCAACCTGCTCGGAGAGCCAGAAGAACAAAGCTGGCCCTGTTTGCCTGCAAGACCGCCAATGGCACA |
| PGDGN_ | GCCATTCCTATTGGCGGCGGAAGCGCCAATGTGTACGTGAACCTGGCTCCTGTGGTCAACGTGGGCCA |
| DG | GAATCTGGTGGTGGACCTGAGCACCCAGATCTTTTGCCACAACGACTACCCCGAGACAATCACCGACT |
| | ACGTGACACTGCAGAGAGGCTCTGCTTACGGCGGCGTGCTGAGCAATTTTTCCGGCACAGTGAAGTAC |
| | AGCGGCAGCAGCTACCCATTTCCTACCACCAGCGAGACACCCAGAGTGGTGTACAACAGCAGAACCG |
| | ACAAGCCCTGGCCTGTGGCTCTGTACCTGACACCTGTTAGTTCTGCCGGCGGAGTGGCCATTAAGGCC |
| | GGATCTCTGATTGCCGTGCTGATCCTGCGGCAGACCAACAACTACAACAGCGACGACTTCCAGTTCGT |
| | GTGGAACATCTACGCCAACAACGACGTGGTGGTGCCCACCTGTGATGTGTCCGCTAGAGATGTGACA |
| | GTGACCCTGCCTGATTACCCCGGCTCTGTGCCTATTCCTCTGACCGTGTACTGCGCCAAGTCTCAGAAC |
| | CTGGGCTACTACCTGAGCGGCACAACAGCCGATGCCGGCAACAGCATCTTTACCAACACCGCCAGCTT |
| | CAGCCCTGCTCAAGGTGTTGGAGTGCAGCTGACCCGGAACGGAACAATCATCCCCGCCAACAATACCG |
| | TGTCTCTGGGAGCTGTGGGCACATCTGCTGTTTCTCTGGGCCTGACAGCCAACTATGCCAGAACAGGC |
| | GGACAAGTGACAGCCGGCAATGTGCAGTCTATCATCGGCGTGACCTTCGTGTATCAGCCTGGCGACG |
| | GAAATGCCGACGTGACCATCACAGTGAATGGCAAGGTGGTGGCCAAAGGCTCTGGACACCACCACCA |
| | TCACCACTG [SEQ ID NO: 93] |
| FimH_ | ATGGAAACCGACACACTGCTGCTGTGGGTGCTGCTTTTGTGGGTGCCAGGCTCTACAGGCGATTTTGC |
| DGG_sl | CTGCAAGACCGCCAACGGCACAGCCATTCCTATTGGCGGAGGCAGCGCCAATGTGTACGTGAACCTG |
| | GCTCCTGTGGTCAACGTGGGCCAGAATCTGGTGGTGGACCTGAGCACCCAGATCTTTTGCCACAACGA |
| | CTACCCCGAGACAATCACCGACTACGTGACACTGCAGAGAGGCTCTGCTTACGGCGGCGTGCTGAGC |
| | AATTTTTCCGGCACAGTGAAGTACAGCGGCAGCAGCTACCCATTTCCTACCACCAGCGAGACACCCAG |
| | AGTGGTGTACAACAGCAGAACCGACAAGCCCTGGCCTGTGGCTCTGTACCTGACACCTGTTAGTTCTG |
| | CCGGCGGAGTGGCCATTAAGGCCGGATCTCTGATTGCCGTGCTGATCCTGCGGCAGACCAACAACTAC |
| | AACAGCGACGACTTCCAGTTCGTGTGGAACATCTACGCCAACAACGACGTGGTGGTGCCCACCTGTGA |
| | TGTGTCCGCTAGAGATGTGACAGTGACCCTGCCTGATTACCCCGGCTCTGTGCCTATTCCTCTGACCGT |
| | GTACTGCGCCAAGAGCCAGAACCTGGGCTACTACCTGTCTGGCACAACAGCCGATGCCGGCAACAGC |
| | ATCTTTACCAACACCGCCAGCTTCAGCCCTGCTCAAGGTGTTGGAGTGCAGCTGACCCGGAACGGAAC |
| | AATCATCCCCGCCAACAATACCGTGTCTCTGGGAGCTGTGGGCACATCTGCTGTTTCTCTGGGCCTGAC |
| | CGCCAATTATGCCAGAACAGGCGGACAAGTGACCGCCGGCAATGTGCAGTCTATCATCGGCGTGACC |
| | TTCGTGTATCAGCCTGGCGACGGAAACGCCGATGTGACCATCACAGTGAATGGCAAGGTGGTGGCCA |
| | AAGGCTCTGGACACCACCACCATCACCACTGA [SEQ ID NO: 94] |
| FimH_ | ATGGAAACCGACACACTGCTGCTGTGGGTGCTGCTTTTGTGGGTGCCAGGCTCTACAGGCGATTTTGC |
| PGDGN_sl | CTGCAAGACCGCCAACGGCACAGCCATTCCTATTGGCGGAGGCAGCGCCAATGTGTACGTGAACCTG |
| | GCTCCTGTGGTCAACGTGGGCCAGAATCTGGTGGTGGACCTGAGCACCCAGATCTTTTGCCACAACGA |
| | CTACCCCGAGACAATCACCGACTACGTGACACTGCAGAGAGGCTCTGCTTACGGCGGCGTGCTGAGC |
| | AATTTTTCCGGCACAGTGAAGTACAGCGGCAGCAGCTACCCATTTCCTACCACCAGCGAGACACCCAG |
| | AGTGGTGTACAACAGCAGAACCGACAAGCCCTGGCCTGTGGCTCTGTACCTGACACCTGTTAGTTCTG |
| | CCGGCGGAGTGGCCATTAAGGCCGGATCTCTGATTGCCGTGCTGATCCTGCGGCAGACCAACAACTAC |
| | AACAGCGACGACTTCCAGTTCGTGTGGAACATCTACGCCAACAACGACGTGGTGGTGCCTACAGGCG |
| | GATGTGATGTGTCCGCCAGAGATGTGACAGTGACCCTGCCTGATTACCCCGGCTCTGTGCCTATTCCTC |
| | TGACCGTGTACTGCGCCAAGAGCCAGAACCTGGGCTACTACCTGTCTGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTTTCTCTGGG |
| | CCTGACAGCCAACTATGCCAGAACAGGCGGACAAGTGACAGCCGGCAATGTGCAGTCTATCATCGGC |
| | GTGACCTTCGTGTATCAGCCTGGCGACGGAAACGCCGATGTGACCATCACAGTGAATGGCAAGGTGG |
| | TGGCCAAAGGCTCTGGACACCACCACCATCACCACTGACTCGAG [SEQ ID NO: 95] |
| FimH_ | ATGGAAACCGACACACTGCTGCTGTGGGTGCTGCTTTTGTGGGTGCCAGGCTCTACAGGCGATTTTGC |
| DNKQ_sl | CTGCAAGACCGCCAACGGCACAGCCATTCCTATTGGCGGAGGCAGCGCCAATGTGTACGTGAACCTG |
| | GCTCCTGTGGTCAACGTGGGCCAGAATCTGGTGGTGGACCTGAGCACCCAGATCTTTTGCCACAACGA |
| | CTACCCCGAGACAATCACCGACTACGTGACACTGCAGAGAGGCTCTGCTTACGGCGGCGTGCTGAGC |
| | AATTTTTCCGGCACAGTGAAGTACAGCGGCAGCAGCTACCCATTTCCTACCACCAGCGAGACACCCAG |
| | AGTGGTGTACAACAGCAGAACCGACAAGCCCTGGCCTGTGGCTCTGTACCTGACACCTGTTAGTTCTG |
| | CCGGCGGAGTGGCCATTAAGGCCGGATCTCTGATTGCCGTGCTGATCCTGCGGCAGACCAACAACTAC |
| | AACAGCGACGACTTCCAGTTCGTGTGGAACATCTACGCCAACAACGACGTGGTGGTGCCTACAGGCG |
| | GATGTGATGTGTCCGCCAGAGATGTGACAGTGACCCTGCCTGATTACCCCGGCTCTGTGCCTATTCCTC |
| | TGACCGTGTACTGCGCCAAGAGCCAGAACCTGGGCTACTACCTGTCTGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTTTCTCTGGG |
| | CCTGACAGCCAACTATGCCAGAACAGGCGGACAAGTGACAGCCGGCAATGTGCAGTCTATCATCGGC |
| | GTGACATTCGTGTATCAGGACAACAAGCAGGCCGACGTGACCATCACCGTGAATGGCAAAGTGGTGG |
| | CCAAAGGCTCTGGCCATCACCACCACCATCACTGACTCGAG [SEQ ID NO: 96] |
| FIMH_ | ATGGAAACCGATACACTGCTGCTGTGGGTGCTGTTGCTCTGGGTTCCAGGCTCTACAGGCGATTTTGC |
| DG_PGDGN_ | CTGCAAGACCGCCAACGGCACAGCCATTCCTATTGGCGGAGGCAGCGCCAATGTGTACGTTAACCTGG |
| 536-MI3 | CTCCTGCCGTGAACGTGGGCCAGAATCTGGTGGTGGATCTGAGCACCCAGATCTTTTGCCACAACGAC |
| | TACCCCGAGACAATCACCGACTACGTGACACTGCAGAGAGGCTCTGCTTACGGCGGCGTGCTGTCTAG |
| | CTTTAGCGGCACCGTGAAGTACAACGGCAGCAGCTACCCATTTCCTACCACCAGCGAGACACCCAGAG |
| | TGGTGTACAACAGCAGAACCGACAAGCCCTGGCCTGTGGCTCTGTACCTGACACCTGTTAGTTCTGCC |
| | GGCGGAGTGGCCATTAAGGCCGGATCTCTGATTGCCGTGCTGATCCTGCGGCAGACCAACAACTACA |
| | ACAGCGACGACTTCCAGTTCGTGTGGAACATCTACGCCAACAACGACGTGGTGGTGCCTACAGGCGG |
| | ATGTGATGTGTCCGCCAGAGATGTGACAGTGACCCTGCCTGATTACCCCGGCTCTGTGCCTATTCCTCT |
| | GACCGTGTACTGCGCCAAGAGCCAGAACCTGGGCTACTACCTGTCTGGCACAACAGCCGATGCCGGC |
| | AACAGCATCTTTACCAACACCGCCAGCTTCAGCCCTGCTCAAGGTGTTGGAGTGCAGCTGACCCGGAA |
| | CGGAACAATCATCCCCGCCAACAATACCGTGTCTCTGGGAGCTGTGGGCACCTCTGCTGTGTCTCTTGG |
| | CCTGACAGCCAACTATGCCAGAACAGGCGGACAAGTGACAGCCGGCAATGTGCAGTCTATCATCGGC |
| | GTGACCTTCGTGTATCAGCCTGGCGACGGAAACGCCGATGTGACCATCACAGTGAATGGCAAGGTGG |
| | TGGCCAAGAGCGGAAGCCACCACCATCATCACCATCACCACGGCGGCAGCATGAAGATGGAAGAACT |
| | GTTCAAGAAGCACAAGATCGTCGCCGTGCTGCGGGCCAATTCTGTGGAAGAGGCCAAAAAAAAGGCC |
| | CTGGCCGTGTTTCTTGGCGGAGTGCACCTGATCGAGATCACCTTTACCGTGCCTGACGCCGACACCGT |
| | GATCAAAGAGCTGAGCTTCCTGAAAGAGATGGGCGCCATCATCGGAGCCGGCACAGTGACATCTGTT |
| | GAGCAGGCCAGAAAGGCCGTGGAATCTGGCGCCGAGTTTATCGTGTCCCCTCACCTGGATGAGGAAA |
| | TCAGCCAGTTCGCCAAAGAAAAGGGCGTGTTCTACATGCCCGGCGTGATGACACCTACAGAGCTGGTC |
| | AAAGCCATGAAGCTGGGCCACACCATCCTGAAGCTGTTTCCAGGCGAAGTCGTGGGCCCTCAGTTCGT |
| | GAAAGCTATGAAGGGCCCATTTCCAAACGTGAAGTTCGTGCCCACTGGCGGCGTGAACCTGGATAAT |
| | GTGTGCGAGTGGTTCAAGGCTGGCGTGCTGGCTGTTGGAGTTGGCTCTGCTCTGGTCAAGGGCACAC |
| | CTGTGGAAGTGGCTGAGAAGGCCAAGGCCTTCGTGGAAAAGATCAGAGGCTGCACCGAGTGA |
| | [SEQ ID NO: 97] |
| HBcFIM | ATGGAAACCGATACACTGCTGCTGTGGGTGCTGTTGCTCTGGGTTCCAGGATCTACCGGCGACGACAT |
| HLJ96 | CGACCCCTACAAAGAGTTTGGCGCCAGCGTCGAGCTGCTGAGCTTCCTGCCTAGCGACTTCTTCCCTTC |
| | CATCCGGGATCTGCTGGATACCGCTAGCGCCCTGTATAGAGAGGCCCTGGAAAGCCCTGAGCACTGCT |
| | CTCCACATCACACAGCCCTGAGACAGGCCATCCTGTGTTGGGGCGAACTGATGAATCTGGCCACCTGG |
| | GTCGGAAGCAACCTGGAAGATCCTGGTTCTGGCGGCGGAGGCTTTGCCTGTAAAACAGCCAATGGCA |
| | CCGCCATTCCTATCGGAGGCGGCAGCGCCAATGTGTACGTTAACCTGGCTCCTGTGGTCAACGTGGGC |
| | CAGAATCTGGTGGTGGACCTGAGCACCCAGATCTTTTGCCACAACGACTACCCCGAGACAATCACCGA |
| | CTACGTGACACTGCAGAGAGGCTCTGCTTACGGCGGCGTGCTGAGCAATTTTTCCGGCACAGTGAAGT |
| | ACAGCGGCAGCAGCTACCCATTTCCTACCACCAGCGAGACACCCAGAGTGGTGTACAACAGCAGAACC |
| | GACAAGCCCTGGCCTGTGGCTCTGTACCTGACACCTGTTAGTTCTGCTGGCGGAGTGGCCATCAAGGC |
| | CGGATCTCTGATTGCCGTGCTGATCCTGCGGCAGACCAACAACTACAACAGCGACGACTTCCAGTTCG |
| | TGTGGAACATCTACGCCAACAACGACGTGGTGGTGCCTACAGGCGGAGGATCTGGCGGAGCTTCTAG |
| | AGAACTGGTCGTGTCCTACGTGAACGTGAACATGGGCCTGAAGATCCGGCAGCTGCTCTGGTTTCACA |
| | TCAGCTGTCTGACCTTCGGCCGGGAAACCGTGCTGGAATACCTGGTGTCCTTCGGCGTGTGGATCAGA |
| | ACCCCTCCTGCCTATAGACCTCCTAACGCTCCCATCCTGAGCACACTGCCTGAGACAACAGTTGTTGGA |
| | AGCGGAGGCGGAGGCCACCACCATCACCATCAT [SEQ ID NO: 98] |
| HBcFIM | ATGGAGACCGACACCCTGCTGCTGTGGGTGCTGCTGCTGTGGGTGCCCGGCAGCACCGGCGACGACA |
| HDGJ96 | TCGACCCCTACAAGGAGTTCGGCGCCAGCGTGGAGCTGCTGAGCTTCCTGCCCAGCGACTTCTTCCCC |
| | AGCATCCGGGACCTGCTGGACACCGCCAGCGCCCTGTACCGGGAGGCCCTGGAGAGCCCCGAGCACT |
| | GCAGCCCCCACCACACCGCCCTGCGGCAGGCCATCCTGTGCTGGGGCGAGCTGATGAACCTGGCCAC |
| | CTGGGTGGGCAGCAACCTGGAGGACCCCGGCAGCGGCGGCGGCGGCTTCGCCTGCAAGACCGCCAA |
| | CGGCACCGCCATCCCCATCGGCGGCGGCAGCGCCAACGTGTACGTGAACCTGGCCCCCGTGGTGAAC |
| | GTGGGCCAGAACCTGGTGGTGGACCTGAGCACCCAGATCTTCTGCCACAACGACTACCCCGAGACCAT |
| | CACCGACTACGTGACCCTGCAGCGGGGCAGCGCCTACGGCGGCGTGCTGAGCAACTTCAGCGGCACC |
| | GTGAAGTACAGCGGCAGCAGCTACCCCTTCCCCACCACCAGCGAGACCCCCCGGGTGGTGTACAACA |
| | GCCGGACCGACAAGCCCTGGCCCGTGGCCCTGTACCTGACCCCCGTGAGCAGCGCCGGCGGCGTGGC |
| | CATCAAGGCCGGCAGCCTGATCGCCGTGCTGATCCTGCGGCAGACCAACAACTACAACAGCGACGACT |
| | TCCAGTTCGTGTGGAACATCTACGCCAACAACGACGTGGTGGTGCCCACCGGCGGCTGCGACGTGAG |
| | CGCCCGGGACGTGACCGTGACCCTGCCCGACTACCCCGGCAGCGTGCCCATCCCCCTGACCGTGTACT |
| | GCGCCAAGAGCCAGAACCTGGGCTACTACCTGAGCGGCACCACCGCCGACGCCGGCAACAGCATCTT |
| | CACCAACACCGCCAGCTTCAGCCCCGCCCAGGGCGTGGGCGTGCAGCTGACCCGGAACGGCACCATC |
| | ATCCCCGCCAACAACACCGTGAGCCTGGGCGCCGTGGGCACCAGCGCCGTGAGCCTGGGCCTGACCG |
| | CCAACTACGCCCGGACCGGCGGCCAGGTGACCGCCGGCAACGTGCAGAGCATCATCGGCGTGACCTT |
| | CGTGTACCAGCCCGGCGACGGCAACGCCGACGTGACCATCACCGTGAACGGCAAGGTGGTGGCCAA |
| | GGGCAGCGGCGGCGGCGGCGCCAGCCGGGAGCTGGTGGTGAGCTACGTGAACGTGAACATGGGCCT |
| | GAAGATCCGGCAGCTGCTGTGGTTCCACATCAGCTGCCTGACCTTCGGCCGGGAGACCGTGCTGGAG |
| | TACCTGGTGAGCTTCGGCGTGTGGATCCGGACCCCCCCCGCCTACCGGCCCCCCAACGCCCCCATCCTG |
| | AGCACCCTGCCCGAGACCACCGTGGTGGGCAGCGGCGGCGGCGGCCACCACCACCACCACCAC |
| | [SEQ ID NO: 99] |

1. A polypeptide having an amino acid sequence comprising:
(a) FimH; or a variant, fragment and/or fusion of FimH, and
(b) a donor-strand complementing amino acid sequence,
wherein (b) is downstream of (a), and wherein (b) comprises an amino acid sequence according to SEQ ID NO: 5.

2. A polypeptide comprising an amino acid sequence X-(a)-L-(b)-Y, wherein “(a)” is a FimH polypeptide, or a variant, fragment and/or fusion of FimH; “L” is a first linker; “(b)” is a donor-strand complementing amino acid sequence, wherein (b) comprises an amino acid sequence according to SEQ ID NO: 5, “X” is an optional N-terminal amino acid sequence; “Y is an optional C-terminal amino acid sequence, wherein “Y” is not derived from FimC or FimH or a fragment thereof.

3. The polypeptide of claim 1, wherein (a) comprises:
(A) the amino acid sequence of SEQ ID NO: 1 (GenbankAccession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (Genbank Accession no: AAN83822.1 (FimH of CFT073)), SEQ ID NO: 103, SEQ ID NO: 104 (Genbank Accession no: AJE58925.1 (FimH of E. coli 789)), SEQ ID NO: 105, SEQ ID NO: 106 (Genbank Accession No. AAC35864.1, corresponding to nucleic acid sequence AF089840.1 (FimH of IHE3034), or SEQ ID NO: 107,
(B) an amino acid sequence comprising from 1 to 10 single amino acid alterations compared to SEQ ID NO: 1 (Genbank Accession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (Genbank Accession no: AAN83822.1 (FimH of CFT073)), SEQ ID NO: 103, SEQ ID NO: 104 (Genbank Accession no: AJE58925.1 (FimH of E. coli 789)), SEQ ID NO: 105, SEQ ID NO: 106 (Genbank Accession No. AAC35864.1, corresponding to nucleic acid sequence AF089840.1 (FimH of IHE3034), or SEQ ID NO: 107,
(C) an amino acid sequence with at least 70% sequence identity with SEQ ID NO: 1 (Genbank Accession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (GenbankAccession no: AAN83822.1 (FimH of CFT073)), SEQ ID NO: 103, SEQ ID NO: 104 (Genbank Accession no: AJE58925.1 (FimH of E. coli 789)), SEQ ID NO: 105, SEQ ID NO: 106 (Genbank Accession No. AAC35864.1, corresponding to nucleic acid sequence AF089840.1 (FimH of IHE3034), or SEQ ID NO: 107, and/or
(D) a fragment of at least 10 consecutive amino acids from SEQ ID NO: 1 (Genbank Accession no: ELL41155.1 (FimH of E. coli J96)), SEQ ID NO: 2, SEQ ID NO: 100 (Genbank Accession no: ABG72591.1 (FimH of UPEC 536)), SEQ ID NO: 101, SEQ ID NO: 102 (Genbank Accession no: AAN83822.1 (FimH of CFT073)), SEQ ID NO: 103, SEQ ID NO: 104 (Genbank Accession no: AJE58925.1 (FimH of E. coli 789)), SEQ ID NO: 105, SEQ ID NO: 106 (Genbank Accession No. AAC35864.1, corresponding to nucleic acid sequence AF089840.1 (FimH of IHE3034), or SEQ ID NO: 107.

4. (canceled)

5. (canceled)

6. (canceled)

7. (canceled)

8. (canceled)

9. (canceled)

10. (canceled)

11. (canceled)

12. The polypeptide of claim 2, wherein the first linker or “L” comprises 2-20 amino acids.

13. The polypeptide of claim 2, wherein the first linker begins with proline.

14. The polypeptide of claim 2, wherein the first linker comprises of polar amino acids.

15. The polypeptide of claim 2, wherein the first linker comprises the amino acid sequence of PGDGN [SEQ ID NO: 7], or a variant or fusion thereof.

16. (canceled)

17. (canceled)

18. (canceled)

19. The polypeptide of claim 1, wherein the polypeptide comprises a nanoparticle domain at the N-terminus or C-terminus, optionally wherein “X” or “Y” comprise a nanoparticle domain.

20. The polypeptide of claim 19, wherein the nanoparticle domain is selected from the group consisting of:
(i) Ferritin, wherein the ferritin comprises the amino acid sequence of [SEQ ID NO: 15], [SEQ ID NO: 109], [SEQ ID NO: 16], any one of [SEQ ID NO: 149]-[SEQ ID NO: 152], or a variant and/or fragment thereof;
(ii) iMX313, wherein the iMX313 comprises the amino acid sequence of [SEQ ID NO: 17], or a variant and/or fragment thereof;
(iii) mI3, wherein the mI3 comprises the amino acid sequence of [SEQ ID NO: 18], or a variant and/or fragment thereof;
(iv) encapsulin, wherein the encapsulin comprises the amino acid sequence of [SEQ ID NO: 19], or a variant and/or fragment thereof; and
(v) Self-assembling viral coat proteins, wherein the self-assembling viral coat protein comprises the amino acid sequence of: Acinetobacter phage AP205 coat protein (NCBI Reference Sequence: NP_085472.1), Hepatitis B virus core protein (HBc) [SEQ ID NO: 110], or bacteriophage Qβ [SEQ ID NO: 111], or a variant and/or fragment thereof.

21. (canceled)

22. (canceled)

23. (canceled)

24. (canceled)

25. (canceled)

26. A polypeptide monomer comprising an amino acid sequence that has:
(i) at least 80% sequence identity to the amino acid sequence SEQ ID NO: 16 and has one or more mutations from the group consisting of: glycine (G) at the position that aligns to residue 34 of SEQ ID NO: 16 (T34G mutation), aspartic acid (D) at the position that aligns to residue 70 of SEQ ID NO: 16 (N70D mutation), isoleucine (I) at the position that aligns to residue 72 of SEQ ID NO: 16 (V72I mutation) and alanine (A) at the position that aligns to residue 124 of SEQ ID NO: 16 (S124A mutation);
(ii) at least 80% sequence identity to the amino acid sequence SEQ ID NO: 16 and has glycine (G) at the position that aligns to residue 34 of SEQ ID NO: 16 (T34G mutation), aspartic acid (D) at the position that aligns to residue 70 of SEQ ID NO: 16 (N70D mutation), isoleucine (I) at the position that aligns to residue 72 of SEQ ID NO: 16 (V72I mutation) and alanine (A) at the position that aligns to residue 124 of SEQ ID NO: 16 (S124A mutation), optionally wherein the polypeptide monomer comprises an amino acid sequence that has at least 80% sequence identity identity to the amino acid sequence SEQ ID NO: 149;
(iii) at least 80% sequence identity to the amino acid sequence SEQ ID NO: 16 and has glycine (G) at the position that aligns to residue 34 of SEQ ID NO: 16 (T34G mutation), isoleucine (I) at the position that aligns to residue 72 of SEQ ID NO: 16 (V72I mutation) and alanine (A) at the position that aligns to residue 124 of SEQ ID NO: 16 (S124A mutation), optionally wherein the polypeptide monomer comprises an amino acid sequence that has at least 80% sequence identity, to the amino acid sequence SEQ ID NO: 150;
(iv) at least 80% sequence identity to the amino acid sequence SEQ ID NO: 16 and has glycine (G) at the position that aligns to residue 34 of SEQ ID NO: 16 (T34G mutation), and alanine (A) at the position that aligns to residue 124 of SEQ ID NO: 16 (S124A mutation), optionally wherein the polypeptide monomer comprises an amino acid sequence that has at least 80% sequence identity to the amino acid sequence SEQ ID NO: 151; or
(v) at least 80% sequence identity to the amino acid sequence SEQ ID NO: 16 and has glycine (G) at the position that aligns to residue 34 of SEQ ID NO: 16 (T34G mutation), optionally wherein the polypeptide monomer comprises an amino acid sequence that has at least 80% sequence identity to the amino acid sequence SEQ ID NO: 152.

27. (canceled)

28. A nanoparticle comprising the polypeptide monomer of claim 26.

29. The nanoparticle of claim 28, wherein the nanoparticle is a homo-oligomer.

30. The nanoparticle claim 28, wherein the exterior surface structure or interior surface structure of the nanoparticle carries one or more antigen and/or immunostimulant.

31. (canceled)

32. (canceled)

33. (canceled)

34. The polypeptide of claim 1, wherein the polypeptide comprises an amino acid sequence with at least 70% sequence identity to SEQ ID NO: 123 or SEQ ID NO:124, for example, 80%, 85%, 90%, 91%, 92%, 93%, 94%, 95%, 96%, 97%, 98% or 99% sequence identity to SEQ ID NO: 123 or SEQ ID NO:124.

35. (canceled)

36. (canceled)

37. (canceled)

38. (canceled)

39. (canceled)

40. (canceled)

41. A nucleic acid encoding the polypeptide of claim 1.

42. (canceled)

43. (canceled)

44. (canceled)

45. (canceled)

46. (canceled)

47. A cell comprising the nucleic acid of claim 41.

48. (canceled)

49. (canceled)

50. (canceled)

51. (canceled)

52. (canceled)

53. (canceled)

54. (canceled)

55. A vaccine comprising the polypeptide of claim 1, or the nucleic acid of claim 41.

56. The vaccine of claim 55 further comprising an adjuvant, optionally wherein the adjuvant comprises 3D-MPL, QS21 and liposomes comprising cholesterol.

57. (canceled)

58. (canceled)

59. (canceled)

60. (canceled)

61. (canceled)

62. (canceled)

63. A method of treating and/or preventing one or more disease in a mammal, the method comprising of administering the mammal with an effective amount of the polypeptide of claim 1, the nucleic acid of claim 41, or the vaccine of claim 56, optionally wherein the disease is a urinary tract infection.

64. A method of raising an immune response in a mammal, the method comprising or consisting of administering the mammal with an effective amount of the polypeptide of claim 1, the nucleic acid of claim 41, or the vaccine of claim 56.
